SEQUENCE LISTING (217 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 3 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVTKSEWQQGFVFSCSVMHEALHNHYTQKSLSLSPGK

SEQ ID NO: 5 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWAVYKTTPPVLDSDGSFFLYSKLTVPKSTWQQGWVFSCSVMHEALHNHYTQKSLSLSPGK

SEQ ID NO: 6 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWAVYQTTPPVLDSDGSFFLYSKLTVPKSTWQQGWVFSCSVMHEALHNHYTQKSLSLSPGK

SEQ ID NO: 7 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWAVYQTTPPVLDSDGSFFLYSKLTVPKSTWQQGWVFSCSVMHEALHNHYTQKSLSLSPGK

SEQ ID NO: 8 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWVNQKTTPPVLDSDGSFFLYSKLTVPKSTWQQGWVFSCSVMHEALHNHYTQKSLSLSPGK

SEQ ID NO: 9 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVNQQTTPPVLDSDGSFFLYSKLTVPKSTWQQGWVFSCSVMHEALHNHYTQKSLSLSPGK

SEQ ID NO: 10 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWVNQQTTPPVLDSDGSFFLYSKLTVPKSTWQQGWVFSCSVMHEALHNHYTQKSLSLSPGK

SEQ ID NO: 11 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKSEWQQGFVFSCSVMHEALHNHYTQKSLSLSPGK

SEQ ID NO: 12 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVTKEEWQQGFVFSCSVMHEALHNHYTQKSLSLSPGK

SEQ ID NO: 13 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 14 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSNYKTTPPVLDSDGSFFLYSKLTVTKSEWQQGFVFSCSVMHEALHNHYTQKSLSLSPGK

SEQ ID NO: 15 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 16 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSNYKTTPPVLDSDGSFFLYSKLTVTKEEWQQGFVFSCSVMHEALHNHYTQKSLSLSPGK

SEQ ID NO: 17 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVTKSEWQQGFVFSCSVMHEALHNHYTQKSLSLSPGK

SEQ ID NO: 18 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYQTTPPVLDSDGSFFLYSKLTVTKSEWQQGFVFSCSVMHEALHNHYTQKSLSLSPGK

SEQ ID NO: 19 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYQTTPPVLDSDGSFFLYSKLTVTKSEWQQGFVFSCSVMHEALHNHYTQKSLSLSPGK

SEQ ID NO: 20 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVTKSEWQQGFVFSCSVMHEALHNHYTQKSLSLSPGK

SEQ ID NO: 21 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTKSEWQQGFVFSCSVMHEALHNHYTQKSLSLSPGK

SEQ ID NO: 22 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFSCSVMHEALHNHYTQKSLSLSPGK

SEQ ID NO: 23 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTGEEWQQGFVFSCSVMHEALHNHYTQKSLSLSPGK

SEQ ID NO: 24 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFSCWVMHEALHNHYTQKSLSLSPGK

SEQ ID NO: 25 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTKEEWQQGFVFSCWVMHEALHNHYTQKSLSLSPGK

SEQ ID NO: 26 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFTCWVMHEALHNHYTQKSLSLSPGK

SEQ ID NO: 27 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFTCGVMHEALHNHYTQKSLSLSPGK

SEQ ID NO: 28 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFECWVMHEALHNHYTQKSLSLSPGK

SEQ ID NO: 29 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFKCWVMHEALHNHYTQKSLSLSPGK

SEQ ID NO: 30 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTPEEWQQGFVFKCWVMHEALHNHYTQKSLSLSPGK

SEQ ID NO: 31 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTREEWQQGFVFSCSVMHEALHNHYTQKSLSLSPGK

SEQ ID NO: 32 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTGEEWQQGFVFSCSVMHEALHNHYTQKSLSLSPGK

SEQ ID NO: 33 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTREEWQQGFVFTCWVMHEALHNHYTQKSLSLSPGK

SEQ ID NO: 34 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTGEEWQQGFVFTCWVMHEALHNHYTQKSLSLSPGK

SEQ ID NO: 35 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTREEWQQGFVFTCGVMHEALHNHYTQKSLSLSPGK

SEQ ID NO: 36 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 37 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTKEEWQQGFVFSCSVMHEALHNHYTQKSLSLSPGK

SEQ ID NO: 38 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 39 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWASYKTTPPVLDSDGSFFLYSKLTVTKEEWQQGFVFSCSVMHEALHNHYTQKSLSLSPGK

SEQ ID NO: 40 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWVSYKTTPPVLDSDGSFFLYSKLTVTKEEWQQGFVFSCSVMHEALHNHYTQKSLSLSPGK

SEQ ID NO: 41 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVSKEEWQQGFVFSCSVMHEALHNHYTQKSLSLSPGK

SEQ ID NO: 42 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWASYKTTPPVLDSDGSFFLYSKLTVTKEEWQQGFVFSCSVMHEALHNHYTQKSLSLSPGK

SEQ ID NO: 43 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWVSYKTTPPVLDSDGSFFLYSKLTVTKEEWQQGFVFSCSVMHEALHNHYTQKSLSLSPGK

SEQ ID NO: 44 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWSSYKTTPPVLDSDGSFFLYSKLTVTKEEWQQGFVFSCSVMHEALHNHYTQKSLSLSPGK

SEQ ID NO: 45 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWASYKTTPPVLDSDGSFFLYSKLTVTKEEWQQGFVFSCSVMHEALHNHYTQKSLSLSPGK

SEQ ID NO: 46 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWVSYKTTPPVLDSDGSFFLYSKLTVTKEEWQQGFVFSCSVMHEALHNHYTQKSLSLSPGK

SEQ ID NO: 47 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVSKEEWQQGFVFSCSVMHEALHNHYTQKSLSLSPGK

SEQ ID NO: 48 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWASYKTTPPVLDSDGSFFLYSKLTVTKEEWQQGFVFSCSVMHEALHNHYTQKSLSLSPGK

SEQ ID NO: 49 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWVSYKTTPPVLDSDGSFFLYSKLTVTKEEWQQGFVFSCSVMHEALHNHYTQKSLSLSPGK

SEQ ID NO: 50 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSNYKTTPPVLDSDGSFFLYSKLTVTKEEWQQGFVFSCSVMHEALHNHYTQKSLSLSPGK

SEQ ID NO: 51 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPGK

SEQ ID NO: 52 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 53 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 54 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWANYKTTPPVLDSDGSFFLYSKLTVTKEEWQQGFVFSCSVMHEALHNHYTQKSLSLSPGK

SEQ ID NO: 55 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWVNYKTTPPVLDSDGSFFLYSKLTVTKEEWQQGFVFSCSVMHEALHNHYTQKSLSLSPGK

SEQ ID NO: 56 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWSNYKTTPPVLDSDGSFFLYSKLTVTKEEWQQGFVFSCSVMHEALHNHYTQKSLSLSPGK

SEQ ID NO: 57 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWANYKTTPPVLDSDGSFFLYSKLTVTKEEWQQGFVFSCSVMHEALHNHYTQKSLSLSPGK

SEQ ID NO: 58 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWVNYKTTPPVLDSDGSFFLYSKLTVTKEEWQQGFVFSCSVMHEALHNHYTQKSLSLSPGK

SEQ ID NO: 59 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSNYKTTPPVLDSDGSFFLYSKLTVSKEEWQQGFVFSCSVMHEALHNHYTQKSLSLSPGK

SEQ ID NO: 60 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWANYKTTPPVLDSDGSFFLYSKLTVTKEEWQQGFVFSCSVMHEALHNHYTQKSLSLSPGK

SEQ ID NO: 61 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWVNYKTTPPVLDSDGSFFLYSKLTVTKEEWQQGFVFSCSVMHEALHNHYTQKSLSLSPGK

SEQ ID NO: 62 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESFGTEWSSYKTTPPVLDSDGSFFLYSKLTVTKEEWQQGFVFSCSVMHEALHNHYTQKSLSLSPGK

SEQ ID NO: 63 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWASYKTTPPVLDSDGSFFLYSKLTVTKEEWQQGFVFSCSVMHEALHNHYTQKSLSLSPGK

SEQ ID NO: 64 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWVSYKTTPPVLDSDGSFFLYSKLTVTKEEWQQGFVFSCSVMHEALHNHYTQKSLSLSPGK

SEQ ID NO: 65 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVSKEEWQQGFVFSCSVMHEALHNHYTQKSLSLSPGK

SEQ ID NO: 66 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESFGTEWASYKTTPPVLDSDGSFFLYSKLTVTKEEWQQGFVFSCSVMHEALHNHYTQKSLSLSPGK

SEQ ID NO: 67 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESFGTEWVSYKTTPPVLDSDGSFFLYSKLTVTKEEWQQGFVFSCSVMHEALHNHYTQKSLSLSPGK

SEQ ID NO: 68 - protein, 217 aa, Artificial Sequence; identical to SEQ ID NO: 17
  Description of Artificial Sequence Synthetic polypeptide

SEQ ID NO: 69 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 70 - protein, 517 aa, Homo sapiens
TDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 71 - protein, 15 aa, Homo sapiens
EPKSCDKTHTCPPCP

SEQ ID NO: 72 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 73 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 74 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 75 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 76 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 77 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 78 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 80 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 81 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVWWESYGTEWSSYKTTPPVLDSDGSFFLVSKLTVTKEEWQQGFVFSCSVMHEALHNHYTQKSLSLSPGK

SEQ ID NO: 82 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVWWESYGTEWSSYKTTPPVLDSDGSFFLVSKLTVTKEEWQQGFVFSCSVMHEALHNHYTQKSLSLSPGK

SEQ ID NO: 83 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVWWESYGTEWSSYKTTPPVLDSDGSFFLVSKLTVTKEEWQQGFVFSCSVMHEALHNHYTQKSLSLSPGK

SEQ ID NO: 84 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVWWESYGTEWSSYKTTPPVLDSDGSFFLVSKLTVTKEEWQQGFVFSCSVMHEALHNHYTQKSLSLSPGK

SEQ ID NO: 85 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 10 aa, Homo sapiens
DKTHTCPPCP

SEQ ID NO: 90 - protein, 525 aa, Homo sapiens
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 91 - protein, 757 aa, Artificial Sequence
  Description of Artificial Sequence Synthetic polypeptide
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 227 aa, Artificial Sequence
  Description of Artificial Sequence Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 93 - protein, 757 aa, Artificial Sequence
  Description of Artificial Sequence Synthetic polypeptide
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 757 aa, Artificial Sequence
  Description of Artificial Sequence Synthetic polypeptide
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 96 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 97 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 98 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 99 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 100 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 101 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 102 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 103 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 104 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 105 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 106 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 107 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 108 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 109 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 110 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 111 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 112 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 113 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 114 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 115 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 116 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 117 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 118 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 119 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 120 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 121 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 122 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 123 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 124 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 125 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 126 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWVNYKTTPPVLDSDGSFFLVSKLTVTKEEWQQGFVFSCSVMHEALHNHYTQKSLSLSPGK

SEQ ID NO: 127 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWVNYKTTPPVLDSDGSFFLVSKLTVTKEEWQQGFVFSCSVMHEALHNHYTQKSLSLSPGK

SEQ ID NO: 128 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWVNYKTTPPVLDSDGSFFLVSKLTVTKEEWQQGFVFSCSVMHEALHNHYTQKSLSLSPGK

SEQ ID NO: 129 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWVNYKTTPPVLDSDGSFFLVSKLTVTKEEWQQGFVFSCSVMHEALHNHYTQKSLSLSPGK

SEQ ID NO: 130 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWVNYKTTPPVLDSDGSFFLVSKLTVTKEEWQQGFVFSCSVMHEALHNHYTQKSLSLSPGK

SEQ ID NO: 131 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWVNYKTTPPVLDSDGSFFLVSKLTVTKEEWQQGFVFSCSVMHEALHNHYTQKSLSLSPGK

SEQ ID NO: 132 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 133 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 134 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 135 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 136 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 137 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 138 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 139 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 140 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 141 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 142 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 143 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 144 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 145 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 146 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 147 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 148 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 149 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 150 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 151 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 152 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 153 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 154 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 155 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 156 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 157 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 158 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 159 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 160 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 161 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 162 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVTKEEWQQGFVFSCSVMHEALHNHYTQKSLSLSPGK

SEQ ID NO: 163 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVTKEEWQQGFVFSCSVMHEALHNHYTQKSLSLSPGK

SEQ ID NO: 164 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWSNYKTTPPVLDSDGSFFLVSKLTVTKEEWQQGFVFSCSVMHEALHNHYTQKSLSLSPGK

SEQ ID NO: 165 - protein, 217 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVTKEEWQQGFVFSCSVMHEALHNHYTQKSLSLSPGK

SEQ ID NO: 166 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVTKEEWQQGFVFSCSVMHEALHNHYTQKSLSLSPGK

SEQ ID NO: 167 - protein, 217 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWSNYKTTPPVLDSDGSFFLVSKLTVTKEEWQQGFVFSCSVMHEALHNHYTQKSLSLSPGK

SEQ ID NO: 168 - protein, 227 aa, Artificial Sequence
  Description of Artificial Sequence Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 169 - protein, 227 aa, Artificial Sequence
  Description of Artificial Sequence Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 170 - protein, 525 aa, Homo sapiens; identical to SEQ ID NO: 90

SEQ ID NO: 171 - protein, 757 aa, Artificial Sequence; identical to SEQ ID NO: 91
  Description of Artificial Sequence Synthetic polypeptide

SEQ ID NO: 172 - protein, 757 aa, Artificial Sequence; identical to SEQ ID NO: 93
  Description of Artificial Sequence Synthetic polypeptide

SEQ ID NO: 173 - protein, 757 aa, Artificial Sequence; identical to SEQ ID NO: 94
  Description of Artificial Sequence Synthetic polypeptide

SEQ ID NO: 174 - protein, 525 aa, Artificial Sequence
  Description of Artificial Sequence Synthetic polypeptide
  MOD_RES (59)..(59): Formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 175 - protein, 757 aa, Artificial Sequence
  Description of Artificial Sequence Synthetic polypeptide
  MOD_RES (59)..(59): Formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 176 - protein, 757 aa, Artificial Sequence
  Description of Artificial Sequence Synthetic polypeptide
  MOD_RES (59)..(59): Formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 177 - protein, 757 aa, Artificial Sequence
  Description of Artificial Sequence Synthetic polypeptide
  MOD_RES (59)..(59): Formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 179 - protein, 5 aa, Artificial Sequence
  Description of Artificial Sequence Synthetic peptide
GGGGS

SEQ ID NO: 180 - protein, 10 aa, Artificial Sequence
  Description of Artificial Sequence Synthetic peptide
GGGGSGGGGS

SEQ ID NO: 181 - protein, 6 aa, Artificial Sequence
  Description of Artificial Sequence Synthetic peptide
HHHHHH

SEQ ID NO: 182 - protein, 7 aa, Artificial Sequence
  Description of Artificial Sequence Synthetic peptide
  MOD_RES (2)..(2): Any amino acid
YXTEWSS

SEQ ID NO: 183 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKEEWQQGFVFSCSVMHEALHNHYTQKSLSLSPG

SEQ ID NO: 185 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 186 - protein, 216 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 187 - protein, 216 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 188 - protein, 216 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 189 - protein, 226 aa, Artificial Sequence
  Description of Artificial Sequence Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 190 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVTKEEWQQGFVFSCSVMHEALHNHYTQKSLSLSPG

SEQ ID NO: 191 - protein, 216 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPG

SEQ ID NO: 192 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPG

SEQ ID NO: 193 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 194 - protein, 216 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 195 - protein, 216 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 196 - protein, 216 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 197 - protein, 216 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 198 - protein, 216 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 199 - protein, 226 aa, Artificial Sequence
  Description of Artificial Sequence Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 200 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWASYKTTPPVLDSDGSFFLYSKLTVTKEEWQQGFVFSCSVMHEALHNHYTQKSLSLSPG

SEQ ID NO: 201 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 202 - protein, 216 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 203 - protein, 216 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 204 - protein, 216 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 205 - protein, 216 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 206 - protein, 216 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 207 - protein, 226 aa, Artificial Sequence
  Description of Artificial Sequence Synthetic polypeptide
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 208 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 209 - protein, 216 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 210 - protein, 216 aa, Artificial Sequence
  Description of Artificial Sequence Synthetic polypeptide
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 211 - protein, 216 aa, Artificial Sequence
  Description of Artificial Sequence Synthetic polypeptide
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 212 - protein, 756 aa, Artificial Sequence
  Description of Artificial Sequence Synthetic polypeptide
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 213 - protein, 756 aa, Artificial Sequence
  Description of Artificial Sequence Synthetic polypeptide
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 214 - protein, 756 aa, Artificial Sequence; identical to SEQ ID NO: 212
  Description of Artificial Sequence Synthetic polypeptide

SEQ ID NO: 215 - protein, 756 aa, Artificial Sequence; identical to SEQ ID NO: 213
  Description of Artificial Sequence Synthetic polypeptide

SEQ ID NO: 216 - protein, 756 aa, Artificial Sequence
  Description of Artificial Sequence Synthetic polypeptide
  MOD_RES (59)..(59): Formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 217 - protein, 756 aa, Artificial Sequence
  Description of Artificial Sequence Synthetic polypeptide
  MOD_RES (59)..(59): Formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

(19) United States
(12) Patent Application Publication
(10) Pub. No.: US 2023/0092681 A1
(43) Pub. Date: Mar. 23, 2023

(54) METHODS FOR THE TREATMENT OF HUNTER SYNDROME

(71) Applicant: DENALI THERAPEUTICS INC., South San Francisco, CA (US)

(72) Inventors: Annie ARGUELLO, San Francisco, CA (US); Heather CAHAN, Foster City, CA (US); Vinay M. DARYANI, San Francisco, CA (US); Anita GROVER, San Mateo, CA (US); Matthew D. TROYER, Piedmont, CA (US)

(73) Assignee: DENALI THERAPEUTICS INC., South San Francisco, CA (US)

(21) Appl. No.: 17/797,644

(22) PCT Filed: Feb. 5, 2021
(86) PCT No.: PCT/US2021/016913

§ 371 Date: Aug. 4, 2022

Related U.S. Application Data
(60) Provisional application No. 62/971,758, filed on Feb. 7, 2020.
(60) Provisional application No. 63/091,704, filed on Oct. 14, 2020.
(60) Provisional application No. 63/111,586, filed on Nov. 9, 2020.
(60) Provisional application No. 63/135,974, filed on Jan. 11, 2021.

(51) Int. Cl. A61K 38/17; A61K 38/46; A61K 9/00; A61P 3/00

(52) U.S. Cl. CPC A61K 38/1774; A61K 38/465; A61K 9/0019; C12Y 301/06013; A61P 3/00

(57) ABSTRACT

Certain embodiments provide a method of treating Hunter syndrome in a subject in need thereof, comprising administering to the subject a therapeutically effective dose of a pharmaceutical composition comprising an ETV:IDS protein.

### CROSS-REFERENCE TO RELATED APPLICATIONS

This application claims priority to U.S. Provisional Application Ser. No. 62/971,758, filed Feb. 7, 2020, U.S. Provisional Application Ser. No. 63/091,704, filed Oct. 14, 2020, U.S. Provisional Application Ser. No. 63/111,586, filed Nov. 9, 2020, and U.S. Provisional Application Ser. No. 63/135,974, filed Jan. 11, 2021. The entire content of the applications referenced above are hereby incorporated by reference herein.

### BACKGROUND

Hunter syndrome, or MPS II, is a rare, X-linked recessive disorder caused by IDS gene mutations. Insufficient iduronate 2-sulfatase (IDS) activity leads to accumulation of the glycosaminoglycans (GAGs) heparan sulfate (HS) and dermatan sulfate (DS) and to lysosomal dysfunction in multiple organs and tissues. Approximately two-thirds of patients display a neuronopathic phenotype (nMPS II). A recombinant form of IDS has been approved to treat Hunter syndrome, but it has little effect on the brain due to difficulties in delivering the recombinant enzyme across the blood-brain barrier (BBB). Accordingly, there is a need for more effective therapies that treat both the peripheral and central nervous system (CNS) symptoms of Hunter syndrome. Additionally, there is a need for new methods and biomarkers for evaluating disease activity and treatment response for such newly developed therapeutics.

### SUMMARY

Accordingly, certain embodiments described herein provide a method of treating Hunter syndrome in a subject in need thereof, comprising administering to the subject a therapeutically effective dose of a pharmaceutical composition comprising an ETV:IDS protein described herein.

Certain embodiments also provide a method of treating Hunter syndrome in a subject in need thereof, comprising administering to the subject a therapeutically effective dose of a pharmaceutical composition comprising an ETV:IDS protein described herein, wherein administration of the pharmaceutical composition reduces levels of a glycosaminoglycan (GAG) in the CSF of the subject to a baseline level measured in a healthy subject or a subject that does not have Hunter syndrome.

In certain embodiments, the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

In certain embodiments, the pharmaceutical composition further comprises a pharmaceutically acceptable excipient (e.g., a pharmaceutically acceptable carrier).

In certain embodiments, the pharmaceutical composition is administered weekly.

In certain embodiments, the pharmaceutical composition is administered to the subject intravenously.

In certain embodiments, the therapeutically effective dose (e.g., a safe and therapeutically effective dose) is evaluated by examining the subject post-treatment using one or more assessments selected from the group consisting of a biomarker assessment, a safety assessment, a neurocognitive assessment, a clinical assessment, a functional assessment and a quality of life assessment.

In certain embodiments, the administration of the pharmaceutical composition treats one or more symptoms of Hunter syndrome (e.g., a neurobehavioral deficit, an auditory deficit and/or a musculoskeletal abnormality) in the subject. Thus, in certain embodiments, the administration of the pharmaceutical composition changes one or more disease associated parameters for the subject. In certain embodiments, the parameter is a parameter described herein. In certain embodiments, the parameter is evaluated using a biomarker assessment, a safety assessment, a neurocognitive assessment, a clinical assessment, a functional assessment or a quality of life assessment. In certain embodiments, the parameter is evaluated using an assessment described herein.

In certain embodiments, the duration of response in relation to the parameter change is at least about 2 weeks, 4 weeks, 6, weeks, 8 weeks, 10 weeks, 12 weeks, 14, weeks, 16 weeks, 18 weeks, 20 weeks, 24 weeks, 26 weeks, 28 weeks, 30 weeks, 32 weeks, 36 weeks or more. In certain embodiments, the duration of response is at least about 24 weeks.

In certain embodiments, the method further comprises adjusting the dose of the pharmaceutical composition based on a parameter described herein.

Certain embodiments also provide method of treating Hunter syndrome in a subject in need thereof, comprising administering to the subject a protein at a weekly dose of about 3 mg/kg, and wherein the protein comprises: (a) a first Fc polypeptide linked to an iduronate 2-sulfatase (IDS) amino acid sequence, an IDS variant amino acid sequence, or a catalytically active fragment thereof, and (b) a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Certain embodiments provide a method of treating Hunter syndrome in a subject in need thereof, comprising administering to the subject a protein at a weekly dose of about 7.5 mg/kg, and wherein the protein comprises: (a) a first Fc polypeptide linked to an iduronate 2-sulfatase (IDS) amino acid sequence, an IDS variant amino acid sequence, or a catalytically active fragment thereof, and (b) a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Certain embodiments provide a method of treating Hunter syndrome in a subject in need thereof, comprising administering to the subject a protein at a weekly dose of about 15 mg/kg, and wherein the protein comprises: (a) a first Fc polypeptide linked to an iduronate 2-sulfatase (IDS) amino acid sequence, an IDS variant amino acid sequence, or a catalytically active fragment thereof, and (b) a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Certain embodiments provide a method of treating Hunter syndrome in a subject in need thereof, comprising administering to the subject a protein at a weekly dose of about 30 mg/kg of protein, and wherein the protein comprises: (a) a first Fc polypeptide linked to an iduronate 2-sulfatase (IDS) amino acid sequence, an IDS variant amino acid sequence, or a catalytically active fragment thereof, and (b) a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

In certain embodiments, the second Fc polypeptide specifically binds to the transferrin receptor.

In certain embodiments, the IDS amino acid sequence has at least 90% identity to SEQ ID NO:70.

In certain embodiments, the IDS amino acid sequence has at least 90% identity to SEQ ID NO:90.

In certain embodiments, the IDS amino acid sequence comprises a sequence selected from the group consisting of SEQ ID NOs:70, 90, 170 and 174.

In certain embodiments, the IDS amino acid sequence comprises a sequence selected from the group consisting of SEQ ID NOs:70, 170 and 174.

In certain embodiments, the IDS amino acid sequence comprises a sequence selected from the group consisting of SEQ ID NOs:90, 170 and 174.

In certain embodiments, the first Fc polypeptide linked to the IDS amino acid sequence comprises the amino acid sequence of SEQ ID NO: 91, 171, 175, 213, 215 or 217. In certain embodiments, the first Fc polypeptide linked to the IDS amino acid sequence comprises the amino acid sequence of SEQ ID NO: 91, 171 or 175. In certain embodiments, the first Fc polypeptide linked to the IDS amino acid sequence comprises the amino acid sequence of SEQ ID NO: 213, 215 or 217.

In certain embodiments, the IDS amino acid sequence is linked to the N-terminus of the first Fc polypeptide.

In certain embodiments, the second Fc polypeptide comprises Leu at position 380; Ala at position 389; and Ser at position 390. In certain embodiments, the second Fc polypeptide comprises Glu at position 380; Ala at position 389; and Asn at position 390. In certain embodiments, the second Fc polypeptide comprises Trp at position 380; Ser at position 389; and Ser at position 390. In certain embodiments, the second Fc polypeptide comprises Leu at position 380; Ser at position 389; and Ser at position 390.

In certain embodiments, the first Fc polypeptide linked to the IDS amino acid sequence comprises SEQ ID NO: 175; and the second Fc polypeptide comprises SEQ ID NO: 92.

In certain embodiments, the first Fc polypeptide linked to the IDS amino acid sequence comprises SEQ ID NO: 217; and the second Fc polypeptide comprises SEQ ID NO: 189.

In certain embodiments, the first Fc polypeptide linked to the IDS amino acid sequence comprises SEQ ID NO: 175; and the second Fc polypeptide comprises SEQ ID NO: 95. In certain embodiments, the second Fc polypeptide further comprises SEQ ID NO: 89, wherein SEQ ID NO: 89 is attached to the N-terminus of SEQ ID NO:95.

In certain embodiments, the first Fc polypeptide linked to the IDS amino acid sequence comprises SEQ ID NO: 217; and the second Fc polypeptide comprises SEQ ID NO: 191. In certain embodiments, the second Fc polypeptide further comprises SEQ ID NO: 89, wherein SEQ ID NO: 89 is attached to the N-terminus of SEQ ID NO: 191.

In certain embodiments, the first Fc polypeptide linked to the IDS amino acid sequence comprises SEQ ID NO: 175; and the second Fc polypeptide comprises SEQ ID NO: 168.

In certain embodiments, the first Fc polypeptide linked to the IDS amino acid sequence comprises SEQ ID NO: 217; and the second Fc polypeptide comprises SEQ ID NO: 207.

In certain embodiments, the first Fc polypeptide linked to the IDS amino acid sequence comprises SEQ ID NO: 175; and the second Fc polypeptide comprises SEQ ID NO: 169.

In certain embodiments, the first Fc polypeptide linked to the IDS amino acid sequence comprises SEQ ID NO: 217; and the second Fc polypeptide comprises SEQ ID NO: 199.

Certain embodiments also provide a pharmaceutical composition comprising a protein for use in a method of treating Hunter syndrome, the method comprising administering a therapeutically effective dose of the pharmaceutical composition to a subject in need thereof, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Certain embodiments also provide the use of a protein in the preparation of a medicament for treating Hunter syndrome by administering a therapeutically effective dose of the medicament to a subject in need thereof, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

### BRIEF DESCRIPTION OF THE FIGURES

FIG. 1. Brain HS/DS (GAG) accumulation in IDS KO mice relative to age matched control. For each age grouping, wildtype (WT) is shown on the left and IDS KO is shown on the right.

FIGS. 2A-2D. Lysosomal lipids (GM2, GM3, BMP and GlcCer) accumulate in the brains of IDS KO mice relative to their age matched controls. In FIGS. 2B-2D, for each age grouping, the bar representing WT is shown on the left and bar representing IDS KO is shown on the right.

FIG. 3. Elevated levels of BMP observed in the serum of IDS KO mice relative to their age matched controls. For each age grouping, the bar representing wildtype (WT) is shown on the left and bar representing IDS KO is shown on the right.

FIG. 4. Elevated levels of lysosomal lipids (Gd1a/b, GM3, BMP and GlcCer) observed in the CSF of IDS KO mice relative to their age matched controls. For each age grouping, the bar representing wildtype (WT) is shown on the left, and bar representing IDS KO is shown on the right.

FIGS. 5A-5B. Peripheral administration of ETV:IDS (4 week treatment) corrects (FIG. 5A) brain and (FIG. 5B) CSF GAG accumulation in IDS KO mice. Graphs display mean±SEM and p values: one-way ANOVA with Dunnett multiple comparison test; * p≤0.05, ** p≤0.01, *** p≤0.001, and **** p≤0.0001.

FIG. 6. Peripheral administration of ETV:IDS (4 week treatment) corrects lysosomal lipid (gangliosides) accumulation in IDS KO brains. Graphs display mean±SEM and p values: one-way ANOVA with Dunnett multiple comparison test; * p≤0.05, ** p≤0.01, *** p≤0.001, and **** p≤0.0001.

FIGS. 7A-7B. Peripheral administration of ETV:IDS (4 week treatment) corrects lysosomal lipid (GlcCer) accumulation in IDS KO brains. Graphs display mean±SEM and p values: one-way ANOVA with Dunnett multiple comparison test; * p≤0.05, ** p≤0.01, *** p≤0.001, and **** p≤0.0001.

FIG. 8. Peripheral administration of ETV:IDS (4 week treatment) corrects lysosomal lipid (BMP) accumulation in IDS KO brains. The graph displays mean±SEM and p values: one-way ANOVA with Dunnett multiple comparison test; * p≤0.05, ** p<0.01, *** p≤0.001, and **** p≤0.0001.

FIG. 9. Heat map illustrating lipid levels in brains of IDS KO mice treated with vehicle, ETV:IDS or Elaprase (idursulfase). Fold change as compared to WT.

FIG. 10. Peripheral administration of ETV:IDS corrects TREM2 accumulation in IDS KO brains. The graph displays mean±SEM and p values: one-way ANOVA with Dunnett multiple comparison test; **** p 0.0001.

FIG. 11. Peripheral administration of ETV:IDS reduces accumulation of CSF soluble TREM2 (sTrem2) levels in IDS KO; TfR^{mu/hu} KI mice.

FIGS. 12A-12C. Serum (FIG. 12A), brain (FIG. 12B) and CSF (FIG. 12C) HS/DS (GAG) accumulation in IDS KO mice treated with vehicle or 3, 10, 20, or 40 mg/kg ETV:IDS.

FIGS. 13A-13C. Peripheral administration of ETV:IDS corrects GM3 (FIG. 13A), GlcCer (FIG. 13B) and BMP (FIG. 13C) accumulation in brains of IDS KO mice treated with 3, 10, 20, or 40 mg/kg ETV:IDS, as compared to IDS KO mice treated with vehicle. Graphs display mean±SEM and p values: one-way ANOVA with Dunnett multiple comparison test; * p≤0.05, ** p≤0.01, *** p≤0.001, and **** p≤0.0001.

FIGS. 14A-14B. Administration of ETV:IDS reduces the levels of individual GAG species (D0S0, D0A0 and D0a4) in brain (FIG. 14A) and CSF (FIG. 14B) from IDS KO mice, as compared to IDS KO mice treated with vehicle. Graphs display mean±SEM and p values: one-way ANOVA with Dunnett multiple comparison test; ** p≤0.01, *** p<0.001, and **** p≤0.0001.

FIG. 15A. GAG levels in microglia, astrocytes, and neurons isolated from IDS KO; TfR^{mu/hu}KI mice relative to TfR^{mu/hu}KI controls (n=3-5 mice per group). All data are displayed as mean±SEM; unpaired student's t-test p≤0.05*, 0.001***.

FIG. 15B. Distribution of ETV:IDS in FACS-enriched neuron, astrocyte, and microglia cell populations at 2-hours post dose in vehicle-treated TfR^{mu/hu}KI and ETV:IDS (40 mg/kg) treated IDS KO; TfR^{mu/hu}KI mice; n=4 mice per group. Graph displays mean±SEM; two-way ANOVA with Sidak's test; ** p≤0.01, *** p≤0.001, and **** p≤0.0001.

FIG. 16. Reduction of GAG accumulation in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, *** p≤0.0001.

FIG. 17. Reduction of ganglioside accumulation in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, **** p≤0.0001.

FIG. 18. Reduction of glucosylceramide accumulation in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, **** p≤0.0001.

FIG. 19. Rescue of BMP alterations in neurons, astrocytes, and microglia from IDS KO; TfR^{mu/hu} KI mice dosed intravenously with 40 mg/kg ETV:IDS once a week for four weeks. Data are displayed as mean±SEM; one-way ANOVA with Tukey's multiple comparison test, * p≤0.05, ** p≤0.01, **** p≤0.0001.

FIG. 20. H&E staining (top panel) and MALDI MS images (middle and bottom panels) of coronal brain sections of wild-type and IDS KO; TfR^{mu/hu} KI mice after four, weekly doses of vehicle, idursulfase or ETV:IDS. The middle panel shows the distribution of the signal at m/z 1382.816, corresponding to GM2 (d36:1); and the bottom panel shows the distribution of the signal at m/z 1179.738, corresponding to GM3 (d36:1). Images depict the relative intensity of each signal from 0-100%.

FIG. 21. Immunofluorescent images of brain tissue from TfR^{mu/hu} KI and IDS KO; TfR^{mu/hu} KI mice that were treated with vehicle, ETV:IDS or idursulfase. The brain tissue was stained for DAPI and CD68. Magnification is 20×, and the images represent hippocampus (top panel), cortex (middle panel), and striatum (bottom panel).

FIG. 22. Dose-dependent reduction in brain GAGs following ETV:IDS treatment. GAG levels in brain from IDS KO; TfR^{mu/hu} KI mice were measured 7 days after the last dose of ETV:IDS or idursulfase and compared to vehicle treatment and TfR^{mu/hu} KI mice; n=5 per group. Graphs display mean±SEM and p values: one-way ANOVA with Dunnett multiple comparison test; **** p<0.0001. Values calculated include fold over TfR^{mu/hu} KI, percent treatment efficiency, and percent reduction from vehicle treated IDS KO; TfR^{mu/hu} KI mice.

FIG. 23. Dose-dependent reduction in CSF GAGs following ETV:IDS treatment. GAG levels in CSF from IDS KO; TfR^{mu/hu} KI mice were measured 7 days after the last dose of ETV:IDS or idursulfase and compared to vehicle treatment and TfR^{mu/hu} KI mice; n=4-5 per group. Graphs display mean±SEM and p values: one-way ANOVA with Dunnett multiple comparison test; **** p<0.0001. Values calculated include fold over TfR^{mu/hu} KI, percent treatment efficiency, and percent reduction from vehicle treated IDS KO; TfR^{mu/hu} KI mice.

FIG. 24. Reduction in liver GAGs following ETV:IDS and Idursulfase treatment. GAG levels in liver from IDS KO; TfR^{mu/hu} KI mice were measured 7 days after the last dose of ETV:IDS or idursulfase and compared to vehicle treatment and TfR^{mu/hu} KI mice; n=5 per group. Graphs display mean±SEM and are represented in log scale. Values calculated include Fold over TfR^{mu/hu} KI, percent treatment efficiency, and percent reduction from vehicle treated IDS KO; TfR^{mu/hu} KI mice.

FIGS. 25A-25C. Reduction of brain lysosomal lipid accumulation following ETV:IDS treatment. Brain levels of (FIG. 25A) glucosylceramide and (FIG. 25C) bis(monoacylglycerol)phosphate (BMP) and brain (top panel) and CSF (bottom panel) levels of gangliosides (FIG. 25B) from IDS KO; TfR^{mu/hu} KI mice were measured 7 days after their last dose of ETV:IDS or idursulfase and compared to vehicle treatment and TfR^{mu/hu} KI mice; n=5 per group. Graphs display mean±SEM and p values: one-way ANOVA with Dunnett multiple comparison test; * p<0.05, ** p<0.01, *** p<0.001, and **** p<0.0001. Values calculated include Fold over TfR^{mu/hu} KI, percent treatment efficiency, and percent reduction from vehicle treated IDS KO; TfR^{mu/hu} KI mice.

FIG. 26. Dose-dependent reduction in brain Trem2 levels following ETV:IDS treatment. Levels of Trem2 were assessed using an MSD-based assay in brains of IDS KO; TfR^{mu/hu} KI mice 7 days after their last dose of ETV:IDS or idursulfase and compared to vehicle treatment and TfR^{mu/hu} KI mice; n=5 per group. Graphs display mean±SEM and p values: one-way ANOVA with Dunnett multiple comparison test; * p<0.05, *** p<0.001, and **** p<0.0001. Values calculated include fold over TfR^{mu/hu} KI, percent treatment efficiency, and percent reduction from vehicle treated IDS KO; TfR^{mu/hu} KI mice.

FIGS. 27A-27B. Reduction in brain and CSF GAG accumulation following ETV:IDS treatment. GAG levels were evaluated in the brain (FIG. 27A) and CSF (FIG. 27B) of mice treated with 1 mg/kg or 3 mg/kg ETV:IDS for 13 weekly doses and compared to vehicle treatment and TfR^{mu/hu} KI mice. Values calculated include fold over TfR^{mu/hu} KI, percent treatment efficiency, and percent reduction from vehicle treated IDS KO; TfR^{mu/hu} KI mice (**p<0.01; ****p<0.0001; error bars=SEM).

FIGS. 28A-28B. Reduction in peripheral GAG accumulation following ETV:IDS treatment. GAG levels were evaluated in the liver (FIG. 28A) and urine (FIG. 28B) of mice treated with 1 mg/kg or 3 mg/kg ETV:IDS for 13 weekly doses and compared to vehicle treatment and TfR^{mu/hu} KI mice. Urine GAG levels were normalized to creatinine. Values calculated include fold over TfR^{mu/hu} KI, percent treatment efficiency, and percent reduction from vehicle treated IDS KO; TfR^{mu/hu} KI mice (**** p<0.0001; error bars=SEM).

FIG. 29. Reduction in brain lysosomal lipid accumulation following ETV:IDS treatment. Lysosomal lipids glucosyl ceramides (left panel), GM3 (middle panel) and BMP (right panel) were evaluated in the brains of mice treated with 1 mg/kg or 3 mg/kg ETV:IDS for 13 weekly doses and compared to vehicle treatment and TfR^{mu/hu} KI mice. Values calculated include fold over TfR^{mu/hu} KI, percent treatment efficiency, and percent reduction from vehicle treated IDS KO; TfR^{mu/hu} KI mice (****p<0.0001; error bars=SEM).

FIG. 30. Reduction in CSF lysosomal lipid accumulation following ETV:IDS treatment. Lysosomal lipids glucosyl ceramides (left panel), GM3 (middle panel) and BMP (right panel) were evaluated in the CSF of mice treated with 1 mg/kg or 3 mg/kg ETV:IDS for 13 weekly doses and compared to vehicle treatment and TfR^{mu/hu} mice. Values calculated include fold over TfR^{mu/hu}, percent treatment efficiency, and percent reduction from vehicle treated IDS KO; TfR^{mu/hu} mice (*p<0.05; ***p<0.001; ****p<0.0001; error bars=SEM).

FIGS. 31A-31C. Scatter plots of Nf-L concentrations in wild-type and IDS knockout mice at 3, 6, and 9 months of age. Nf-L levels in serum and CSF of 9-month old mice are illustrated in FIGS. 31A and 31B, respectively. Nf-L levels in CSF of mice cohorts at different ages are illustrated in FIG. 31C. Data are displayed as mean+/−SEM with p values obtained by unpaired t test analysis.

FIG. 32. Reduction in CSF Neurofilament Light Chain (Nf-L) following administration of ETV:IDS. Levels of Neurofilament Light chain (Nf-L) were evaluated in the CSF of mice treated with 1 mg/kg or 3 mg/kg ETV:IDS for 13 weekly doses and compared to vehicle treatment and TfR^{mu/hu} KI mice. Values calculated include Fold over TfR^{mu/hu} KI, percent treatment efficiency, and percent reduction from vehicle treated IDS KO; TfR^{mu/hu} KI mice. One outlier was removed from TfR^{mu/hu} vehicle treated and 3 mg/kg ETV:IDS treated groups as determined by Grubbs test or ROUT method (*p<0.05; ***p<0.001; error bars=SEM).

FIG. 33. ETV:IDS reduces peripheral and CNS GAGs. GAG levels were evaluated in the liver, brain and CSF of Ids KO; TfR^{mu/hu} mice and compared to vehicle treatment and non-diseased TfR^{mu/hu} mice. Graphs display mean±SEM: one-way ANOVA with Tukey's multiple comparison test; * p<0.05, ** p<0.01, *** p<0.001, and **** p<0.0001.

FIG. 34. ETV:IDS reduces serum and CSF Nf-L levels. Nf-L levels were evaluated in the serum (left panel) and CSF (right panel) of Ids KO; TfR^{mu/hu} mice and compared to vehicle treatment and non-diseased TfR^{mu/hu} mice. Graphs display mean±SEM: one-way ANOVA with Tukey's multiple comparison test; * p<0.05, ** p<0.01, *** p<0.001, and **** p<0.0001.

FIG. 35. ETV:IDS improves certain neurobehavioral deficits in Ids KO; TfR^{mu/hu} mice. Proportion of mice that successfully completed the running trials on the accelerated treadmill (Fisher exact or Chi-square test). Comparison of vehicle treated TfR^{mu/hu} mice and Ids KO; TfR^{mu/hu} mice, or as indicated by brackets; * p<0.05, ** p<0.001, *** p<0.0005, and **** p<0.0001.

FIG. 36. ETV:IDS improves certain neurobehavioral deficits in Ids KO; TfR^{mu/hu} mice. Latency to descend the pole averaged across 3 trials (linear mixed-effects model). Graph displays mean±SEM. Comparison of vehicle treated TfR^{mu/hu} mice and Ids KO; TfR^{mu/hu} mice, or as indicated by brackets; * p<0.05, ** p<0.001, *** p<0.0005, and **** p<0.0001.

FIG. 37. ETV:IDS improves certain neurobehavioral deficits in Ids KO; TfR^{mu/hu} mice. Levels of startle inhibition were calculated in the pre-pulse inhibition assay (linear model). Graph displays mean±SEM. Comparison of vehicle treated TfR^{mu/hu} mice and Ids KO; TfR^{mu/hu} mice, or as indicated by brackets; * p<0.05, ** p<0.001, *** p<0.0005, and **** p<0.0001.

FIG. 38. ETV:IDS normalizes certain neurobehavioral deficits in Ids KO; TfR^{mu/hu} mice. In the active place avoidance assay, mice are trained to avoid an unmarked aversive zone (orange) where a mild foot shock was presented upon entrance in the zone. The number of entrances in the aversive zone was measured during each phase of testing (Linear mixed-effects model to assess treatment effect in Ids KO; TfR^{mu/hu} mice). Graph displays mean±SEM. Comparison of vehicle treated TfR^{mu/hu} mice and Ids KO; TfR^{mu/hu} mice, or as indicated by brackets; * p<0.05, ** p<0.001, *** p<0.0005, and **** p<0.0001.

FIG. 39. ETV:IDS normalizes certain neurobehavioral deficits in Ids KO; TfR^{mu/hu} mice. Number of entrances in the aversive zone from the third trial and the reinstatement trial (Wilcoxon sum rank test). Graphs display mean±SEM except. Comparison of vehicle treated TfR^{mu/hu} mice and Ids KO; TfR^{mu/hu} mice, or as indicated by brackets; * p<0.05, ** p<0.001, *** p<0.0005, and **** p<0.0001.

FIG. 40. ETV:IDS normalizes certain neurobehavioral deficits in Ids KO; TfR^{mu/hu} mice. Classification of performance based on number of entrances in the aversive zone during the reinstatement trial; poor (>1), good (1), top (0). Fisher's exact test. Comparison of vehicle treated TfR^{mu/hu} mice and Ids KO; TfR^{mu/hu} mice, or as indicated by brackets; * p<0.05, ** p<0.001, *** p<0.0005, and **** p<0.0001.

FIG. 41. The three mice treatment cohorts were observed to have similar pain response in the hot plate assay and proper avoidance of the aversive location in the visual cliff assay. The hind paw withdrawal was measured in the hot plate test to assess supraspinal nociception (Wilcoxon rank sum test, ns) (left panel). Visual function of the mice was assessed by evaluating their preference toward the shallow over the deep zones in the visual cliff test (Chi-square test, ns) (right panel). Graphs display mean±SEM. Comparison of vehicle treated TfR^{mu/hu} mice and Ids KO; TfR^{mu/hu} mice, or as indicated by brackets; * p<0.05, ** p<0.001, *** p<0.0005, and **** p<0.0001.

FIG. 42. ETV:IDS improves auricular abnormalities of Ids KO; TfR^{mu/hu} mice. Representative H&E photomicrographs of the ear canal from vehicle-treated TfR^{mu/hu}, vehicle-treated Ids KO; TfR^{mu/hu} mice, and ETV:IDS-treated Ids KO; TfR^{mu/hu} mice. The tympanic bulla (TB) in the examined Ids KO; TfR^{mu/hu} mice had effusion and/or chronic otitis media as evidenced by the presence of cellular debris and exudate (black arrowhead) and expansion of the tympanic membrane (TM) by inflammatory cell infiltrates (higher magnification in panel a). Higher magnification of the lining of the TB (panel b) showed the submucosa (SM) of the TB epithelium (EP) was expanded by edema or dense connective tissue (fibrosis); EAM=external auditory canal; BM=bone marrow cells. Fibrous polyps were also evident (yellow arrowheads).

FIG. 43. ETV:IDS improves the facial morphology of Ids KO; TfR^{mu/hu} mice. Qualitative assessment of the severity of an animal's shortened, broadened snout (arrowhead). Representative images of each facial score is shown on the left panel. Graphs display the number of mice within each category: Fisher's exact test; **** p<0.0001.

FIG. 44. ETV:IDS improves skeletal abnormalities in Ids KO; TfR^{mu/hu} mice. Representative image of the femur defining the regions of interest for analysis of cortical and trabecular bone; representative cross sectional images from vehicle-treated TfR^{mu/hu} (n=5), vehicle-treated Ids KO; TfR^{mu/hu} mice (n=5), and ETV:IDS-treated Ids KO; TfR^{mu/hu} mice (n=5).

FIG. 45. ETV:IDS improves skeletal abnormalities in Ids KO; TfR^{mu/hu} mice. Quantitative analysis from the micro-CT scans of the femur trabecular. Graphs display mean SEM: one-way ANOVA with Tukey's multiple comparison test; * p≤0.05, *** p≤0.001, and **** p≤0.0001.

FIG. 46. ETV:IDS improves skeletal abnormalities in Ids KO; TfR^{mu/hu} mice. (Quantitative analysis from the micro-CT scans of the femur cortical bone. Graphs display mean±SEM: one-way ANOVA with Tukey's multiple comparison test; * p≤0.05, *** p≤0.001, and **** p≤0.0001.

FIG. 47. Scheme showing assessments for a nonrandomized, multicenter, prospective, observational study of patients diagnosed with MPS II.

FIG. 48. Scheme showing dosing escalation and assessments for a Phase 1/2 study of patients diagnosed with MPS II. To Be Determined¹ (TBD¹): Subjects within sub-cohorts B1 and B2 may undergo dose escalation based on Cohort A and Cohort B safety and biomarker results; however, dosing will not exceed 30 mg/kg. TBD²: Dosing for Cohort C will be determined based on Cohort A and Cohort B safety and biomarker results.

FIG. 49. ETV:IDS statistically reduces CSF GAG (heparan sulfate, dermatan sulfate) levels in MPS II patients after four weekly doses. P-values were determined by T-test comparison of mean percent change from baseline.

FIGS. 50A-50B. Heparan sulfate (FIG. 50A) and dermatan sulfate (FIG. 50B) levels in CSF from MPS II patients after 12 weekly doses of ETV:IDS. Normal range (black circle) determined using 30 adult CSF samples (range 10-90 percentile); adult and child CSF GAG levels are similar (Hendriksz et al., Mol. Genet. Metab. Rep., 5:103-106 (2015)). P-values were determined by T-test comparison of mean percent change from baseline.

FIGS. 51A-51B. Heparan sulfate (FIG. 51A) and dermatan sulfate (FIG. 51B) levels in urine from MPS II patients after 8 weekly doses of ETV:IDS. P-values were determined by T-test comparison of mean percent change from baseline.

FIGS. 52A-52B. GM3 (FIG. 52A) and BMP (FIG. 52B) levels in CSF from MPS II patients after 8 weekly doses of ETV:IDS. Individual patient data is shown on the left and Group Mean % change from baseline (Δ BL) is shown on the right. P-values were determined by T-test comparison of mean percent change from baseline.

### DETAILED DESCRIPTION

Hunter syndrome, specifically the neurocognitive phenotype, remains a significant unmet medical need. Described herein is the evaluation of a specific enzyme replacement therapy termed ETV:IDS, which has the capability of crossing the BBB and treating both the peripheral and CNS manifestations of Hunter syndrome, including neurobehavioral deficits, auditory deficits and musculoskeletal abnormalities. Exemplary ETV:IDS proteins are described herein, and disease activity and therapeutic response to ETV:IDS may be evaluated using a series of assessments (e.g., one or more assessments), including biomarker, safety, neurocognitive, clinical, functional, and quality of life assessments. For example, exploration of CSF, serum, and urine biomarkers described herein, including glycosaminoglycan (GAG) species, indicators of CNS lysosomal lipid accumulation (sphingolipids, gangliosides, and bis(monoacylglycerol)phosphate (BMP) species), microglial activation and neuroinflammation (sTREM2; cytokines), and neuroaxonal neurofilament light chain (Nf-L) may be used to identify and evaluate safe and therapeutically effective doses of ETV:IDS. In some embodiments, changes in the CSF, serum, and urine biomarkers described herein can occur after switching to treatment with ETV:IDS from an intravenous idursulfase treatment (e.g. Elaprase).

Also described herein is an exploration of therapeutically effective doses of ETV:IDS, or a pharmaceutical composition including the same, which significantly reduce CSF GAG levels in patients with Hunter syndrome. In some embodiments, the administered doses of ETV:IDS result in normalization of CSF GAG levels. Furthermore, as described herein, the administered doses can have a beneficial impact on CSF lysosomal lipid levels, as well as urine GAG levels in the patients.

### Biomarkers Assessments

Disease activity and therapeutic response to ETV:IDS may be evaluated using a series of biomarkers described herein. In particular, these biomarkers include the accumulation of glycosaminoglycans (GAGs) and specific lipids (i.e., sphingolipids, gangliosides, and BMPs) in a subject having Hunter syndrome, as well as the accumulation of Nf-L and TREM2, which may be measured based on sTREM2 levels. Additionally, increased concentrations of cytokines may also be used as a biomarker for neuroinflammation. Thus, the concentration of one or more of these molecules/lipids/proteins may be evaluated and compared relative to a baseline level or control (e.g., a baseline pre-treatment level as described herein). In certain embodiments, the assessment is performed, e.g., 6 months, 12 months and/or 18 months after treatment initiation.

In certain embodiments, administration of a pharmaceutical composition comprising ETV:IDS reduces levels of one or more analytes in a sample from the subject as compared to a control, wherein the one or more analytes are selected from the group consisting of a GAG, Nf-L, sTREM2, a cytokine, a BMP, a ganglioside and a sphingolipid. In certain embodiments, administration of a pharmaceutical composition comprising ETV:IDS reduces levels of one or more analytes in a sample from the subject by at least about 10%, 15%, 20%, 25%, or 30% as compared to a control, wherein the one or more analytes are selected from the group consisting of a GAG, Nf-L, sTREM2, a cytokine, a BMP, a ganglioside and a sphingolipid. In certain embodiments, the control is a Hunter syndrome patient that was not administered the pharmaceutical composition. In certain embodiments, the control is the subject prior to treatment.

In certain embodiments, administration of the pharmaceutical composition reduces levels of one or more analytes in a sample from the subject to baseline levels, wherein the one or more analytes are selected from the group consisting of a GAG, Nf-L, sTREM2, a cytokine, a BMP, a ganglioside and a sphingolipid. In certain embodiments, the baseline levels are measured in a sample from a healthy individual. In certain embodiments, the baseline levels are measured in a sample from a subject that does not have Hunter syndrome.

As used herein, the phrase “sample” or “physiological sample” is meant to refer to a biological sample obtained from a subject that contains an analyte of interest, such as a polysaccharide, protein and/or lipid. Thus, the sample may be evaluated at, e.g., the molecular, lipid or protein level. In certain embodiments, the physiological sample comprises tissue, cerebrospinal fluid (CSF), urine, blood, serum, or plasma. In certain embodiments, the sample comprises tissue, such as brain, liver, kidney, lung or spleen. The sample may include a fluid. In certain embodiments, the sample comprises CSF. In certain embodiments, the sample comprises blood and/or plasma. In certain embodiments, the sample comprises serum. In certain embodiments, the sample comprises CNS tissue (e.g., brain tissue).

In certain embodiments, administration of the pharmaceutical composition reduces levels of one or more analytes in an organ, tissue or fluid (e.g., blood, plasma, serum, CSF or urine) of the subject, as compared to a control, wherein the one or more analytes are selected from the group consisting of a GAG, Nf-L, sTREM2, a cytokine, a BMP, a ganglioside and a sphingolipid. In certain embodiments, administration of the pharmaceutical composition reduces levels of one or more analytes in an organ, tissue or fluid (e.g., blood, plasma, serum, CSF or urine) of the subject by at least about 10%, 15%, 20%, 25%, or 30%, as compared to a control, wherein the one or more analytes are selected from the group consisting of a GAG, Nf-L, sTREM2, a cytokine, a BMP, a ganglioside and a sphingolipid. In certain embodiments, the control is a Hunter syndrome patient that was not administered the pharmaceutical composition. In certain embodiments, the control is the subject prior to treatment. For example, in certain embodiments, administration of the pharmaceutical composition reduces levels of one or more analytes in the CSF of the subject by at least about 10%, 15%, 20%, 25%, or 30%, wherein the reduction is relative to the level of the corresponding one or more analytes in the subject prior to the administration, wherein the one or more analytes are selected from the group consisting of a GAG, Nf-L, sTREM2, a cytokine, a BMP, a ganglioside and a sphingolipid.

In certain embodiments, administration of the pharmaceutical composition reduces levels of one or more analytes in an organ, tissue or fluid (e.g., CSF, blood, plasma, serum or urine) of the subject to baseline levels, wherein the one or more analytes are selected from the group consisting of a GAG, Nf-L, sTREM2, a cytokine, a BMP, a ganglioside, and a sphingolipid. In certain embodiments, the baseline levels are measured in a healthy individual. In certain embodiments, the baseline levels are measured in a subject that does not have Hunter syndrome.

### Glycosaminoglycans (GAGs)

Insufficient or absent IDS enzyme activity leads to the accumulation of the glycosaminoglycans (GAGs) heparan sulfate (HS; D0A0 and D0S0) and dermatan sulfate (DS; D0a4) in the CNS and periphery, as well as lysosome dysfunction in multiple organs and tissues. Accordingly, the concentration of total GAGs or the concentration of one or more GAG species may be evaluated in a subject having Hunter syndrome (e.g., in a post-treatment sample from a subject administered ETV:IDS). Quantification of GAG concentration levels may be performed using methods known in the art, for example, using a liquid chromatography mass spectrometry (LCMS) assay (see, e.g., the Examples).

In certain embodiments, the administration of the pharmaceutical composition reduces levels of a GAG in an organ, tissue or fluid of the subject, as compared to a control. In certain embodiments, levels of a GAG in an organ, tissue or fluid of the subject are reduced by at least about 30%, 40%, 50%, 60%, 70%, 80%, 90%, 95% or more, as compared to a control. In certain embodiments, the administration of the pharmaceutical composition reduces levels of a GAG in an organ, tissue or fluid of the subject, to baseline levels (e.g. levels measured in a healthy subject or a subject that does not have Hunter syndrome).

In certain embodiments, the administration of the pharmaceutical composition reduces levels of a GAG in the CNS of the subject, as compared to a control. In certain embodiments, levels of a GAG in the CNS of the subject is reduced by at least about 30%, 40%, 50%, 60%, 70%, 80%, 90%, 95% or more, as compared to a control. In certain embodiments, the administration of the pharmaceutical composition reduces levels of a GAG in the CNS of the subject to baseline levels (e.g. levels measured in a healthy subject or a subject that does not have Hunter syndrome).

In certain embodiments, the administration of the pharmaceutical composition reduces levels of a GAG in the CSF of the subject, as compared to a control. In certain embodiments, levels of a GAG in the CSF of the subject is reduced by at least about 30%, 40%, 50%, 60%, 70%, 80%, 90%, 95% or more, as compared to a control. In certain embodiments, levels of a GAG in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a GAG in the CSF of the subject is relative to the CSF levels of the GAG in the subject prior to the administration. For example, in certain embodiments, the administration of the pharmaceutical composition reduces levels of a GAG in the CSF of the subject by at least 50%, 60%, 70%, 75%, 80%, 85%, or 90% after at least 4 weekly doses, wherein the reduction is relative to the CSF levels of the GAG in the subject prior to the administration. For example, in certain embodiments, the administration of the pharmaceutical composition reduces levels of heparan sulfate in the CSF of the subject by at least 80% after at least 4 weekly doses, wherein the reduction is relative to the CSF heparan sulfate levels in the subject prior to administration. In certain embodiments, the administration of the pharmaceutical composition reduces levels of the glycosaminoglycan (GAG) in the CSF of the subject by at least 50%, 60%, 70%, 75%, 80%, 85%, or 90% after 4 weekly doses. In certain embodiments, the administration of the pharmaceutical composition reduces levels of a glycosaminoglycan (GAG) in the CSF of the subject by at least 50%, 60%, 70%, 75%, 80%, 85%, or 90% after at least 8 weekly doses, wherein the reduction is relative to the CSF levels of the GAG in the subject prior to the administration. In certain embodiments, the administration of the pharmaceutical composition reduces levels of the glycosaminoglycan (GAG) in the CSF of the subject by at least 50%, 60%, 70%, 75%, 80%, 85%, or 90% after 8 weekly doses. In certain embodiments, the administration of the pharmaceutical composition reduces levels of a GAG in the CSF of the subject by at least about 50%, 60%, 70%, 75%, 80%, 85%, or 90% after at least 12 weekly doses, wherein the reduction is relative to the CSF levels of the GAG in the subject prior to the administration. For example, in certain embodiments, the administration of the pharmaceutical composition reduces levels heparan sulfate in the CSF of the subject by at least 70% after at least 12 weekly doses, wherein the reduction is relative to the CSF heparan sulfate levels in the subject prior to administration. In certain embodiments, the administration of the pharmaceutical composition reduces levels of the GAG in the CSF of the subject by at least about 50%, 60%, 70%, 75%, 80%, 85%, or 90% after 12 weekly doses. In certain embodiments, the administration of the pharmaceutical composition normalizes levels of a GAG in the CSF of the subject, i.e. reduces levels of a GAG in the CSF of the subject to baseline levels measured in a healthy subject or a subject that does not have Hunter syndrome. In certain embodiments, the reduction or normalization of a GAG in the CSF of the subject occurs after switching the subject's administered therapy from intravenous idursulfase (e.g. Elaprase) to the pharmaceutical composition comprising ETV:IDS.

In certain embodiments, the administration of the pharmaceutical composition reduces levels of a GAG in the serum of the subject, as compared to a control. In certain embodiments, levels of a GAG in the serum of the subject are reduced by at least about 30%, 40%, 50%, 60%, 70%, 80%, 90%, 95% or more, as compared to a control. In certain embodiments, the reduction of a GAG in the serum of the subject is relative to the serum levels of the GAG in the subject prior to the administration. In certain embodiments, the administration of the pharmaceutical composition reduces levels of a GAG in the serum of the subject to baseline levels (e.g. levels measured in a healthy subject or a subject that does not have Hunter syndrome).

In certain embodiments, the administration of the pharmaceutical composition reduces levels of a GAG in the urine of the subject, as compared to a control. In certain embodiments, levels of a GAG in the urine of the subject are reduced by at least about 30%, 40%, 50%, 60%, 70%, 80%, 90%, 95% or more, as compared to a control. In certain embodiments, levels of a GAG in the urine of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a GAG in the urine of the subject is relative to the urine levels of the GAG in the subject prior to the administration. For example, in certain embodiments, the administration of the pharmaceutical composition reduces levels of a GAG in the urine of the subject by at least about 50%, 60%, 70%, 75%, 80%, 85%, or 90% after at least 4 weekly doses, wherein the reduction is relative to the urine levels of the GAG in the subject prior to the administration. In certain embodiments, the administration of the pharmaceutical composition reduces levels of the GAG in the urine of the subject by at least about 50%, 60%, 70%, 75%, 80%, 85%, or 90% after 4 weekly doses. In certain embodiments, the administration of the pharmaceutical composition reduces levels of a GAG in the urine of the subject by at least about 50%, 60%, 70%, 75%, 80%, 85%, or 90% after at least 8 weekly doses, wherein the reduction is relative to the urine levels of the GAG in the subject prior to the administration. In certain embodiments, the administration of the pharmaceutical composition reduces levels of the GAG in the urine of the subject by at least about 50%, 60%, 70%, 75%, 80%, 85%, or 90% after 8 weekly doses. In certain embodiments, the administration of the pharmaceutical composition reduces levels of a GAG in the urine of the subject to baseline levels measured in a healthy subject or a subject that does not have Hunter syndrome. In certain embodiments, the reduction of a GAG in the urine of the subject occurs after switching the subject's administered therapy from intravenous idursulfase (e.g. Elaprase) to the pharmaceutical composition comprising ETV:IDS.

In certain embodiments, total GAG levels are reduced. In certain embodiments, heparan sulfate levels are reduced (e.g., D0A0 and/or D0S0 levels are reduced). In certain embodiments, dermatan sulfate levels are reduced (e.g., D0a4 levels are reduced).

Neurofilament light (Nf-L)

As used herein, the term “Nf-L” refers to neurofilament light chain (also referred to as neurofilament light chain polypeptide, neurofilament light polypeptide, and neurofilament light protein) that is encoded by the gene NEFL. As used herein, an “Nf-L protein” refers to a native (i.e., wild-type) Nf-L protein. In some embodiments, an Nf-L protein is a human Nf-L protein having the sequence identified in UniprotKB accession number P07196.

As described herein, increased levels of Nf-L are indicative of downstream pathology in subjects having Hunter syndrome. Accordingly, the concentration of Nf-L may be evaluated in a subject having Hunter syndrome (e.g., in a post-treatment sample from a subject administered ETV:IDS). Nf-L levels can be measured using an assay known in the art or described herein. For example, assays for detecting and measuring levels of protein expression include, e.g., western blot analysis, immunofluorescence, immunohistochemistry (e.g., of tissue arrays), MesoScale Discovery (MSD) method, etc.

In certain embodiments, the administration of the pharmaceutical composition reduces levels of Nf-L in an organ, tissue or fluid of the subject, as compared to a control. In certain embodiments, levels of Nf-L in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of Nf-L in an organ, tissue or fluid of the subject, to baseline levels (e.g. levels measured in a healthy subject or a subject that does not have Hunter syndrome).

In certain embodiments, the administration of the pharmaceutical composition reduces levels of Nf-L in the CNS of the subject, as compared to a control. In certain embodiments, levels of Nf-L in the CNS of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of Nf-L in the CNS of the subject, to baseline levels (e.g. levels measured in a healthy subject or a subject that does not have Hunter syndrome).

In certain embodiments, the administration of the pharmaceutical composition reduces levels of Nf-L in the CSF of the subject, as compared to a control. In certain embodiments, levels of Nf-L in the CSF of the subject are reduced by at least about 10%, 15%, 20%, 25%, 30%, 40%, 50%, 60%, 70%, 80%, 90%, 95% or more, as compared to a control. In certain embodiments, levels of Nf-L in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of Nf-L in the CSF of the subject is relative to the CSF Nf-L level in the subject prior to the administration. In certain embodiments, the administration of the pharmaceutical composition reduces levels of Nf-L in the CSF of the subject to baseline levels (e.g. levels measured in a healthy subject or a subject that does not have Hunter syndrome).

In certain embodiments, the administration of the pharmaceutical composition reduces levels of Nf-L in the serum of the subject, as compared to a control. In certain embodiments, levels of Nf-L in the serum of the subject are reduced by at least about 10%, 15%, 20%, 25%, 30%, 40%, 50%, 60%, 70%, 80%, 90%, 95% or more, as compared to a control. In certain embodiments, levels of Nf-L in the serum of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of Nf-L in the serum of the subject is relative to the serum Nf-L level in the subject prior to the administration. In certain embodiments, the administration of the pharmaceutical composition reduces levels of Nf-L in the serum of the subject to baseline levels (e.g. levels measured in a healthy subject or a subject that does not have Hunter syndrome).

### TREM2

As used herein, the term “TREM2 protein” refers to a triggering receptor expressed on myeloid cells 2 protein that is encoded by the gene Trem2. As used herein, a “TREM2 protein” refers to a native (i.e., wild-type) TREM2 protein. In some embodiments, a TREM2 protein is a human TREM2 protein having the sequence identified in UniprotKB accession number Q9NZC2.

The TREM2 gene encodes a 230 amino acid-length protein that includes an extracellular domain, a transmembrane region and a short cytoplasmic tail (see, UniProtKB Q9NZC2; NCBI Reference Sequence: NP_061838.1). The extracellular region, encoded by exon 2, is composed of a single type V Ig-SF domain, containing three potential N-glycosylation sites. The putative transmembrane region contains a charged lysine residue. The cytoplasmic tail of TREM2 lacks signaling motifs and is thought to signal through the signaling adaptor molecule DAP12/TYROBP and through DAP10. TREM2 is found on the surface of osteoclasts, immature dendritic cells, and macrophages. In the central nervous system, TREM2 is exclusively expressed in microglia.

TREM2 may be cleaved by a disintegrin and metalloproteinase (ADAM) proteases (e.g., ADAM10 and ADAM17), which results in the release of soluble TREM2 (sTREM2) into the extracellular environment. As described herein, increased levels of TREM2 are indicative of downstream pathology (e.g., microglial activation and neuroinflammation) in subjects having Hunter syndrome. Accordingly, the concentration of TREM2/sTREM2 may be evaluated in a subject having Hunter syndrome (e.g., in a post-treatment sample from a subject administered ETV:IDS). TREM2 or sTREM2 levels can be measured using an assay known in the art or described herein. For example, assays for detecting and measuring levels of protein expression include, e.g., western blot analysis, immunofluorescence, immunohistochemistry (e.g., of tissue arrays), MesoScale Discovery (MSD) method, etc.

In certain embodiments, the administration of the pharmaceutical composition reduces levels of TREM2 or sTREM2 in an organ, tissue or fluid of the subject, as compared to a control. In certain embodiments, levels of TREM2 or sTREM2 in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of TREM2 or sTREM2 in an organ, tissue or fluid of the subject, to baseline levels (e.g. levels measured in a healthy subject or a subject that does not have Hunter syndrome).

In certain embodiments, the administration of the pharmaceutical composition reduces levels of TREM2 or sTREM2 in the CSF of the subject, as compared to a control. In certain embodiments, levels of TREM2 or sTREM2 in the CSF of the subject are reduced by at least about 10%, 15%, 20%, 25%, 30%, 40%, 50%, 60%, 70%, 80%, 90%, 95% or more, as compared to a control. In certain embodiments, the reduction of TREM2 or sTREM2 in the CSF of the subject is relative to the CSF TREM2 or sTREM2 level in the subject prior to the administration. In certain embodiments, the administration of the pharmaceutical composition reduces levels of TREM2 or sTREM2 in the CSF of the subject to baseline levels (e.g. levels measured in a healthy subject or a subject that does not have Hunter syndrome).

### Lysosomal Lipids

As described herein, increased levels of BMPs, sphingolipids (e.g., GlcCer) and gangliosides (e.g., GD3, GD1a/b, GM2 and GM3) are indicative of downstream pathology in subjects having Hunter syndrome. Accordingly, the concentration of one or more of these lysosomal lipids may be evaluated in a subject having Hunter syndrome (e.g., in a post-treatment sample from a subject administered ETV:IDS). The concentration of these lipids may be measured using an assay known in the art or described herein (e.g., by mass spectrometry).

In certain embodiments, the administration of the pharmaceutical composition reduces levels of one or more of a BMP, a sphingolipid or a ganglioside in an organ, tissue or fluid of the subject, as compared to a control. In certain embodiments, levels of one or more of a BMP, a sphingolipid or a ganglioside in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of one or more of a BMP, a sphingolipid or a ganglioside in an organ, tissue or fluid of the subject, to baseline levels (e.g. levels measured in a healthy subject or a subject that does not have Hunter syndrome).

BMPs

Bis(monoacylglycero)phosphates (BMPs) refer to a class of an anionic phospholipids. BMPs are enriched in internal membranes of multivesicular endosomes and lysosomes and are thought to play a role in glycosphingolipid degradation and cholesterol transport (see, Kobayashi et al., Nat. Cell Biol. 1 (1999) 113-118). Particular BMP species are described herein, and include but are not limited to, BMP (44:12), BMP (36:2), BMP (di20:4), BMP (di22:6) and BMP (di18:1) (see also, e.g., the Examples and Figures).

In certain embodiments, the administration of the pharmaceutical composition reduces levels of a BMP in an organ, tissue or fluid of the subject, as compared to a control. In certain embodiments, levels of a BMP in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a BMP in an organ, tissue or fluid of the subject, to baseline levels (e.g. levels measured in a healthy subject or a subject that does not have Hunter syndrome).

In certain embodiments, the administration of the pharmaceutical composition reduces levels of a BMP in the CSF of the subject, as compared to a control. In certain embodiments, levels of a BMP in the CSF of the subject are reduced by at least about 10%, 15%, 20%, 25%, 30%, 40%, 50%, 60%, 70%, 80%, 90%, 95% or more, as compared to a control. In certain embodiments, levels of a BMP in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a BMP in the CSF of the subject is relative to the CSF levels of the BMP in the subject prior to the administration. For example, in certain embodiments, the administration of the pharmaceutical composition reduces levels of a BMP in the CSF of the subject by at least about 10%, 15%, 20%, 25%, 30%, 40%, 50%, 60%, 70%, 80%, or 90% after at least 8 weekly doses, wherein the reduction is relative to the CSF levels of the BMP in the subject prior to administration. In certain embodiments, the administration of the pharmaceutical composition reduces levels of the BMP in the CSF of the subject by at least about 10%, 15%, 20%, 25%, 30%, 40%, 50%, 60%, 70%, 80%, or 90% after 8 weekly doses. In certain embodiments, the administration of the pharmaceutical composition reduces levels of a BMP in the CSF of the subject to baseline levels (e.g. levels measured in a healthy subject or a subject that does not have Hunter syndrome). In certain embodiments, the reduction of a BMP in the CSF of the subject occurs after switching the subject's administered therapy from intravenous idursulfase (e.g. Elaprase) to the pharmaceutical composition comprising ETV:IDS. In certain embodiments, the BMP is a BMP di 18:1 species.

Sphingolipids

As described herein, increased levels of certain sphingolipids, such as glucosylceramide (GlcCer), are indicative of downstream pathology in subjects having Hunter syndrome. GlcCer is a glycosphingolipid (ceramide and oligosaccharide) or oligoglycosylceramide with one or more sialic acids linked on the sugar chain. Particular GlcCer species, include but are not limited to, GlcCer (d34:0), GlcCer (d34:1), GlcCer (d36:1), GlcCer (d42:1), GlcCer (d18:1, 16:0), GlcCer (d18:1, 18:0), GlcCer (d18:2, 18:0), GlcCer (d18:1, 20:0), GlcCer (d18:2, 20:0), GlcCer (d18:1, 22:0), GlcCer (d18:1, 22:1), GlcCer (d18:2, 22:0), GlcCer (d18:1, 24:1) or GlcCer (d18:1, 24:0) (see also, e.g., the Examples and Figures).

In certain embodiments, the administration of the pharmaceutical composition reduces levels of a sphingolipid (e.g., a GlcCer) in an organ, tissue or fluid of the subject, as compared to a control. In certain embodiments, levels of a sphingolipid (e.g., a GlcCer) in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a sphingolipid (e.g., a GlcCer) in an organ, tissue or fluid of the subject, to baseline levels (e.g. levels measured in a healthy subject or a subject that does not have Hunter syndrome).

In certain embodiments, the administration of the pharmaceutical composition reduces levels of a sphingolipid (e.g., a GlcCer) in the CSF of the subject, as compared to a control. In certain embodiments, levels of a sphingolipid (e.g., a GlcCer) in the CSF of the subject are reduced by at least about 10%, 15%, 20%, 25%, 30%, 40%, 50%, 60%, 70%, 80%, 90%, 95% or more, as compared to a control. In certain embodiments, levels of a sphingolipid (e.g., a GlcCer) in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a sphingolipid (e.g. a GlcCer) in the CSF of the subject is relative to the CSF sphingolipid level in the subject prior to the administration. In certain embodiments, the administration of the pharmaceutical composition reduces levels of a sphingolipid (e.g., a GlcCer) in the CSF of the subject to baseline levels (e.g. levels measured in a healthy subject or a subject that does not have Hunter syndrome).

Gangliosides

Gangliosides are a type of glycosphingolipid. As described herein, increased levels of certain gangliosides, such as (GD3, GD1a/b, GM2 and GM3), are indicative of downstream pathology in subjects having Hunter syndrome.

In certain embodiments, the administration of the pharmaceutical composition reduces levels of a ganglioside (e.g., a GD3, GD1a/b, GM2 or GM3 species) in an organ, tissue or fluid of the subject, as compared to a control. In certain embodiments, levels of a ganglioside in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a ganglioside in an organ, tissue or fluid of the subject, to baseline levels (e.g. levels measured in a healthy subject or a subject that does not have Hunter syndrome).

In certain embodiments, the administration of the pharmaceutical composition reduces levels of a ganglioside (e.g., a GD3, GD1a/b, GM2 or GM3 species) in the CSF of the subject, as compared to a control. In certain embodiments, levels of a ganglioside in the CSF of the subject are reduced by at least about 10%, 15%, 20%, 25%, 30%, 40%, 50%, 60%, 70%, 80%, 90%, 95% or more, as compared to a control. In certain embodiments, levels of a ganglioside (e.g., a GD3, GD1a/b, GM2 or GM3 species) in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a ganglioside in the CSF of the subject is relative to the CSF levels of the ganglioside in the subject prior to the administration. For example, in certain embodiments, the administration of the pharmaceutical composition reduces levels of a ganglioside in the CSF of the subject by at least about 10%, 15%, 20%, 25%, 30%, 40%, 50%, 60%, 70%, 80%, or 90% after at least 8 weekly doses, wherein the reduction is relative to the CSF levels of the ganglioside in the subject prior to administration. In certain embodiments, the administration of the pharmaceutical composition reduces levels of the ganglioside in the CSF of the subject by at least about 10%, 15%, 20%, 25%, 30%, 40%, 50%, 60%, 70%, 80%, or 90% after 8 weekly doses. In certain embodiments, the administration of the pharmaceutical composition reduces levels of a ganglioside in the CSF of the subject to baseline levels (e.g. levels measured in a healthy subject or a subject that does not have Hunter syndrome). In certain embodiments, the reduction of a ganglioside in the CSF of the subject occurs after switching the subject's administered therapy from intravenous idursulfase (e.g. Elaprase) to the pharmaceutical composition comprising ETV:IDS.

In certain embodiments, the administration of the pharmaceutical composition reduces levels of a GD3 species. Particular GD3 species, include but are not limited to, e.g., GD3 (d34:1), GD3 (d36:1), GD3 (d38:1), GD3 (d39:1), GD3 (d40:1), GD3 (d42:2) or GD3 (d42:1). (see also, e.g., the Examples and Figures).

In certain embodiments, the administration of the pharmaceutical composition reduces levels of a GD3 species in an organ, tissue or fluid of the subject, as compared to a control. In certain embodiments, levels of a GD3 species in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a GD3 species in an organ, tissue or fluid of the subject, to baseline levels (e.g. levels measured in a healthy subject or a subject that does not have Hunter syndrome).

In certain embodiments, the administration of the pharmaceutical composition reduces levels of a GD3 species in the CSF of the subject, as compared to a control. In certain embodiments, levels of a GD3 species in the CSF of the subject are reduced by at least about 10%, 15%, 20%, 25%, 30%, 40%, 50%, 60%, 70%, 80%, 90%, 95% or more, as compared to a control. In certain embodiments, levels of a GD3 species in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a GD3 in the CSF of the subject is relative to the CSF levels of the GD3 in the subject prior to the administration. In certain embodiments, the administration of the pharmaceutical composition reduces levels of a GD3 species in the CSF of the subject to baseline levels (e.g. levels measured in a healthy subject or a subject that does not have Hunter syndrome).

Gangliosides GD1a and GD1b are glycosphingolipids. Particular GD1a/b species are described herein, such as GD1a/b (d36:1) and GD1a/b (d38:1) (see also, e.g., the Examples and Figures).

In certain embodiments, the administration of the pharmaceutical composition reduces levels of a GD1a/b species in an organ, tissue or fluid of the subject, as compared to a control. In certain embodiments, levels of a GD1a/b species in an organ, tissue or fluid of the subject is reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a GD1a/b species in an organ, tissue or fluid of the subject, to baseline levels (e.g. levels measured in a healthy subject or a subject that does not have Hunter syndrome).

In certain embodiments, the administration of the pharmaceutical composition reduces levels of a GD1a/b species in the CSF of the subject, as compared to a control. In certain embodiments, levels of a GD1a/b species in the CSF of the subject is reduced by at least about 10%, 15%, 20%, 25%, 30%, 40%, 50%, 60%, 70%, 80%, 90%, 95% or more, as compared to a control. In certain embodiments, levels of a GD1a/b species in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a GD1a/b in the CSF of the subject is relative to the CSF levels of the GD1a/b in the subject prior to the administration. In certain embodiments, the administration of the pharmaceutical composition reduces levels of a GD1a/b species in the CSF of the subject to baseline levels (e.g. levels measured in a healthy subject or a subject that does not have Hunter syndrome).

Ganglioside Monosialic 2 (GM2) is a glycosphingolipid. Particular GM2 species are described herein, such as GM2 (d38:1) or GM2 (d36:1) (see also, e.g., the Examples and Figures).

In certain embodiments, the administration of the pharmaceutical composition reduces levels of a GM2 species in an organ, tissue or fluid of the subject, as compared to a control. In certain embodiments, levels of a GM2 species in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a GM2 species in an organ, tissue or fluid of the subject, to baseline levels (e.g. levels measured in a healthy subject or a subject that does not have Hunter syndrome).

In certain embodiments, the administration of the pharmaceutical composition reduces levels of a GM2 species in the CSF of the subject, as compared to a control. In certain embodiments, levels of a GM2 species in the CSF of the subject are reduced by at least about 10%, 15%, 20%, 25%, 30%, 40%, 50%, 60%, 70%, 80%, 90%, 95% or more, as compared to a control. In certain embodiments, levels of a GM2 species in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a GM2 in the CSF of the subject is relative to the CSF levels of the GM2 in the subject prior to the administration. In certain embodiments, the administration of the pharmaceutical composition reduces levels of a GM2 species in the CSF of the subject to baseline levels (e.g. levels measured in a healthy subject or a subject that does not have Hunter syndrome).

Similar to GM2, Ganglioside Monosialic 3 (GM3) is also a class of glycosphingolipids. Particular GM3 species, include but are not limited to, (d34:1), GM3 (d36:1), GM3 (d38:1), GM3 (d40:1), GM3 (d41:1), GM3 (d42:2), GM3 (d42:1), GM3 (d43:0), GM3 (d44:1) or GM3 (d44:2) (see also, e.g., the Examples and Figures).

In certain embodiments, the administration of the pharmaceutical composition reduces levels of a GM3 species in an organ, tissue or fluid of the subject, as compared to a control. In certain embodiments, levels of a GM3 species in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a GM3 species in an organ, tissue or fluid of the subject, to baseline levels (e.g. levels measured in a healthy subject or a subject that does not have Hunter syndrome).

In certain embodiments, the administration of the pharmaceutical composition reduces levels of a GM3 species in the CSF of the subject, as compared to a control. In certain embodiments, levels of a GM3 species in the CSF of the subject are reduced by at least about 10%, 15%, 20%, 25%, 30%, 40%, 50%, 60%, 70%, 80%, 90%, 95% or more, as compared to a control. In certain embodiments, levels of a GM3 species in the CSF of the subject is reduced by at least about 30%, 40%, 50%, 60%, 70%, 80%, 90%, 95% or more, as compared to a control, after at least 1, 2, 3, 4, 5, 6, 7, 8, 9, 10, 15, 20 or more weekly doses. In certain embodiments, the reduction of a GM3 in the CSF of the subject is relative to the CSF levels of the GM3 in the subject prior to the administration. For example, in certain embodiments, the administration of the pharmaceutical composition reduces levels of a GM3 species in the CSF of the subject by at least about 10%, 15%, 20%, 25%, 30%, 40%, 50%, 60%, 70%, 80%, or 90% after at least 8 weekly doses, wherein the reduction is relative to the CSF levels of the GM3 species in the subject prior to administration. In certain embodiments, the administration of the pharmaceutical composition reduces levels of the GM3 species in the CSF of the subject by at least about 10%, 15%, 20%, 25%, 30%, 40%, 50%, 60%, 70%, 80%, or 90% after 8 weekly doses. In certain embodiments, the administration of the pharmaceutical composition reduces levels of a GM3 species in the CSF of the subject to baseline levels (e.g. levels measured in a healthy subject or a subject that does not have Hunter syndrome). In certain embodiments, the reduction of a GM3 species in the CSF of the subject occurs after switching the subject's administered therapy from intravenous idursulfase (e.g. Elaprase) to the pharmaceutical composition comprising ETV:IDS.

### Cytokines

As described herein, increased levels of certain cytokines are indicative of neuroinflammation. Accordingly, the concentration of one or more of these cytokines may be evaluated in a subject having Hunter syndrome (e.g., in a post-treatment sample from a subject administered ETV:IDS). For example, cytokines such as interleukin (IL) 1-b, tumor necrosis factor alpha (TNF-α), monocyte chemoattractant protein 1 (MCP-1), stromal cell-derived factor 1 alpha (SDF-1α), IL-Ra, macrophage inflammatory protein 1 beta (MIP-1β), IL-8, and vascular endothelial growth factor (VEGF) may be evaluated. The concentration of these cytokines may be measured using an assay known in the art or described herein.

In certain embodiments, the administration of the pharmaceutical composition reduces levels of a cytokine in an organ, tissue or fluid of the subject, as compared to a control. In certain embodiments, levels of a cytokine in an organ, tissue or fluid of the subject are reduced by at least about 10%, 15%, 20%, 25%, 30%, 40%, 50%, 60%, 70%, 80%, 90%, 95% or more, as compared to a control. In certain embodiments, the administration of the pharmaceutical composition reduces levels of a cytokine in an organ, tissue or fluid of the subject, to baseline levels (e.g. levels measured in a healthy subject or a subject that does not have Hunter syndrome).

In certain embodiments, the administration of the pharmaceutical composition reduces levels of a cytokine in the CSF of the subject, as compared to a control. In certain embodiments, levels of a cytokine in the CSF of the subject are reduced by at least about 10%, 15%, 20%, 25%, 30%, 40%, 50%, 60%, 70%, 80%, 90%, 95% or more, as compared to a control. In certain embodiments, the reduction of a cytokine in the CSF of the subject is relative to the CSF levels of the cytokine in the subject prior to the administration. In certain embodiments, the administration of the pharmaceutical composition reduces levels of a cytokine in the CSF of the subject to baseline levels (e.g. levels measured in a healthy subject or a subject that does not have Hunter syndrome).

In certain embodiments, the administration of the pharmaceutical composition reduces levels of a cytokine in the serum of the subject, as compared to a control. In certain embodiments, levels of a cytokine in the serum of the subject are reduced by at least about 10%, 15%, 20%, 25%, 30%, 40%, 50%, 60%, 70%, 80%, 90%, 95% or more, as compared to a control. In certain embodiments, the reduction of a cytokine in the serum of the subject is relative to the serum levels of the cytokine in the subject prior to the administration. In certain embodiments, the administration of the pharmaceutical composition reduces levels of a cytokine in the serum of the subject to baseline levels (e.g. levels measured in a healthy subject or a subject that does not have Hunter syndrome).

In certain embodiments, the administration of the pharmaceutical composition reduces levels of a cytokine in the urine of the subject, as compared to a control. In certain embodiments, levels of a cytokine in the urine of the subject are reduced by at least about 10%, 15%, 20%, 25%, 30%, 40%, 50%, 60%, 70%, 80%, 90%, 95% or more, as compared to a control. In certain embodiments, the reduction of a cytokine in the urine of the subject is relative to the urine levels of the cytokine in the subject prior to the administration. In certain embodiments, the administration of the pharmaceutical composition reduces levels of a cytokine in the urine of the subject to baseline levels (e.g. levels measured in a healthy subject or a subject that does not have Hunter syndrome).

### Certain Manifestations of Disease

In addition to the correction of certain biomarkers, a therapeutically effective dose of a pharmaceutical composition described herein may also be used for the treatment of particular manifestations of Hunter syndrome, including neurobehavioral deficits, auditory deficits and/or musculoskeletal abnormalities associated with the disease (see, e.g., Example 9).

Accordingly, certain embodiments of the invention provide a method of treating a neurobehavioral deficit, an auditory deficit and/or a musculoskeletal abnormality in a subject with Hunter syndrome, comprising administering to the subject a therapeutically effective dose of a pharmaceutical composition comprising a protein, wherein the protein comprises: (a) a first Fc polypeptide linked to an iduronate 2-sulfatase (IDS) amino acid sequence, an IDS variant amino acid sequence, or a catalytically active fragment thereof; and (b) a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Changes in, or stabilization of, a manifestation of Hunter syndrome (e.g., a neurobehavioral deficit, an auditory deficit, and/or a musculoskeletal abnormality) may also be used to evaluate disease activity and therapeutic response to ETV:IDS. For example, one or more assessments, such as one or more assessments described herein, may be used to evaluate a parameter(s) associated with a given manifestation. In certain embodiments, the parameter is measured relative to a baseline level. In certain embodiments, the baseline level of a parameter is the parameter level for the subject prior to administration of the pharmaceutical composition.

### Neurobehavioral Deficits

Approximately two-thirds of Hunter syndrome patients display the neuronopathic form of the disease, which in addition to earlier presentation of the somatic disease, is characterized by progressive, debilitating neurobehavioral deficits, which include but are not limited to, motor skill deficits, cognitive deficits (e.g., learning and memory deficits) and sensorimotor gating deficits (e.g., attention and inhibitory function deficits).

In certain embodiments, a therapeutically effective dose of a pharmaceutical composition comprising ETV:IDS may be used to treat a neurobehavioral deficit in a subject having Hunter syndrome. In certain embodiments, the administration of the pharmaceutical composition changes the subject's neurobehavioral capabilities relative to a baseline level, e.g., as measured by an assessment described herein. In certain embodiments, the administration of the pharmaceutical composition prevents further deterioration of the subject's neurobehavioral capabilities, relative to a baseline level (e.g., as measured by an assessment described herein).

In certain embodiments, the neurobehavioral deficit is a motor skill deficit, such as a gross and/or fine motor skill deficit. Thus, in certain embodiments, a therapeutically effective dose of a pharmaceutical composition comprising ETV:IDS may be used to treat a gross and/or fine motor deficit in a subject having Hunter syndrome. In certain embodiments, the administration of the pharmaceutical composition changes the subject's gross and/or fine motor capabilities relative to a baseline level, e.g., as measured by an assessment described herein. In certain embodiments, the administration of the pharmaceutical composition prevents further deterioration of the subject's gross and/or fine motor capabilities, relative to a baseline level (e.g., as measured by an assessment described herein).

In certain embodiments, the neurobehavioral deficit is an agility deficit. Thus, in certain embodiments, a therapeutically effective dose of a pharmaceutical composition comprising ETV:IDS may be used to treat an agility deficit in a subject having Hunter syndrome. In certain embodiments, the administration of the pharmaceutical composition changes the subject's agility capabilities relative to a baseline level, e.g., as measured by an assessment described herein. In certain embodiments, the administration of the pharmaceutical composition prevents further deterioration of the subject's agility capabilities, relative to a baseline level (e.g., as measured by an assessment described herein).

In certain embodiments, the neurobehavioral deficit is a cognitive deficit, such as a learning deficit (e.g., spatial learning deficit) or a memory deficit. Thus, in certain embodiments, a therapeutically effective dose of a pharmaceutical composition comprising ETV:IDS may be used to treat a cognitive deficit in a subject having Hunter syndrome. In certain embodiments, the administration of the pharmaceutical composition changes the subject's cognitive capabilities relative to a baseline level, e.g., as measured by an assessment described herein. In certain embodiments, the administration of the pharmaceutical composition prevents further deterioration of the subject's cognitive capabilities, relative to a baseline level (e.g., as measured by an assessment described herein).

In certain embodiments, the neurobehavioral deficit is a sensorimotor gating deficit, which is associated with both attention and inhibitory function in a subject. Thus, in certain embodiments, a therapeutically effective dose of a pharmaceutical composition comprising ETV:IDS may be used to treat a sensorimotor gating deficit in a subject having Hunter syndrome. In certain embodiments, the administration of the pharmaceutical composition changes the subject's sensorimotor gating capabilities relative to a baseline level, e.g., as measured by an assessment described herein. In certain embodiments, the administration of the pharmaceutical composition prevents further deterioration of the subject's sensorimotor gating capabilities, relative to a baseline level (e.g., as measured by an assessment described herein).

### Auditory Deficits

Progressive conductive and sensorineural hearing loss, resulting from GAG accumulation in the middle ear and recurrent otitis, are common in Hunter syndrome patients.

In certain embodiments, a therapeutically effective dose of a pharmaceutical composition comprising ETV:IDS may be used to treat an auditory deficit, such as conductive or sensorineural hearing loss, in a subject having Hunter syndrome. In certain embodiments, the administration of the pharmaceutical composition changes the subject's auditory capabilities relative to a baseline level, e.g., as measured by an assessment described herein. In certain embodiments, the administration of the pharmaceutical composition prevents further deterioration of the subject's auditory capabilities, relative to a baseline level (e.g., as measured by an assessment described herein).

In certain embodiments, a therapeutically effective dose of a pharmaceutical composition comprising ETV:IDS may be used to reduce middle ear effusion and/or otitis media in a subject having Hunter syndrome. In certain embodiments, the administration of the pharmaceutical composition changes the amount of middle ear effusion and/or otitis media in a subject relative to a baseline level, e.g., as measured by an assessment described herein.

In certain embodiments, a therapeutically effective dose of a pharmaceutical composition comprising ETV:IDS may be used to treat an auricular abnormality in a subject having Hunter syndrome. In certain embodiments, the administration of the pharmaceutical composition changes the subject's auricular features relative to a baseline level, e.g., as measured by an assessment described herein. In certain embodiments, the administration of the pharmaceutical composition prevents further deterioration of the subject's auricular features, relative to a baseline level (e.g., as measured by an assessment described herein).

### Musculoskeletal Abnormalities

A series of musculoskeletal abnormalities are commonly found in Hunter syndrome patients, including disproportional short stature, joint stiffness/contractures, thoracolumbar kyphosis, hip dysplasia, and dysostosis multiplex.

In certain embodiments, a therapeutically effective dose of a pharmaceutical composition comprising ETV:IDS may be used to treat a musculoskeletal abnormality (e.g., a skeletal abnormality) in a subject having Hunter syndrome. In certain embodiments, the administration of the pharmaceutical composition changes a musculoskeletal feature(s) in the subject relative to a baseline level, e.g., as measured by an assessment described herein. In certain embodiments, the administration of the pharmaceutical composition prevents further deterioration of the subject's musculoskeletal abnormality, relative to a baseline level (e.g., as measured by an assessment described herein).

### Neurocognitive Assessments

Disease activity and therapeutic response to ETV:IDS may be evaluated using one or more neurocognitive assessments, such as the Vineland Adaptive Behavior Scales, Second Edition (Vineland-II), the Bayley Scales of Infant and Toddler Development, Third Edition (BSID-III), and the Kaufman Assessment Battery for Children, Second Edition (KABC-II).

In certain embodiments, changes in a neurocognitive assessment are relative to a baseline (e.g., from a subject who has not undergone treatment; from a subject prior to treatment; or from an age-matched control subject that does not have Hunter syndrome). In certain embodiments, the baseline is from the subject prior to treatment and the assessment is performed, e.g., at 2 months, 6 months, 12 months and/or 18 months after treatment initiation.

The Vineland-II is a standardized assessment tool used to aid in diagnosing and classifying an individual's intellectual and developmental disabilities and other disorders (Sparrow et al., Vineland adaptive behavior scales: interview edition, survey form manual. Circle Pines, Minn.: American Guidance Service; 1984; Sparrow et al., Vineland adaptive behavior scales: second edition (Vineland II), survey interview form/caregiver rating form. Livonia, Minn.: Pearson Assessments; 2005). The content and scales of Vineland-II are organized within a 3-domain structure: Communication, Daily Living, and Socialization. In addition, Vineland-II offers a Motor Skills domain. Within this assessment, the caregiver-reported scale may be used to measure behaviors in the domains associated with typical development, such as communication and daily living, socialization, and motor skills.

In certain embodiments, the administration of the pharmaceutical composition changes the subject's intellectual and/or developmental disabilities/disorders relative to a baseline level, as measured by Vineland-II. In certain embodiments, the administration of the pharmaceutical composition prevents the subject's intellectual and/or developmental disabilities/disorders, as measured by Vineland-II, from deteriorating relative to a baseline level.

The BSID-III is a standard series of measurements used to assess the development of infants and toddlers aged 1 to 42 months (Bayley, Bayley scales of infant and toddler development: administration manual. San Antonio, Tex.: Harcourt Assessment; 2006). Measurements may include 3 main subtests: the Cognitive Scale, the Language Scale, and the Motor Scale. Raw age-normed and age-equivalent scores may be used to calculate a DQ. In certain embodiments, the BSID-III is performed for patients who have a developmental age of 42 months or younger based on the Vineland-II.

In certain embodiments, the administration of the pharmaceutical composition changes the subject's DQ, as measured by BSID-III, relative to a baseline level. In certain embodiments, the administration of the pharmaceutical composition prevents the subject's DQ, as measured by BSID-III, from deteriorating relative to a baseline level.

The KABC-II is an individually administered measure of the cognitive processing abilities of children and adolescents aged 3 through 18 years. Raw age-normed and age-equivalent scores (e.g., nonverbal ability score) may be used to calculate a DQ. In certain embodiments, the KABC-II is performed for patients who have a developmental age of >42 months based on the Vineland-II.

In certain embodiments, the administration of the pharmaceutical composition changes the subject's DQ, as measured by KABC-II, relative to a baseline level. In certain embodiments, the administration of the pharmaceutical composition prevents the subject's DQ, as measured by KABC-II, from deteriorating relative to a baseline level.

### Clinical Assessments

Disease activity and therapeutic response to ETV:IDS may also be evaluated using patient diaries and/or one or more clinical assessments described herein. Additionally, as described below, these tools may also be used to evaluate the safety and tolerability of the therapeutic (e.g., to identify adverse events (AEs) associated with ETV:IDS administration). In certain embodiments, the baseline is from, e.g., the subject prior to treatment, from a subject who has not undergone treatment; or from an age-matched control subject that does not have Hunter syndrome. In certain embodiments, the assessment is performed at, e.g., 2 weeks, 3 weeks, 1 month, 2 months, 4 months, 6 months, 12 months, 18 months, and/or 24 months or longer after treatment initiation.

### Medical Histories and Patient Diaries

Medical histories may be reviewed to identify and collect information about the subject's presentation, diagnosis, comorbidities, disease-related medical events and symptoms, and medications used to manage symptoms. For example, medical histories may include but are not limited to histories of acute, chronic, and infectious disease; surgical and oncological histories; reproductive status; any conditions affecting major body systems; any cognitive or behavioral assessments or interventions; and demographic data, including year of birth, sex, race, and ethnicity. This information may be considered in conjunction with a patient diary.

### Physical Examinations

Physical examinations, including age-appropriate neurologic examinations, may also be performed to evaluate disease activity and therapeutic response to ETV:IDS, as well as safety/tolerability to the composition. For example, symptom-oriented physical/neurological examination may be performed (e.g., to assess patient symptoms or safety laboratory abnormalities). Physical examinations may also include measurement of vital signs (e.g., blood pressure, pulse rate, respiratory rate and body temperature) body weight and height, and liver and spleen volume.

In certain embodiments, the physical and/or neurologic examination does not identify new symptoms post treatment in the subject, as compared to pre-treatment symptoms in the subject. In certain embodiments, the physical and/or neurologic examination identifies a reduced number of symptoms post treatment in the subject, as compared to pre-treatment symptoms in the subject.

In certain embodiments, the administration of the pharmaceutical composition does not change the subject's vital signs, relative to a baseline level. In certain embodiments, the administration of the pharmaceutical composition improves the subject's vital signs, relative to a baseline level. In certain embodiments, the subject's post-treatment vital signs are within a normal range (e.g., relative to a healthy subject that does not have Hunter syndrome).

In certain embodiments, the administration of the pharmaceutical composition changes the subject's liver volume relative to a baseline level, as assessed by MRI or ultrasound. In certain embodiments, the administration of the pharmaceutical composition reduces the subject's liver volume by at least about 10%, 15%, 20%, 25%, 30%, 40%, 50%, 60%, 70%, 80%, 90%, 95% or more, relative to a baseline level, as assessed by MRI or ultrasound.

In certain embodiments, the administration of the pharmaceutical composition changes the subject's spleen volume relative to a baseline level, as assessed by MRI or ultrasound. In certain embodiments, the administration of the pharmaceutical composition reduces the subject's spleen volume by at least about 10%, 15%, 20%, 25%, 30%, 40%, 50%, 60%, 70%, 80%, 90%, 95% or more, relative to a baseline level, as assessed by MRI or ultrasound.

### Laboratory Tests

Standard laboratory tests (chemistry, hematology, and urinalysis) may be performed at baseline and/or at varying time-points post-treatment initiation. For example, a coagulation panel; a complete blood count (for platelet count); CSF cell counts, protein, and glucose from CSF samples; and urinalysis may be performed.

In certain embodiments, the administration of the pharmaceutical composition does not change the subject's results for one or more of these standard tests, relative to a baseline level. In certain embodiments, the subject's post-treatment results for one or more of these standard tests are within a normal range (e.g., relative to a healthy subject that does not have Hunter syndrome).

### Functional Assessments

Disease activity and therapeutic response to ETV:IDS may also be evaluated using one or more functional assessments described herein. For example, Individualized Educational Plans (IEPs) and/or Individualized Family Service Plan (IFSPS) may be performed at baseline and/or at varying time-points post-treatment initiation. An audiology assessment may also be performed at baseline and/or at varying time-points post-treatment (e.g., an auditory brainstem response (ABR) assessment or a standard hearing test). Additionally, a subject's toileting abilities percentage (TAP) may also be assessed at baseline and/or at varying time-points post-treatment. In certain embodiments, the baseline is from, e.g., the subject prior to treatment, from a subject who has not undergone treatment; or from an age-matched control subject that does not have Hunter syndrome. In certain embodiments, the assessment is performed, e.g., at 2 months, 6 months, 12 months and/or 18 months after treatment initiation.

In certain embodiments, the administration of the pharmaceutical composition improves or maintains the subject's qualitative IEP trajectories in cognitive categories or behavioral categories relative to a baseline level. In certain embodiments, the administration of the pharmaceutical composition improves or maintains the subject's qualitative IFSPS trajectories in cognitive categories or behavioral categories relative to a baseline level.

In certain embodiments, the subject's qualitative IEP trajectories in cognitive categories or behavioral categories relative to a baseline level do not significantly worsen post-treatment with the pharmaceutical composition. In certain embodiments, the administration of the pharmaceutical composition does not significantly worsen the subject's qualitative IFSPS trajectories in cognitive categories or behavioral categories relative to a baseline level do not significantly worsen post-treatment with the pharmaceutical composition.

In certain embodiments, the administration of the pharmaceutical composition improves or maintains the subject's toileting abilities percentage (TAP) relative to a baseline level. In certain embodiments, the subject's TAP relative to a baseline level does not significantly worsen post-treatment with the pharmaceutical composition.

In certain embodiments, the administration of the pharmaceutical composition improves or maintains the subject's hearing relative to a baseline level, as assessed by ABR or a standard hearing test. In certain embodiments, the subject's hearing relative to a baseline level does not significantly worsen post-treatment with the pharmaceutical composition, as assessed by ABR or a standard hearing test.

### Quality of Life Assessments

Disease activity and therapeutic response to ETV:IDS may also be evaluated using one or more quality of life assessments described herein. For example, the Child Health Questionnaire Parent Form 28 (CHQ-PF28), the Infant and Toddler Quality of Life™ (ITQOL), and/or the Pediatric Quality of Life Inventory™ Family Impact Module (PedsQL™-FIM) assessments may be performed at baseline and/or at varying time-points post-treatment initiation. In certain embodiments, the baseline is from, e.g., the subject prior to treatment, from a subject who has not undergone treatment; or from an age-matched control subject that does not have Hunter syndrome. In certain embodiments, the assessment is performed, e.g., at 2 months, 6 months, 12 months and/or 18 months after treatment initiation.

The CHQ-PF28 is a generic person-reported outcome measurement that may be used to assess health-related quality of life for children and adolescents from 5 through 18 years of age. The ITQOL is a generic person-reported outcome measurement to assess health-related quality of life for children aged 2 months to 5 years (Raat et al. Reliability and validity of the Infant and Toddler Quality of Life Questionnaire (ITQOL) in a general population and respiratory disease sample. Qual Life Res. 2007; 16(3):445-60). This instrument is an extension of the CHQ-PF28 for younger patients. The ITQOL may be used for patients under age 5 years. The PedsQL-FIM is a multidimensional instrument that was designed to measure the impact of pediatric chronic health conditions on parents and the family. The PedsQL-FIM measures parent self-reported physical, emotional, social, and cognitive functioning, communication, and worry. The module also measures parent-reported family daily activities and family relationships.

In certain embodiments, the administration of the pharmaceutical composition improves or maintains the subject's quality of life, e.g., as assessed by CHQ-PF28, ITQOL and/or PedsQL-FIM, relative to a baseline level. In certain embodiments, the subject's quality of life, e.g., as assessed by CHQ-PF28, ITQOL and/or PedsQL-FIM, relative to a baseline level, does not significantly worsen post-treatment with the pharmaceutical composition.

### Safety and Tolerability Assessments

The safety and tolerability of ETV:IDS at a given dosage may be evaluated using certain assessments described herein. For example, certain biomarker and clinical assessments may be used to evaluate subjects that have been administered a pharmaceutical composition comprising ETV:IDS. Additionally, the frequency and severity of adverse events (AEs) may also be monitored.

For example, in certain embodiments, a subject administered the pharmaceutical composition may be evaluated using a combination of clinical assessments, including but not limited to, vital sign measurement, physical examination, including neurological examination, diagnostic testing, laboratory assessments (e.g., hematology, serum clinical chemistry, urinalysis, and coagulation), and characterization of immunogenicity of the protein in serum (measured by the incidence of anti-drug antibodies (ADAs) relative to baseline). Additionally, the total GAG concentration (normalized to creatinine) in urine may also be monitored as a safety parameter.

In certain embodiments the pharmacokinetics (PK) of the ETV:IDS are evaluated. For example, PK parameters may include, but are not be limited to, the following: Cmax; Trough concentration (Cmin); Tmax; Area under the concentration-time curve from time zero to the time of last quantifiable concentration (_{AUC0-last}); Area under the concentration-time curve over a dosing interval (AUC_{0-T}); Apparent terminal elimination rate constant (λz); Apparent terminal elimination t½; and Accumulation ratio.

In certain embodiments, the urine total GAG concentration (normalized to creatinine) does not increase relative to a baseline level after administration of the pharmaceutical composition (e.g., the urine total GAG concentration does not increase relative to pre-treatment levels in the subject). In certain embodiments, the urine total GAG concentration (normalized to creatinine) increases by less than about 20%, 19%, 18%, 17%, 16%, 15%, 14%, 13%, 12%, 11%, 10%, 9%, 8%, 7%, 6%, 5%, 4%, 3%, 2%, or 1% relative to a baseline level after administration of the pharmaceutical composition. In certain embodiments, the urine total GAG concentration (normalized to creatinine) decreases relative to a baseline level after administration of the pharmaceutical composition. In certain embodiments, the stabilization or decrease in urine total GAG concentration in the subject supports safety of the administered dose.

In certain embodiments, prior to administration of the pharmaceutical composition, the subject had received recombinant idursulfase enzyme replacement therapy. In certain embodiments, the subject has pre-existing ADAs against IDS prior to administration of the pharmaceutical composition. In certain embodiments, the subject's pre-existing titer of anti-drug antibodies against IDS is greater than 100, 150, 200, 300, 400, 500, 1,000, 5,000, 25,000, 50,000, 75,000, 100,000, 500,000, 1,000,000, 10,000,000 or more. In certain embodiments, the subject's pre-existing titer of anti-drug antibodies against IDS ranges from 189 to greater than 11 million. In certain embodiments, the subject's pre-existing titer of anti-drug antibodies against IDS is greater than 11 million.

In certain embodiments, the incidence of ADAs does not increase relative to a baseline level after administration of the pharmaceutical composition (e.g., the incidence of ADAs does not increase relative to pre-treatment levels in the subject). In certain embodiments, the incidence of ADAs increases by less than about 10%, 9%, 8%, 7%, 6%, 5%, 4%, 3%, 2%, or 1% relative to a baseline level after administration of the pharmaceutical composition. In certain embodiments, the formation of new ADAs or increase in existing ADAs relative to a baseline level does not significantly diminish the efficacy of treatment with the pharmaceutical composition.

As used herein, an adverse event refers to any untoward medical occurrence associated the use of the pharmaceutical composition, and includes, e.g., any unfavorable and unintended signs, symptoms or disease; any new disease or exacerbation of an existing disease; recurrence of an intermittent medical condition not present at baseline; and any deterioration in a laboratory value or other clinical test that is associated with symptoms or leads to a change in concomitant treatment. For example, AEs include infusion related reactions (IRRs), such as allergic reactions and anaphylaxis.

As used herein, a “serious adverse event (SAE)” is any AE that meets any of the following criteria: is fatal; is life-threatening; requires or prolongs patient hospitalization; results in persistent or significant disability/incapacity (i.e., the AE results in substantial disruption of the patient's ability to conduct normal life functions); or is a significant medical event (e.g., may jeopardize the patient or may require medical/surgical intervention to prevent one or more outcomes listed above).

AEs may also be categorized by severity (i.e., the intensity of the AE). For example, an AE may be considered: mild (i.e., easily tolerated, causes minimal discomfort, and does not interfere with everyday activities); moderate (i.e., causes sufficient discomfort to interfere with normal everyday activities; or severe (i.e., medically significant event that causes marked limitation or inability to perform daily activities).

In certain embodiments, a subject administered the pharmaceutical composition does not experience an adverse event (e.g., an AE associated with the administration or the composition). In certain embodiments, adverse events (AEs) in subjects administered the pharmaceutical composition are uncommon. In certain embodiments, AEs in subjects administered the pharmaceutical composition are rare or very rare. For example, in certain embodiments, the frequency of AEs in subjects administered the pharmaceutical composition are less than about 1/100, or less than about 1/1,000, or less than about 1/10,000.

In certain embodiments, a subject administered the pharmaceutical composition does not experience a serious adverse event (SAE), such as an untoward medical occurrence that, e.g., results in death, is life threatening, requires inpatient hospitalization or prolongation of existing hospitalization, or results in significant disability or incapacity. In certain embodiments, serious adverse events (SAEs) in subjects administered the pharmaceutical composition are uncommon. In certain embodiments, serious adverse events (SAEs) in subjects administered the pharmaceutical composition are rare or very rare. For example, in certain embodiments, the frequency of SAEs in subjects administered the pharmaceutical composition are less than about 1/100, or less than about 1/1,000, or less than about 1/10,000.

In certain embodiments, a subject administered the pharmaceutical composition does not experience a severe AE. In certain embodiments, severe AEs in subjects administered the pharmaceutical composition are uncommon. In certain embodiments, severe AEs in subjects administered the pharmaceutical composition are rare or very rare. For example, in certain embodiments, the frequency of severe AEs in subjects administered the pharmaceutical composition are less than about 1/100, or less than about 1/1,000, or less than about 1/10,000.

In certain embodiments, a subject does not experience an IRR, such as an allergic reaction or anaphylaxis, upon administration of the pharmaceutical composition. In certain embodiments, the subject does not experience an allergic reaction. In certain embodiments, the subject does not experience anaphylaxis. In certain embodiments, IRRs in subjects administered the pharmaceutical composition are uncommon. In certain embodiments, IRRs in subjects administered the pharmaceutical composition are rare or very rare. For example, in certain embodiments, the frequency of IRRs in subjects administered the pharmaceutical composition are less than about 1/100, or less than about 1/1,000, or less than about 1/10,000.

In certain embodiments, the pharmaceutical composition is administered to the subject without pretreatment or co-administration of medication for an infusion-related reaction (IRR). Such medications include, but are not limited to, an anti-histamine, an anti-pyretic, and a corticosteroid.

In certain embodiments, a subject experiences an IRR upon administration of the pharmaceutical composition. In certain embodiments, the pharmaceutical composition is administered to the subject with pretreatment or co-administration of medication for an infusion-related reaction (IRR). In certain embodiments, a medication for an IRR is administered to the subject post-treatment of the pharmaceutical composition.

Certain embodiments also provide a method of resolving an infusion-related reaction (IRR) in a subject receiving treatment for Hunter syndrome, comprising administering one or more agents useful for treating an IRR (e.g., selected from the group consisting of an anti-histamine, an anti-pyretic, and a corticosteroid), wherein the subject is being administered or was administered a therapeutically effective dose of the pharmaceutical composition (e.g., at least about 7.5 mg/kg). In certain embodiments, the IRR is a fever. In certain embodiments, the method of resolving the IRR comprises administering to the subject acetaminophen and diphenhydramine.

### Subjects

The term “subject,” “individual,” and “patient,” as used interchangeably herein, refers to a mammal, including but not limited to humans, non-human primates, rodents (e.g., rats, mice, and guinea pigs), rabbits, cows, pigs, horses, and other mammalian species. In certain embodiments described herein, the subject is a human subject.

In certain embodiments, the subject is a male subject.

In certain embodiments, the subject is from about 1 month to 30 years of age. In certain embodiments, the subject is from about 6 months to 30 years of age. In certain embodiments, the subject is from about 1 to 30 years of age. In certain embodiments, the subject is from about 1 to 25 years of age. In certain embodiments, the subject is from about 1 to 18 years of age. In certain embodiments, the subject is from about 2 to 18 years of age. In certain embodiments, the subject is from about 2 to 15 years of age. In certain embodiments, the subject is from about 2 to years of age. In certain embodiments, the subject is from about 5 to 10 years of age. In certain embodiments, the subject is less than 4 years of age. In certain embodiments, the subject is less than 2 years of age. In certain embodiments, the subject is less than 1 year of age. In certain embodiments, the subject is more than 1 year of age. In certain embodiments, the subject is about 1, 2, 3, 4, 5, 6, 7, 8, 9, 10, 11, 12, 13, 14, 15, 16, 17 or 18 years of age.

In certain embodiments, the subject has a weight of ≥5 kg. In certain embodiments, the subject has a weight of ≥9 kg. In certain embodiments, the subject has a weight of ≥15 kg. In certain embodiments, the subject has a weight of ≥19 kg. In certain embodiments, the subject has a weight of ≥23 kg. In certain embodiments, the subject has a weight of ≥27 kg. In certain embodiments, the subject has a weight of ≥32 kg. In certain embodiments, the subject has a weight of ≥36 kg. In certain embodiments, the subject has a weight of ≥41 kg. In certain embodiments, the subject has a weight of ≥45 kg. In certain embodiments, the subject has a weight of ≥50 kg.

In certain embodiments, the subject has neuronopathic Hunter syndrome, also referred to as neuronopathic MPSII (nMPSII). In certain embodiments, the subject has non-neuronopathic Hunter syndrome. In certain embodiments, the subject has Hunter syndrome with an unknown neuronopathic phenotype.

In certain embodiments, prior to administration of the pharmaceutical composition, the subject had received recombinant idursulfase enzyme replacement therapy (e.g., for more than 4 months, more than 6 months, more than 1 year, more than 18 months, more than 2 years, or longer).

In certain embodiments, the subject has a neurobehavioral deficit. In certain embodiments, the subject has a motor skills deficit. In certain embodiments, the subject has a cognitive deficit. In certain embodiments, the subject has a sensorimotor deficit.

In certain embodiments, the subject has impaired or delayed development (e.g., mental or physical development). In certain embodiments, the subject has a development quotient (DQ)<85. In certain embodiments, the subject has a DQ<80. In certain embodiments, the subject has a DQ<75. In certain embodiments, the subject has a DQ<70. In certain embodiments, the subject has a DQ<65. In certain embodiments, the subject has a DQ<60. In certain embodiments, the subject has a DQ<65. In certain embodiments, the subject has a DQ<60. In certain embodiments, the subject has a DQ<55. In certain embodiments, the subject has a DQ<50.

In certain embodiments, the subject has a decline of at least 5 points in DQ within a period of about 6 months or longer. In certain embodiments, the subject has a decline of at least 7.5 points in DQ within a period of about 6 months or longer. In certain embodiments, the subject has a decline of at least 10 points in DQ within a period of about 6 months or longer. In certain embodiments, the subject has a decline of at least 15 points in DQ within a period of about 6 months or longer. In certain embodiments, the subject has a decline of at least 20 points in DQ within a period of about 6 months or longer. In certain embodiments, the subject has a decline of at least 25 points in DQ within a period of about 6 months or longer. In certain embodiments, DQ declines within a period of about 9, 12, 18, or 24 mo or longer.

In certain embodiments, the subject has an auditory deficit.

In certain embodiments, the subject has a musculoskeletal abnormality.

In certain embodiments, the subject has a documented mutation in the IDS gene. In certain embodiments, the subject has been diagnosed as having reduced IDS enzyme activity. In certain embodiments, subject has been diagnosed with Hunter syndrome based on reduced IDS enzyme activity and a documented mutation in the IDS gene. In certain embodiments, the subject has a same genetic mutation in the IDS gene as a blood relative with confirmed neuronopathic Hunter Syndrome/nMPS II. In certain embodiments, the subject has neuronopathic Hunter syndrome/nMPS II or has a same genetic mutation in the IDS gene as a blood relative with confirmed neuronopathic Hunter Syndrome/nMPS II.

In certain embodiments, a subject administered the pharmaceutical composition, or a sample therefrom, is evaluated using an assessment described herein. In certain embodiments, parameters or certain assessment results are compared to a control or baseline levels. Depending on the type of parameter being evaluated or the assessment being performed, the control or baseline level may vary. For example, in certain embodiments, the term “control” may refer to a heathy subject or a subject that does not have Hunter syndrome (or a sample therefrom). Alternatively, the term “control” may refer to a Hunter syndrome patient that was not administered the pharmaceutical composition or to the subject prior to treatment (or a sample therefrom). Similarly, a “baseline level” may refer to a level or a range of levels that is measured in, e.g., a healthy individual or in a subject that does not have Hunter syndrome. In certain other embodiments described herein, a “baseline level” may also refer to a level or a range of levels in a Hunter syndrome patient that was not administered the pharmaceutical composition or in the subject prior to administration of the pharmaceutical composition.

In certain embodiments, a control value or baseline level may be established using data from a population of control subjects. In some embodiments, the population of subjects is matched to a test subject according to one or more patient characteristics such as age, sex, ethnicity, or other criteria. In some embodiments, the control value is established using the same type of sample from the population of subjects (e.g., a sample comprising blood or PBMCs) as is used for assessing the level of lipids/proteins in the test subject.

It is within the skill of the art to identify the appropriate control or baseline level depending on the parameter being evaluated.

### Administration and Therapeutically Effective Doses

A pharmaceutical composition described herein may be administered to a subject at a therapeutically effective amount or dose, e.g., a safe and therapeutically effect amount or dose.

In certain embodiments, a therapeutically effective dose comprises an amount of the protein that is activity equivalent to a standard of care dose for recombinant idursulfase. In certain embodiments, a therapeutically effective dose comprises an amount of the protein that is activity equivalent to a standard of care dose for recombinant idursulfase beta. In certain embodiments, the therapeutically effective dose is from about 1 mg/kg to about 40 mg/kg of protein. In certain embodiments, the therapeutically effective dose is from about 1 mg/kg to about 30 mg/kg of protein. In certain embodiments, the therapeutically effective dose is about 1 mg/kg of protein. In certain embodiments, the therapeutically effective dose is about 3 mg/kg of protein. In certain embodiments, the therapeutically effective dose is about 7.5 mg/kg of protein. In certain embodiments, the therapeutically effective dose is about 10 mg/kg of protein. In certain embodiments, the therapeutically effective dose is about 15 mg/kg of protein. In certain embodiments, the therapeutically effective dose is about 20 mg/kg of protein. In certain embodiments, the therapeutically effective dose is about 30 mg/kg of protein. In certain embodiments, the therapeutically effective dose is about 40 mg/kg of protein.

In certain embodiments, such a dose described herein is a safe and therapeutically effective dose (e.g., as determined using an assessment described herein, such as stabilization or decrease in urine total GAG concentration in the subject).

In certain embodiments, the pharmaceutical composition is administered weekly.

In some embodiments, a protein molecule described herein has an enzymatic activity of at least about 500 units (U)/mg, about 1,000 U/mg, or at least about 1,500, 2,000, 2,500, 3,000, 3,500, 4,000, 4,500, 5,000, 6,000, 7,000, 8,000, 9,000, or 10,000 U/mg. In some embodiments, the enzymatic activity is at least about 11,000 U/mg, or at least about 12,000, 13,000, 14,000, 15,000, 16,000, 17,000, 18,000, 19,000, 20,000, 25,000, 30,000, 35,000, 40,000, 45000, or 50,000 U/mg; or anywhere in a range of about 500 U/mg to about 50,000 U/mg.

Dosages may be varied according to several factors, including the chosen route of administration, the formulation of the composition, patient response, the severity of the condition, the subject's weight, the subject's age, the subject's head size and/or ratio of head size to height, and the judgment of the prescribing physician. The dosage can be increased or decreased over time, as required by an individual patient. In some embodiments, a patient initially is given a low dose, which is then increased to an efficacious dosage tolerable to the patient. Determination of an effective amount is well within the capability of those skilled in the art.

In various embodiments, a pharmaceutical composition described herein is administered parenterally. In some embodiments, the pharmaceutical composition is administered intravenously. Intravenous administration can be by infusion, e.g., over a period of from about to about 30 minutes, or over a period of at least 1 hour, 2 hours, 3 hours, 4 hours, 6 hours, 8 hours, or 10 hours. In some embodiments, the pharmaceutical composition is administered intravenously over a period of from about 20 minutes to 6 hours, or from about 30 minutes to 4 hours. In some embodiments, the pharmaceutical composition is administered as an intravenous bolus. Combinations of infusion and bolus administration may also be used.

### ETV:IDS Protein Molecules

Certain methods described herein provide a method of treating Hunter syndrome in a subject in need thereof, comprising administering to the subject a therapeutically effective dose of a pharmaceutical composition comprising a fusion protein described herein. These fusion proteins may be referenced herein as an enzyme transport vehicle (ETV) in conjunction with the an iduronate 2-sulfatase (IDS) enzyme, or ETV:IDS. ETV:IDS proteins, which may be used in a method described herein, are discussed below and are described in WO 2019/070577, which is incorporated by reference herein for all purposes.

### Certain Fusion Proteins Comprising IDS Enzymes

Described below are certain embodiments of fusion proteins that include an IDS enzyme, or a catalytically active variant or fragment of a wild-type IDS (e.g., a wild-type human IDS), linked to an Fc polypeptide; these fusion proteins may be included in a pharmaceutical composition described herein for the treatment of Hunter syndrome. In some cases, the protein includes a dimeric Fc polypeptide, where one of the Fc polypeptide monomers is linked to the IDS enzyme. The Fc polypeptides can increase enzyme half-life and, in some cases, can be modified to confer additional functional properties onto the protein. Also described herein are fusion proteins that facilitate delivery of an IDS enzyme across the blood-brain barrier (BBB). These proteins comprise an Fc polypeptide and a modified Fc polypeptide that form a dimer, and an IDS enzyme linked to the Fc region and/or the modified Fc region. The modified Fc region can specifically bind to a BBB receptor such as a transferrin receptor (TfR).

In some aspects, a fusion protein described herein comprises: (i) an Fc polypeptide, which may contain modifications (e.g., one or more modifications that promote heterodimerization) or may be a wild-type Fc polypeptide; and an IDS enzyme; and (ii) an Fc polypeptide, which may contain modifications (e.g., one or more modifications that promote heterodimerization) or may be a wild-type Fc polypeptide; and optionally an IDS enzyme. In some embodiments, one or both Fc polypeptides may contain modifications that result in binding to a blood-brain barrier (BBB) receptor, e.g., a TfR. An IDS enzyme incorporated into the fusion protein is catalytically active, i.e., it retains the enzymatic activity that is deficient in Hunter syndrome.

In some embodiments, a fusion protein comprising an IDS enzyme and optionally a modified Fc polypeptide that binds to a BBB receptor, e.g., a TfR-binding Fc polypeptide, comprises a catalytically active fragment or variant of a wild-type IDS. In some embodiments, the IDS enzyme is a variant or a catalytically active fragment of an IDS protein that comprises the amino acid sequence of any one of SEQ ID NOS: 69, 70, 90, 170, and 174. In some embodiments, a catalytically active variant or fragment of an IDS enzyme has at least 50%, at least 55%, at least 60%, at least 65%, at least 70%, at least 75%, at least 80%, at least 85%, at least 90%, at least 95%, or greater of the activity of the wild-type IDS enzyme.

In some embodiments, an IDS enzyme, or a catalytically active variant or fragment thereof, that is present in a fusion protein described herein, retains at least 25% of its activity compared to its activity when not joined to an Fc polypeptide or a TfR-binding Fc polypeptide. In some embodiments, an IDS enzyme, or a catalytically active variant or fragment thereof, retains at least 10%, or at least 15%, 20%, 25%, 30%, 35%, 40%, 4%, 50%, 55%, 60%, 65%, 70%, 75%, 80%, 85%, 90%, or 95%, of its activity compared to its activity when not joined to an Fc polypeptide or a TfR-binding Fc polypeptide. In some embodiments, an IDS enzyme, or a catalytically active variant or fragment thereof, retains at least 80%, 85%, 90%, or 95% of its activity compared to its activity when not joined to an Fc polypeptide or a TfR-binding Fc polypeptide. In some embodiments, fusion to an Fc polypeptide does not decrease the activity of the IDS enzyme, or catalytically active variant or fragment thereof. In some embodiments, fusion to a TfR-binding Fc polypeptide does not decrease the activity of the IDS enzyme.

I. Fc Polypeptide Modifications for Blood-Brain Barrier (BBB) Receptor Binding

In some aspects, the fusion proteins are capable of being transported across the blood-brain barrier (BBB). Such a protein comprises a modified Fc polypeptide that binds to a BBB receptor. BBB receptors are expressed on BBB endothelia, as well as other cell and tissue types. In some embodiments, the BBB receptor is transferrin receptor (TfR).

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein.

A modified (e.g., enhancing heterodimerization and/or BBB receptor-binding) Fc polypeptide present in a fusion protein described herein can have at least 70% identity, at least 75% identity, at least 80% identity, at least 85% identity, at least 90% identity, at least 95% identity, at least 96% identity, at least 97% identity, at least 98% identity, or at least 99% identity to a native Fc region sequence or a fragment thereof, e.g., a fragment of at least 50 amino acids or at least 100 amino acids, or greater in length. In some embodiments, the native Fc amino acid sequence is the Fc region sequence of SEQ ID NO:1. In some embodiments, the native Fc amino acid sequence is the Fc region sequence of SEQ ID NO:183. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 1-110 of SEQ ID NO:1; to amino acids 1-110 of SEQ ID NO:183; to amino acids 111-217 of SEQ ID NO:1, to amino acids 111-216 of SEQ ID NO:183, or a fragment thereof, e.g., a fragment of at least 50 amino acids or at least 100 amino acids, or greater in length.

In some embodiments, a modified (e.g., enhancing heterodimerization and/or BBB receptor-binding) Fc polypeptide comprises at least 50 amino acids, or at least 60, 65, 70, 75, 80, 85, 90, or 95 or more, or at least 100 amino acids, or more, that correspond to a native Fc region amino acid sequence. In some embodiments, the modified Fc polypeptide comprises at least 25 contiguous amino acids, or at least 30, 35, 40, or 45 contiguous amino acids, or 50 contiguous amino acids, or at least 60, 65, 70, 75, 80 85, 90, or 95 or more contiguous amino acids, or 100 or more contiguous amino acids, that correspond to a native Fc region amino acid sequence, such as SEQ ID NO:1 or 183.

In some embodiments, the domain that is modified for BBB receptor-binding activity is a human Ig CH3 domain, such as an IgG1 CH3 domain. The CH3 domain can be of any IgG subtype, i.e., from IgG1, IgG2, IgG3, or IgG4. In the context of IgG1 antibodies, a CH3 domain refers to the segment of amino acids from about position 341 to about position 447 as numbered according to the EU numbering scheme.

In some embodiments, a modified (e.g., BBB receptor-binding) Fc polypeptide present in a fusion protein described herein comprises at least one, two, or three substitutions; and in some embodiments, at least four, five, six, seven, eight, or nine substitutions at amino acid positions 384, 386, 387, 388, 389, 390, 413, 416, and 421, according to the EU numbering scheme.

### FcRn Binding Sites

In certain aspects, modified (e.g., BBB receptor-binding) Fc polypeptides, or Fe polypeptides present in a fusion protein described herein that do not specifically bind to a BBB receptor, can also comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart modified Fc polypeptide having the wild-type residues at the mutated positions when assayed under the same conditions). In some embodiments, an FcRn binding site has one or more amino acid residues that are substituted at positions 250-256, 307, 380, 428, and 433-436, according to the EU numbering scheme.

In some embodiments, one or more residues at or near an FcRn binding site are mutated, relative to a native human IgG sequence, to extend serum half-life of the modified polypeptide. In some embodiments, mutations are introduced into one, two, or three of positions 252, 254, and 256. In some embodiments, the mutations are M252Y, S254T, and T256E. In some embodiments, a modified Fc polypeptide further comprises the mutations M252Y, S254T, and T256E. In some embodiments, a modified Fc polypeptide comprises a substitution at one, two, or all three of positions T307, E380, and N434, according to the EU numbering scheme. In some embodiments, the mutations are T307Q and N434A. In some embodiments, a modified Fc polypeptide comprises mutations T307A, E380A, and N434A. In some embodiments, a modified Fc polypeptide comprises substitutions at positions T250 and M428, according to the EU numbering scheme. In some embodiments, the modified Fc polypeptide comprises mutations T250Q and/or M428L. In some embodiments, a modified Fc polypeptide comprises substitutions at positions M428 and N434, according to the EU numbering scheme. In some embodiments, the modified Fc polypeptide comprises mutations M428L and N434S. In some embodiments, a modified Fc polypeptide comprises an N434S or N434A mutation.

II. Transferrin Receptor-Binding Fc Polypeptides

This section describes generation of modified Fc polypeptides described herein that bind to transferrin receptor (TfR) and are capable of being transported across the blood-brain barrier (BBB).

### TfR-Binding Fc Polypeptides Comprising Mutations in the CH3 Domain

In some embodiments, a modified Fc polypeptide that specifically binds to TfR comprises substitutions in a CH3 domain. In some embodiments, a modified Fc polypeptide comprises a human Ig CH3 domain, such as an IgG CH3 domain, that is modified for TfR-binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

In some embodiments, a modified Fc polypeptide that specifically binds to TfR comprises at least two, three, four, five, six, seven, eight, or nine substitutions at positions 384, 386, 387, 388, 389, 390, 413, 416, and 421, according to the EU numbering scheme. Illustrative substitutions that may be introduced at these positions are shown in Tables 4 and 5. In some embodiments, the amino acid at position 388 and/or 421 is an aromatic amino acid, e.g., Trp, Phe, or Tyr. In some embodiments, the amino acid at position 388 is Trp. In some embodiments, the aromatic amino acid at position 421 is Trp or Phe.

In some embodiments, at least one position as follows is substituted: Leu, Tyr, Met, or Val at position 384; Leu, Thr, His, or Pro at position 386; Val, Pro, or an acidic amino acid at position 387; an aromatic amino acid, e.g., Trp at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four, five, six, seven, or all eight of positions 384, 386, 387, 388, 389, 413, 416, and 421 have an amino acid substitution as specified in this paragraph.

In some embodiments, an Fc polypeptide that is modified as described in the preceding two paragraphs comprises a native Asn at position 390. In some embodiments, the modified Fc polypeptide comprises Gly, His, Gln, Leu, Lys, Val, Phe, Ser, Ala, or Asp at position 390. In some embodiments, the modified Fc polypeptide further comprises one, two, three, or four substitutions at positions comprising 380, 391, 392, and 415, according to the EU numbering scheme. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the modified Fc polypeptide comprises two, three, four, five, six, seven, eight, nine, ten, or eleven positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the modified Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the modified Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the modified Fc polypeptide further comprises Ser, Thr, Gln, or Phe at position 391. In some embodiments, the modified Fc polypeptide further comprises Trp, Tyr, Leu, or Gln at position 380 and/or Gln, Phe, or His at position 392. In some embodiments, Trp is present at position 380 and/or Gln is present at position 392. In some embodiments, the modified Fc polypeptide does not have a Trp at position 380.

In other embodiments, the modified Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the modified Fc polypeptide comprises a native Asn at position 390. In certain embodiments, the modified Fc polypeptide further comprises Trp, Tyr, Leu, or Gln at position 380; and/or Glu at position 415. In some embodiments, the modified Fc polypeptide further comprises Trp at position 380 and/or Glu at position 415.

In additional embodiments, the modified Fc polypeptide further comprises one, two, or three substitutions at positions comprising 414, 424, and 426, according to the EU numbering scheme. In some embodiments, position 414 is Lys, Arg, Gly, or Pro; position 424 is Ser, Thr, Glu, or Lys; and/or position 426 is Ser, Trp, or Gly.

In some embodiments, the modified Fc polypeptide comprises one or more of the following substitutions: Trp at position 380; Thr at position 386; Trp at position 388; Val at position 389; Thr or Ser at position 413; Glu at position 415; and/or Phe at position 421, according to the EU numbering scheme.

In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-217 of any one of SEQ ID NOS:4-68, 73-76, 81-84, and 95-167 (e.g., SEQ ID NOS:12-16, 36, and 38-68). In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-216 of any one of SEQ ID NOS: 184-198 and 200-206 (e.g., SEQ ID NOS: 184, 190, 192 and 200). In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to any one of SEQ ID NOS:4-68, 73-76, 81-84, 95-167, 184-198 and 200-206 (e.g., SEQ ID NOS:12-16, 36, 38-68, 184, 190, 192 and 200). In some embodiments, the modified Fc polypeptide comprises the amino acids at EU index positions 384-390 and/or 413-421 of any one of SEQ ID NOS:4-68, 73-76, 81-84, 95-167, 184-198 and 200-206 (e.g., SEQ ID NOS:12-16, 36, 38-68, 184, 190, 192 and 200). In some embodiments, the modified Fc polypeptide comprises the amino acids at EU index positions 380-390 and/or 413-421 of any one of SEQ ID NOS:4-68, 73-76, 81-84, 95-167, 184-198 and 200-206 (e.g., SEQ ID NOS:12-16, 36, 38-68, 184, 190, 192 and 200). In some embodiments, the modified Fc polypeptide comprises the amino acids at EU index positions 380-392 and/or 413-426 of any one of SEQ ID NOS:4-68, 73-76, 81-84, 95-167, 184-198 and 200-206 (e.g., SEQ ID NOS:12-16, 36, 38-68, 184, 190, 192 and 200).

In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to any one of SEQ ID NOS:4-68, 73-76, 81-84, 95-167, 184-198 and 200-206 (e.g., SEQ ID NOS:12-16, 36, 38-68, 184, 190, 192 and 200), and further comprises at least five, six, seven, eight, nine, ten, eleven, twelve, thirteen, fourteen, fifteen, or sixteen of the positions, numbered according to the EU index, as follows: Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426.

In some embodiments, the modified Fc polypeptide comprises the amino acid sequence of any one of SEQ ID NOS:12-16, 36, 38-68, 184, 190, 192 and 200. In other embodiments, the modified Fc polypeptide comprises the amino acid sequence of any one of SEQ ID NOS: 12-16, 36, 38-68, 184, 190, 192 and 200, but in which one, two, or three amino acids are substituted.

In some embodiments, the modified Fc polypeptide comprises additional mutations such as the mutations described below, including, but not limited to, a knob mutation (e.g., T366W as numbered with reference to EU numbering), hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and/or mutations that increase serum stability or serum half-life (e.g., (i) M252Y, S254T, and T256E as numbered with reference to EU numbering, or (ii) N434S with or without M428L as numbered according to the EU numbering scheme). By way of illustration, SEQ ID NOS:73-76, 81-84, 92, 95-169, 185-189, 191, 193-199, and 201-207 provide non-limiting examples of modified Fc polypeptides with mutations in the CH3 domain (e.g., clones CH3C.35.20.1, CH3C.35.23.2, CH3C.35.23.3, CH3C.35.23.4, CH3C.35.21.17.2, and CH3C.35.23) comprising one or more of these additional mutations.

In some embodiments, the modified Fc polypeptide comprises a knob mutation (e.g., T366W as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS:73, 96, 108, 120, 132, 144, 156, 185, 193, 201. In some embodiments, the modified Fc polypeptide comprises the sequence of any one of SEQ ID NOS:73, 96, 108, 120, 132, 144, 156, 185, 193 and 201.

In some embodiments, the modified Fc polypeptide comprises a knob mutation (e.g., T366W as numbered with reference to EU numbering) and mutations that modulate effector function (e.g., L234A, L235A, and/or P329G (e.g., L234A and L235A) as numbered with reference to EU numbering), and has at least 85% identity, at least 90% identity, at least 95% identity, at least 96% identity, at least 97% identity, at least 98% identity, or at least 99% identity to the sequence of any one of SEQ ID NOS:74, 92, 97, 98, 109, 110, 121, 122, 133, 134, 145, 146, 157, 158, 168, 169, 186, 191, 194, 195, 199, 202, 203, and 207. In some embodiments, the modified Fc polypeptide comprises the sequence of any one of SEQ ID NOS:74, 92, 97, 98, 109, 110, 121, 122, 133, 134, 145, 146, 157, 158, 168, 169, 186, 191, 194, 195, 199, 202, 203, and 207.

In some embodiments, the modified Fc polypeptide comprises a knob mutation (e.g., T366W as numbered with reference to EU numbering) and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, at least 95% identity, at least 96% identity, at least 97% identity, at least 98% identity, or at least 99% identity to the sequence of any one of SEQ ID NOS:75, 99, 111, 123, 135, 147, 159, 187, 196, and 204. In some embodiments, the modified Fc polypeptide comprises the sequence of any one of SEQ ID NOS:75, 99, 111, 123, 135, 147, 159, 187, 196 and 204.

In some embodiments, the modified Fc polypeptide comprises a knob mutation (e.g., T366W as numbered with reference to EU numbering), mutations that modulate effector function (e.g., L234A, L235A, and/or P329G (e.g., L234A and L235A) as numbered with reference to EU numbering), and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, at least 95% identity, at least 96% identity, at least 97% identity, at least 98% identity, or at least 99% identity to the sequence of any one of SEQ ID NOS:76, 100, 101, 112, 113, 124, 125, 136, 137, 148, 149, 160, 161, 188, 197, 198, 205, and 206. In some embodiments, the modified Fc polypeptide comprises the sequence of any one of SEQ ID NOS:76, 100, 101, 112, 113, 124, 125, 136, 137, 148, 149, 160, 161, 188, 197, 198, 205, and 206.

In some embodiments, the modified Fc polypeptide comprises hole mutations (e.g., T366S, L368A, and Y407V as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS:81, 102, 114, 126, 138, 150, and 162. In some embodiments, the modified Fc polypeptide comprises the sequence of any one of SEQ ID NOS:81, 102, 114, 126, 138, 150, and 162.

In some embodiments, the modified Fc polypeptide comprises hole mutations (e.g., T366S, L368A, and Y407V as numbered with reference to EU numbering) and mutations that modulate effector function (e.g., L234A, L235A, and/or P329G (e.g., L234A and L235A) as numbered with reference to EU numbering), and has at least 85% identity, at least 90% identity, at least 95% identity, at least 96% identity, at least 97% identity, at least 98% identity, or at least 99% identity to the sequence of any one of SEQ ID NOS:82, 103, 104, 115, 116, 127, 128, 139, 140, 151, 152, 163, and 164. In some embodiments, the modified Fc polypeptide comprises the sequence of any one of SEQ ID NOS:82, 103, 104, 115, 116, 127, 128, 139, 140, 151, 152, 163, and 164.

In some embodiments, the modified Fc polypeptide comprises hole mutations (e.g., T366S, L368A, and Y407V as numbered with reference to EU numbering) and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, at least 95%, at least 96% identity, at least 97% identity, at least 98% identity, or at least 99% identity to the sequence of any one of SEQ ID NOS:83, 105, 117, 129, 141, 153, and 165. In some embodiments, the modified Fc polypeptide comprises the sequence of any one of SEQ ID NOS:83, 105, 117, 129, 141, 153, and 165.

In some embodiments, the modified Fc polypeptide comprises hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and mutations that increase serum stability or serum half-life (e.g., (i) M252Y, S254T, and T256E as numbered with reference to EU numbering, or (ii) N434S with or without M428L as numbered according to the EU numbering scheme), and has at least 85% identity, at least 90% identity, at least 95% identity, at least 96% identity, at least 97% identity, at least 98% identity, or at least 99% identity to the sequence of any one of SEQ ID NOS:84, 106, 107, 118, 119, 130, 131, 142, 143, 154, 155, 166, and 167. In some embodiments, the modified Fc polypeptide comprises the sequence of any one of SEQ ID NOS:84, 106, 107, 118, 119, 130, 131, 142, 143, 154, 155, 166, and 167.

In some embodiments, a modified Fc polypeptide that specifically binds to TfR comprises at least two, three, four, five, six, seven, or eight substitutions at positions 345, 346, 347, 349, 437, 438, 439, and 440, according to the EU numbering scheme. In some embodiments, the modified Fc polypeptide comprises Gly at position 437; Phe at position 438; and/or Asp at position 440. In some embodiments, Glu is present at position 440. In certain embodiments, the modified Fc polypeptide comprises at least one substitution at a position as follows: Phe or Ile at position 345; Asp, Glu, Gly, Ala, or Lys at position 346; Tyr, Met, Leu, Ile, or Asp at position 347; Thr or Ala at position 349; Gly at position 437; Phe at position 438; His Tyr, Ser, or Phe at position 439; or Asp at position 440. In some embodiments, two, three, four, five, six, seven, or all eight of positions 345, 346, 347, 349, 437, 438, 439, and 440 and have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

III. Additional Fc Polypeptide Mutations

In some aspects, a fusion protein described herein comprises two Fc polypeptides that may each comprise independently selected modifications or may be a wild-type Fc polypeptide, e.g., a human IgG1 Fc polypeptide. In some embodiments, one or both Fc polypeptides contains one or more modifications that confer binding to a blood-brain barrier (BBB) receptor, e.g., transferrin receptor (TfR). Non-limiting examples of other mutations that can be introduced into one or both Fc polypeptides include, e.g., mutations to increase serum stability or serum half-life, to modulate effector function, to influence glycosylation, to reduce immunogenicity in humans, and/or to provide for knob and hole heterodimerization of the Fc polypeptides.

In some embodiments, the Fc polypeptides present in the fusion protein independently have an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

In some embodiments, the Fc polypeptides present in the fusion protein include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the fusion protein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a fusion protein described herein has the T366W knob mutation and the other Fc polypeptide has the Y407V mutation, which is typically accompanied by the T366S and L368A hole mutations.

In some embodiments, modifications to enhance serum half-life may be introduced. For example, in some embodiments, one or both Fc polypeptides present in a fusion protein described herein may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, as numbered according to the EU numbering scheme. Alternatively, one or both Fc polypeptides may have an N434S or N434A substitution.

In some embodiments, one or both Fc polypeptides present in a fusion protein described herein may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in a fusion protein described herein may also be engineered to contain other modifications for heterodimerization, e.g., electrostatic engineering of contact residues within a CH3-CH3 interface that are naturally charged or hydrophobic patch modifications.

In some embodiments, one or both Fc polypeptides present in a fusion protein described herein may include additional modifications that modulate effector function.

In some embodiments, one or both Fc polypeptides present in a fusion protein described herein may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

In some embodiments, the C′terminal Lys residue is removed or not present in an Fc polypeptide described herein (i.e., the Lys residue at position 447, according to the EU numbering scheme).

### Illustrative Fc Polypeptides Comprising Additional Mutations

By way of non-limiting example, one or both Fc polypeptides present in a fusion protein described herein may comprise additional mutations including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and/or mutations that increase serum stability or serum half-life (e.g., (i) M252Y, S254T, and T256E as numbered with reference to EU numbering, or (ii) N434S with or without M428L as numbered according to the EU numbering scheme).

In some embodiments, an Fc polypeptide may have a knob mutation (e.g., T366W as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS:1, 4-68, 183, 184, 190, 192, and 200. In some embodiments, an Fc polypeptide having the sequence of any one of SEQ ID NOS: 1, 4-68, 183, 184, 190, 192, and 200 may be modified to have a knob mutation.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 4-68, 183, 184, 190, 192, and 200. In some embodiments, an Fc polypeptide having the sequence of any one of SEQ ID NOS:1, 4-68, 183, 184, 190, 192, and 200 may be modified to have a knob mutation and mutations that modulate effector function.

In some embodiments, an Fc polypeptide may have a knob mutation (e.g., T366W as numbered according to the EU numbering scheme), mutations that increase serum stability or serum half-life (e.g., (i) M252Y, S254T, and T256E as numbered with reference to EU numbering, or (ii) N434S with or without M428L as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 4-68, 183, 184, 190, 192, and 200. In some embodiments, an Fc polypeptide having the sequence of any one of SEQ ID NOS:1, 4-68, 183, 184, 190, 192, and 200 may be modified to have a knob mutation and mutations that increase serum stability or serum half-life.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), mutations that increase serum stability or serum half-life (e.g., (i) M252Y, S254T, and T256E as numbered with reference to EU numbering, or (ii) N434S with or without M428L as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 4-68, 183, 184, 190, 192, and 200. In some embodiments, an Fc polypeptide having the sequence of any one of SEQ ID NOS:1, 4-68, 183, 184, 190, 192, and 200 may be modified to have a knob mutation, mutations that modulate effector function, and mutations that increase serum stability or serum half-life.

In some embodiments, an Fc polypeptide may have hole mutations (e.g., T366S, L368A, and Y407V as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS: 1, 4-68, 183, 184, 190, 192, and 200. In some embodiments, an Fc polypeptide having the sequence of any one of SEQ ID NOS: 1, 4-68, 183, 184, 190, 192, and 200 may be modified to have hole mutations.

In some embodiments, an Fc polypeptide may have hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 4-68, 183, 184, 190, 192, and 200. In some embodiments, an Fc polypeptide having the sequence of any one of SEQ ID NOS: 1, 4-68, 183, 184, 190, 192, and 200 may be modified to have hole mutations and mutations that modulate effector function.

In some embodiments, an Fc polypeptide may have hole mutations (e.g., T366S, L368A, and Y407V as numbered according to the EU numbering scheme), mutations that increase serum or serum half-life (e.g., (i) M252Y, S254T, and T256E as numbered with reference to EU numbering, or (ii) N434S with or without M428L as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 4-68, 183, 184, 190, 192, and 200. In some embodiments, an Fc polypeptide having sequence of any one of SEQ ID NOS: 1, 4-68, 183, 184, 190, 192, and 200 may be modified to have hole mutations and mutations that increase serum stability or serum half-life.

In some embodiments, an Fc polypeptide may have hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), mutations that increase serum stability or serum half-life (e.g., (i) M252Y, S254T, and T256E as numbered with reference to EU numbering, or (ii) N434S with or without M428L as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 4-68, 183, 184, 190, 192, and 200. In some embodiments, an Fc polypeptide having the sequence of any one of SEQ ID NOS: 1, 4-68, 183, 184, 190, 192, and 200 may be modified to have hole mutations, mutations that modulate effector function, and mutations that increase serum stability or serum half-life.

IV. Illustrative Fusion Proteins Comprising an IDS Enzyme

In some aspects, a fusion protein described herein comprises a first Fc polypeptide that is linked to an IDS enzyme, an IDS enzyme variant, or a catalytically active fragment thereof, and a second Fc polypeptide that forms an Fc dimer with the first Fc polypeptide. In some embodiments, the first Fc polypeptide and/or the second Fc polypeptide does not include an immunoglobulin heavy and/or light chain variable region sequence or an antigen-binding portion thereof. In some embodiments, the first Fc polypeptide is a modified Fc polypeptide and/or the second Fc polypeptide is a modified Fc polypeptide. In some embodiments, the second Fc polypeptide is a modified Fc polypeptide. In some embodiments, the modified Fc polypeptide contains one or more modifications that promote its heterodimerization to the other Fc polypeptide. In some embodiments, the modified Fc polypeptide contains one or more modifications that reduce effector function. In some embodiments, the modified Fc polypeptide contains one or more modifications that extend serum half-life. In some embodiments, the modified Fc polypeptide contains one or more modifications that confer binding to a blood-brain barrier (BBB) receptor, e.g., transferrin receptor (TfR).

In other aspects, a fusion protein described herein comprises a first polypeptide chain that comprises a modified Fc polypeptide that specifically binds to a BBB receptor, e.g., TfR, and a second polypeptide chain that comprises an Fc polypeptide which dimerizes with the modified Fc polypeptide to form an Fe dimer. An IDS enzyme may be linked to either the first or the second polypeptide chain. In some embodiments, the IDS enzyme is linked to the second polypeptide chain. In some embodiments, the protein comprises two IDS enzymes, each linked to one of the polypeptide chains. In some embodiments, the Fc polypeptide may be a BBB receptor-binding polypeptide that specifically binds to the same BBB receptor as the modified Fc polypeptide in the first polypeptide chain. In some embodiments, the Fc polypeptide does not specifically bind to a BBB receptor.

In some embodiments, a fusion protein described herein comprises a first polypeptide chain comprising a modified Fc polypeptide that specifically binds to TfR and a second polypeptide chain that comprises an Fc polypeptide, wherein the modified Fc polypeptide and the Fc polypeptide dimerize to from an Fc dimer. In some embodiments, the IDS enzyme is linked to the first polypeptide chain. In some embodiments, the IDS enzyme is linked to the second polypeptide chain. In some embodiments, the Fc polypeptide does not specifically bind to a BBB receptor, e.g., TfR.

In some embodiments, a fusion protein described herein comprises a first polypeptide chain that comprises a modified Fc polypeptide that binds to TfR and comprises a T366W (knob) substitution; and a second polypeptide chain that comprises an Fc polypeptide comprising T366S, L368A, and Y407V (hole) substitutions. In some embodiments, the modified Fc polypeptide and/or the Fc polypeptide further comprises L234A and L235A (LALA) substitutions. In some embodiments, the modified Fc polypeptide and/or the Fc polypeptide further comprises M252Y, S254T, and T256E (YTE) substitutions. In some embodiments, the modified Fc polypeptide and/or the Fc polypeptide further comprises L234A and L235A (LALA) substitutions and M252Y, S254T, and T256E (YTE) substitutions. In some embodiments, the modified Fc polypeptide and/or the Fc polypeptide comprises human IgG1 wild-type residues at positions 234, 235, 252, 254, 256, and 366.

In some embodiments, the modified Fc polypeptide comprises the knob, LALA, and/or YTE mutations as specified for any one of SEQ ID NOS:73-76, 95-101, 108-113, 120-125, 132-137, 144-149, 156-161, 185-188, 191, 193-198, and 201-206, and has at least 85% identity, at least 90% identity, at least 95% identity, at least 96% identity, at least 97% identity, at least 98% identity, or at least 99% identity to the respective sequence; or comprises the sequence of any one of SEQ ID NOS: 73-76, 95-101, 108-113, 120-125, 132-137, 144-149, 156-161, 185-188, 191, 193-198, and 201-206. In some embodiments, the Fc polypeptide comprises the hole, LALA, and/or YTE mutations as specified for any one of SEQ ID NOS:77-80 and 208-211 and has at least 85% identity, at least 90% identity, at least 95% identity, at least 96% identity, at least 97% identity, at least 98% identity, or at least 99% identity to the respective sequence; or comprises the sequence of any one of SEQ ID NOS: 77-80 and 208-211. In some embodiments, the modified Fc polypeptide comprises any one of SEQ ID NOS:73-76, 95-101, 108-113, 120-125, 132-137, 144-149, and 156-161, and the Fc polypeptide comprises any one of SEQ ID NOS:77-80. In some embodiments, the modified Fc polypeptide comprises any one of SEQ ID NOS: 185-188, 191, 193-198 and 201-206, and the Fc polypeptide comprises any one of SEQ ID NOS: 208-211. In some embodiments, the N-terminus of the modified Fc polypeptide and/or the Fc polypeptide includes a portion of an IgG1 hinge region (e.g., DKTHTCPPCP; SEQ ID NO:89). In some embodiments, the modified Fc polypeptide has at least 85%, at least 90%, at least 95% identity, at least 96% identity, at least 97% identity, at least 98% identity, or at least 99% identity to any one of SEQ ID NOS:92, 168, and 169, or comprises the sequence of any one of SEQ ID NOS:92, 168, and 169. In some embodiments, the modified Fc polypeptide has at least 85%, at least 90%, or at least 95% identity, at least 96% identity, at least 97% identity, at least 98% identity, or at least 99% identity to any one of SEQ ID NOS:189, 199, and 207, or comprises the sequence of any one of SEQ ID NOS: 189, 199, and 207.

In some embodiments, a fusion protein described herein comprises a first polypeptide chain that comprises a modified Fc polypeptide that binds to TfR and comprises T366S, L368A, and Y407V (hole) substitutions; and a second polypeptide chain that comprises an Fc polypeptide comprising a T366W (knob) substitution. In some embodiments, the modified Fc polypeptide and/or the Fc polypeptide further comprises L234A and L235A (LALA) substitutions. In some embodiments, the modified Fc polypeptide and/or the Fc polypeptide further comprises M252Y, S254T, and T256E (YTE) substitutions. In some embodiments, the modified Fc polypeptide and/or the Fc polypeptide further comprises L234A and L235A (LALA) substitutions and M252Y, S254T, and T256E (YTE) substitutions. In some embodiments, the modified Fc polypeptide and/or the Fc polypeptide comprises human IgG1 wild-type residues at positions 234, 235, 252, 254, 256, and 366.

In some embodiments, the modified Fc polypeptide comprises the hole, LALA, and/or YTE mutations as specified for any one of SEQ ID NOS:81-84, 102-107, 114-119, 126-131, 138-143, 150-155, and 162-167 and has at least 85% identity, at least 90% identity, at least 95% identity, at least 96% identity, at least 97% identity, at least 98% identity, or at least 99% identity to the respective sequence; or comprises the sequence of any one of SEQ ID NOS: 81-84, 102-107, 114-119, 126-131, 138-143, 150-155, and 162-167. In some embodiments, the Fc polypeptide comprises the knob, LALA, and/or YTE mutations as specified for any one of SEQ ID NOS:85-88 and has at least 85% identity, at least 90% identity, at least 95% identity, at least 96% identity, at least 97% identity, at least 98% identity, or at least 99% identity to the respective sequence; or comprises the sequence of any one of SEQ ID NOS:85-88. In some embodiments, the modified Fc polypeptide comprises any one of SEQ ID NOS: 81-84, 102-107, 114-119, 126-131, 138-143, 150-155, and 162-167, and the Fc polypeptide comprises any one of SEQ ID NOS:85-88. In some embodiments, the N-terminus of the modified Fc polypeptide and/or the Fc polypeptide includes a portion of an IgG1 hinge region (e.g., DKTHTCPPCP; SEQ ID NO:89).

In some embodiments, an IDS enzyme present in a fusion protein described herein is linked to a polypeptide chain that comprises an Fc polypeptide having at least 85%, at least 90%, at least 95% identity, at least 96% identity, at least 97% identity, at least 98% identity, or at least 99% identity to any one of SEQ ID NOS: 77-80 and 208-211, or comprises the sequence of any one of SEQ ID NOS: 77-80 and 208-211 (e.g., as a fusion polypeptide). In some embodiments, the IDS enzyme is linked to the Fc polypeptide by a linker, such as a flexible linker, and/or a hinge region or portion thereof (e.g., DKTHTCPPCP; SEQ ID NO:89). In some embodiments, the IDS enzyme comprises a sequence having at least 85%, at least 90%, at least 95% identity, at least 96% identity, at least 97% identity, at least 98% identity, or at least 99% identity to any one of SEQ ID NOS:90, 170, and 174, or comprises the sequence of any one of SEQ ID NOS:90, 170, and 174. In some embodiments, the IDS sequence linked to the Fc polypeptide has at least 85%, at least 90%, at least 95% identity, at least 96% identity, at least 97% identity, at least 98% identity, or at least 99% identity to any one of SEQ ID NOS:91, 93, 171, 172, 175, 176, and 212-217 or comprises the sequence of any one of SEQ ID NOS: 91, 93, 171, 172, 175, 176, and 212-217. In some embodiments, the fusion protein comprises a modified Fc polypeptide having at least 85%, at least 90%, at least 95% identity, at least 96% identity, at least 97% identity, at least 98% identity, or at least 99% identity to any one of SEQ ID NOS: 73-76, 95-101, 108-113, 120-125, 132-137, 144-149, 156-161, 185-188, 191, 193-198, and 201-206, or comprises the sequence of any one of SEQ ID NOS: 73-76, 95-101, 108-113, 120-125, 132-137, 144-149, 156-161, 185-188, 191, 193-198, and 201-206. In some embodiments, the N-terminus of the Fc polypeptide and/or the modified Fc polypeptide includes a portion of an IgG1 hinge region (e.g., DKTHTCPPCP; SEQ ID NO:89). In some embodiments, the modified Fc polypeptide has at least 85%, at least 90%, at least 95% identity, at least 96% identity, at least 97% identity, at least 98% identity, or at least 99% identity to any one of SEQ ID NOS:92, 168, and 169, or comprises the sequence of any one of SEQ ID NOS:92, 168, and 169. In some embodiments, the modified Fc polypeptide has at least 85%, at least 90%, or at least 95% identity, at least 96% identity, at least 97% identity, at least 98% identity, or at least 99% identity to any one of SEQ ID NOS:189, 199, and 207, or comprises the sequence of any one of SEQ ID NOS: 189, 199, and 207.

In some embodiments, the fusion protein comprises an IDS-Fc fusion polypeptide comprising the sequence of SEQ ID NO:91, and a modified Fc polypeptide comprising the sequence of any one of SEQ ID NOS: 145 and 168 (e.g., SEQ ID NO:168). In other embodiments, the fusion protein comprises an IDS-Fc fusion polypeptide comprising the sequence of SEQ ID NO:91, and a modified Fc polypeptide comprising the sequence of any one of SEQ ID NOS:109 and 169 (e.g., SEQ ID NO:169).

In some embodiments, the fusion protein comprises an IDS-Fc fusion polypeptide comprising the sequence of SEQ ID NO:215, and a modified Fc polypeptide comprising the sequence of any one of SEQ ID NOS:202 and 207 (e.g., SEQ ID NO:207). In other embodiments, the fusion protein comprises an IDS-Fc fusion polypeptide comprising the sequence of SEQ ID NO:215, and a modified Fc polypeptide comprising the sequence of any one of SEQ ID NOS:194 and 199 (e.g., SEQ ID NO:199).

In some embodiments, the fusion protein comprises an IDS-Fc fusion polypeptide comprising the sequence of SEQ ID NO:171, and a modified Fc polypeptide comprising the sequence of any one of SEQ ID NOS: 145 and 168 (e.g., SEQ ID NO:168). In other embodiments, the fusion protein comprises an IDS-Fc fusion polypeptide comprising the sequence of SEQ ID NO:171, and a modified Fc polypeptide comprising the sequence of any one of SEQ ID NOS:109 and 169 (e.g., SEQ ID NO:169).

In some embodiments, the fusion protein comprises an IDS-Fc fusion polypeptide comprising the sequence of SEQ ID NO:213, and a modified Fc polypeptide comprising the sequence of any one of SEQ ID NOS:202 and 207 (e.g., SEQ ID NO:207). In other embodiments, the fusion protein comprises an IDS-Fc fusion polypeptide comprising the sequence of SEQ ID NO:213, and a modified Fc polypeptide comprising the sequence of any one of SEQ ID NOS:194 and 199 (e.g., SEQ ID NO:199).

In some embodiments, the fusion protein comprises an IDS-Fc fusion polypeptide comprising the sequence of SEQ ID NO:175, and a modified Fc polypeptide comprising the sequence of any one of SEQ ID NOS:145 and 168 (e.g., SEQ ID NO:168). In other embodiments, the fusion protein comprises an IDS-Fc fusion polypeptide comprising the sequence of SEQ ID NO:175, and a modified Fc polypeptide comprising the sequence of any one of SEQ ID NOS:109 and 169 (e.g., SEQ ID NO:169).

In some embodiments, the fusion protein comprises an IDS-Fc fusion polypeptide comprising the sequence of SEQ ID NO:217, and a modified Fc polypeptide comprising the sequence of any one of SEQ ID NOS:202 and 207 (e.g., SEQ ID NO:207). In other embodiments, the fusion protein comprises an IDS-Fc fusion polypeptide comprising the sequence of SEQ ID NO:217, and a modified Fc polypeptide comprising the sequence of any one of SEQ ID NOS:194 and 199 (e.g., SEQ ID NO:199).

In some embodiments, an IDS enzyme present in a fusion protein described herein is linked to a polypeptide chain that comprises an Fc polypeptide having at least 85%, at least 90%, at least 95% identity, at least 96% identity, at least 97% identity, at least 98% identity, or at least 99% identity to any one of SEQ ID NOS:85-88, or comprises the sequence of any one of SEQ ID NOS:85-88 (e.g., as a fusion polypeptide). In some embodiments, the IDS enzyme is linked to the Fc polypeptide by a linker, such as a flexible linker, and/or a hinge region or portion thereof (e.g., DKTHTCPPCP; SEQ ID NO:89). In some embodiments, the IDS enzyme comprises a sequence having at least 85%, at least 90%, at least 95% identity, at least 96% identity, at least 97% identity, at least 98% identity, or at least 99% identity to any one of SEQ ID NOS:90, 170, and 174, or comprises the sequence of any one of SEQ ID NOS:90, 170, and 174. In some embodiments, the IDS sequence linked to the Fc polypeptide has at least 85%, at least 90%, at least 95% identity, at least 96% identity, at least 97% identity, at least 98% identity, or at least 99% identity to any one of SEQ ID NOS:94, 173, and 177, or comprises the sequence of any one of SEQ ID NOS:94, 173, and 177. In some embodiments, the fusion protein comprises a modified Fc polypeptide having at least 85%, at least 90%, at least 95% identity, at least 96% identity, at least 97% identity, at least 98% identity, or at least 99% identity to any one of SEQ ID NOS: 81-84, 102-107, 114-119, 126-131, 138-143, 150-155, and 162-167, or comprises the sequence of any one of SEQ ID NOS: 81-84, 102-107, 114-119, 126-131, 138-143, 150-155, and 162-167. In some embodiments, the N-terminus of the Fc polypeptide and/or the modified Fc polypeptide includes a portion of an IgG1 hinge region (e.g., DKTHTCPPCP; SEQ ID NO:89).

In some embodiments, an IDS enzyme present in a fusion protein described herein is linked to a polypeptide chain that comprises a modified Fc polypeptide having at least 85%, at least 90%, at least 95% identity, at least 96% identity, at least 97% identity, at least 98% identity, or at least 99% identity to any one of SEQ ID NOS: 73-76, 95-101, 108-113, 120-125, 132-137, 144-149, 156-161, 185-188, 191, 193-198, and 201-206, or comprises the sequence of any one of SEQ ID NOS: 73-76, 95-101, 108-113, 120-125, 132-137, 144-149, 156-161, 185-188, 191, 193-198, and 201-206 (e.g., as a fusion polypeptide). In some embodiments, the IDS enzyme is linked to the modified Fc polypeptide by a linker, such as a flexible linker, and/or a hinge region or portion thereof (e.g., DKTHTCPPCP; SEQ ID NO:89). In some embodiments, the IDS enzyme comprises a sequence having at least 85%, at least 90%, at least 95% identity, at least 96% identity, at least 97% identity, at least 98% identity, or at least 99% identity to any one of SEQ ID NOS:90, 170, and 174, or comprises the sequence of any one of SEQ ID NOS:90, 170, and 174. In some embodiments, the fusion protein comprises an Fc polypeptide having at least 85%, at least 90%, at least 95% identity, at least 96% identity, at least 97% identity, at least 98% identity, or at least 99% identity to any one of SEQ ID NOS:77-80, or comprises the sequence of any one of SEQ ID NOS:77-80. In some embodiments, the fusion protein comprises an Fc polypeptide having at least 85%, at least 90%, at least 95% identity, at least 96% identity, at least 97% identity, at least 98% identity, or at least 99% identity to any one of SEQ ID NOS:208-211, or comprises the sequence of any one of SEQ ID NOS:208-211. In some embodiments, the N-terminus of the modified Fc polypeptide and/or the Fc polypeptide includes a portion of an IgG1 hinge region (e.g., DKTHTCPPCP; SEQ ID NO:89).

In some embodiments, an IDS enzyme present in a fusion protein described herein is linked to a polypeptide chain that comprises a modified Fc polypeptide having at least 85%, at least 90%, at least 95% identity, at least 96% identity, at least 97% identity, at least 98% identity, or at least 99% identity to any one of SEQ ID NOS: 81-84, 102-107, 114-119, 126-131, 138-143, 150-155, and 162-167, or comprises the sequence of any one of SEQ ID NOS: 81-84, 102-107, 114-119, 126-131, 138-143, 150-155, and 162-167 (e.g., as a fusion polypeptide). In some embodiments, the IDS enzyme is linked to the modified Fc polypeptide by a linker, such as a flexible linker, and/or a hinge region or portion thereof (e.g., DKTHTCPPCP; SEQ ID NO:89). In some embodiments, the IDS enzyme comprises a sequence having at least 85%, at least 90%, at least 95% identity, at least 96% identity, at least 97% identity, at least 98% identity, or at least 99% identity to any one of SEQ ID NOS:90, 170, and 174, or comprises the sequence of any one of SEQ ID NOS:90, 170, and 174. In some embodiments, the fusion protein comprises an Fc polypeptide having at least 85%, at least 90%, at least 95% identity, at least 96% identity, at least 97% identity, at least 98% identity, or at least 99% identity to any one of SEQ ID NOS:85-88, or comprises the sequence of any one of SEQ ID NOS:85-88. In some embodiments, the N-terminus of the modified Fc polypeptide and/or the Fc polypeptide includes a portion of an IgG1 hinge region (e.g., DKTHTCPPCP; SEQ ID NO:89).

V. IDS Enzymes Linked to Fc Polypeptides

In some embodiments, a fusion protein described herein comprises two Fc polypeptides as described herein and one or both of the Fc polypeptides may further comprise a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:71) or a portion thereof (e.g., DKTHTCPPCP; SEQ ID NO:89). In some embodiments, the hinge region is at the N-terminal region of the Fc polypeptide.

In some embodiments, an Fc polypeptide is joined to the IDS enzyme by a linker, e.g., a peptide linker. In some embodiments, the Fc polypeptide is joined to the IDS enzyme by a peptide bond or by a peptide linker, e.g., is a fusion polypeptide. The peptide linker may be configured such that it allows for the rotation of the IDS enzyme relative to the Fc polypeptide to which it is joined; and/or is resistant to digestion by proteases. Peptide linkers may contain natural amino acids, unnatural amino acids, or a combination thereof. In some embodiments, the peptide linker may be a flexible linker, e.g., containing amino acids such as Gly, Asn, Ser, Thr, Ala, and the like. Such linkers are designed using known parameters and may be of any length and contain any number of repeat units of any length (e.g., repeat units of Gly and Ser residues). For example, the linker may have repeats, such as two, three, four, five, or more Gly4-Ser (SEQ ID NO:179) repeats or a single Gly4-Ser (SEQ ID NO:179). In some embodiments, the peptide linker may include a protease cleavage site, e.g., that is cleavable by an enzyme present in the central nervous system.

In some embodiments, the IDS enzyme is joined to the N-terminus of the Fc polypeptide, e.g., by a Gly₄-Ser linker (SEQ ID NO:179) or a (Gly₄-Ser)₂ linker (SEQ ID NO:180). In some embodiments, the Fc polypeptide may comprise a hinge sequence or partial hinge sequence at the N-terminus that is joined to the linker or directly joined to the IDS enzyme.

In some embodiments, the IDS enzyme is joined to the C-terminus of the Fc polypeptide, e.g., by a Gly₄-Ser linker (SEQ ID NO:179) or a (Gly₄-Ser)₂ linker (SEQ ID NO:180). In some embodiments, the C-terminus of the Fc polypeptide is directly joined to the IDS enzyme.

In some embodiments, the IDS enzyme is joined to the Fc polypeptide by a chemical cross-linking agent. Such conjugates can be generated using well-known chemical cross-linking reagents and protocols. For example, there are a large number of chemical cross-linking agents that are known to those skilled in the art and useful for cross-linking the polypeptide with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl) aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate. 2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers.

### CERTAIN EMBODIMENTS

Embodiment 1. A method of treating Hunter syndrome in a subject in need thereof, comprising administering to the subject a therapeutically effective dose of a pharmaceutical composition comprising a protein, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 2. A method of treating Hunter syndrome in a subject in need thereof, comprising administering to the subject a therapeutically effective dose (e.g., a safe and therapeutically effective dose) of a pharmaceutical composition comprising a protein, wherein administration of the pharmaceutical composition reduces levels of a glycosaminoglycan (GAG) in the CSF of the subject to a baseline level measured in a healthy subject or a subject that does not have Hunter syndrome, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 3. The method of any one of embodiments 1-2, wherein the therapeutically effective dose (e.g., safe and therapeutically effective dose) is from about 3 mg/kg to about 30 mg/kg of protein.

Embodiment 4. The method of embodiment 3 or 115, wherein the therapeutically effective dose (e.g., a safe and therapeutically effective dose) is about 3 mg/kg of protein.

Embodiment 5. The method of embodiment 3 or 115, wherein the therapeutically effective dose (e.g., a safe and therapeutically effective dose) is about 7.5 mg/kg of protein.

Embodiment 6. The method of embodiment 3 or 115, wherein the therapeutically effective dose (e.g., a safe and therapeutically effective dose) is about 15 mg/kg of protein.

Embodiment 7. The method of embodiment 3 or 115, wherein the therapeutically effective dose (e.g., a safe and therapeutically effective dose) is about 30 mg/kg of protein.

Embodiment 8. The method of any one of embodiments 1-7 or 112-150, wherein the pharmaceutical composition is administered weekly.

Embodiment 9. The method of any one of embodiments 1-8, wherein the pharmaceutical composition further comprises a pharmaceutically acceptable excipient (e.g., a pharmaceutically acceptable carrier).

Embodiment 10. The method of any one of embodiments 1-9, wherein the second Fc polypeptide specifically binds to the transferrin receptor.

Embodiment 11. The method of any one of embodiments 1-10, wherein the IDS amino acid sequence comprises an amino acid sequence having at least 90% identity to SEQ ID NO:70.

Embodiment 12. The method of embodiment 11, wherein the IDS amino acid sequence comprises a sequence selected from the group consisting of SEQ ID NOs:70, 170 and 174.

Embodiment 13. The method of embodiment 11, wherein the IDS amino acid sequence comprises a sequence selected from the group consisting of SEQ ID NOs:70, 90, 170 and 174.

Embodiment 14. The method of any one of embodiments 1-13, wherein the first Fc polypeptide linked to the IDS amino acid sequence comprises the amino acid sequence of SEQ ID NO: 91, 171 or 175.

Embodiment 15. The method of any one of embodiments 1-13, wherein the first Fc polypeptide linked to the IDS amino acid sequence comprises the amino acid sequence of SEQ ID NO: 213, 215, or 217.

Embodiment 16. The method of any one of embodiments 1-15, wherein the IDS amino acid sequence is linked to the N-terminus of the first Fc polypeptide.

Embodiment 17. The method of any one of embodiments 1-16, wherein the second Fc polypeptide comprises:
a. Leu at position 380;
b. Ala at position 389; and
c. Ser at position 390.

Embodiment 18. The method of any one of embodiments 1-16, wherein the second Fc polypeptide comprises:
a. Glu at position 380;
b. Ala at position 389; and
c. Asn at position 390.

Embodiment 19. The method of any one of embodiments 1-16, wherein the second Fc polypeptide comprises:
a. Trp at position 380;
b. Ser at position 389; and
c. Ser at position 390.

Embodiment 20. The method of any one of embodiments 1-16, wherein the second Fc polypeptide comprises:
a. Leu at position 380;
b. Ser at position 389; and
c. Ser at position 390.

Embodiment 21. The method of any one of embodiments 1-10, wherein the first Fc polypeptide linked to the IDS amino acid sequence comprises SEQ ID NO: 175; and the second Fc polypeptide comprises SEQ ID NO: 92.

Embodiment 22. The method of any one of embodiments 1-10, wherein the first Fc polypeptide linked to the IDS amino acid sequence comprises SEQ ID NO: 217; and the second Fc polypeptide comprises SEQ ID NO: 189.

Embodiment 23. The method of any one of embodiments 1-10, wherein the first Fc polypeptide linked to the IDS amino acid sequence comprises SEQ ID NO: 175; and the second Fc polypeptide comprises SEQ ID NO: 95.

Embodiment 24. The method of embodiment 23, wherein the second Fc polypeptide further comprises SEQ ID NO: 89, wherein SEQ ID NO: 89 is attached to the N-terminus of SEQ ID NO:95.

Embodiment 25. The method of any one of embodiments 1-10, wherein the first Fc polypeptide linked to the IDS amino acid sequence comprises SEQ ID NO: 217; and the second Fc polypeptide comprises SEQ ID NO: 191.

Embodiment 26. The method of embodiment 25, wherein the second Fc polypeptide further comprises SEQ ID NO: 89, wherein SEQ ID NO: 89 is attached to the N-terminus of SEQ ID NO:191.

Embodiment 27. The method of any one of embodiments 1-10, wherein the first Fc polypeptide linked to the IDS amino acid sequence comprises SEQ ID NO: 175; and the second Fc polypeptide comprises SEQ ID NO: 168.

Embodiment 28. The method of any one of embodiments 1-10, wherein the first Fc polypeptide linked to the IDS amino acid sequence comprises SEQ ID NO: 217; and the second Fc polypeptide comprises SEQ ID NO: 207.

Embodiment 29. The method of any one of embodiments 1-10, wherein the first Fc polypeptide linked to the IDS amino acid sequence comprises SEQ ID NO: 175; and the second Fc polypeptide comprises SEQ ID NO: 169.

Embodiment 30. The method of any one of embodiments 1-10, wherein the first Fc polypeptide linked to the IDS amino acid sequence comprises SEQ ID NO: 217; and the second Fc polypeptide comprises SEQ ID NO: 199.

Embodiment 31. The method of any one of embodiments 1-30 or 112-150, wherein the administration of the pharmaceutical composition reduces levels of one or more analytes in the CSF of the subject to baseline levels measured in a healthy subject or in a subject that does not have Hunter syndrome, wherein the one or more analytes are selected from the group consisting of a glycosaminoglycan (GAG), neurofilament light (Nf-L), soluble triggering receptor expressed on myeloid cells 2 (sTREM2), a bis(monoacylglycerol) phosphate (BMP), a ganglioside and a sphingolipid.

Embodiment 32. The method of embodiment 31, wherein the administration of the pharmaceutical composition reduces levels of a GAG in the CSF of the subject to baseline levels.

Embodiment 33. The method of embodiment 31 or 32, wherein the administration of the pharmaceutical composition reduces levels of Nf-L in the CSF of the subject to baseline levels.

Embodiment 34. The method of any one of embodiments 31-33, wherein the administration of the pharmaceutical composition reduces levels of sTREM2 in the CSF of the subject to baseline levels.

Embodiment 35. The method of any one of embodiments 31-34, wherein the administration of the pharmaceutical composition reduces levels of a BMP in the CSF of the subject to baseline levels.

Embodiment 36. The method of any one of embodiments 31-35, wherein the administration of the pharmaceutical composition reduces levels of a ganglioside in the CSF of the subject to baseline levels.

Embodiment 37. The method of any one of embodiments 31-36, wherein the administration of the pharmaceutical composition reduces levels of a sphingolipid in the CSF of the subject to baseline levels.

Embodiment 38. The method of any one of embodiments 1-30 or 112-150, wherein the administration of the pharmaceutical composition reduces levels of one or more analytes in the CSF of the subject by at least about 10%, 15%, 20%, 25%, or 30%, wherein the reduction is relative to the level of the corresponding one or more analytes in the subject prior to the administration, wherein the one or more analytes are selected from the group consisting of neurofilament light (Nf-L), soluble triggering receptor expressed on myeloid cells 2 (sTREM2), a bis(monoacylglycerol) phosphate (BMP), a ganglioside, and a sphingolipid.

Embodiment 39. The method of embodiment 38, wherein the administration of the pharmaceutical composition reduces levels of the one or more analytes in the CSF of the subject to baseline levels, measured in a healthy subject or in a subject that does not have Hunter syndrome.

Embodiment 40. The method of any one of embodiments 1-39 or 112-150, wherein the administration of the pharmaceutical composition reduces levels of one or more analytes in the serum of the subject by at least about 30%, 40%, 50%, 60%, or 70% relative to the level of the corresponding one or more analytes in the serum of the subject prior to the administration, wherein the one or more analytes are selected from the group consisting of a GAG and Nf-L.

Embodiment 41. The method of embodiment 40, wherein the administration of the pharmaceutical composition reduces levels of a GAG in the serum of the subject to baseline levels (e.g., as measured in a healthy subject or in a subject that does not have Hunter syndrome).

Embodiment 42. The method of embodiment 40 or 41, wherein the administration of the pharmaceutical composition reduces levels of Nf-L in the serum of the subject to baseline levels (e.g., as measured in a healthy subject or in a subject that does not have Hunter syndrome).

Embodiment 43. The method of any one of embodiments 1-42 or 112-150, wherein the administration of the pharmaceutical composition reduces levels of a GAG in the urine of the subject by at least about 30%, 40%, 50%, 60%, or 70% relative to the level of the GAG in the urine of the subject prior to the administration.

Embodiment 44. The method of embodiment 43, wherein the administration of the pharmaceutical composition reduces levels of a GAG in the urine of the subject to baseline levels.

Embodiment 45. The method of any one of embodiments 1-44 or 112-150, wherein the administration of the pharmaceutical composition improves or maintains the subject's toileting abilities percentage (TAP) relative to a baseline level measured for the subject prior to administration of the pharmaceutical composition.

Embodiment 46. The method of any one of embodiments 1-45 or 112-150, wherein the administration of the pharmaceutical composition improves or maintains the subject's qualitative individualized educational plan (IEP) trajectories in cognitive categories or behavioral categories relative to a baseline level measured for the subject prior to administration of the pharmaceutical composition.

Embodiment 47. The method of any one of embodiments 1-46 or 112-150, wherein the administration of the pharmaceutical composition improves or maintains the subject's hearing relative to a baseline level measured for the subject prior to administration of the pharmaceutical composition, as assessed by an auditory brainstem response (ABR) assessment or a standard hearing test.

Embodiment 48. The method of any one of embodiments 1-47 or 112-150, wherein the administration of the pharmaceutical composition changes the subject's liver volume relative to a baseline level measured for the subject prior to administration of the pharmaceutical composition, as assessed by MRI or ultrasound.

Embodiment 49. The method of any one of embodiments 1-48 or 112-150, wherein the administration of the pharmaceutical composition changes the subject's spleen volume relative to a baseline level measured for the subject prior to administration of the pharmaceutical composition, as assessed by MRI or ultrasound.

Embodiment 50. The method of any one of embodiments 1-49 or 112-150, wherein the subject is a male subject.

Embodiment 51. The method of any one of embodiments 1-50 or 112-150, wherein the subject is from about 2 to 18 years of age.

Embodiment 52. The method of any one of embodiments 1-50 or 112-150, wherein the subject is from about 2 to 10 years of age.

Embodiment 53. The method of any one of embodiments 1-50 or 112-150, wherein the subject is from about 5 to 10 years of age.

Embodiment 54. The method of any one of embodiments 1-50 or 112-150, wherein the subject is less than 4 years of age.

Embodiment 55. The method of any one of embodiments 1-54 or 112-150, wherein the subject has a weight of ≥15 kg.

Embodiment 56. The method of any one of embodiments 1-54 or 112-150, wherein the subject has a weight of ≥19 kg.

Embodiment 57. The method of any one of embodiments 1-56 or 112-150, wherein the subject has a development quotient (DQ)<85.

Embodiment 58. The method of any one of embodiments 1-57 or 112-150, wherein the subject has a decline of at least 10 points in development quotient (DQ) within a period of about 6 months or longer.

Embodiment 59. The method of any one of embodiments 1-58 or 112-150, wherein the subject has a same genetic mutation in the IDS gene as a blood relative with confirmed neuronopathic Hunter syndrome (nMPS II).

Embodiment 60. The method of any one of embodiments 1-58 or 112-150, wherein the subject has neuronopathic Hunter syndrome (nMPS II) or has a same genetic mutation in the IDS gene as a blood relative with confirmed neuronopathic Hunter syndrome (nMPS II).

Embodiment 61. The method of any one of embodiments 1-60 or 112-150, wherein the pharmaceutical composition is administered to the subject intravenously.

Embodiment 62. The method of embodiment 61, wherein the subject does not experience an infusion-related reaction (IRR) upon administration of the pharmaceutical composition.

Embodiment 63. The method of embodiment 62, wherein IRR is an allergic reaction.

Embodiment 64. The method of embodiment 62, wherein IRR is anaphylaxis.

Embodiment 65. The method of any one of embodiments 62-64, wherein the pharmaceutical composition is administered to the subject without pretreatment or co-administration of medication for an infusion-related reaction (IRR).

Embodiment 66. The method of embodiment 61, wherein the pharmaceutical composition is administered to the subject with pretreatment or co-administration of medication for an infusion-related reaction (IRR).

Embodiment 67. The method of embodiment 65 or 66, wherein the medication for infusion-related reaction is one or more selected from the group consisting of: an anti-histamine, an anti-pyretic, and a corticosteroid.

Embodiment 68. The method of any one of embodiments 1-67 or 112-150, wherein the urine total GAG concentration (normalized to creatinine) increases by less than 20% relative to a baseline level after administration of the pharmaceutical composition, wherein the baseline level is measured for the subject prior to administration of the pharmaceutical composition.

Embodiment 69. The method of any one of embodiments 1-67 or 112-150, wherein the urine total GAG concentration (normalized to creatinine) does not increase relative to a baseline level after administration of the pharmaceutical composition, wherein the baseline level is measured for the subject prior to administration of the pharmaceutical composition.

Embodiment 70. The method of any one of embodiments 1-67 or 112-150, wherein the urine total GAG concentration (normalized to creatinine) decreases relative to a baseline level after administration of the pharmaceutical composition, wherein the baseline level is measured for the subject prior to administration of the pharmaceutical composition.

Embodiment 71. The method of any one of embodiments 69 or 70, wherein the stabilization or decrease in urine total GAG concentration in the subject supports safety of the administered dose.

Embodiment 72. The method of any one of embodiments 1-71 or 112-150, wherein the incidence of anti-drug antibodies (ADAs) does not increase relative to a baseline level after administration of the pharmaceutical composition, wherein the baseline level is measured for the subject prior to administration of the pharmaceutical composition.

Embodiment 73. The method of any one of embodiments 1-71 or 112-150, wherein the incidence of anti-drug antibodies (ADAs) increases by less than 10% relative to a baseline level after administration of the pharmaceutical composition, wherein the baseline level is measured for the subject prior to administration of the pharmaceutical composition.

Embodiment 74. The method of any one of embodiments 1-73 or 112-150, wherein the frequency of serious adverse events (SAEs) is less than about 1/1000.

Embodiment 75. The method of any one of embodiments 1-73 or 112-150, wherein the incidence of severe SAEs is less than about 1/1000.

Embodiment 76. The method of any one of embodiments 1-75 or 112-150, wherein the therapeutically effective dose comprises an amount of the protein that is activity equivalent to a standard of care dose for recombinant idursulfase.

Embodiment 77. The method of any one of embodiments 1-75 or 112-150, wherein the therapeutically effective dose comprises an amount of the protein that is activity equivalent to a standard of care dose for recombinant idursulfase-beta.

Embodiment 78. The method of any one of embodiments 1-77 or 112-150, wherein the administration of the pharmaceutical composition reduces levels of a glycosaminoglycan (GAG) in the CSF of the subject by at least 50%, 60%, 70%, 75%, 80%, 85%, or 90% after at least 4 weekly doses, wherein the reduction is relative to the CSF levels of the GAG in the subject prior to administration.

Embodiment 79. The method of embodiment 78, wherein the administration of the pharmaceutical composition reduces levels of the glycosaminoglycan (GAG) in the CSF of the subject by at least 50%, 60%, 70%, 75%, 80%, 85%, or 90% after 4 weekly doses.

Embodiment 80. The method of any one of embodiments 1-77 or 112-150, wherein the administration of the pharmaceutical composition reduces levels of a glycosaminoglycan (GAG) in the CSF of the subject by at least 50%, 60%, 70%, 75%, 80%, 85%, or 90% after at least 8 weekly doses, wherein the reduction is relative to the CSF levels of the GAG in the subject prior to administration.

Embodiment 81. The method of embodiment 80, wherein the administration of the pharmaceutical composition reduces levels of the glycosaminoglycan (GAG) in the CSF of the subject by at least 50%, 60%, 70%, 75%, 80%, 85%, or 90% after 8 weekly doses.

Embodiment 82. The method of any one of embodiments 1-77 or 112-150, wherein the administration of the pharmaceutical composition reduces levels of a glycosaminoglycan (GAG) in the CSF of the subject by at least 50%, 60%, 70%, 75%, 80%, 85%, or 90% after at least 12 weekly doses, wherein the reduction is relative to the CSF levels of the GAG in the subject prior to administration.

Embodiment 83. The method of embodiment 82, wherein the administration of the pharmaceutical composition reduces levels of the glycosaminoglycan (GAG) in the CSF of the subject by at least 50%, 60%, 70%, 75%, 80%, 85%, or 90% after 12 weekly doses.

Embodiment 84. The method of any one of embodiments 78-83, wherein administration of the pharmaceutical composition reduces levels of the glycosaminoglycan (GAG) in the CSF of the subject to a baseline level measured in a healthy subject or a subject that does not have Hunter syndrome.

Embodiment 85. The method of any one of embodiments 1-84 or 112-150, wherein the administration of the pharmaceutical composition reduces levels of a glycosaminoglycan (GAG) in the urine of the subject by at least 50%, 60%, 70%, 75%, 80%, 85%, or 90% after at least 4 weekly doses, wherein the reduction is relative to the urine levels of the GAG in the subject prior to administration.

Embodiment 86. The method of embodiment 85, wherein the administration of the pharmaceutical composition reduces levels of the glycosaminoglycan (GAG) in the urine of the subject by at least 50%, 60%, 70%, 75%, 80%, 85%, or 90% after 4 weekly doses.

Embodiment 87. The method of any one of embodiments 1-84 or 112-150, wherein the administration of the pharmaceutical composition reduces levels of a glycosaminoglycan (GAG) in the urine of the subject by at least 50%, 60%, 70%, 75%, 80%, 85%, or 90% after at least 8 weekly doses, wherein the reduction is relative to the urine levels of the GAG in the subject prior to administration.

Embodiment 88. The method of embodiment 87, wherein the administration of the pharmaceutical composition reduces levels of the glycosaminoglycan (GAG) in the urine of the subject by at least 50%, 60%, 70%, 75%, 80%, 85%, or 90% after 8 weekly doses.

Embodiment 89. The method of any one of embodiments 85-88, wherein administration of the pharmaceutical composition reduces levels of the glycosaminoglycan (GAG) in the urine of the subject to a baseline level measured in a healthy subject or a subject that does not have Hunter syndrome.

Embodiment 90. The method of any one of embodiments 78-89, wherein the GAG is heparan sulfate.

Embodiment 91. The method of embodiment 90, wherein administration of the pharmaceutical composition reduces levels heparan sulfate in the CSF of the subject by at least 80% after at least 4 weekly doses, wherein the reduction is relative to the CSF heparan sulfate levels in the subject prior to administration.

Embodiments 92. The method of embodiment 90, wherein administration of the pharmaceutical composition reduces levels heparan sulfate in the CSF of the subject by at least 70% after at least 12 weekly doses, wherein the reduction is relative to the CSF heparan sulfate levels in the subject prior to administration.

Embodiment 93. The method of any one of embodiments 78-89, wherein the GAG is dermatan sulfate.

Embodiment 94. The method of any one of embodiments 1-93 or 112-150, wherein the administration of the pharmaceutical composition reduces levels of a ganglioside in the CSF of the subject by at least 10%, 15%, 20%, 25%, 30%, 40%, 50%, 60%, 70%, 80%, or 90% after at least 8 weekly doses, wherein the reduction is relative to the CSF levels of the ganglioside in the subject prior to administration.

Embodiment 95. The method of embodiment 94, wherein the administration of the pharmaceutical composition reduces levels of the ganglioside in the CSF of the subject by at least 10%, 15%, 20%, 25%, 30%, 40%, 50%, 60%, 70%, 80%, or 90% after 8 weekly doses.

Embodiment 96. The method of embodiment 94 or 95, wherein the ganglioside is GM3.

Embodiment 97. The method of any one of embodiments 1-96 or 112-150, wherein the administration of the pharmaceutical composition reduces levels of a BMP in the CSF of the subject by at least 10%, 15%, 20%, 25%, 30%, 40%, 50%, 60%, 70%, 80%, or 90% after at least 8 weekly doses, wherein the reduction is relative to the CSF levels of the BMP in the subject prior to administration.

Embodiment 98. The method of embodiment 97, wherein the administration of the pharmaceutical composition reduces levels of the BMP in the CSF of the subject by at least 10%, 15%, 20%, 25%, 30%, 40%, 50%, 60%, 70%, 80%, or 90% after 8 weekly doses.

Embodiment 99. The method of embodiment 97 or 98, wherein the BMP is a BMP di 18:1 species.

Embodiment 100. The method of any one of embodiments 1-99 or 112-150, wherein prior to the administration of the pharmaceutical composition, the subject had received recombinant idursulfase enzyme replacement therapy.

Embodiment 101. The method of any one of embodiments 1-100 or 112-150, wherein the subject has pre-existing anti-drug antibodies against IDS prior to administration of the pharmaceutical composition.

Embodiment 102. The method of embodiment 101, wherein the subject's titer of anti-drug antibodies against IDS ranges from 189 to greater than 11 million.

Embodiment 103. The method of embodiment 102, wherein the subject's titer of anti-drug antibodies against IDS is greater than 11 million.

Embodiment 104. A method of treating Hunter syndrome in a subject in need thereof, comprising administering to the subject a protein at a weekly dose of about 3 mg/kg, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 105. A method of treating Hunter syndrome in a subject in need thereof, comprising administering to the subject a protein at a weekly dose of about 7.5 mg/kg, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 106. A method of treating Hunter syndrome in a subject in need thereof, comprising administering to the subject a protein at a weekly dose of about 15 mg/kg, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 107. A method of treating Hunter syndrome in a subject in need thereof, comprising administering to the subject a protein at a weekly dose of about 30 mg/kg, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 108. The method of any one of embodiments 104-107, wherein the protein is comprised in a pharmaceutical composition that further comprises a pharmaceutically acceptable excipient.

Embodiment 109. A method of resolving an infusion-related reaction (IRR) in a subject receiving treatment for Hunter syndrome, comprising administering one or more agents selected from the group consisting of an anti-histamine, an anti-pyretic, and a corticosteroid, wherein the subject is being administered or was administered a pharmaceutical composition comprising a protein at a dose of at least about 7.5 mg/kg of protein, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 110. The method of embodiment 109, wherein the IRR is a fever.

Embodiment 111. The method of embodiment 109 or 110, wherein method of resolving the IRR comprises administering to the subject acetaminophen and diphenhydramine.

Embodiment 112. A method of treating a neurobehavioral deficit, an auditory deficit and/or a musculoskeletal abnormality in a subject with Hunter syndrome, comprising administering to the subject a therapeutically effective dose of a pharmaceutical composition comprising a protein, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 113. The method of embodiment 112, wherein the therapeutically effective dose comprises an amount of the protein that is activity equivalent to a standard of care dose for recombinant idursulfase.

Embodiment 114. The method of embodiment 112, wherein the therapeutically effective dose comprises an amount of the protein that is activity equivalent to a standard of care dose for recombinant idursulfase-beta.

Embodiment 115. The method of embodiment 112 or 113, wherein the therapeutically effective dose is from about 3 mg/kg to about 30 mg/kg of protein.

Embodiment 116. The method of any one of embodiments 112-115, wherein the pharmaceutical composition further comprises a pharmaceutically acceptable excipient (a pharmaceutically acceptable carrier).

Embodiment 117. The method of any one of embodiments 112-116, wherein the second Fc polypeptide specifically binds to the transferrin receptor.

Embodiment 118. The method of any one of embodiments 112-117, wherein the IDS amino acid sequence comprises an amino acid sequence having at least 90% identity to SEQ ID NO:70.

Embodiment 119. The method of embodiment 118, wherein the IDS amino acid sequence comprises a sequence selected from the group consisting of SEQ ID NOs:70, 90, 170 and 174.

Embodiment 120. The method of any one of embodiments 112-119, wherein the first Fc polypeptide linked to the IDS amino acid sequence comprises the amino acid sequence of SEQ ID NO: 91, 171 or 175.

Embodiment 121. The method of any one of embodiments 112-119, wherein the first Fc polypeptide linked to the IDS amino acid sequence comprises the amino acid sequence of SEQ ID NO: 213, 215, or 217.

Embodiment 122. The method of any one of embodiments 112-121, wherein the IDS amino acid sequence is linked to the N-terminus of the first Fc polypeptide.

Embodiment 123. The method of any one of embodiments 112-122, wherein the second Fc polypeptide comprises:
d. Leu at position 380;
e. Ala at position 389; and
f. Ser at position 390.

Embodiment 124. The method of any one of embodiments 112-122, wherein the second Fc polypeptide comprises:
d. Glu at position 380;
e. Ala at position 389; and
f. Asn at position 390.

Embodiment 125. The method of any one of embodiments 112-122, wherein the second Fc polypeptide comprises:
d. Trp at position 380;
e. Ser at position 389; and
f. Ser at position 390.

Embodiment 126. The method of any one of embodiments 112-122, wherein the second Fc polypeptide comprises:
d. Leu at position 380;
e. Ser at position 389; and
f. Ser at position 390.

Embodiment 127. The method of any one of embodiments 112-117, wherein the first Fc polypeptide linked to the IDS amino acid sequence comprises SEQ ID NO: 175; and the second Fc polypeptide comprises SEQ ID NO: 92.

Embodiment 128. The method of any one of embodiments 112-117, wherein the first Fc polypeptide linked to the IDS amino acid sequence comprises SEQ ID NO: 217; and the second Fc polypeptide comprises SEQ ID NO: 189.

Embodiment 129. The method of any one of embodiments 112-117, wherein the first Fc polypeptide linked to the IDS amino acid sequence comprises SEQ ID NO: 175; and the second Fc polypeptide comprises SEQ ID NO: 95.

Embodiment 130. The method of embodiment 129, wherein the second Fc polypeptide further comprises SEQ ID NO: 89, wherein SEQ ID NO: 89 is attached to the N-terminus of SEQ ID NO:95.

Embodiment 131. The method of any one of embodiments 112-117, wherein the first Fc polypeptide linked to the IDS amino acid sequence comprises SEQ ID NO: 217; and the second Fc polypeptide comprises SEQ ID NO: 191.

Embodiment 132. The method of embodiment 131, wherein the second Fc polypeptide further comprises SEQ ID NO: 89, wherein SEQ ID NO: 89 is attached to the N-terminus of SEQ ID NO:191.

Embodiment 133. The method of any one of embodiments 112-117, wherein the first Fc polypeptide linked to the IDS amino acid sequence comprises SEQ ID NO: 175; and the second Fc polypeptide comprises SEQ ID NO: 168.

Embodiment 134. The method of any one of embodiments 112-117, wherein the first Fc polypeptide linked to the IDS amino acid sequence comprises SEQ ID NO: 217; and the second Fc polypeptide comprises SEQ ID NO: 207.

Embodiment 135. The method of any one of embodiments 112-117, wherein the first Fc polypeptide linked to the IDS amino acid sequence comprises SEQ ID NO: 175; and the second Fc polypeptide comprises SEQ ID NO: 169.

Embodiment 136. The method of any one of embodiments 112-117, wherein the first Fc polypeptide linked to the IDS amino acid sequence comprises SEQ ID NO: 217; and the second Fc polypeptide comprises SEQ ID NO: 199.

Embodiment 137. The method of any one of embodiments 112-136, which is a method of treating a neurobehavioral deficit.

Embodiment 138. The method of embodiment 137, wherein the neurobehavioral deficit is a motor skill deficit.

Embodiment 139. The method of embodiment 138, wherein the motor skill deficit is a fine motor skill deficit.

Embodiment 140. The method of embodiment 138, wherein the motor skill deficit is a gross motor skill deficit.

Embodiment 141. The method of embodiment 137, wherein the neurobehavioral deficit is an agility deficit.

Embodiment 142. The method of embodiment 137, wherein the neurobehavioral deficit is a cognitive deficit.

Embodiment 143. The method of embodiment 142, wherein the cognitive deficit is a learning or memory deficit.

Embodiment 144. The method of embodiment 137, wherein the neurobehavioral deficit is a sensorimotor gating deficit.

Embodiment 145. The method of any one of embodiments 112-136, which is a method of treating an auditory deficit.

Embodiment 146. The method of embodiment 145, wherein administration of the pharmaceutical composition reduces middle ear effusion and/or otitis media in the subject, relative to a baseline level.

Embodiment 147. The method of embodiment 146, wherein the baseline level of a parameter is the parameter level for the subject prior to administration of the pharmaceutical composition.

Embodiment 148. The method of any one of embodiments 112-136, which is a method of treating a musculoskeletal abnormality.

Embodiment 149. The method of embodiment 148, wherein the musculoskeletal abnormality is a skeletal abnormality.

Embodiment 150. The method of any one of embodiments 112-149, wherein the administration of the pharmaceutical composition reduces levels of one or more analytes in the CSF of the subject by at least about 10%, 15%, 20%, 25%, or 30%, wherein the reduction is relative to the level of the corresponding one or more analytes in the subject prior to the administration, wherein the one or more analytes are selected from the group consisting of a glycosaminoglycan (GAG), neurofilament light (Nf-L), soluble triggering receptor expressed on myeloid cells 2 (sTREM2), a bis(monoacylglycerol) phosphate (BMP), a ganglioside, and a sphingolipid.

Embodiment 151. An agent for use in resolving an infusion-related reaction (IRR), the method comprising administering the agent to a subject in need thereof, wherein the agent is selected from the group consisting of an anti-histamine, an anti-pyretic, and a corticosteroid, wherein the subject is being administered or was administered a pharmaceutical composition comprising a protein at a dose of at least about 7.5 mg/kg of protein, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 152. Use of an agent in the preparation of a medicament for resolving an infusion-related reaction (IRR), by administering the medicament to a subject in need thereof, wherein the agent is selected from the group consisting of an anti-histamine, an anti-pyretic, and a corticosteroid, wherein the subject is being administered or was administered a pharmaceutical composition comprising a protein at a dose of at least about 7.5 mg/kg of protein, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 153. A therapeutically effective dose of a pharmaceutical composition comprising a protein for use in treating Hunter syndrome in a subject in need thereof, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 154. The use of a therapeutically effective dose of a pharmaceutical composition comprising a protein in the preparation of a medicament for treating Hunter syndrome, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 155. A therapeutically effective dose of a pharmaceutical composition comprising a protein for use in treating a neurobehavioral deficit, an auditory deficit and/or a musculoskeletal deficit in a subject with Hunter syndrome, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 156. The use of a therapeutically effective dose of a pharmaceutical composition comprising a protein in the preparation of a medicament for treating a neurobehavioral deficit, an auditory deficit and/or a musculoskeletal deficit in a subject with Hunter syndrome, wherein the protein comprises:

a. a first Fc polypeptide linked to an iduronate 2-sulfatase (IDS) amino acid sequence, an IDS variant amino acid sequence, or a catalytically active fragment thereof, and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 157. A pharmaceutical composition comprising a protein for use in a method of treating Hunter syndrome, the method comprising administering a therapeutically effective dose of the pharmaceutical composition to a subject in need thereof, wherein administration of the pharmaceutical composition reduces levels of a glycosaminoglycan (GAG) in the CSF of the subject to a baseline level measured in a healthy subject or a subject that does not have Hunter syndrome, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 158. Use of a protein in the preparation of a medicament for treating Hunter syndrome, by administering a therapeutically effective dose of the medicament to a subject in need thereof, wherein administration of the pharmaceutical composition reduces levels of a glycosaminoglycan (GAG) in the CSF of the subject to a baseline level measured in a healthy subject or a subject that does not have Hunter syndrome, and wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 159. The pharmaceutical composition of embodiment 157 or the use of embodiment 158, wherein the therapeutically effective dose is from about 3 mg/kg to about 30 mg/kg of protein.

Embodiment 160. A pharmaceutical composition comprising a protein for use in a method of treating a neurobehavioral deficit, an auditory deficit and/or a musculoskeletal deficit associated with Hunter syndrome, the method comprising administering a therapeutically effective dose of the pharmaceutical composition to the subject, wherein the protein comprises:

a. a first Fc polypeptide linked to an iduronate 2-sulfatase (IDS) amino acid sequence, an IDS variant amino acid sequence, or a catalytically active fragment thereof; and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 161. Use of a protein in the preparation of a medicament for treating a neurobehavioral deficit, an auditory deficit and/or a musculoskeletal deficit associated with Hunter syndrome, by administering a therapeutically effective dose of the medicament to a subject in need thereof, wherein the protein comprises:

a. a first Fc polypeptide linked to an iduronate 2-sulfatase (IDS) amino acid sequence, an IDS variant amino acid sequence, or a catalytically active fragment thereof, and

b. a second Fc polypeptide comprising the following amino acid residues, according to EU numbering: Trp, Leu, or Glu at position 380; Tyr at position 384; Thr at position 386; Glu at position 387; Trp at position 388; Ser or Ala at position 389; Ser or Asn at position 390; Thr at position 413; Glu at position 415; Glu at position 416; and Phe at position 421.

Embodiment 162. The pharmaceutical composition of embodiment 160 or the use of embodiment 161, wherein the therapeutically effective dose is from about 3 mg/kg to about 30 mg/kg of protein.

### Certain Definitions

The terms “control” or “control sample” refer to any sample appropriate to the detection technique employed. The control sample may contain the products of the detection technique employed or the material to be tested. Further, the controls may be positive or negative controls.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The term “pharmaceutically acceptable excipient” refers to a non-active pharmaceutical ingredient that is biologically or pharmacologically compatible for use in humans or animals, such as but not limited to a buffer, carrier, or preservative.

The term “administer” refers to a method of delivering agents (e.g., an LSD therapeutic agent, such as an ETV therapy described herein), compounds, or compositions (e.g, pharmaceutical composition) to the desired site of biological action. These methods include, but are not limited to, oral, topical delivery, parenteral delivery, intravenous delivery, intradermal delivery, intramuscular delivery, intrathecal delivery, colonic delivery, rectal delivery, or intraperitoneal delivery. In one embodiment, the polypeptides described herein are administered intravenously.

As used herein, “treatment” (and grammatical variations thereof such as “treat” or “treating”) refers to clinical intervention to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

A “therapeutically effective amount” of a substance/molecule disclosed herein may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The terms “obtaining a sample from a patient”, “obtained from a patient” and similar phrasing, is used to refer to obtaining the sample directly from the patient, as well as obtaining the sample indirectly from the patient through an intermediary individual (e.g., obtaining the sample from a courier who obtained the sample from a nurse who obtained the sample from the patient).

An “iduronate sulfatase,” “iduronate-2-sulfatase,” or “IDS” as used herein refers to iduronate 2-sulfatase (EC 3.1.6.13), which is an enzyme involved in the lysosomal degradation of the glycosaminoglycans heparan sulfate and dermatan sulfate. Deficiency of IDS is associated with Mucopolysaccharidosis II, also known as Hunter syndrome. The term “IDS” as used herein as a component of a protein that comprises an Fc polypeptide is catalytically active and encompasses functional variants, including allelic and splice variants, of a wild-type IDS or a fragment thereof. The sequence of human IDS isoform I, which is the human sequence designated as the canonical sequence, is available under UniProt entry P22304 and is encoded by the human IDS gene at Xq28. The full-length sequence is provided as SEQ ID NO:69. A “mature” IDS sequence as used herein refers to a form of a polypeptide chain that lacks the signal and propeptide sequences of the naturally occurring full-length polypeptide chain. The amino acid sequence of a mature human IDS polypeptide is provided as SEQ ID NO:70, which corresponds to amino acids 34-550 of the full-length human sequence. A “truncated” IDS sequence as used herein refers to a catalytically active fragment of the naturally occurring full-length polypeptide chain. The amino acid sequence of an exemplary truncated human IDS polypeptide is provided as SEQ ID NO:90, which corresponds to amino acids 26-550 of the full-length human sequence. The structure of human IDS has been well-characterized. An illustrative structure is available under PDB accession code 5FQL. The structure is also described in Nat. Comm. 8:15786 doi: 10.1038/ncomms15786, 2017. Non-human primate IDS sequences have also been described, including chimpanzee (UniProt entry K7BKV4) and rhesus macaque (UniProt entry H9FTX2). A mouse IDS sequence is available under Uniprot entry Q08890. An IDS variant has at least 50%, at least 55%, at least 60%, at least 65%, at least 70%, at least 75%, at least 80%, at least 85%, at least 90%, or at least 95% of the activity of the corresponding wild-type IDS or fragment thereof, e.g., when assayed under identical conditions. A catalytically active IDS fragment has at least 50%, at least 55%, at least 60%, at least 65%, at least 70%, at least 75%, at least 80%, at least 85%, at least 90%, or at least 95% of the activity of the corresponding full-length IDS or variant thereof, e.g., when assayed under identical conditions.

An “IDS enzyme variant” refers to a functional variant, including allelic and splice variants, of a wild-type IDS enzyme or a fragment thereof, where the IDS enzyme variant has at least 50%, at least 55%, at least 60%, at least 65%, at least 70%, at least 75%, at least 80%, at least 85%, at least 90%, or at least 95% of the activity of the corresponding wild-type IDS enzyme or fragment thereof, e.g., when assayed under identical conditions.

A “transferrin receptor” or “TfR” as used herein refers to transferrin receptor protein 1. The human transferrin receptor 1 polypeptide sequence is set forth in SEQ ID NO:72. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:178.

A “fusion protein” or “[IDS enzyme]-Fc fusion protein” as used herein refers to a dimeric protein comprising a first Fc polypeptide that is linked (e.g., fused) to an IDS enzyme, an IDS enzyme variant, or a catalytically active fragment thereof (i.e., an “[IDS]-Fc fusion polypeptide”); and a second Fc polypeptide that forms an Fe dimer with the first Fc polypeptide. The second Fc polypeptide may also be linked (e.g., fused) to an IDS enzyme, an IDS enzyme variant, or a catalytically active fragment thereof. The first Fc polypeptide and/or the second Fc polypeptide may be linked to the IDS enzyme, IDS enzyme variant, or catalytically active fragment thereof by a peptide bond or by a polypeptide linker. The first Fc polypeptide and/or the second Fc polypeptide may be a modified Fc polypeptide that contains one or more modifications that promote its heterodimerization to the other Fc polypeptide. The first Fc polypeptide and/or the second Fc polypeptide may be a modified Fc polypeptide that contains one or more modifications that confer binding to a transferrin receptor. The first Fc polypeptide and/or the second Fc polypeptide may be a modified Fc polypeptide that contains one or more modifications that reduce effector function. The first Fc polypeptide and/or the second Fc polypeptide may be a modified Fc polypeptide that contains one or more modifications that extend serum half-life.

A “fusion polypeptide” or “[IDS enzyme]-Fc fusion polypeptide” as used herein refers to an Fc polypeptide that is linked (e.g., fused) to an IDS enzyme, an IDS enzyme variant, or a catalytically active fragment thereof. The Fc polypeptide may be linked to the IDS enzyme, IDS enzyme variant, or catalytically active fragment thereof by a peptide bond or by a polypeptide linker. The Fc polypeptide may be a modified Fc polypeptide that contains one or more modifications that promote its heterodimerization to another Fc polypeptide. The Fe polypeptide may be a modified Fc polypeptide that contains one or more modifications that confer binding to a transferrin receptor. The Fc polypeptide may be a modified Fc polypeptide that contains one or more modifications that reduce effector function. The Fc polypeptide may be a modified Fc polypeptide that contains one or more modifications that extend serum half-life.

As used herein, the term “Fe polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a 02-microglobulin of about 15 kDa in size.

As used herein, an “FcRn binding site” refers to the region of an Fc polypeptide that binds to FcRn. In human IgG, the FcRn binding site, as numbered using the EU index, includes T250, L251, M252, 1253, S254, R255, T256, T307, E380, M428, H433, N434, H435, and Y436.

These positions correspond to positions 20 to 26, 77, 150, 198, and 203 to 206 of SEQ ID NO:1.

As used herein, a “native FcRn binding site” refers to a region of an Fc polypeptide that binds to FcRn and that has the same amino acid sequence as the region of a naturally occurring Fc polypeptide that binds to FcRn.

The terms “CH3 domain” and “CH2 domain” as used herein refer to immunoglobulin constant region domain polypeptides. For purposes of this application, a CH3 domain polypeptide refers to the segment of amino acids from about position 341 to about position 447 as numbered according to EU, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:71).

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation. For example, the terms “wild-type,” “native,” and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO:1. The polypeptide that is aligned to the reference sequence need not be the same length as the reference sequence.

The term “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

A “binding affinity” as used herein refers to the strength of the non-covalent interaction between two molecules, e.g., a single binding site on a polypeptide and a target, e.g., transferrin receptor, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

As used herein, the term “specifically binds” or “selectively binds” to a target, e.g., TfR, when referring to an engineered TfR-binding polypeptide, TfR-binding peptide, or TfR-binding antibody as described herein, refers to a binding reaction whereby the engineered TfR-binding polypeptide, TfR-binding peptide, or TfR-binding antibody binds to the target with greater affinity, greater avidity, and/or greater duration than it binds to a structurally different target. In typical embodiments, the engineered TfR-binding polypeptide, TfR-binding peptide, or TfR-binding antibody has at least 5-fold, 10-fold, 50-fold, 100-fold, 1,000-fold, 10,000-fold, or greater affinity for a specific target, e.g., TfR, compared to an unrelated target when assayed under the same affinity assay conditions. The term “specific binding,” “specifically binds to,” or “is specific for” a particular target (e.g., TfR), as used herein, can be exhibited, for example, by a molecule having an equilibrium dissociation constant K_{D} for the target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, an engineered TfR-binding polypeptide, TfR-binding peptide, or TfR-binding antibody specifically binds to an epitope on TfR that is conserved among species, (e.g., structurally conserved among species), e.g., conserved between non-human primate and human species (e.g., structurally conserved between non-human primate and human species). In some embodiments, an engineered TfR-binding polypeptide, TfR-binding peptide, or TfR-binding antibody may bind exclusively to a human TfR.

The term “variable region” or “variable domain” refers to a domain in an antibody heavy chain or light chain that is derived from a germline Variable (V) gene, Diversity (D) gene, or Joining (J) gene (and not derived from a Constant (C and CS) gene segment), and that gives an antibody its specificity for binding to an antigen. Typically, an antibody variable region comprises four conserved “framework” regions interspersed with three hypervariable “complementarity determining regions.”

The terms “antigen-binding portion” and “antigen-binding fragment” are used interchangeably herein and refer to one or more fragments of an antibody that retains the ability to specifically bind to an antigen via its variable region. Examples of antigen-binding fragments include, but are not limited to, a Fab fragment (a monovalent fragment consisting of the VL, VH, CL, and CH1 domains), a F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), a VL (light chain variable region), and a VH (heavy chain variable region).

The following Examples are intended to be non-limiting.

### Example 1: Effect of Peripheral Administration of ETV:IDS on Brain GAG and Lysosomal Lipids in IDS KO×TfR^{muhu} Mice

As described below, the effects of peripheral administration of ETV:IDS on brain GAG and lysosomal lipids in IDS KO×TfR^{muhu} mice was investigated.

### Materials and Methods

### Animal Care

Mice were housed under a 12-hour light/dark cycle and had access to water and standard rodent diet (LabDiet® #25502, Irradiated) ad libitum.

### Mouse Strains

A previously described, IDS KO mice on a B6N background were obtained from The Jackson Laboratories (JAX strain 024744). Development and characterization of the TfR^{mu/hu} KI mouse line harboring the human TfR apical domain knocked into the mouse receptor was previously described (U.S. Pat. No. 10,143,187). TfR^{mu/hu} male mice were bred to female IDS heterozygous mice to generate IDS KO×TfR^{mu/hu} mice. All mice used in this study were males.

### Administration and Tissue Collection

2 month old IDS KO×TfR^{mu/hu} mice were injected i.v. with idursulfase (14.2 mg/kg body weight), or ETV:IDS (40 mg/kg body weight) once every week for 4 weeks (n=8) (see, Example 6 for ETV:IDS). 2 month-old littermate TfR^{mu/hu} mice, injected i.v. with saline once every week for 4 weeks (n=5) were used as controls. For the 7-day cohort, animals were sacrificed 7 days following the first. For the 28-day cohort, animals were sacrificed 7 days following fourth weekly dose.

For terminal sample collection, animals were deeply anesthetized via intraperitoneal (i.p.) injection of 2.5% Avertin. For CSF collection, a sagittal incision was made at the back of the animal's skull, subcutaneous tissue and muscle was separated to expose the cisterna magna and a pre-pulled glass capillary tube was used to puncture the cisterna magna to collect CSF. CSF was transferred to a Low Protein LoBind Eppendorf tube and centrifuged at 12,700 rpm for 10 minutes at 4° C. CSF was transferred to a fresh tube and snap frozen on dry ice. Lack of blood contamination in mouse CSF was confirmed by measuring the absorbance of the samples at 420 nm. Blood was collected via cardiac puncture for serum collection. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice. Animals were transcardially perfused with ice-cold PBS using a peristaltic pump (Gilson Inc. Minipuls Evolution). The brain and liver were dissected and flash-frozen on dry ice.

### Tissue Preparation: GAG Extraction and Processing

Brain and liver tissue samples and CSF were collected as described above. Brain and liver tissue were homogenized using a TissueLyser from Qiagen. Tissue homogenate was then transferred to a 96-well deep plate and sonicated using a 96-tip sonicator (Q Sonica). A BCA protein assay was performed to quantify total protein, and 20 μg liver and 100 μg brain per sample were subjected to LC-MS/MS sample preparation. 3 μL was used per sample for CSF. Briefly, heparan and dermatan sulfate derived disaccharides were generated by digesting tissue homogenates using a combination of Heparinases I, II, III and Chondriotinase B. Digests were mixed with acetonitrile and subjected to LC-MS/MS as described below.

### LCMS Assay for GAG

Quantification of GAG was performed by liquid chromatography (Shimadzu Nexera X2 system, Shimadzu Scientific Instrument, Columbia, Md., USA) coupled to electrospray mass spectrometry (Sciex QTRAP 6500+, Sciex, Framingham, Mass., USA). For each analysis, sample was injected on a ACQUITY UPLC BEH Amide 1.7 mm, 2.1×150 mm column (Waters Corporation) using a flow rate of 0.55 mL/minute with a column temperature of 55° C. Mobile phases A and B consisted of water with 10 mM ammonium formate and 0.1% formic acid, and acetonitrile with 0.1% formic acid, respectively. A gradient was programmed as follows: 0.0-0.5 minutes at 80% B, 0.5-3.5 minutes from 80% B to 50% B, 3.5-4.0 minutes 50% B to 80% B, 4.0-4.5 minutes hold at 80% B. Electrospray ionization was performed in the negative-ion mode applying the following settings: curtain gas at 25; collision gas was set at medium; ion spray voltage at −4500; temperature at 600° C.; ion source Gas 1 at 50; ion source Gas 2 at 60. Data acquisition was performed using Analyst 1.6.3 (Sciex) in multiple reaction monitoring mode (MRM), with dwell time 50 (msec) for each species. collision energy (CE) was set at −30; declustering potential (DP) at −80; entrance potential (EP) at −10; collision cell exit potential (CXP) at −10. GAGs were detected as [M-H]− using the following MRM transitions: D0A0 at m/z 378.1>87.0; D0S0 at m/z 416.1>138.0; D0a4 at m/z 458.1>300.0; D4UA-2S-GlcNCOEt-6S (Iduron Ltd, Manchester, UK) at m/z 472.0 (in source fragment ion)>97.0 was used as internal standard. Individual disaccharide species were identified based on their retention times and MRM transitions using commercially available reference standards (Iduron Ltd). GAGs were quantified by the peak area ratio of D0A0, D0S0 and D0a4 to the internal standard using MultiQuant 3.0.2 (Sciex). Reported GAG amounts were normalized to total protein levels as measured by a BCA assay (Pierce).

Tissue Preparation: Lipid Extraction from Brain Tissue

Frozen brain tissues (20±2 mg) were transferred into dry ice 2 mL Safe-Lock —Eppendorf tube (Eppendorf Cat #022600044) and kept in dry ice containing a 5 mm stainless steel bead (QIAGEN Cat #69989) and 400 μl of MS-grade methanol containing internal standards. The tissues were homogenized with Tissuelyser for 30 sec at 25 Hz (in the cold room). The samples were then centrifuged for 20 min at 21,000×g at 4° C. The methanol supernatant was transferred into new eppendorf vials and were left at −20° C. for 1 hour to allow for further precipitation of proteins. The samples were then centrifuged for 10 min at 21,000×g at 4° C. 200 uL of the methanol supernatant was transferred into a LCMS 96 well-plate and dry down under nitrogen and then resuspended in 100 uL of ACN/IPA/H2O (92.5/5/2.5) with 5 mM ammonium formate and 0.5% formic acid for GlcCer analysis. The rest of the supernatant was transferred, without disturbing pellet, into a separate LCMS 96 well-plate for analysis of BMP and ganglioside species. The samples were either directly run on LCMS or stored at −80° C.

### LGS Assay for BMP and Gangliosides

BMP and gangliosides analyses were performed by liquid chromatography (Shimadzu Nexera X2 system, Shimadzu Scientific Instrument, Columbia, Md., USA) coupled to electrospray mass spectrometry (Sciex QTRAP 6500+, Sciex, Framingham, Mass., USA). For each analysis, 5 μL of sample was injected on a BEH C18 1.7 μm, 2.1×100 mm column (Waters Corporation Milford, Mass., USA) using a flow rate of 0.25 mL/mirin at 55° C. Mobile phase A consisted of 60:40 acetonitrile/water (v/v) with 10 mM ammonium acetate Mobile phase B consisted of 90:10 isopropyl alcohol/acetonitrile (v/v) with 10 mM ammonium acetate. The gradient was programmed as follows: 0.0-0.01 min from 45% 13 to 99% 13, 0.1-3.0 min at 99% B, 3.0-3.01 min to 45% B, and 3.01-3.50 min at 45% B. Electrospray ionization was performed in negative ion mode applying the following settings: curtain gas at 30; collision gas was set at medium; ion spray voltage at −4500; temperature at 600° C.; ion source Gas 1 at 50; ion source Gas 2 at 60. Data acquisition was performed using Analyst 1.63 (Sciex) in multiple reaction monitoring mode (MRM), with the following parameters: dwell time (msec) for each species reported in the Table A, collision energy (CE) at −50, declustering potential (DP) at −80; entrance potential (EP) at −10; and collision cell exit (CXP) potential at −15. BMP and gangliosides species were quantified using the non endogenous internal standards BMP di14:0 and GM3 (d36:1 (d5)). Quantification was performed using MultiQuant 3.02 (Sciex). BMP and gangliosides concentration were normalized to either total protein amount, tissue weight or volume. Protein concentration was measured using the bicinchoninic acid (BCA) assay (Pierce, Rockford, Ill., USA).

TABLE A
Acquisition parameters information for the BMP and gangliosides assay.

| | | | Q1 | Q2 | Time | | |
|---|---|---|---|---|---|---|---|
| Lipid | Internal standard | Group | mass | mass | (msec) | DP | CE |
| BMP (14:0/14:0) | Internal standard | BMP | 665.3 | 227.2 | 20 | −80 | −50 |
| BMP (20:4/20:4) | BMP (14:0/14:0) | BMP | 817.5 | 303.3 | 200 | −80 | −50 |
| BMP (22:6/22:6) | BMP (14:0/14:0) | BMP | 865.5 | 327.3 | 800 | −80 | −50 |
| BMP (18:1/18:1) | BMP (14:0/14:0) | BMP | 773.5 | 281.3 | 50 | −80 | −50 |
| GM3(d18:1/18:0(d5)) | Internal standard | GM | 1184.8 | 290.1 | 10 | −60 | −65 |
| GM3(d34:1) | GM3(d18:1/18:0(d5)) | GM3 | 1151.7 | 290.1 | 10 | −60 | −65 |
| GM3(d36:1) | GM3(d18:1/18:0(d5)) | GM3 | 1179.8 | 290.1 | 10 | −60 | −65 |
| GM3(d38:1) | GM3(d18:1/18:0(d5)) | GM3 | 1207.8 | 290.1 | 10 | −60 | −65 |
| GM3(d40:1) | GM3(d18:1/18:0(d5)) | GM3 | 1235.8 | 290.1 | 10 | −60 | −65 |
| GM3(d41:1) | GM3(d18:1/18:0(d5)) | GM3 | 1249.8 | 290.1 | 10 | −60 | −65 |
| GM3(d42:2) | GM3(d18:1/18:0(d5)) | GM3 | 1261.8 | 290.1 | 10 | −60 | −65 |
| GM3(d42:1) | GM3(d18:1/18:0(d5)) | GM3 | 1263.8 | 290.1 | 10 | −60 | −65 |
| GM3(d43:0) | GM3(d18:1/18:0(d5)) | GM3 | 1279.8 | 290.1 | 10 | −60 | −65 |
| GM3(d44:1) | GM3(d18:1/18:0(d5)) | GM3 | 1291.8 | 290.1 | 10 | −60 | −65 |
| GM3(d44:2) | GM3(d18:1/18:0(d5)) | GM3 | 1289.8 | 290.1 | 10 | −60 | −65 |
| GM1(d36:1) | GM3(d18:1/18:0(d5)) | GM1 | 1544.9 | 290.1 | 10 | −275 | −75 |
| GM1(d38:1) | GM3(d18:1/18:0(d5)) | GM1 | 1572.9 | 290.1 | 10 | −275 | −75 |
| GM2(d38:1) | GM3(d18:1/18:0(d5)) | GM2 | 1410.7 | 290.1 | 10 | −60 | −65 |
| GM2(d36:1) | GM3(d18:1/18:0(d5)) | GM2 | 1382.8 | 290.1 | 10 | −60 | −65 |
| GD3(d34:1) | GM3(d18:1/18:0(d5)) | GM3 | 720.9 | 290.1 | 8 | −60 | −40 |
| GD3(d36:1) | GM3(d18:1/18:0(d5)) | GM3 | 734.9 | 290.1 | 8 | −60 | −40 |
| GD3(d38:1) | GM3(d18:1/18:0(d5)) | GM3 | 748.9 | 290.1 | 8 | −60 | −40 |
| GD3(d40:1) | GM3(d18:1/18:0(d5)) | GM3 | 762.9 | 290.1 | 8 | −60 | −40 |
| GD3(d42:2) | GM3(d18:1/18:0(d5)) | GM3 | 775.9 | 290.1 | 8 | −60 | −40 |
| GD3(d42:1) | GM3(d18:1/18:0(d5)) | GM3 | 776 | 290.1 | 8 | −60 | −40 |
| GD1a/b(d36:1) | GM3(d18:1/18:0(d5)) | GM3 | 917.5 | 290.1 | 8 | −60 | −52 |
| GD1a/b(d38:1) | GM3(d18:1/18:0(d5)) | GM3 | 931.5 | 290.1 | 8 | −60 | −52 |
| GT1b(d36:1) | GM3(d18:1/18:0(d5)) | GM3 | 1063 | 290.1 | 8 | −60 | −35 |
| GT1b(d38:1) | GM3(d18:1/18:0(d5)) | GM3 | 1077 | 290.1 | 8 | −60 | −35 |
| GQ1b(d36:1) | GM3(d18:1/18:0(d5)) | GM3 | 1208.6 | 290.1 | 8 | −60 | −55 |
| GQ1b(d38:1) | GM3(d18:1/18:0(d5)) | GM3 | 1222.6 | 290.1 | 8 | −60 | −55 |

### LCMS Assay for GlcCer and GalCer

Glucosylceramide and galactosylceramide analyses were performed by liquid chromatography (Shimadzu Nexera X2 system, Shimadzu Scientific Instrument, Columbia, Md., USA) coupled to electrospray mass spectrometry (Sciex QTRAP 6500+ Sciex, Framingham, Mass., USA). For each analysis, 10 μL of sample was injected on a HALO HILIC 2.0 μm, 3.0×150 mm column (Advanced Materials Technology, PN 91813-701) using a flow rate of 0.45 mL/min at 45° C. Mobile phase A consisted of 92.5/5/2.5 ACN/IPA/H2O with 5 mM ammonium formate and 0.5% formic Acid. Mobile phase B consisted of 92.5/5/2.5 H2O/IPA/ACN with 5 mM ammonium formate and 0.5% formic Acid. The gradient was programmed as follows: 0.0-3.1 min at 100% B, 3.2 min at 95% B, 5.7 min at 85% B, hold to 7.1 min at 85% B, drop to 0% B at 7.25 min and hold to 8.75 min, ramp back to 100% at 10.65 min and hold to 11 min. Electrospray ionization was performed in the positive-ion mode applying the following settings: curtain gas at 25; collision gas was set at medium; ion spray voltage at 5500; temperature at 350° C.; ion source Gas 1 at 55; ion source Gas 2 at 60. Data acquisition was performed using Analyst 1.6 (Sciex) in multiple reaction monitoring mode (MRM) with the following parameters: dwell time (msec) and collision energy (CE) for each species reported in Table B; declustering potential (DP) at 45; entrance potential (EP) at 10; and collision cell exit potential (CXP) at 12.5. Lipids were quantified using a mixture of isotope labeled internal standards as reported in Table B. Glucosylceramide and Galactosylceramide were identified based on their retention times and MRM properties of commercially available reference standards (Avanti Polar Lipids, Birmingham, Ala., USA). Quantification was performed using MultiQuant 3.02 (Sciex). Metabolites were normalized to either total protein amount, tissue weight or volume.

TABLE B
Acquisition parameters information for GlcCer and GalCer Assay.

| | | | Q1 | Q2 | Time | | |
|---|---|---|---|---|---|---|---|
| Lipid | Internal Standard | Group | mass | mass | (msec) | DP | CE |
| GlcCer(d18:1/16:0) | GlcCer (d18:1(d5)/18:0) | GlcCer | 700.6 | 264.3 | 50 | 45 | 45 |
| GlcCer(d18:1/18:0) | GlcCer (d18:1(d5)/18:0) | GlcCer | 728.6 | 264.3 | 50 | 45 | 45 |
| GlcCer(d18:2/18:0) | GlcCer (d18:1(d5)/18:0) | GlcCer | 726.6 | 262.3 | 50 | 45 | 45 |
| GlcCer(d18:1/20:0) | GlcCer (d18:1(d5)/18:0) | GlcCer | 756.6 | 264.3 | 50 | 45 | 50 |
| GlcCer (d18:2/20:0) | GlcCer (d18:1(d5)/18:0) | GlcCer | 754.6 | 262.3 | 50 | 45 | 50 |
| GlcCer (d18:1/22:0) | GlcCer (d18:1(d5)/18:0) | GlcCer | 784.6 | 264.3 | 50 | 45 | 50 |
| GlcCer (d18:1/22:1) | GlcCer (d18:1(d5)/18:0) | GlcCer | 782.6 | 264.3 | 50 | 45 | 50 |
| GlcCer (d18:2/22:0) | GlcCer (d18:1(d5)/18:0) | GlcCer | 782.6 | 262.3 | 50 | 45 | 50 |
| GlcCer (d18:1/24:1) | GlcCer (d18:1(d5)/18:0) | GlcCer | 810.7 | 264.3 | 50 | 45 | 50 |
| GlcCer (d18:1/24:0) | GlcCer (d18:1(d5)/18:0) | GlcCer | 812.7 | 264.3 | 50 | 45 | 50 |
| Glucosyl Sphingosine | Glucosyl Sphingosine-d5 | GlcCer | 462.2 | 264.3 | 200 | 45 | 16 |
| GlcCer (d18:1/18:0)-d5 | Internal standard | GlcCer | 733.6 | 269.3 | 7 | 45 | 45 |
| Glucosyl Sphingosine-d5 | Internal standard | GlcCer | 467.2 | 269.3 | 15 | 16 | 30 |
| GalCer (d18:1/16:0) | GlcCer (d18:1(d5)/18:0) | GalCer | 700.6 | 264.3 | 50 | 45 | 45 |
| GalCer (d18:1/18:0) | GlcCer (d18:1(d5)/18:0) | GalCer | 728.6 | 264.3 | 50 | 45 | 45 |
| GalCer (d18:2/18:0) | GlcCer (d18:1(d5)/18:0) | GalCer | 726.6 | 262.3 | 50 | 45 | 45 |
| GalCer (d18:1/20:0) | GlcCer (d18:1(d5)/18:0) | GalCer | 756.6 | 264.3 | 50 | 45 | 50 |
| GalCer (d18:2/20:0) | GlcCer (d18:1(d5)/18:0) | GalCer | 754.6 | 262.3 | 50 | 45 | 50 |
| GalCer (d18:1/22:0) | GlcCer (d18:1(d5)/18:0) | GalCer | 784.6 | 264.3 | 50 | 45 | 50 |
| GalCer (d18:1/22:1) | GlcCer (d18:1(d5)/18:0) | GalCer | 782.6 | 264.3 | 50 | 45 | 50 |
| GalCer (d18:2/22:0) | GlcCer (d18:1(d5)/18:0) | GalCer | 782.6 | 262.3 | 50 | 45 | 50 |
| GalCer (d18:1/24:1) | GlcCer (d18:1(d5)/18:0) | GalCer | 810.7 | 264.3 | 50 | 45 | 50 |
| GalCer (d18:1/24:0) | GlcCer (d18:1(d5)/18:0) | GalCer | 812.7 | 264.3 | 50 | 45 | 50 |
| Gal-Sph 18:1 | Glucosyl Sphingosine-d5 | GalCer | 462.2 | 282.3 | 200 | 45 | 30 |

### Mass Spectrometry Analysis of Eicosanoids

Eicosanoid analyses were performed by liquid chromatography (Shimadzu Nexera X2 system, Shimadzu Scientific Instrument, Columbia, Md., USA) coupled to electrospray mass spectrometry (Sciex QTRAP 6500+, Sciex, Framingham, Mass., USA). For each analysis, 5 μL of sample was injected on a BEH C18 1.7 μm, 2.1×100 mm column (Waters Corporation, Milford, Mass., USA) using a flow rate of 0.6 mL/min at 40° C. Mobile phases were composed as follows: A=water+0.1% acetic acid, and B=90:10 acetonitrile/isopropyl alcohol (v/v). The gradient was programmed as follows: 0.0-1.0 min at 25% B; 1.0-8.5 min to 95% B; 8.50-8.51 min at 95% B; 8.51-10.00 min at 25% B. Electrospray ionization was performed in negative ion mode applying the following settings: curtain gas at 30; collision gas was set at medium; ion spray voltage at −4500; temperature at 600° C.; ion source Gas 1 at 50; ion source Gas 2 at 60. Data acquisition was performed using Analyst 1.6.3 (Sciex) in multiple reaction monitoring mode (MRM), with the following parameters: dwell time (msec), collision energy (CE), and declustering potential (DP) for each species reported in Table C; entrance potential (EP) at −10; and collision cell exit potential (CXP) at −12. Eicosanoids were quantified using a mixture of non endogenous, deuterated internal standards as reported in Table C. Eicosanoids were identified based on their retention times and MRM properties of commercially available reference standards (Avanti Polar Lipids, Birmingham, Ala., USA). Quantification was performed using MultiQuant 3.02 (Sciex) and Skyline. Metabolites were normalized to either total protein amount, tissue weight or volume. Protein concentration was measured using the bicinchoninic acid (BCA) assay (Pierce, Rockford, Ill., USA).

TABLE C
Acquisition parameters information for Eicosanoid Assay.

| | | Q1 | Q2 | Time | | |
|---|---|---|---|---|---|---|
| Lipid | Internal Standard | mass | mass | (msec) | DP | CE |
| 12-HETE-d8 | N/A | 327.3 | 184.1 | 20 | −65 | −20 |
| 15-HETE-d8 | N/A | 327.3 | 226.1 | 20 | −65 | −20 |
| 5-HETE-d8 | N/A | 327.3 | 116.1 | 20 | −65 | −20 |
| (d4) PGE2 | N/A | 355.31 | 275.1 | 20 | −35 | −23 |
| PGF2alpha-d4 | N/A | 357.2 | 197.1 | 20 | −60 | −35 |
| 6-k-PGF1alpha-d4 | N/A | 373.2 | 249.1 | 20 | −45 | −35 |
| Arachidonic Acid-d8 | N/A | 311.2 | 267.1 | 10 | −110 | −18 |
| 5-HETE | 5-HETE-d8 | 319.2 | 115.1 | 20 | −50 | −18 |
| 12-HETE | 12-HETE-d8 | 319.2 | 179.1 | 20 | −50 | −18 |
| 15-HETE | 15-HETE-d8 | 319.2 | 219.1 | 20 | −50 | −18 |
| 6k PGF1α | 6-k-PGF1alpha-d4 | 369.2 | 245.1 | 20 | −45 | −34 |
| TxB2 | 15-HETE-d8 | 369.2 | 169.1 | 20 | −50 | −22 |
| PGF2a/8-iso PGF2a | PGF2alpha-d4 | 353.2 | 193.1 | 20 | −50 | −35 |
| PGE2/D2 | (d4) PGE2 | 351.2 | 271.1 | 40 | −30 | −28 |
| Arachidonic Acid | Arachidonic Acid-d8 | 303.2 | 259.1 | 10 | −110 | −18 |
| 5,15-diHETE | 15-HETE-d8 | 335.2 | 115.1 | 20 | −35 | −18 |
| 17 HDoHE | 15-HETE-d8 | 343.2 | 229.1 | 20 | −35 | −15 |
| 5-iso PGF2a | PGF2alpha-d4 | 353.2 | 115.1 | 20 | −60 | −28 |
| PGE2 | (d4) PGE2 | 351.2 | 175.1 | 20 | −30 | −28 |
| DHA | Arachidonic Acid-d8 | 327.2 | 229.1 | 10 | −110 | −17 |
| PGD2 | (d4) PGE2 | 351.2 | 233.2 | 20 | −30 | −28 |
| (d4) PGE2 | N/A | 355.31 | 193.1 | 20 | −35 | −23 |
| LTB4 | 15-HETE-d8 | 335.3 | 195.2 | 20 | −45 | −21 |
| EPA | (d8) Arachidonic Acid | 301.3 | 257.1 | 20 | −110 | −18 |
| LTE4 | 15-HETE-d8 | 438 | 235 | 20 | −60 | −28 |
| 9-HOTrE | 15-HETE-d9 | 293 | 171 | 20 | −45 | −15 |
| 13-HOTrE | 15-HETE-d10 | 293 | 195 | 20 | −45 | −15 |
| 9-oxoODE | 15-HETE-d11 | 293 | 185 | 20 | −70 | −20 |
| 13-oxoODE | 15-HETE-d12 | 293 | 113 | 20 | −70 | −20 |
| 9,10 EpOME | 15-HETE-d13 | 295 | 171.1 | 20 | −70 | −20 |
| 9-HODE | 15-HETE-d14 | 295 | 171 | 20 | −115 | −23 |
| 13-HODE | 15-HETE-d15 | 295 | 195 | 20 | −115 | −23 |
| Leukotriene E4 (LTE4) | 15-HETE-d16 | 438 | 333 | 20 | −80 | −25 |
| 8,15 diHETE | 15-HETE-d8 | 335 | 127 | 20 | −45 | −15 |

### Lipidomics Analysis

Lipid analyses were performed by liquid chromatography (Shimadzu Nexera X2 system, Shimadzu Scientific Instrument, Columbia, Md., USA) coupled to electrospray mass spectrometry (QTRAP 6500+, Sciex, Framingham, Mass., USA). For each analysis, 5 μL of sample was injected on a BEH C18 1.7 μm, 2.1×100 mm column (Waters Corporation, Milford, Mass., USA) using a flow rate of 0.25 mL/min at 55° C. For positive ionization mode, mobile phase A consisted of 60:40 acetonitrile/water (v/v) with 10 mM ammonium formate+0.1% formic acid; mobile phase B consisted of 90:10 isopropyl alcohol/acetonitrile (v/v) with 10 mM ammonium formate+0.1% formic acid. For negative ionization mode, mobile phase A consisted of 60:40 acetonitrile/water (v/v) with 10 mM ammonium acetate; mobile phase B consisted of 90:10 isopropyl alcohol/acetonitrile (v/v) with 10 mM ammonium acetate. The gradient was programmed as follows: 0.0-8.0 min from 45% B to 99% B, 8.0-9.0 min at 99% B, 9.0-9.1 min to 45% B, and 9.1-10.0 min at 45% B. Electrospray ionization was performed in either positive or negative ion mode applying the following settings: curtain gas at 30; collision gas was set at medium; ion spray voltage at 5500 (positive mode) or 4500 (negative mode); temperature at 250° C. (positive mode) or 600° C. (negative mode); ion source Gas 1 at 50; ion source Gas 2 at 60. Data acquisition was performed using Analyst 1.6.3 (Sciex) in multiple reaction monitoring mode (MRM), with the following parameters: dwell time (msec) and collision energy (CE) for each species reported in Table D (negative mode) or Table E (positive mode); declustering potential (DP) at 80 (positive mode) and at −80 (negative mode); entrance potential (EP) at 10 (positive mode) or −10 (negative mode): and collision cell exit potential (CXP) at 12.5 (positive mode) or −12.5 (negative mode). Lipids were quantified using a mixture of non-endogenous internal standards as reported in Tables D and E. Lipids were identified based on their retention times and MRM properties of commercially available reference standards (Avanti Polar Lipids, Birmingham, Ala., USA). Quantification was performed using MultiQuant 3.02 (Sciex). Metabolites were normalized to either total protein amount or cell number.

TABLE D
Acquisition parameters information for Lipidomics Assay in negative mode.

| | | Q1 | Q2 | Time | |
|---|---|---|---|---|---|
| Lipid | Internal Standard | mass | mass | (msec) | CE |
| PA(15:0/18:1(d7)) | N/A | 666.52 | 241.3 | 5 | −50 |
| PA(16:0_18:1) | PA(15:0/18:1(d7)) | 673.5 | 255.3 | 5 | −50 |
| PA(18:0_18:1) | PA(15:0/18:1(d7)) | 701.5 | 283.3 | 5 | −50 |
| PA(18:1_18:1) | PA(15:0/18:1(d7)) | 699.5 | 281.3 | 5 | −50 |
| PA(18:0_20:4) | PA(15:0/18:1(d7)) | 723.5 | 283.3 | 5 | −50 |
| PA(18:1_22:6) | PA(15:0/18:1(d7)) | 745.5 | 281.3 | 5 | −50 |
| PA(18:0_22:6) | PA(15:0/18:1(d7)) | 747.5 | 283.3 | 5 | −50 |
| PE(15:0/18:1(d7)) | N/A | 709.56 | 241.3 | 5 | −50 |
| PE(P-18:0/18:1) | PE(15:0/18:1(d7)) | 728.6 | 283.3 | 5 | −50 |
| PE(P-18:0/18:2) | PE(15:0/18:1(d7)) | 726.6 | 281.3 | 5 | −50 |
| PE(P-16:0/20:4) | PE(15:0/18:1(d7)) | 722.6 | 303.3 | 5 | −50 |
| PE(P-18:0/20:4) | PE(15:0/18:1(d7)) | 750.6 | 303.3 | 5 | −50 |
| PE(P-16:0/22:6) | PE(15:0/18:1(d7)) | 746.6 | 327.3 | 5 | −50 |
| PE(P-18:0/22:6) | PE(15:0/18:1(d7)) | 774.6 | 327.3 | 5 | −50 |
| (3-O-sulfo)Gal- | N/A | 722.5 | 97 | 8 | −150 |
| Cer(d18:1/12:0) | | | | | |
| (3-O-sulfo)Gal- | (3-O-sulfo)Gal- | 778.5 | 97 | 8 | −150 |
| Cer(d18:1/16:0) | Cer(d18:1/12:0) | | | | |
| (3-O-sulfo)Gal- | (3-O-sulfo)Gal- | 806.6 | 97 | 8 | −150 |
| Cer(d18:1/18:0) | Cer(d18:1/12:0) | | | | |
| (3-O-sulfo)Gal- | (3-O-sulfo)Gal- | 822.6 | 97 | 8 | −150 |
| Cer(d18:1/18:0(2OH) | Cer(d18:1/12:0) | | | | |
| (3-O-sulfo)Gal- | (3-O-sulfo)Gal- | 890.7 | 97 | 8 | −150 |
| Cer(d18:1/24:0) | Cer(d18:1/12:0) | | | | |
| (3-O-sulfo)Gal- | (3-O-sulfo)Gal- | 906.7 | 97 | 8 | −150 |
| Cer(d18:1/24:0(2OH)) | Cer(d18:1/12:0) | | | | |
| (3-O-sulfo)Gal- | (3-O-sulfo)Gal- | 888.7 | 97 | 8 | −150 |
| Cer(d18:1/24:1) | Cer(d18:1/12:0) | | | | |
| (3-O-sulfo)Gal- | (3-O-sulfo)Gal- | 904.7 | 97 | 8 | −150 |
| Cer(d18:1/24:1(2OH)) | Cer(d18:1/12:0) | | | | |
| LPE(P-16:0) | LPE(18:1(d7)) | 436.3 | 196.1 | 5 | −50 |
| LPE(P-18:0) | LPE(18:1(d7)) | 464.3 | 196.1 | 5 | −50 |
| LPE(P-18:1) | LPE(18:1(d7)) | 462.3 | 196.1 | 5 | −50 |
| LPE(18:1(d7)) | N/A | 485.3 | 288.3 | 5 | −50 |
| LPE(16:0) | LPE(18:1(d7)) | 452.278 | 255.3 | 5 | −50 |
| LPE(18:0) | LPE(18:1(d7)) | 480.31 | 283.3 | 5 | −50 |
| LPE(18:1) | LPE(18:1(d7)) | 478.3 | 281.3 | 5 | −50 |
| LPI(16:0) | LPE(18:1(d7)) | 571.3 | 241.1 | 5 | −50 |
| LPI(18:0) | LPE(18:1(d7)) | 599.3 | 241.1 | 5 | −50 |
| LPI(20:4) | LPE(18:1(d7)) | 619.3 | 241.1 | 5 | −50 |
| LPG(16:0) | LPE(18:1(d7)) | 483.3 | 255.3 | 5 | −50 |
| LPG(18:0) | LPE(18:1(d7)) | 511.3 | 283.3 | 5 | −50 |
| LPG(18:1) | LPE(18:1(d7)) | 509.3 | 281.3 | 5 | −50 |
| LPG(20:4) | LPE(18:1(d7)) | 531.3 | 303.3 | 5 | −50 |
| CL(14:0/14:0/14:0/14:0) | N/A | 555.3 | 327.3 | 5 | −50 |
| CL(72:8) | CL(14:0/14:0/14:0/14:0) | 619.5 | 227.2 | 5 | −50 |
| Cholesterol Sulfate | N/A | 465.3 | 96.7 | 5 | −80 |
| PG(15:0/18:1(d7)) | N/A | 740.55 | 241.3 | 5 | −50 |
| PG(16:0_18:1) | PG(15:0/18:1(d7)) | 747.5 | 255.3 | 5 | −50 |
| PG(18:0_18:1) | PG(15:0/18:1(d7)) | 775.5 | 283.3 | 5 | −50 |
| PG(18:1/18:1) | PG(15:0/18:1(d7)) | 773.5 | 281.3 | 5 | −50 |
| PG(18:0_20:4) | PG(15:0/18:1(d7)) | 797.6 | 283.3 | 5 | −50 |
| PI(15:0/18:1(d7)) | N/A | 828.6 | 241.3 | 5 | −50 |
| PI(18:0_18:1) | PI(15:0/18:1(d7)) | 863.6 | 283.3 | 5 | −50 |
| PI(18:1/18:1) | PI(15:0/18:1(d7)) | 861.6 | 281.3 | 5 | −50 |
| PI(16:0_20:4) | PI(15:0/18:1(d7)) | 857.6 | 255.3 | 5 | −50 |
| PI(18:0_20:4) | PI(15:0/18:1(d7)) | 885.6 | 283.3 | 5 | −50 |
| PI(16:0_22:6) | PI(15:0/18:1(d7)) | 881.6 | 255.3 | 5 | −50 |
| PI(18:0_22:6) | PI(15:0/18:1(d7)) | 909.6 | 283.3 | 5 | −50 |
| PI(20:4/20:4) | PI(15:0/18:1(d7)) | 905.6 | 303.3 | 5 | −50 |
| PS(15:0/18:1(d7)) | N/A | 753.55 | 241.3 | 5 | −50 |
| PS(18:0_18:1) | PS(15:0/18:1(d7)) | 788.6 | 283.3 | 5 | −50 |
| PS(18:0_20:4) | PS(15:0/18:1(d7)) | 810.6 | 283.3 | 5 | −50 |
| PS(16:0_22:6) | PS(15:0/18:1(d7)) | 806.6 | 255.3 | 5 | −50 |
| PS(18:1_22:6) | PS(15:0/18:1(d7)) | 832.6 | 281.3 | 5 | −50 |
| PS(18:0_22:6) | PS(15:0/18:1(d7)) | 834.6 | 283.3 | 5 | −50 |
| PS(22:6/22:6) | PS(15:0/18:1(d7)) | 878.5 | 327.3 | 5 | −50 |
| Arachidonic acid(d8) | N/A | 311.3 | 311.3 | 5 | −10 |
| Palmitic acid | Arachidonic acid(d8) | 255.1 | 255.1 | 5 | −10 |
| Palmitoleic acid | Arachidonic acid(d8) | 253.1 | 253.1 | 5 | −10 |
| Stearic acid | Arachidonic acid(d8) | 283.2 | 283.2 | 5 | −10 |
| Oleic acid | Arachidonic acid(d8) | 281.2 | 281.2 | 5 | −10 |
| Linoleic acid | Arachidonic acid(d8) | 279.2 | 279.2 | 5 | −10 |
| Linolenic acid | Arachidonic acid(d8) | 277.2 | 277.2 | 5 | −10 |
| Arachidonic acid | Arachidonic acid(d8) | 303.2 | 303.2 | 5 | −10 |
| EPA | Arachidonic acid(d8) | 301.2 | 301.2 | 5 | −10 |
| DHA | Arachidonic acid(d8) | 327.2 | 327.2 | 5 | −10 |

TABLE E
Acquisition parameters information for Lipidomics Assay in positive mode.

| | | Q1 | Q3 | Time | |
|---|---|---|---|---|---|
| Lipid | Internal Standard | mass | mass | (msec) | CE |
| Sphingosine(d17:1) | N/A | 286.2 | 268.3 | 5 | 20 |
| Sphingosine | Sphingosine(d17:1) | 300.2 | 282.2 | 5 | 20 |
| Sphinganine | Sphingosine(d17:1) | 302.2 | 284.2 | 5 | 20 |
| Glucosyl | N/A | 467.2 | 269.3 | 5 | 16 |
| sphingosine(d5) | | | | | |
| Hexosyl sphingosine | Glucosyl sphingosine | 462.3 | 282.2 | 5 | 16 |
| | (d5) | | | | |
| Lactosyl sphingosine | Glucosyl sphingosine | 624.4 | 282.3 | 5 | 16 |
| | (d5) | | | | |
| Cer(d18:1/17:0) | N/A | 552.4 | 264.3 | 5 | 40 |
| Cer(d18:1/16:0) | Cer(d18:1/17:0) | 538.5 | 264.4 | 5 | 40 |
| Cer(d18:1/18:0) | Cer(d18:1/17:0) | 566.6 | 264.4 | 5 | 40 |
| Cer(d18:1/24:0) | Cer(d18:1/17:0) | 650.6 | 264.4 | 5 | 40 |
| Cer(d18:1/24:1) | Cer(d18:1/17:0) | 648.6 | 264.4 | 5 | 40 |
| SM(d18:1(d9)/18:1) | N/A | 738.7 | 184.1 | 5 | 40 |
| SM(d18:1/16:0) | SM(d18:1(d9)/18:1) | 703.6 | 184.1 | 5 | 40 |
| SM(d18:1/18:0) | SM(d18:1(d9)/18:1) | 731.6 | 184.1 | 5 | 40 |
| SM(d18:1/24:0) | SM(d18:1(d9)/18:1) | 815.7 | 184.1 | 5 | 40 |
| SM(d18:1/24:1) | SM(d18:1(d9)/18:1) | 813.7 | 184.1 | 5 | 40 |
| HexCer(d18:1/12:0) | N/A | 644.5 | 264.3 | 5 | 40 |
| HexCer(d18:1/16:0) | HexCer(d18:1/12:0) | 700.6 | 264.6 | 5 | 40 |
| HexCer(d18:1/18:0) | HexCer(d18:1/12:0) | 728.6 | 264.4 | 5 | 40 |
| HexCer(d18:1/24:0) | HexCer(d18:1/12:0) | 812.7 | 264.4 | 5 | 40 |
| HexCer(d18:1/24:1) | HexCer(d18:1/12:0) | 810.7 | 264.4 | 5 | 40 |
| LacCer(d18:1/16:0) | HexCer(d18:1/12:0) | 862.6 | 264.6 | 5 | 40 |
| LacCer(d18:1/18:0) | HexCer(d18:1/12:0) | 890.7 | 264.4 | 5 | 40 |
| LacCer(d18:1/24:0) | HexCer(d18:1/12:0) | 974.8 | 264.4 | 5 | 40 |
| LacCer(d18:1/24:1) | HexCer(d18:1/12:0) | 972.7 | 264.4 | 5 | 40 |
| LPC(18:1(d7)) | N/A | 529.3 | 184.1 | 5 | 40 |
| LPC(16:1) | LPC(18:1(d7)) | 494.5 | 184.1 | 5 | 40 |
| LPC(16:0) | LPC(18:1(d7)) | 496.3 | 184.1 | 5 | 40 |
| LPC(18:0) | LPC(18:1(d7)) | 524.3 | 184.1 | 5 | 40 |
| LPC(18:1) | LPC(18:1(d7)) | 522.3 | 184.1 | 5 | 40 |
| LPC(20:4) | LPC(18:1(d7)) | 544.3 | 184.1 | 5 | 40 |
| LPC(22:6) | LPC(18:1(d7)) | 568.3 | 184.1 | 5 | 40 |
| LPC(24:1) | LPC(18:1(d7)) | 606.5 | 184.1 | 5 | 40 |
| LPC(24:0) | LPC(18:1(d7)) | 608.5 | 184.1 | 5 | 40 |
| LPC(26:1) | LPC(18:1(d7)) | 634.5 | 104.1 | 5 | 40 |
| LPC(26:0) | LPC(18:1(d7)) | 636.5 | 104.1 | 5 | 40 |
| LSM | LPC(18:1(d7)) | 465.5 | 184.1 | 5 | 40 |
| PC(15:0/18:1(d7)) | N/A | 754.6 | 184.1 | 5 | 40 |
| PC(36:1) | PC(15:0/18:1(d7)) | 788.6 | 184.1 | 5 | 40 |
| PC(36:2) | PC(15:0/18:1(d7)) | 786.6 | 184.1 | 5 | 40 |
| PC(36:4) | PC(15:0/18:1(d7)) | 782.6 | 184.1 | 5 | 40 |
| PC(38:4) | PC(15:0/18:1(d7)) | 810.6 | 184.1 | 5 | 40 |
| PC(38:6) | PC(15:0/18:1(d7)) | 806.6 | 184.1 | 5 | 40 |
| PC(40:6) | PC(15:0/18:1(d7)) | 834.6 | 184.1 | 5 | 40 |
| PC(O-18:0/2:0) | LPC(18:1(d7)) | 524.3 | 184.1 | 5 | 40 |
| PE(15:0/18:1(d7)) | N/A | 711.6 | 570.5 | 5 | 40 |
| PE(36:1) | PE(15:0/18:1(d7)) | 746.6 | 605.5 | 5 | 40 |
| PE(36:2) | PE(15:0/18:1(d7)) | 744.6 | 603.5 | 5 | 40 |
| PE(36:4) | PE(15:0/18:1(d7)) | 740.6 | 599.5 | 5 | 40 |
| PE(38:4) | PE(15:0/18:1(d7)) | 768.6 | 627.5 | 5 | 40 |
| PE(38:6) | PE(15:0/18:1(d7)) | 764.6 | 623.5 | 5 | 40 |
| PE(40:6) | PE(15:0/18:1(d7)) | 792.6 | 651.5 | 5 | 40 |
| Cholesterol(d7) | N/A | 376.2 | 376.2 | 5 | 10 |
| Cholesterol | Cholesterol(d7) | 369.3 | 369.3 | 5 | 10 |
| CE(18:1(d7)) | N/A | 675.2 | 369.4 | 5 | 26 |
| CE(16:1) | CE(18:1(d7)) | 640.6 | 369.3 | 5 | 26 |
| CE(18:1) | CE(18:1(d7)) | 668.6 | 369.3 | 5 | 26 |
| CE(18:2) | CE(18:1(d7)) | 666.6 | 369.3 | 5 | 26 |
| CE(20:4) | CE(18:1(d7)) | 690.6 | 369.3 | 5 | 26 |
| CE(20:5) | CE(18:1(d7)) | 688.6 | 369.3 | 5 | 26 |
| CE(22:6) | CE(18:1(d7)) | 714.6 | 369.3 | 5 | 26 |
| TG(15:0/18:1(d7)/15:0) | N/A | 829.4 | 523.5 | 5 | 40 |
| TG 52:4/18:1 | TG(15:0/18:1(d7)/15:0) | 872.7 | 573.4 | 5 | 40 |
| TG 52:3/18:1 | TG(15:0/18:1(d7)/15:0) | 874.7 | 575.4 | 5 | 40 |
| TG 54:2/18:0 | TG(15:0/18:1(d7)/15:0) | 904.7 | 603.4 | 5 | 40 |
| TG(52:5/20:4) | TG(15:0/18:1(d7)/15:0) | 870.6 | 549.3 | 5 | 40 |
| TG 54:6/20:4 | TG(15:0/18:1(d7)/15:0) | 896.6 | 575.3 | 5 | 40 |
| TG 54:7/20:4 | TG(15:0/18:1(d7)/15:0) | 894.6 | 573.3 | 5 | 40 |
| TG 56:4/20:4 | TG(15:0/18:1(d7)/15:0) | 928.8 | 607.5 | 5 | 40 |
| TG 56:6/20:4 | TG(15:0/18:1(d7)/15:0) | 924.7 | 603.4 | 5 | 40 |
| TG 56:7/20:4 | TG(15:0/18:1(d7)/15:0) | 922.7 | 601.4 | 5 | 40 |
| TG(58:5/20:4) | TG(15:0/18:1(d7)/15:0) | 954.7 | 633.4 | 5 | 40 |
| TG(58:7/20:4) | TG(15:0/18:1(d7)/15:0) | 950.7 | 629.4 | 5 | 40 |
| TG 58:8/22:6 | TG(15:0/18:1(d7)/15:0) | 948.7 | 603.4 | 5 | 40 |
| TG 60:7/22:6 | TG(15:0/18:1(d7)/15:0) | 978.7 | 633.4 | 5 | 40 |
| TG 60:8/22:6 | TG(15:0/18:1(d7)/15:0) | 976.7 | 631.4 | 5 | 40 |
| Sphingosine-1- | N/A | 366.3 | 250.3 | 5 | 25 |
| phosphate(d17:1) | | | | | |
| Sphingosine-1- | Sphingosine(d17:1) | 380.3 | 264.3 | 5 | 25 |
| phosphate | | | | | |
| Sphinganine-1- | Sphingosine(d17:1) | 382.3 | 266.3 | 5 | 18 |
| phosphate | | | | | |
| GB3(d18:1/16:0) | HexCer(d18:1/12:0) | 1025 | 520.5 | 5 | 40 |
| GB3(d18:1/18:0) | HexCer(d18:1/12:0) | 1053 | 548.6 | 5 | 40 |
| GB3(d18:1/24:0) | HexCer(d18:1/12:0) | 1137 | 632.6 | 5 | 40 |
| GB3(d18:1/24:1) | HexCer(d18:1/12:0) | 1135 | 630.6 | 5 | 40 |
| GlcCer(d18:1/18:0(d5)) | N/A | 733.6 | 269.3 | 5 | 45 |
| Cer(d18:1/16:0(d7)) | N/A | 545.5 | 271.4 | 5 | 40 |
| LPC(26:0) | LPC(18:1(d7)) | 636.5 | 104.1 | 5 | 40 |
| LPC(24:0) | LPC(18:1(d7)) | 608.5 | 184.1 | 5 | 40 |
| LPC(26:1) | LPC(18:1(d7)) | 634.5 | 104.1 | 5 | 40 |
| LPC(24:1) | LPC(18:1(d7)) | 606.5 | 184.1 | 5 | 40 |
| LPC(16:1) | LPC(18:1(d7)) | 494.5 | 184.1 | 5 | 40 |
| Cer(d18:0/16:0) | Cer(d18:1/17:0) | 540.6 | 522.3 | 5 | 40 |
| Cer(d18:0/18:0) | Cer(d18:1/17:0) | 568.7 | 550.4 | 5 | 40 |
| Cer(d18:0/24:0) | Cer(d18:1/17:0) | 652.9 | 634.4 | 5 | 40 |
| Cer(d18:0/24:1) | Cer(d18:1/17:0) | 650.9 | 632.4 | 5 | 40 |
| Glc-Cholesterol | CE(18:1(d7)) | 566.6 | 369.3 | 5 | 17 |
| Glc-Sitosterol | CE(18:1(d7)) | 594.6 | 397.4 | 5 | 17 |
| 7-HOCA | CE(18:1(d7)) | 431.3 | 367.3 | 5 | 25 |
| 24-hydroxy- | N/A | 392.4 | 374.4 | 5 | 15 |
| cholesterol(d7) | | | | | |
| hydroxy-cholesterol | CE(18:1(d7)) | 385.3 | 367.3 | 5 | 30 |
| DG(16:0_18:1) | DG(15:0/18:1(d7)) | 614.4 | 313.4 | 5 | 30 |
| DG(18:0_18:1) | DG(15:0/18:1(d7)) | 640.4 | 341.3 | 5 | 30 |
| DG(18:1/18:1) | DG(15:0/18:1(d7)) | 638.4 | 339.3 | 5 | 30 |
| DG(16:0_20:4) | DG(15:0/18:1(d7)) | 634.5 | 313.3 | 5 | 30 |
| DG(18:0_20:4) | DG(15:0/18:1(d7)) | 662.5 | 341.3 | 5 | 30 |
| DG(18:0_22:6) | DG(15:0/18:1(d7)) | 686.6 | 341.3 | 5 | 30 |
| DG(18:1_20:4) | DG(15:0/18:1(d7)) | 660.5 | 339.3 | 5 | 30 |
| DG(15:0/18:1(d7)) | N/A | 605.6 | 346.5 | 5 | 30 |
| MG(18:1(d7)) | N/A | 381.3 | 272.5 | 5 | 22 |
| MG(16:0) | MG(18:1(d7)) | 348.3 | 239.3 | 5 | 22 |
| MG(20:4) | MG(18:1(d7)) | 396.3 | 287.3 | 5 | 22 |
| MG(18:0) | MG(18:1(d7)) | 376.3 | 267.3 | 5 | 22 |
| MG(18:1) | MG(18:1(d7)) | 374.3 | 265.3 | 5 | 22 |
| MG(22:6) | MG(18:1(d7)) | 403.3 | 311.3 | 5 | 22 |
| MG(16:1) | MG(18:1(d7)) | 346.3 | 237.3 | 5 | 22 |
| 7-keto-cholesterol | CE(18:1(d7)) | 401.3 | 383.3 | 5 | 15 |
| 4-beta- | CE(18:1(d7)) | 420.3 | 385.3 | 5 | 15 |
| hydroxycholesterol | | | | | |
| CE oxoODE | CE(18:1(d7)) | 680.6 | 369.2 | 5 | 25 |
| CE HODE | CE(18:1(d7)) | 682.6 | 369.2 | 5 | 25 |
| CE HpODE | CE(18:1(d7)) | 698.6 | 369.2 | 5 | 25 |
| Glc Sphingosine(d5) | N/A | 467.2 | 269.3 | 5 | 16 |
| PC(15:0/18:1(d7)) | N/A | 754.6 | 184.1 | 5 | 40 |
| PC(36:1) | PC(15:0/18:1(d7)) | 788.6 | 184.1 | 5 | 40 |
| PC(36:2) | PC(15:0/18:1(d7)) | 786.6 | 184.1 | 5 | 40 |
| PC(36:4) | PC(15:0/18:1(d7)) | 782.6 | 184.1 | 5 | 40 |
| PC(38:4) | PC(15:0/18:1(d7)) | 810.6 | 184.1 | 5 | 40 |
| PC(38:6) | PC(15:0/18:1(d7)) | 806.6 | 184.1 | 5 | 40 |
| PC(40:4) | PC(15:0/18:1(d7)) | 838.6 | 184.1 | 5 | 40 |
| PC(40:6) | PC(15:0/18:1(d7)) | 834.6 | 184.1 | 5 | 40 |
| PC(O-18:0/2:0) | LPC(18:1(d7)) | 524.3 | 184.1 | 5 | 40 |
| PE(15:0/18:1(d7)) | N/A | 711.6 | 570.5 | 5 | 40 |
| PE(36:1) | PE(15:0/18:1(d7)) | 746.6 | 605.5 | 5 | 40 |
| PE(36:2) | PE(15:0/18:1(d7)) | 744.6 | 603.5 | 5 | 40 |
| PE(36:4) | PE(15:0/18:1(d7)) | 740.6 | 599.5 | 5 | 40 |
| PE(38:4) | PE(15:0/18:1(d7)) | 768.6 | 627.5 | 5 | 40 |
| PE(38:6) | PE(15:0/18:1(d7)) | 764.6 | 623.5 | 5 | 40 |
| PE(40:4) | PE(15:0/18:1(d7)) | 796.6 | 655.5 | 5 | 40 |
| PE(40:6) | PE(15:0/18:1(d7)) | 792.6 | 651.5 | 5 | 40 |
| POV-PC | PC(15:0/18:1(d7)) | 594.5 | 184.1 | 5 | 40 |
| PC(16:0/9:0(CHO)) | PC(15:0/18:1(d7)) | 650.4 | 184.1 | 5 | 40 |
| PC(16:0/9:0(COOH)) | PC(15:0/18:1(d7)) | 666.4 | 184.1 | 5 | 40 |
| LysoPAF(16:0) | PC(15:0/18:1(d7)) | 482.3 | 184.1 | 5 | 40 |
| PC(O-16:0/2:0) | PC(15:0/18:1(d7)) | 524.3 | 184.1 | 5 | 40 |
| PC(18:0/20:4(OH[S])) | PC(15:0/18:1(d7)) | 826.6 | 184.1 | 5 | 40 |
| PC(18:0/20:4(OOH[S])) | PC(15:0/18:1(d7)) | 842.6 | 184.1 | 5 | 40 |
| PE(18:0/20:4(OH[S])) | PE(15:0/18:1(d7)) | 784.5 | 643.4 | 5 | 40 |
| PE(18:0/20:4(OOH[S])) | PE(15:0/18:1(d7)) | 800.5 | 659.4 | 5 | 40 |
| Coenzyme Q10 | TG(15:0/18:1(d7)/15:0) | 863.3 | 197.2 | 5 | 35 |

### Homogenization of ETV:IDS Brain Tissue and Trem2 Analysis of ETV:IDS Brain Tissue and CSF

50 mg tissue was homogenized in 500 μL 1×CST buffer (Cell Signaling Technology 9803S) made with complete Protease Inhibitor (Roche #04693132001) and PhosStop (Roche 04906837001) using the Qiagen TissueLyzer II (Cat No./ID: 85300) for 2 rounds of 3 minutes at 30 Hz. Homogenate was incubated on ice for 20 minutes and spun at 21,100 g for 30 minutes at 4° C. Subsequent lysate was transferred to a clean 96-well deep plate, and a BCA was performed to quantify total protein amounts. Samples were then stored at −80° C. until assay use.

For Trem2 analysis in brain tissue and soluble Trem2 (sTrem2) analysis in CSF, an MSD GOLD 96w small spot streptavidin plate (MSD L45SA) was prepared for Trem2 assay by coating with 1 ug/mL biotinylated sheep anti-mouse antibody (R&D Systems BAF1729) overnight at 4° C. The next day, the MSD plate was rinsed with tris buffered saline with triton (TBST) and blocked for two hours using 3% bovine serum albumin in TBST, while shaking at 600 rpm. The MSD plate was again rinsed again with TBST, and brain lysates were diluted 5× in blocking solution and added to the MSD plate to incubate for 1 hour at 600 rpm. Following the next TBST rinse, sulfotagged sheep anti-mouse antibody (R&D Systems AF1729) was added to the plate and incubated for 1 hour, again at 600 rpm, and a final rinse was conducted before adding 2×MSD read buffer diluted in water. The plate was then read using the MSD Meso Sector S600. The Trem2 signal was normalized to the protein concentration and plotted with GraphPad Prism.

### Abbreviations

BMP=bis(monoacylglycerol)phosphate; ETV:IDS=enzyme transport vehicle: iduronate 2-sulfatase; GlcCer=glucosylceramide; GalCer=galactosyl ceramide; IDS=iduronate 2-sulfatase; KI=knock-in; KO=knockout; TfR^{mu/hu}=chimeric human/mouse transferrin receptor.

### Results

To determine whether the robust GAG reduction observed in brain translated to correction of downstream disease-relevant pathology, the ability of ETV:IDS to correct secondary lysosomal storage was assessed. Four weekly activity-equivalent doses of ETV:IDS or idursulfase were intravenously administered to IDS KO; TfR^{mu/hu} KI mice, and the levels of a panel of lysosomal lipids, including gangliosides, glucosylceramide, and bis(monoacylglycerol)phosphate, were measured using liquid chromatography-tandem mass spectrometry (LCMS). Significant accumulation of lysosomal lipids was observed in the brains of IDS KO; TfR^{mu/hu} KI mice compared with wild-type controls. Following 4 weekly doses of 40 mg/kg, ETV:IDS was highly effective in lowering lysosomal lipids in the brain, completely reducing levels of these lipids to that seen in wild-type mice. Treatment with an activity-equivalent dose of idursulfase, however, failed to reduce levels of these lysosomal lipids in the brain. Together, these data demonstrate that ETV:IDS effectively corrects secondary lysosomal storage in addition to its proximal effects on GAG accumulation.

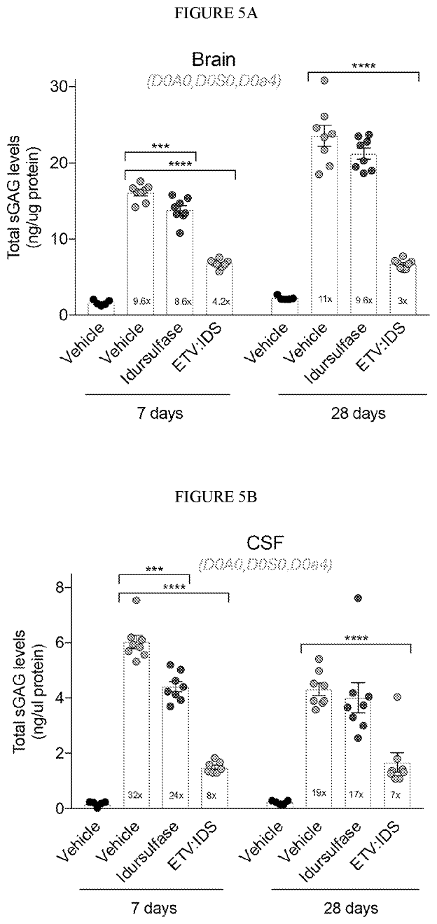

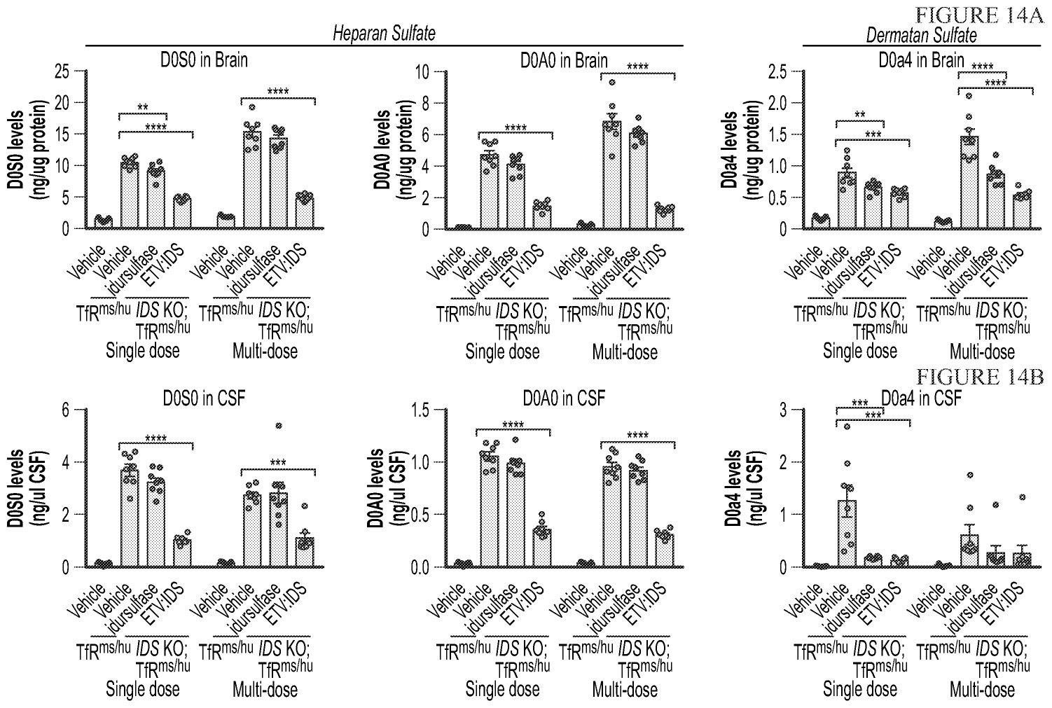

In particular FIGS. 5A-5B show peripheral administration of ETV:IDS (4 week treatment) corrects (FIG. 5A) brain and (FIG. 5B) CSF GAG accumulation in IDS KO mice. A 58% reduction in brain GAG was observed with peripheral administration of ETV:IDS compared to a 14% reduction with an activity equivalent dose of Elaprase. Additionally, a 66% reduction in CSF GAG was observed with peripheral administration of ETV:IDS compared to a 27% reduction with an activity equivalent dose of Elaprase (one outlier omitted for ETV:IDS group). FIGS. 14A-14B illustrate the reduction of individual GAG species (DOOSO, D00A0, D00a4) in the brain and CSF. In a follow-up study, CSF GAGs were reduced three times over WT at a dosing of 40 mg/kg of ETV:IDS.

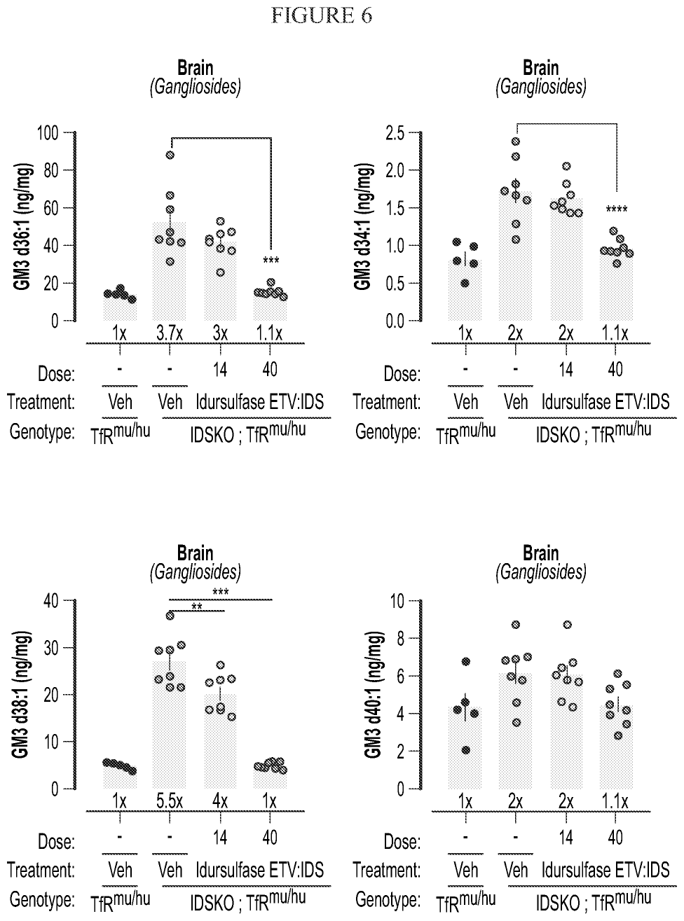

FIG. 6 shows peripheral administration of ETV:IDS (4 week treatment) corrected lysosomal lipid (gangliosides) accumulation in IDS KO brains.

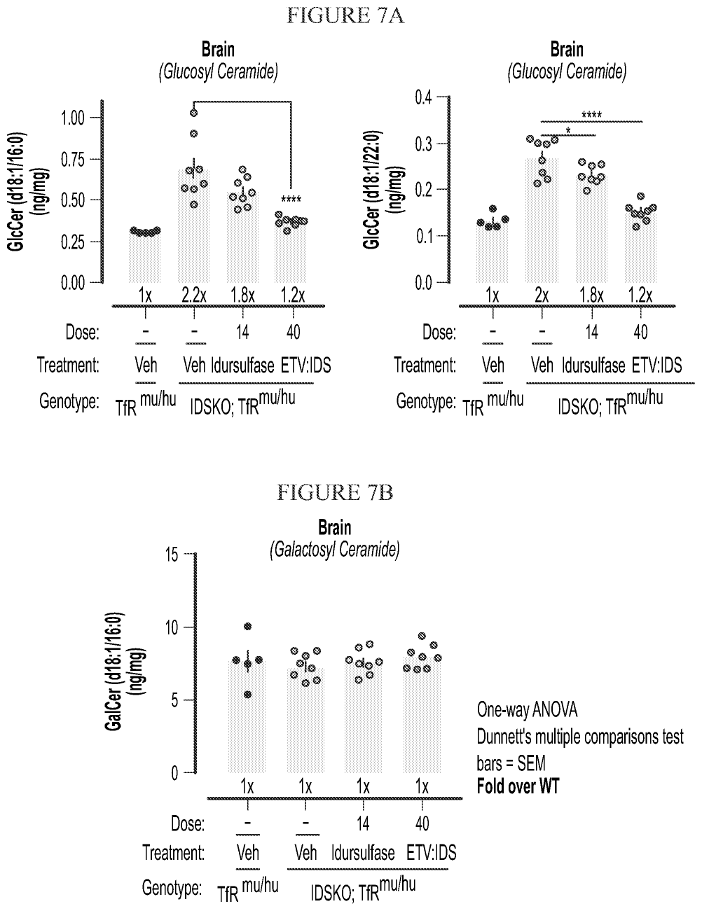

FIGS. 7A-7B show that peripheral administration of ETV:IDS (4 week treatment) corrected lysosomal lipid (GlcCer) accumulation in IDS KO brains. Correction of brain GlcCer accumulation with peripheral administration of ETV:IDS was observed, while no change was observed in galactosyl ceramides (GalCer).

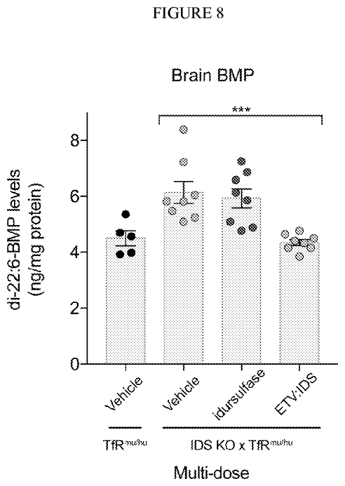

FIG. 8 shows that peripheral administration of ETV:IDS (4 week treatment) corrected lysosomal lipid (BMP) accumulation in IDS KO brains.

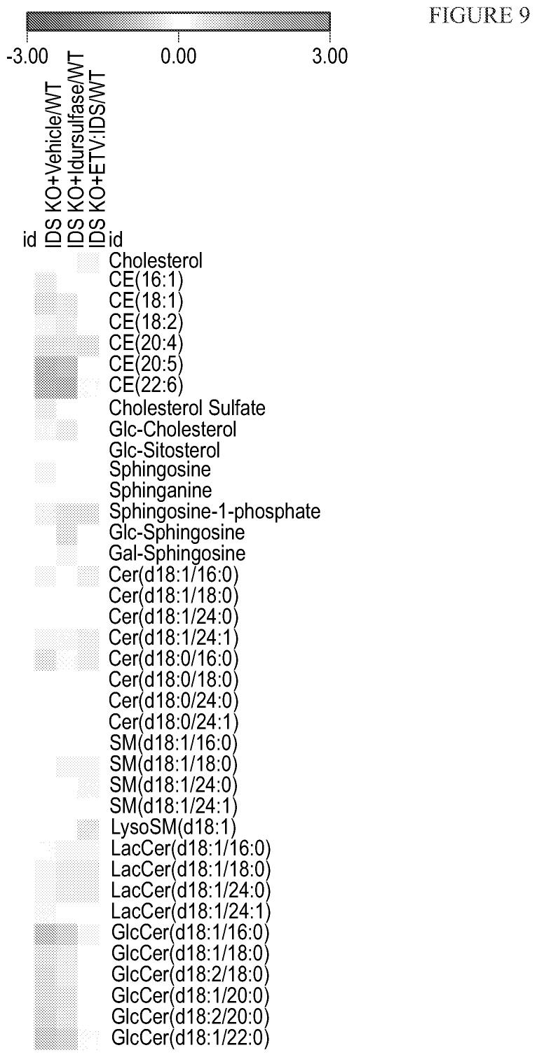

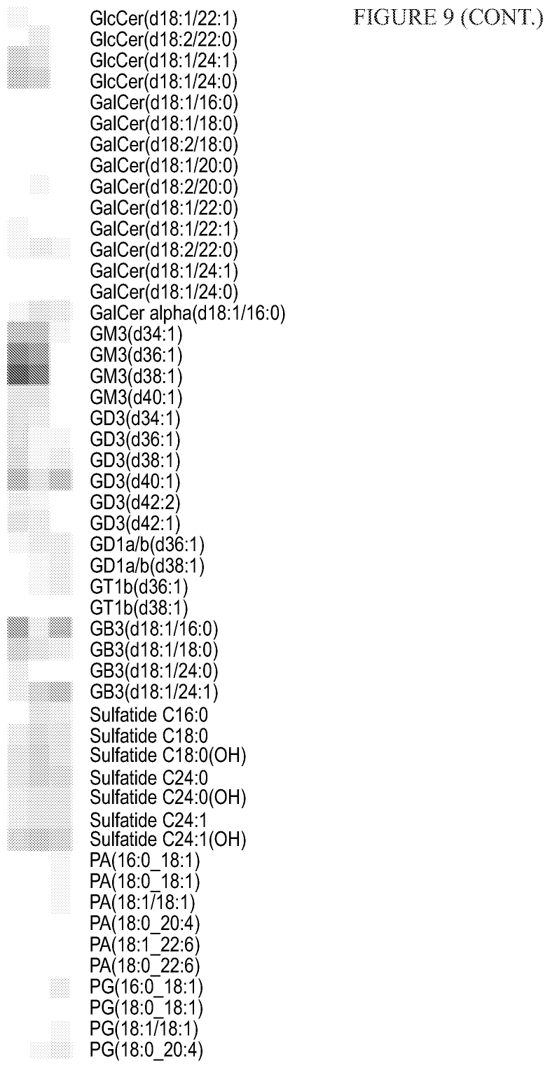

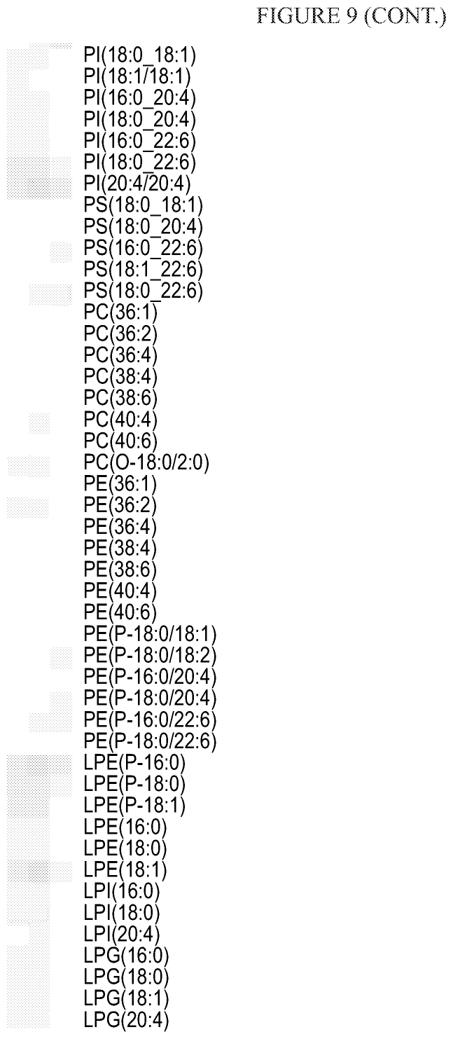

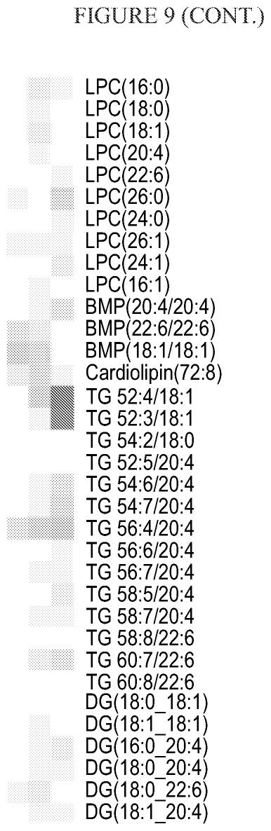

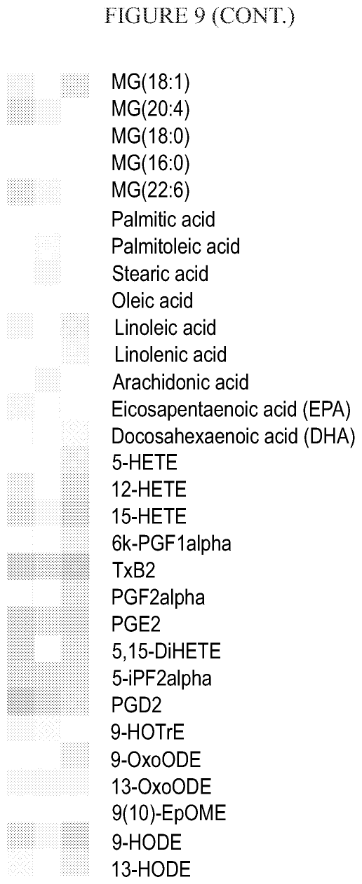

FIG. 9 shows a heat map illustrating lipid levels in brains of IDS KO mice treated with vehicle, ETV:IDS or Elaprase (idursulfase). This heat map was generated using modified data from Table 2. Specifically, the data in Table 2 was modified with a cutoff, converting values between 0.9 and 1.1 to 1, in order to show differences greater than 10%.

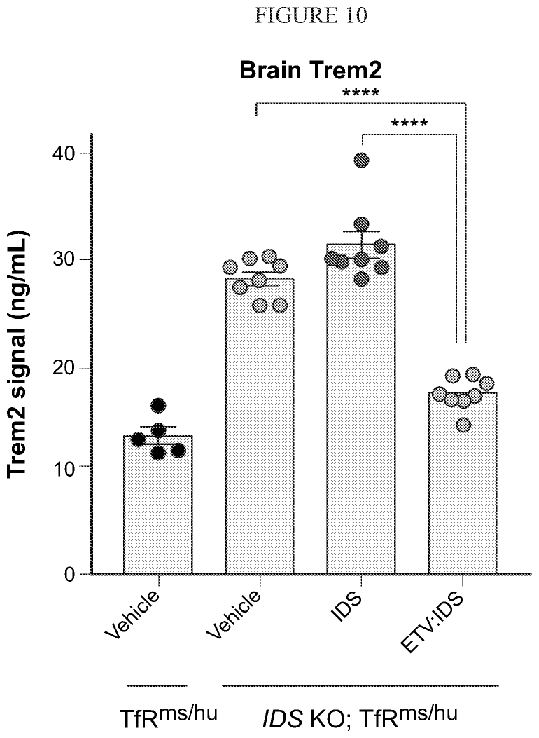

FIG. 10 shows that peripheral administration of ETV:IDS corrected TREM2 accumulation in IDS KO brains.

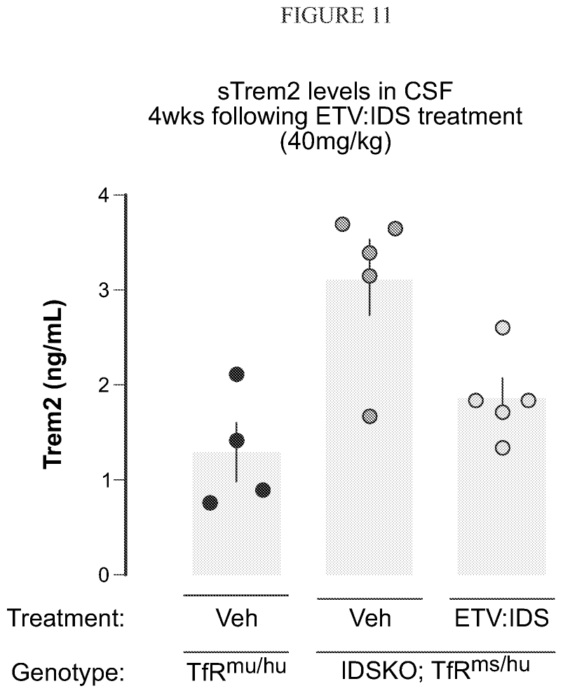

FIG. 11 illustrates that an increase in CSF sTrem2 levels was observed in IDS KO; TfR^{mu/hu} KI mice relative to a TfR^{mu/hu} KI mice cohort. Treatment with ETV:IDS reduced the accumulation of CSF sTrem2 levels in the IDS KO; TfR^{mu/hu} KI mice.

Table 1 provides a summary of the GlcCer species analyzed, with the levels represented as fold over WT.

Table 2 provides data for lipid levels in brains of IDS KO mice treated with vehicle, ETV:IDS or Elaprase (fold changes over WT).

### Example 2. Measurement of Brain GAG, Lysosomal Lipids, and Neurofilament Light Chain (Nf-L) in IDS KO Mice at 3, 6 and 9 Months of Age

As described below, brain GAG, lysosomal lipid, and neurofilament light chain (Nf-L) levels were investigated in WT and IDS KO mice at 3, 6 and 9 months of age.

### Materials and Methods

Animals used in this study were cared for as described herein (Example 1). Tissues were sampled, and lipid and GAG levels were measured using methods described herein (Example 1). Nf-L levels were measured as described below.

### Methods for CSF and Serum Analysis of Nf-L

Using Quanterix Simoa Neurofilament-Light (NF-L) Sample Diluent (Quanterix 102252), cerebrospinal fluid (CSF) was diluted 100× and serum was diluted 4× before being added to Simoa 96-well microplate (Quanterix 101457). NF-light assay was carried out according to Simoa NF-Light Advantage Kit (Quanterix 1031086) instructions using Simoa detector reagent and bead reagent (Quanterix 103159 and 102246, respectively). After incubation of samples with detector and bead reagent at 30° C., 800 rpm for 30 minutes, the sample plate was washed with Simoa Wash Buffer A (Quanterix 103078) on Simoa Microplate Washer according to Quanterix two step protocol. SBG reagent (Quanterix 102250) was subsequently added, and samples were incubated at 30° C., 800 rpm for 10 minutes. The 2-step washer protocol was continued, with the sample beads being twice resuspended in Simoa Wash Buffer B (Quanterix 103079) before final aspiration of buffer. Sample NF-L levels were measured using the NF-Light analysis protocol on the Quanterix SR-X instrument and interpolated against a calibration curve provided with the Quanterix assay kit.

### Results

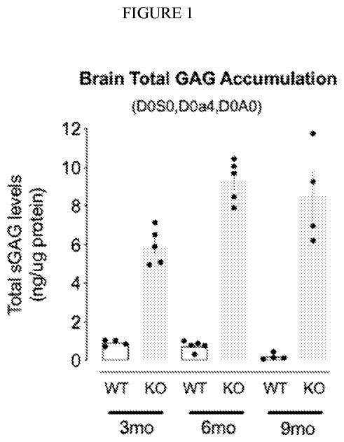

Brain HS/DS (GAG) accumulation at ages 3, 6, and 9 months in IDS KO mice relative to age-matched WT controls was measured (FIG. 1). GAG accumulation was significantly higher in the IDS KO mice at all ages tested.

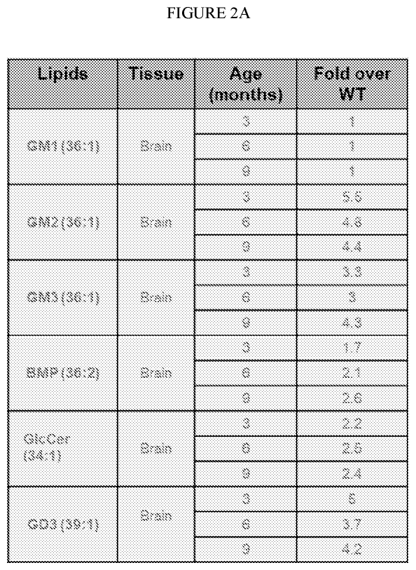

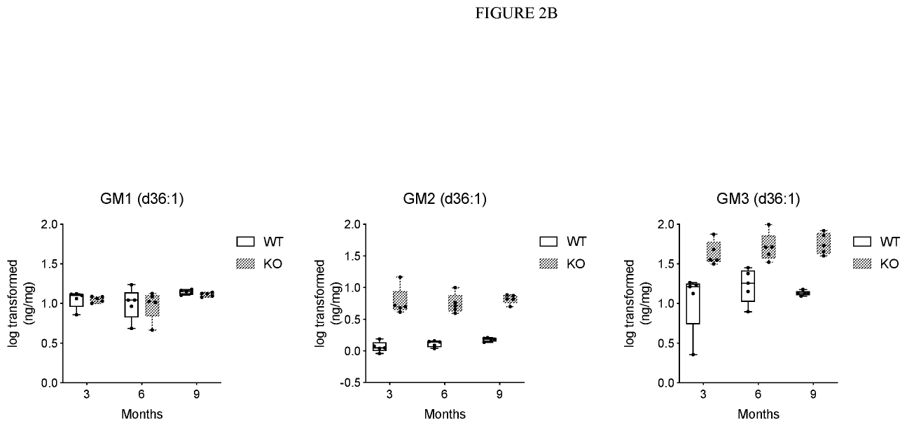

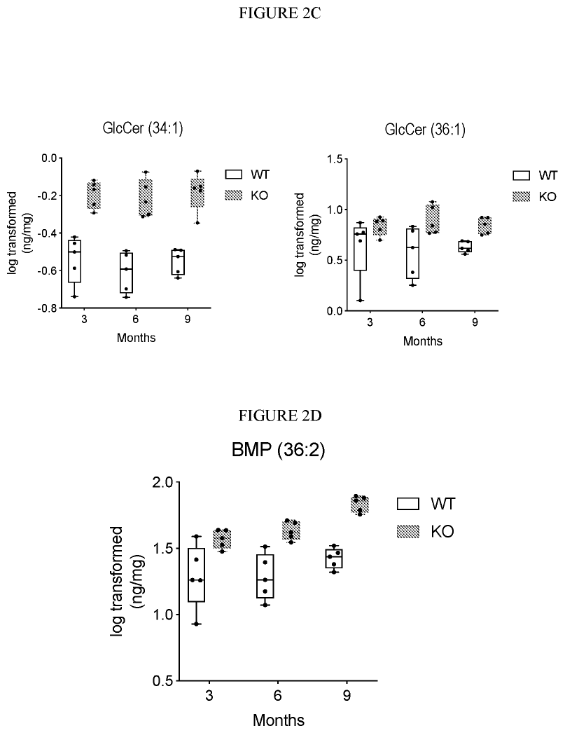

Lysosomal lipid accumulation (GM1, GM2, GM3, BMP, GlcCer and GD3) in the brains of IDS KO mice relative to age-matched WT controls was also measured (FIGS. 2A-2D). GM1 (d36:1) showed similar levels in both IDS KO and WT mice (FIGS. 2A and 2B), whereas levels of GM2 (36:1), GM3 (36:1), BMP (36:2), GlcCer (34:1), and GD (39:1) all showed between 1.7 and 5.5 fold increases over WT animals (FIGS. 2A-2D). Similar to GD3 (39:1), GD3 (36:1) also is increased in the IDS KO (2 fold on average).

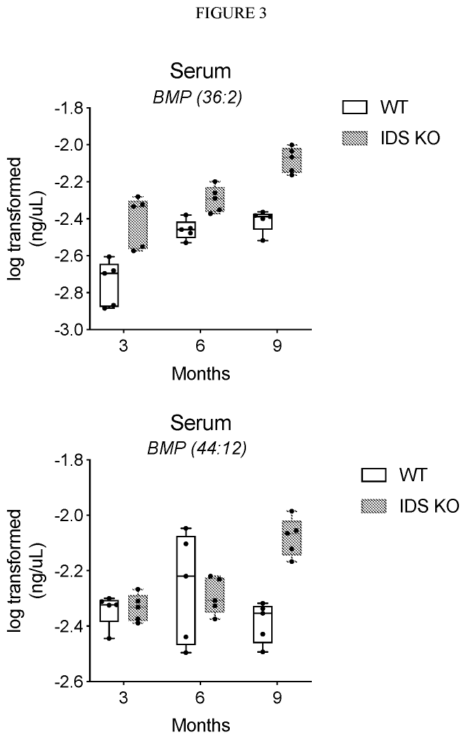

Elevated levels of BMP in the serum of IDS KO mice relative to their age-matched controls were also observed. In particular, BMP (36:2) and BMP (44:12) were increased particularly in the 9 month serum samples taken in IDS KO mice as compared to WT controls (FIG. 3).

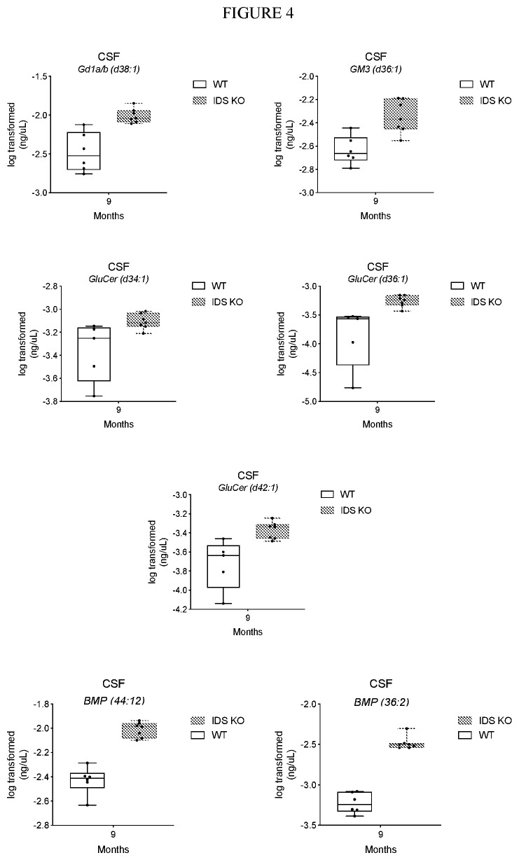

Elevated levels of lysosomal lipids (Gd1a/b, GM3, BMP and GlcCer) were observed in the CSF of 9 month old IDS KO mice relative to WT age-matched controls (FIG. 4).

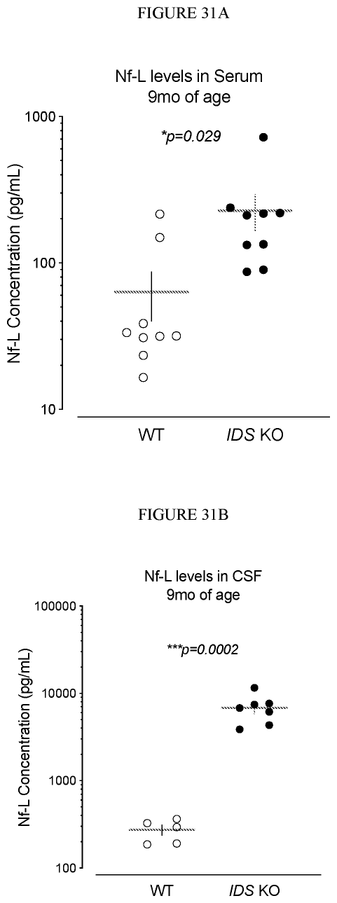

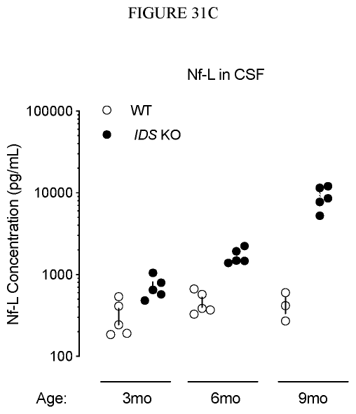

Nf-L is a useful marker for neurodegeneration (Norgren et al. 2003. Brain Research 987(1):25-31), but it has not previously been associated with Hunter syndrome/MPSII. Nf-L concentrations in serum and CSF of 9-month old IDS KO mice were elevated relative to a same-age cohort of wild-type mice (FIGS. 31A, 31B). The relative difference in Nf-L levels in CSF increased with the age of the mice cohorts, with the largest difference in Nf-L levels observed in the 9-month old mice cohort groups (FIG. 31C). These results indicate that Nf-L can also be useful as a disease-related and treatment-responsive biomarker in mouse models of Hunter syndrome.

### Example 3. Effect of Peripheral Administration of ETV:IDS on GAG and Lysosomal Lipids in IDS KO×TfR^{muhu} Mice

The effect of varying doses of ETV:IDS on GAG and lysosomal lipids in IDS KO×TfR^{muhu} mice was examined.

### Materials and Methods

### Animal Care

Mice were housed under a 12-hour light/dark cycle and had access to water and standard rodent diet (LabDiet® #25502, Irradiated) ad libitum.

### Mouse Strains

The IDS KO×TfR^{mu/hu} mice used in this study are described in Example 1 above. All mice used in this study were males.

### Administration and Sample Collection

2-3 month old IDS KO×TfR^{mu/hu} mice were injected i.v. with saline, idursulfase (14.2 mg/kg body weight), or ETV:IDS (3, 10, 20, or 40 mg/kg body weight) once every week for 4 weeks (n=5-8) (see, Example 6 for ETV:IDS). 2-3 month-old littermate TfR^{mu/hu} mice, injected i.v. with saline once every week for 4 weeks (n=5) were used as controls. Animals were sacrificed 7 days following last 4-week dose.

For terminal sample collection, animals were deeply anesthetized via intraperitoneal (i.p.) injection of 2.5% Avertin. For CSF collection, a sagittal incision was made at the back of the animal's skull, subcutaneous tissue and muscle was separated to expose the cisterna magna and a pre-pulled glass capillary tube was used to puncture the cisterna magna to collect CSF. CSF was transferred to a Low Protein LoBind Eppendorf tube and centrifuged at 12,700 rpm for 10 minutes at 4° C. CSF was transferred to a fresh tube and snap frozen on dry ice. Lack of blood contamination in mouse CSF was confirmed by measuring the absorbance of the samples at 420 nm. Blood was collected via cardiac puncture for serum collection. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice. Animals were transcardially perfused with ice-cold PBS using a peristaltic pump (Gilson Inc. Minipuls Evolution). The brain was dissected and flash-frozen on dry ice.

### Tissue Preparation and LCMS Assay

Tissue preparation and LCMS assays were performed using methods similar to those described in Example 1.

### Results

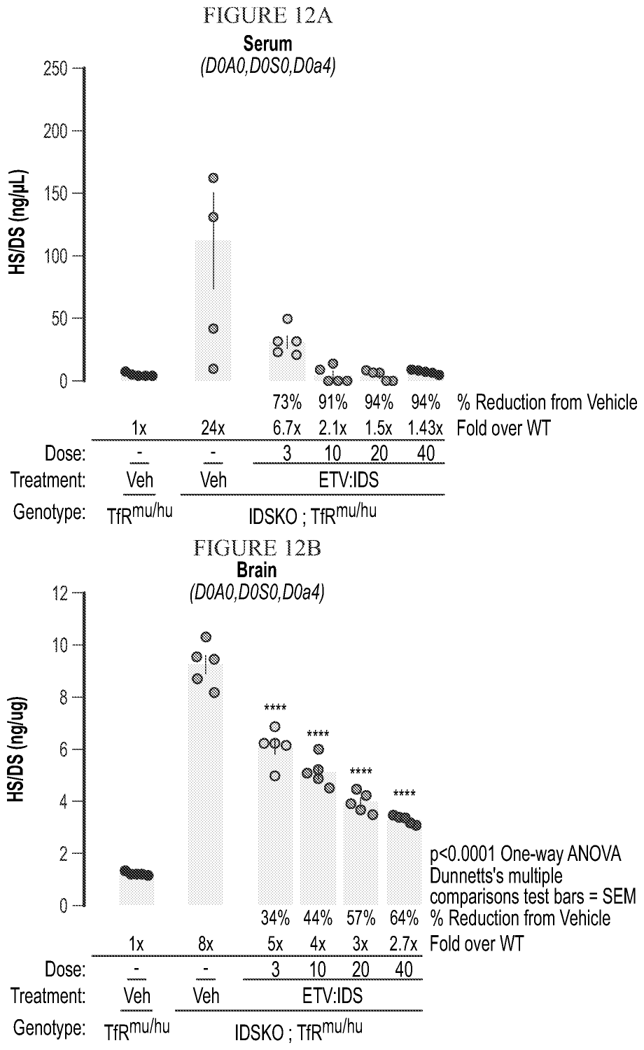

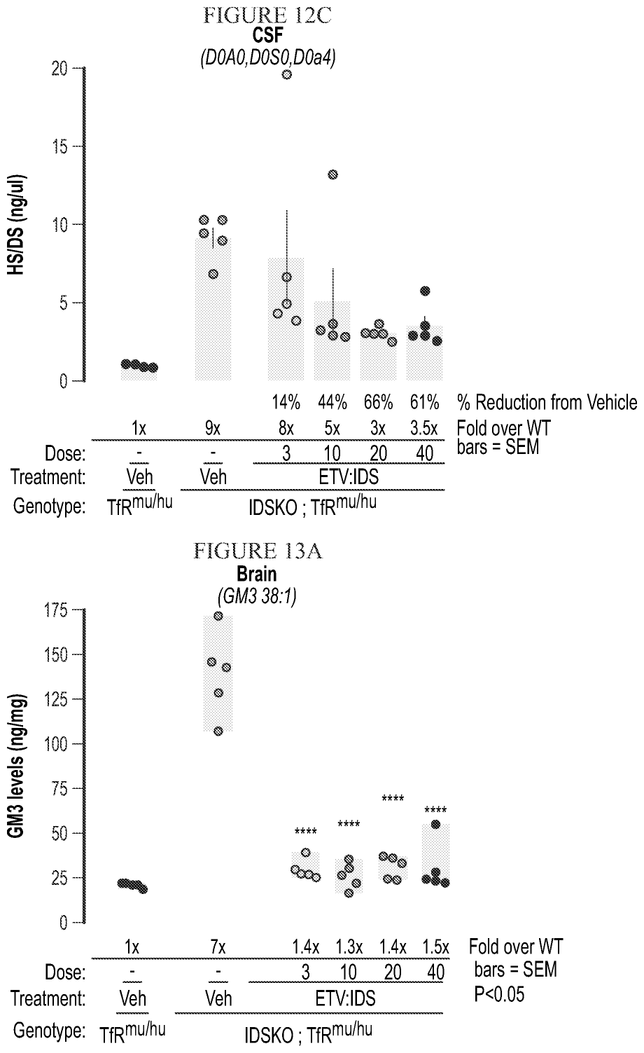

A dose-dependent decrease in serum brain and CSF GAGs was observed (FIGS. 12A-12C) for mice receiving ETV:IDS. Notably, serum GAGs were corrected down to wildtype levels.

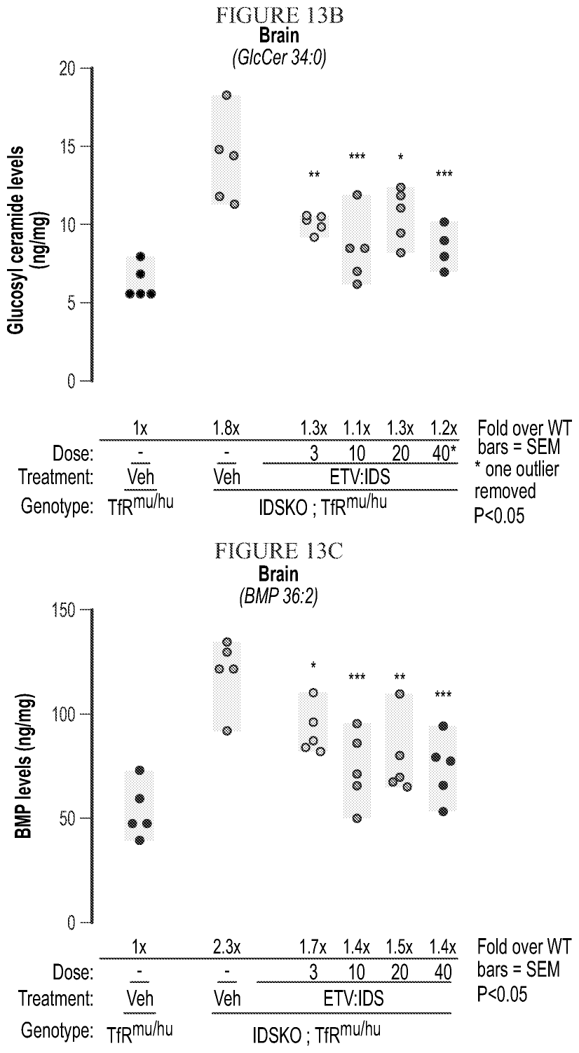

Importantly, there was a substantial reduction in brain lysosomal lipids (GM3, GlcCer and BMP), even at the lowest ETV:IDS dose (3 mg/kg) tested (FIGS. 13A-13C). Because lipid accumulation in IDS-deficient mice is believed to be a functional result of the GAG accumulation, these results may suggest that functional restoration of lysosomal function in the brain can be achieved even with GAG levels that are elevated relative to WT mice.

### Example 4. Effect of Peripheral Administration of ETV:IDS on GAG and Lysosomal Lipids in Specific CNS Cell Types of IDS KO×TfR^{muhu} Mice

A protocol was developed to isolate enriched populations of neurons, astrocytes, and microglial cells from brain tissue. The enriched populations from IDS KO×TfR^{muhu} mice were then used to investigate the effect of varying doses of ETV:IDS on GAG and lysosomal.

### Materials and Methods

### Animal Care

Mice were housed under a 12-hour light/dark cycle and had access to water and standard rodent diet (LabDiet® #25502, Irradiated) ad libitum.

### Mouse Strains

The TfR^{mu/hu} mice and IDS KO×TfR^{m/hu} mice used in this study are described in Example 1 above. All mice used in this study were males.

### Administration and Sample Collection

2-3 month old IDS KO×TfR^{mu/hu} mice were injected i.v. with saline or ETV:IDS (40 mg/kg body weight) once every week for 4 weeks (n=4-6 per treatment) (see, Example 6 for ETV:IDS). 2-3 month-old littermate TfR^{mu/hu} mice, injected i.v. with saline once every week for 4 weeks (n=4-6) were used as controls. Animals were sacrificed 7 days following last 4-week dose.

For terminal sample collection, animals were deeply anesthetized via intraperitoneal (i.p.) injection of 2.5% Avertin. For CSF collection, a sagittal incision was made at the back of the animal's skull, subcutaneous tissue and muscle was separated to expose the cisterna magna and a pre-pulled glass capillary tube was used to puncture the cisterna magna to collect CSF. CSF was transferred to a Low Protein LoBind Eppendorf tube and centrifuged at 12,700 rpm for 10 minutes at 4° C. CSF was transferred to a fresh tube and snap frozen on dry ice. Lack of blood contamination in mouse CSF was confirmed by measuring the absorbance of the samples at 420 nm. Blood was collected via cardiac puncture for serum collection. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice. Animals were transcardially perfused with ice-cold PBS using a peristaltic pump (Gilson Inc. Minipuls Evolution). The brain was dissected and flash-frozen on dry ice.

### CNS Cell Type Isolation

To prepare a single cell suspension for sorting CNS cells, mice were perfused with PBS, brains dissected and processed into a single cell suspension according to the manufacturers' protocol using the adult brain dissociation kit (Miltenyi Biotec 130-107-677). Cells were Fc blocked (Biolegend #101320, 1:100) and stained for flow cytometric analysis with Fixable Viability Stain BV510 (BD Biosciences #564406, 1:100) to exclude dead cells, CD11b-BV421 (BD Biosciences 562605, 1:100), CD31-PerCP Cy5.5 (BD Biosciences #562861, 1:100), 01-488 (Thermo/eBio #14-6506-82, 1:37.5), Thy1-PE (R&D #FAB7335P, 1:100), and EAAT2-633 (Alomone #AGC-022-FR, 1:50). Cells were washed with PBS/1% BSA and strained through a 100 μm filter before sorting CD11b+ microglia, EAAT2+ astrocytes, and Thy1+ neurons on a FACS Aria III (BD Biosciences) with a 100 μm nozzle. In order to achieve pure populations of astrocytes, microglia, and neurons negative gates were set to remove O1+ and CD31+ cells which are predominantly oligodendrocytes and endothelial cells respectively. Sorted cells were either pelleted or collected directly into lysis buffers, and then processed for downstream analysis including qRT-PCR, RNAseq, or glycomics as described in the relevant methods disclosed herein. Cell numbers were used to calculate pg GAG/cell.

Cell lysate preparation and LCMS assays for measurement of GAGs, BMPs, gangliosides, GlcCer, and GalCer were performed using methods similar to those described in Example 1.

### Distribution Analysis of ETV:IDS Across CNS Cell Types

Live cells in sheath fluid (˜1.5 ml) were sorted directly into 150 μL 5% CHAPS buffer for lysis, final concentration of CHAPS 0.5%. Samples were concentrated with Amicon Ultra 30 KDa filters. Five (5) μL of sample or recombinant ETV:IDS dilution series was run with an IgG (human) AlphaLISA Detection Kit (PerkinElmer #AL205C) per the manufacturer's instructions and read on an EnVision™ plate reader. Sample concentrations were interpolated from the standard curve generated using ETV:IDS and normalized to total cell input number.

### Results

To confirm the enrichment and purity of isolated cell populations, gene expression profiles for each of the sorted populations were analyzed using RNA-Seq and qRT-PCR and compared to known profiles (data not shown). A strong enrichment of corresponding cell-type specific genes in isolated populations and a reduction in gene markers of endothelial cells and oligodendrocytes was observed (data not shown). This collective data demonstrates that highly pure populations of neurons, astrocytes, and microglia were obtained.

The cell-type specific distribution and efficacy of ETV:IDS in the brains of IDS KO; TfR^{mu/hu}KI mice were subsequently assessed.

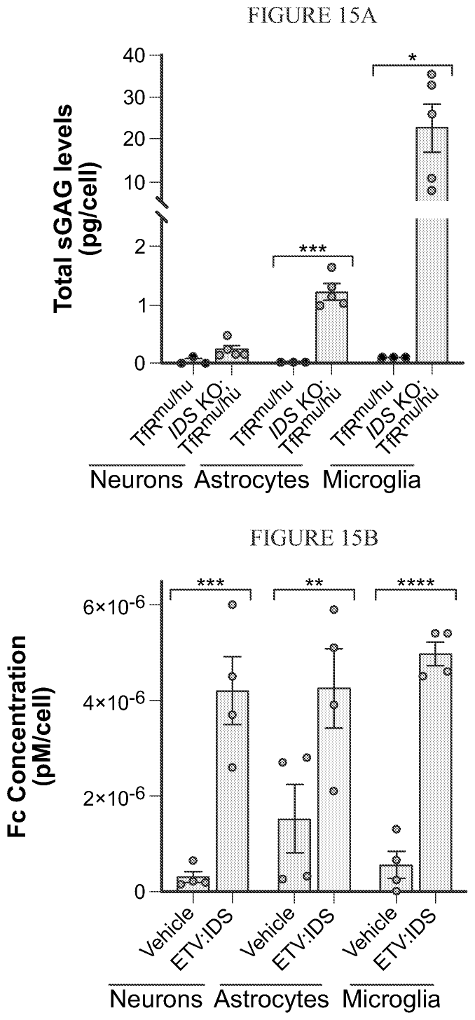

GAG levels in enriched CNS cell populations were quantified as described by LC-MS/MS. FIG. 15A illustrates that GAG levels were elevated in microglia, astrocytes, and neurons isolated from IDS KO; TfR^{mu/hu}KI mice relative to TfR^{mu/hu} KI controls, demonstrating substrate accumulation across all three CNS cell types when IDS expression was knocked-out (n=3-5 mice per group). The IDS KO; TfR^{mu/hu}KI mice (n=4 mice per group) were dosed intravenously with 40 mg/kg ETV:IDS, and enzyme concentration was assessed across CNS cell populations two hours post-dose. FIG. 15B illustrates that significant accumulation of ETV:IDS was observed in neurons, astrocytes, and microglia, demonstrating that ETV:IDS effectively distributes to the brain parenchyma and is taken up by key CNS cell types. All data are displayed as mean±SEM; unpaired student's t-test p≤0.05*, 0.001***.

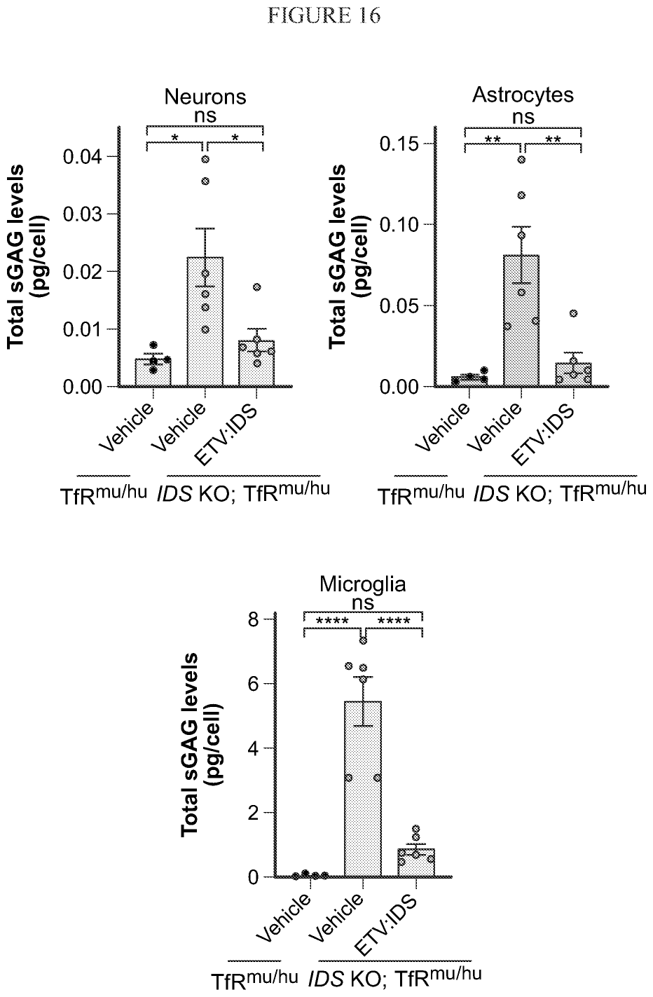

Next, IDS KO; TfR^{mu/hu} KI mice were dosed with 40 mg/kg ETV:IDS intravenously once a week for four weeks, and LC-MS/MS was used to assess the ability of ETV:IDS to reduce GAG accumulation in all three CNS cell types. FIG. 16 illustrates that ETV:IDS treatment reduced GAG levels in neurons, astrocytes, and microglia to levels comparable to that seen in wild-type mice following repeated administration. Data are displayed as mean±SEM; one-way ANOVA with Tukey's multiple comparison test, * p≤0.05, ** p≤0.01. The data demonstrates that ETV:IDS enables the delivery of IDS past the cerebral endothelium to brain cells and that this delivery is sufficient for efficacy across key CNS cell types.

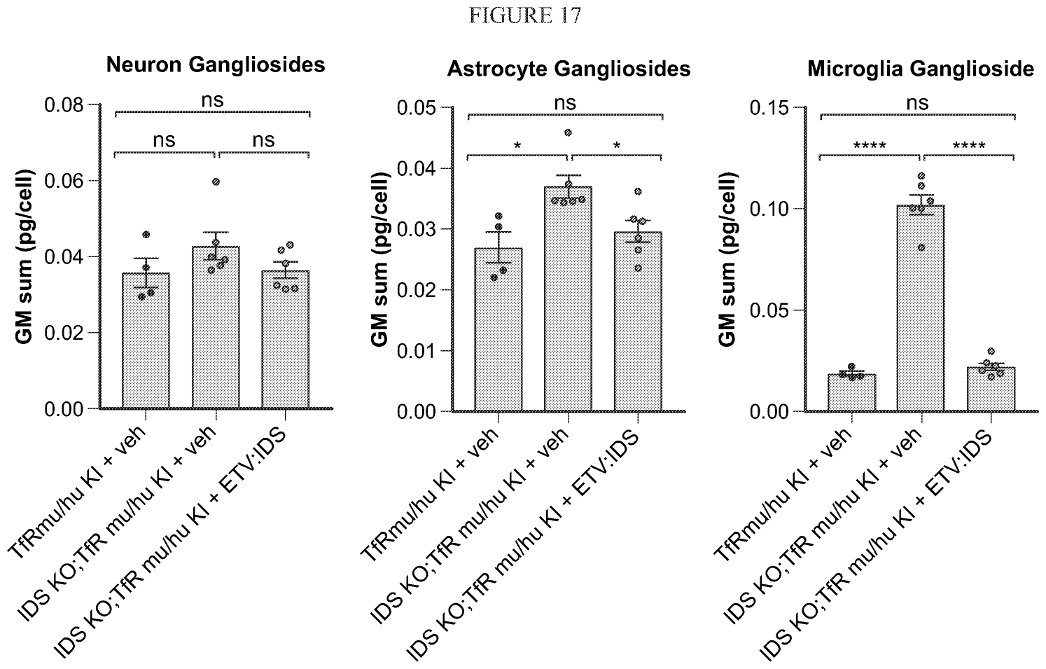

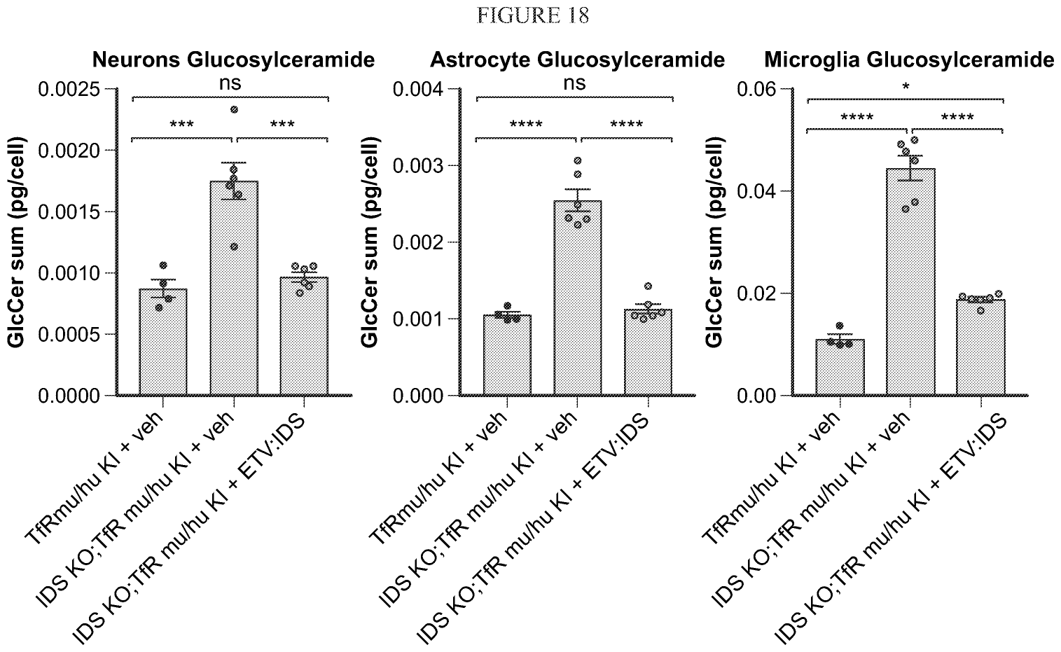

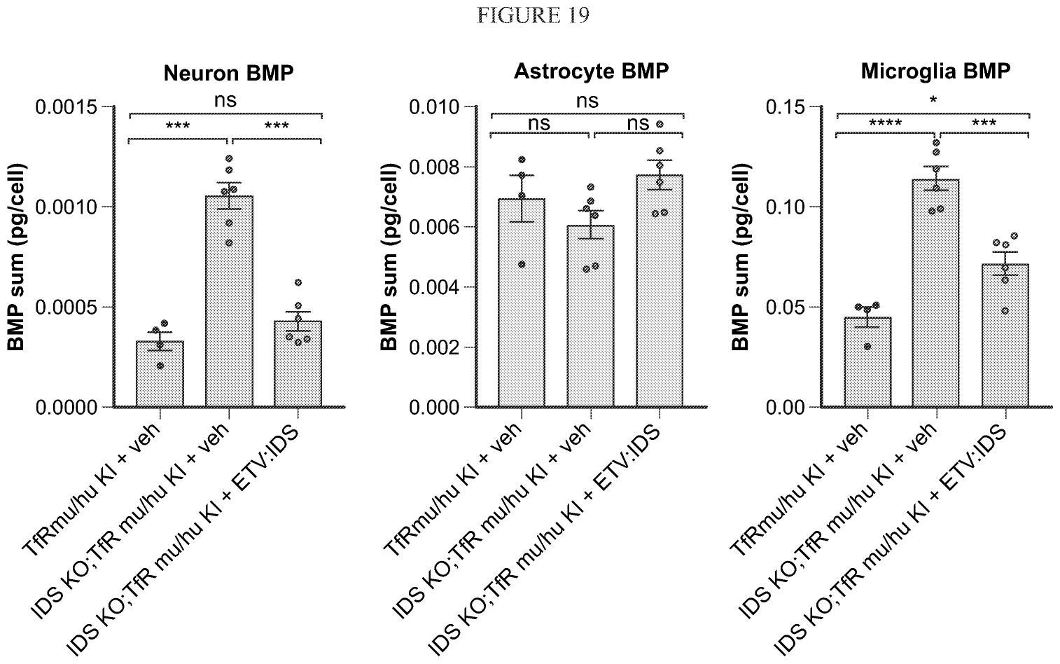

ETV:IDS treatment also reduced the secondary accumulation of the lysosomal lipids including gangliosides (FIG. 17), glucosylceramides (FIG. 18), and bis(monoacylglycerol)phosphate (BMP) (FIG. 19) in CNS cell types of interest (e.g., neurons, astrocytes, microglial cells). These data illustrate that treatment with ETV:IDS corrects secondary lysosomal dysfunction in addition to primary GAG storage in CNS cell types.

### Example 5. Spatial Distribution of Accumulated Lipid Species and Correction of Lipid Accumulation by ETV:IDS

The effect of varying doses of ETV:IDS on the spatial distribution of lysosomal lipids and on microglial activation in IDS KO×TfR^{muhu} mice was examined.

### Mouse Strains and Administration

The TfR^{mu/hu} mice and IDS KO×TfR^{mu/hu} mice used in this study are described in Example 1 above. All mice used in this study were males. The mice were administered saline, idursulfase, or ETV:IDS (40 mg/kg body weight) and sacrificed for tissue analysis as described in Example 1 (see, Example 6 for ETV:IDS).

### Mass-Spectrometry Based Imaging of Ganglioside Levels

Brain tissue was flash frozen on aluminum foil that was slowly lowered into liquid nitrogen for approximately 10 seconds. Frozen brains were stored at −80° C. until ready for use. Prior to sectioning, the brains were placed in a cryostat chamber to equilibrate the tissues to −20° C. Brains were cut on a cryostat (Leica Biosystems, Buffalo Grove, Ill.) into 12 μm thick sections and thaw-mounted onto indium-tix oxide (ITO) coated slides (Delta Technologies, Loveland, Colo.). Two brain levels were collected at approximately +0.72 mm and −1.82 mm from Bregma. Plates with sections designated for IMS were washed three times with chilled (about 4° C.) 50 mM ammonium formate and allowed to dry at room temperature prior to matrix application. Additional sections were obtained for H&E staining. After staining, digital micrographs were obtained via a slide scanner (Leica Biosystems, Buffalo Grove, Ill.). For matrix application, the plates were coated with 1,5-diaminonaphthalene (DAN) MALDI matrix via sublimation (Hankin et al. 2007. Journal of the American Society for Mass Spectrometry 18:1646-1652; Thomas et al. 2012. Analytical Chemistry 84:2048-2054). Briefly, 100 mg of recrystallized DAN was placed in the bottom of a glass sublimation apparatus (Chemglass Life Sciences, Vineland, N.J.). The apparatus was placed on a metal heating block set to 130° C., and DAN was sublimated onto the tissue surface for 4 minutes at a pressure of less than 25 mTorr. Approximately 1.8 mg of DAN was applied to each slide, determined by weighing the slide before and after matrix application. The coated plates were then placed in a Petri dish, flushed with nitrogen gas, and stored at −80° C. for two days prior to MS analysis (Yang et al. 2019. International Journal of Mass Spectrometry 437:3-9).

For imaging mass spectrometry, the plates were allowed to equilibrate to room temperature prior to removal from the sealed Petri dish. The brain sections were imaged on a Solarix 15T FT-ICR MS (Bruker Daltonics, Billerica, Mass.), equipped with a SmartBeam II 2 kHz frequency tripled Nd:YAG laser (355 nm). Images were acquired at 100 μm spatial resolution in negative ion mode. Each pixel is the average of 1000 laser shots using the small laser focus setting and random-walking within the 100 μm pixel. The mass spectrometer was externally calibrated with a series of phosphorus clusters (Sládková et al. 2009. Rapid Communications in Mass Spectrometry 23:3114-3118). Data were collected from m/z 600-3,000 with a time-domain file size of 1 M (FID length=1.3631 sec), resulting in a resolving power of 153,000 at m/z 1041. Images were generated using FlexImaging 3.0 (Bruker Daltonics, Billerica, Mass.). Gangliosides were identified by accurate mass, with the mass accuracies typically better than 1 ppm.

### Immunohistochemistry

Fresh frozen mouse brain tissue was sectioned coronally at 10 micron thickness using a Leica Cryostat (Leica CM 1950). Sections were directly mounted onto Fisherbrand Superfrost Plus microscope slides and stored at −80° C. until processed for immunohistochemistry. Sections were rinsed in 1×PBS for 3 rounds of 5 minutes then fixed in 4% Paraformaldehyde for 15 minutes. Sections were then rinsed in 1×PBS for 3 rounds of 5 minutes and incubated in Blocking Solution (1×PBS/5% normal goat serum/0.3% Triton X-100) for 1 hour at room temperature. Sections were then incubated in primary antibody (BioRad: Rat anti-Cd68, 1:500) prepared in Blocking Solution for 2 hours at room temperature. Sections were rinsed in 1×PBS/0.3% Triton X-100 for 3 rounds of 5 minutes followed by incubation in secondary antibody (Invitrogen: Goat anti-rat Alexa Fluor 488, 1:500) and DAPI (Invitrogen Molecular Probes D1306: 1:10,000 from 5 mg/mL stock) prepared in Blocking Solution for 1 hour at room temperature in the dark. Sections were then rinsed in 1×PBS/0.3% Triton X-100 for 3 rounds of 5 minutes, quickly rinsed in 1×PBS, and cover slipped with polyvinyl alcohol mounting medium with DABCO antifading (Sigma 10981). Fluorescent images were taken at 20× magnification using a Zeiss Axio Scan Z1. Each fluorophore was individually imaged with appropriate single-channel filter sets, using identical exposure times per fluorophore across all tissue samples imaged. Individual images were then tiled and stitched with shading correction using Zeiss Zen software.

### Results

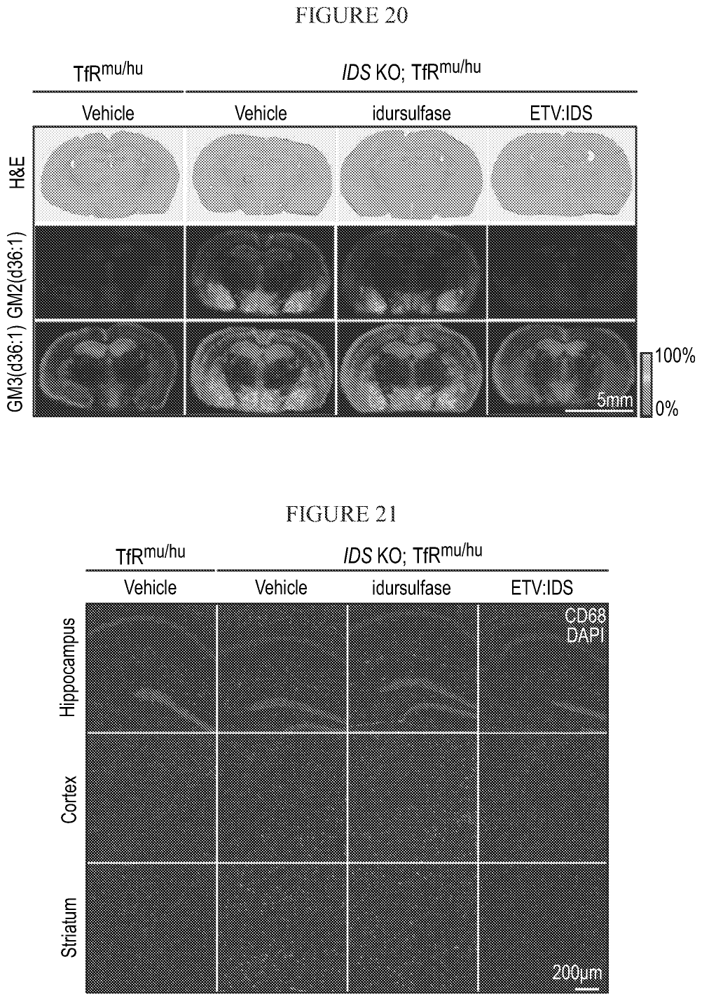

Imaging mass-spectrometry (IMS) was carried out to determine the spatial distribution of lipid accumulation in brains of in IDS KO; TfR^{mu/hu} KI mice and to assess whether ETV:IDS administration could correct lysosomal lipid accumulation throughout the brain. MALDI MS images were acquired from coronal brain sections of wild-type and IDS KO; TfR^{mu/hu} KI mice after four, weekly doses of vehicle, idursulfase or ETV:IDS. Representative images for select ganglioside species are illustrated in FIG. 20. The middle panel shows the distribution of the signal at m/z 1382.816, corresponding to GM2 (d36:1), while the bottom panel shows the distribution of the signal at m/z 1179.738, corresponding to GM3 (d36:1). Images depict the relative intensity of each signal from 0-100%.

As illustrated in FIG. 20, an enrichment of several species of gangliosides in the brains of IDS KO; TfR^{mu/hu}KI mice was observed compared with wild-type controls. This was similar to results observed in analysis of homogenized tissues (FIG. 6). Ganglioside accumulation was region specific and was concentrated in the hypothalamus and amygdala brain regions. Four weekly doses of ETV:IDS reduced the accumulation of these ganglioside species throughout all brain regions assessed to levels seen in wild-type mice, while only modest reductions in gangliosides were observed with idursulfase treatment. Taken together, these data demonstrate that ETV:IDS can correct lysosomal dysfunction downstream of GAG accumulation in the CNS.

Neuroinflammation represents a common hallmark of many neuronopathic LSDs, and there is an emerging consensus that glial activation, commonly reported in mouse models of MPS II disease as well as in MPS patients, may contribute to progressive degeneration throughout the brain in MPS disorders. The levels of two markers of microglia activity, CD68 and triggering receptor expressed on myeloid cells 2 (Trem2) were assessed through immunohistochemical analysis of brain tissue sections or biochemical analysis of brain lysates, respectively. FIG. 21 is a representative image of an immunofluorescent stain for DAPI and CD68 from the brains of TfR^{mu/hu} KI and IDS KO; TfR^{mu/hu} KI mice treated with vehicle, ETV:IDS or idursulfase. Magnification is 20×, and the images represent hippocampus (top panel), cortex (middle panel), and striatum (bottom panel). FIG. 10 illustrates levels of Trem2 in the treated mice.

The levels of CD68 and Trem2 were both elevated in the brains of IDS KO; TfR^{mu/hu} KI mice compared to TfR^{mu/hu}KI controls (FIGS. 10, 21). Four weekly doses of 40 mg/kg ETV:IDS effectively reduced the CD68 signal throughout the brains of IDS KO; TfR^{mu/hu} KI mice (FIG. 18). Levels of Trem2 in brain were completely reduced to levels seen in wild-type mice following repeated systemic administration of ETV:IDS (FIG. 10). Four weekly activity-equivalent doses of idursulfase, however, failed to lower levels of CD68 and Trem2. Administration of ETV:IDS also reduced accumulation of soluble Trem2 (sTrem2) in the CSF of treated IDS KO; TfR^{mu/hu} KI mice (FIG. 11). These data demonstrate that ETV:IDS can attenuate microglia activity in addition to GAG and secondary lysosomal lipid accumulation in the brain.

### Example 6. Construction of Fusion Proteins Comprising IDS

### Design and Cloning

IDS-Fc fusion proteins were designed that contain (i) a fusion polypeptide where a mature, human IDS enzyme is fused to a human IgG1 fragment that includes the Fc region (an “IDS-Fc fusion polypeptide”), and (ii) a modified human IgG1 fragment which contains mutations in the Fc region that confer transferrin receptor (TfR) binding (a “modified Fc polypeptide”). In particular, IDS-Fc fusion polypeptides were created in which IDS fragments were fused to either the N- or C-terminus of the human IgG1 Fc region. In some cases, a linker was placed between the IDS and IgG1 fragments to alleviate any steric hindrance between the two fragments. In all constructs, the signal peptide from the kappa chain V-III, amino acids 1-(UniProtKB ID—P01661) was inserted upstream of the fusion to facilitate secretion, and IDS was truncated to consist of amino acids S26-P550 (UniProtKB ID—P22304). The fragment of the human IgG1 Fc region used corresponds to amino acids D104-K330 of the sequence in UniProtKB ID P01857 (positions 221-447, EU numbering, which includes 10 amino acids of the hinge (positions 221-230)). In some embodiments, a second Fc polypeptide derived from human IgG1 residues D104-K330 but lacking the IDS fusion was co-transfected with the IDS-Fc fusion polypeptide in order to generate heterodimeric fusion proteins with one IDS enzyme (a “monozyme”). In some constructs, the IgG1 fragments contained additional mutations to facilitate heterodimerization of the two Fc regions. Control IDS-Fc fusion proteins that lack the mutations that confer TfR binding were designed and constructed analogously, with the difference being that these proteins lacked the mutations that confer TfR binding. As an additional control, IDS (amino acids S26-P550) with a C-terminal hexahistidine tag (SEQ ID NO:181) was generated to facilitate detection and purification.

The TfR-binding IDS-Fc fusion proteins used in the examples are dimers formed by an IDS-Fc fusion polypeptide and a modified Fc polypeptide that binds to TfR. For dimers where the IDS enzyme is linked to the N-terminus of the Fc region, the IDS-Fc fusion polypeptide may have the sequence of any one of SEQ ID NOS:91, 171, and 175. In these sequences, the IDS sequence is underlined and contains a cysteine at position 59 (double underlined) modified to formylglycine. The IDS was joined to the Fc polypeptide by a GGGGS linker (SEQ ID NO:179). A portion of an IgG1 hinge region (DKTHTCPPCP; SEQ ID NO:89) was included at the N-terminus of the Fc polypeptide. The CH2 domain sequence starts at position 541 of SEQ ID NOS:91, 171, and 175.

The IDS-Fc fusion protein ETV:IDS 35.21 is a dimer formed by an IDS-Fc fusion polypeptide having the sequence of any one of SEQ ID NOS:91, 171, and 175 and a modified Fc polypeptide that binds to TfR having the sequence of SEQ ID NO:92. The IDS-Fc fusion protein ETV:IDS 35.21 may also be further processed during cell culture production, such that the IDS-Fc fusion polypeptide has the sequence of any one of SEQ ID NOS:213, 215, and 217 and/or the modified Fc polypeptide that binds to TfR has the sequence of SEQ ID NO:189. Thus, as used herein, the term ETV:IDS 35.21 may be used to refer to protein molecules having unprocessed sequences (i.e., SEQ ID NOs:91, 171, 175, and 92); protein molecules comprising one or more processed sequences (i.e., selected from SEQ ID NOs: 213, 215, 217 and 189); or to a mixture comprising processed and unprocessed protein molecules. The first 10 amino acids are a portion of an IgG1 hinge region. The CH2 domain sequence starts at position 11 of SEQ ID NOs:92 and 189, respectively.

The IDS-Fc fusion protein ETV:IDS 35.21.17.2 is a dimer formed by an IDS-Fc fusion polypeptide having the sequence of any one of SEQ ID NOS:91, 171, and 175 and a modified Fc polypeptide that binds to TfR having the sequence of SEQ ID NO: 168. The IDS-Fc fusion protein ETV:IDS 35.21.17.2 may also be further processed during cell culture production, such that the IDS-Fc fusion polypeptide has the sequence of any one of SEQ ID NOS: 213, 215 and 217 and/or the modified Fc polypeptide that binds to TfR has the sequence of SEQ ID NO:207. Thus, as used herein, the term ETV:IDS 35.21.17.2 may be used to refer to protein molecules having unprocessed sequences (i.e., SEQ ID NOs:91, 171, 175, and 168); protein molecules comprising one or more processed sequences (i.e., selected from SEQ ID NOs: 213, 215, 217 and 207); or to a mixture comprising processed and unprocessed protein molecules. The first 10 amino acids are a portion of an IgG1 hinge region. The CH2 domain sequence starts at position 11 of SEQ ID NOs:168 and 207, respectively.

The IDS-Fc fusion protein ETV:IDS 35.23.2 is a dimer formed by an IDS-Fc fusion polypeptide having the sequence of any one of SEQ ID NOS:91, 171, and 175 and a modified Fc polypeptide that binds to TfR having the sequence of SEQ ID NO:169. The IDS-Fc fusion protein ETV:IDS 35.23.2 may also be further processed during cell culture production, such that the IDS-Fc fusion polypeptide has the sequence of any one of SEQ ID NOS: 213, 215 and 217 and/or the modified Fc polypeptide that binds to TfR has the sequence of SEQ ID NO:199. Thus, as used herein, the term ETV:IDS 35.23.2 may be used to refer to protein molecules having unprocessed sequences (i.e., SEQ ID NOs:91, 171, 175, and 169); protein molecules comprising one or more processed sequences (i.e., selected from SEQ ID NOs: 213, 215, 217 and 199); or to a mixture comprising processed and unprocessed protein molecules. The first 10 amino acids are a portion of an IgG1 hinge region. The CH2 domain sequence starts at position 11 of SEQ ID NOs:169 and 199, respectively.

The IDS-Fc fusion protein ETV:IDS 35.21.17 is a dimer formed by an IDS-Fc fusion polypeptide having the sequence of any one of SEQ ID NOS:91, 171, and 175 and a modified Fc polypeptide that binds to TfR having the sequence of SEQ ID NO:95. The IDS-Fc fusion protein ETV:IDS 35.21.17 may also be further processed during cell culture production, such that the IDS-Fc fusion polypeptide has the sequence of any one of SEQ ID NOS: 213, 215 and 217 and/or the modified Fc polypeptide that binds to TfR has the sequence of SEQ ID NO:191. Thus, as used herein, the term ETV:IDS 35.21.17 may be used to refer to protein molecules having unprocessed sequences (i.e., SEQ ID NOs:91, 171, 175, and 95); protein molecules comprising one or more processed sequences (i.e., selected from SEQ ID NOs: 213, 215, 217 and 191); or to a mixture comprising processed and unprocessed protein molecules. The N-terminus of the modified Fc polypeptide may include a portion of an IgG1 hinge region (e.g., SEQ ID NO:89).

### Recombinant Protein Expression and Purification

To express recombinant IDS enzyme fused to an Fc region, ExpiCHO cells (Thermo Fisher Scientific) were transfected with relevant DNA constructs using Expifectamine™ CHO transfection kit according to manufacturer's instructions (Thermo Fisher Scientific). Cells were grown in ExpiCHO™ Expression Medium at 37° C., 6% C02 and 120 rpm in an orbital shaker (Infors HT Multitron). In brief, logarithmic growing ExpiCHO™ cells were transfected at 6×10⁶ cells/ml density with 0.8 μg of DNA plasmid per mL of culture volume. After transfection, cells were returned to 37° C. and transfected cultures were supplemented with feed as indicated 18-22 hrs post transfection. Transfected cell culture supernatants were harvested 120 hrs post transfection by centrifugation at 3,500 rpm from 20 mins. Clarified supernatants were filtered (0.22 μM membrane) and stored at 4° C. Expression of an epitope-tagged IDS enzyme (used as a control) was carried out as described above with minor modifications. In brief, an IDS enzyme harboring a C-terminal hexahistidine tag (SEQ ID NO:181) was expressed in ExpiCHO cells.

IDS-Fc fusion proteins with (or without) engineered Fc regions conferring TfR binding were purified from cell culture supernatants using Protein A affinity chromatography. Supernatants were loaded onto a HiTrap MabSelect SuRe Protein A affinity column (GE Healthcare Life Sciences using an Akta Pure System). The column was then washed with >20 column volumes (CVs) of PBS. Bound proteins were eluted using 100 mM citrate/NaOH buffer pH 3.0 containing 150 mM NaCl. Immediately after elution, fractions were neutralized using 1 M arginine-670 mM succinate buffer pH 5.0 (at a 1:5 dilution). Homogeneity of IDS-Fc fusion proteins in eluted fractions was assessed by reducing and non-reducing SDS-PAGE.

To purify hexahistadine-tagged (SEQ ID NO:181) IDS enzyme, transfected supernatants were exhaustively dialyzed against 15 L of 20 mM HEPES pH 7.4 containing 100 mM NaCl overnight. Dialyzed supernatants were bound to a HisTrap column (GE Healthcare Life Sciences using an Akta Pure System). After binding, the column was washed with 20 CV of PBS. Bound proteins were eluted using PBS containing 500 mM imidazole. Homogeneity of IDS enzyme in eluted fractions was assessed by reducing and non-reducing SDS-PAGE. Pooled fractions containing IDS enzyme were diluted 1:10 in 50 mM Tris pH 7.5 and further purified using Q Sepharose High Performance (GE Healthcare). After binding, the column was washed with 10 CV of 50 mM Tris pH 7.5. Bound proteins were eluted using a linear gradient to 50 mM Tris pH 7.5 and 0.5 M NaCl and collected in 1 CV fractions. Fraction purity was assessed by non-reducing SDS-PAGE. Purification yielded homogeneous IDS-Fc fusion proteins and hexahistidine-tagged (SEQ ID NO:181) IDS enzyme.

### Example 7. Effect of Peripheral Administration of ETV:IDS on Substrate Accumulation, Lysosomal Lipid Storage, and Neuroinflammation after 4 Weeks of Treatment

The objective of this study was to assess the dose-dependent reduction of HS/DS accumulation and downstream markers of disease in brain and CSF following 4 weeks of ETV:IDS treatment in IDS KO; TfR^{mu/hu} KI mice compared to a 1 mg/kg dose of idursulfase.

### Materials and Methods

### Animal Care

Mice were housed under a 12-hour light/dark cycle and had access to water and standard rodent diet (LabDiet® #25502, Irradiated) ad libitum.

### Mouse Strains

The IDS KO×TfR^{mu/hu} mice used in this study are described in Example 1 above. All mice used in this study were males.

### Administration and Sample Collection

8-week old IDS KO; TfR^{mu/hu} KI mice were administered idursulfase at 1 mg/kg or ETV:IDS at 0 (Vehicle), 1 mg/kg, 3 mg/kg or 10 mg/kg IV via the tail vein (see, Example 6 for ETV:IDS). 8-week old TfR^{mu/hu} KI mice, injected with Vehicle, were used as controls (n=5). All animals were humanely euthanized either 2 hours after their first dose (Day 1; n=4) or 7 days following their last dose (Day 28; n=5).

In-life blood samples were collected by submandibular bleed. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice. For serum samples collected at necropsy, animals were deeply anesthetized via IP injection of 2.5% Avertin. Blood was collected via cardiac puncture and processed for serum as described above. Samples were stored in a freezer, set to maintain −60 to −80° C.

For terminal sample collection, animals were deeply anesthetized via intraperitoneal injection of 2.5% Avertin. For CSF collection, a sagittal incision was made at the back of the animal's skull, subcutaneous tissue and muscle was separated to expose the cisterna magna and a prepulled glass capillary tube was used to puncture the cisterna magna to collect CSF. CSF was transferred to a Low Protein LoBind Eppendorf tube and centrifuged at 12,700 rpm for 10 minutes at 4° C. CSF was transferred to a fresh tube and snap frozen on dry ice. Blood was collected via cardiac puncture for serum collection. Animals were transcardially perfused with ice-cold PBS using a peristaltic pump (Gilson Inc. Minipuls Evolution).

Approximately 50 mg of brain and liver tissue were placed into individually labeled tubes (one tube for each animal), flash frozen on dry ice, and placed on dry ice until transferred to a freezer, set to maintain −60 to −80° C. Leftover brain tissue was placed into individually labeled tubes (one tube for each animal), flash frozen on dry ice and placed on dry ice until transferred to a freezer, set to maintain −60 to −80° C., for GAG, Lipidomics, and Trem2 analysis.

### Tissue Preparation and LCMS Assay

Tissue preparation and LCMS assays were performed using methods similar to those described in Example 1.

### Tissue Preparation and Trem2 Analysis by Mesoscale Discovery (MSD)

Tissue preparation and Trem2 analysis by MSD was performed using methods similar to those described in Example 1.

### Results

### Pharmacodynamics

### Brain, CSF, and Liver GAGs

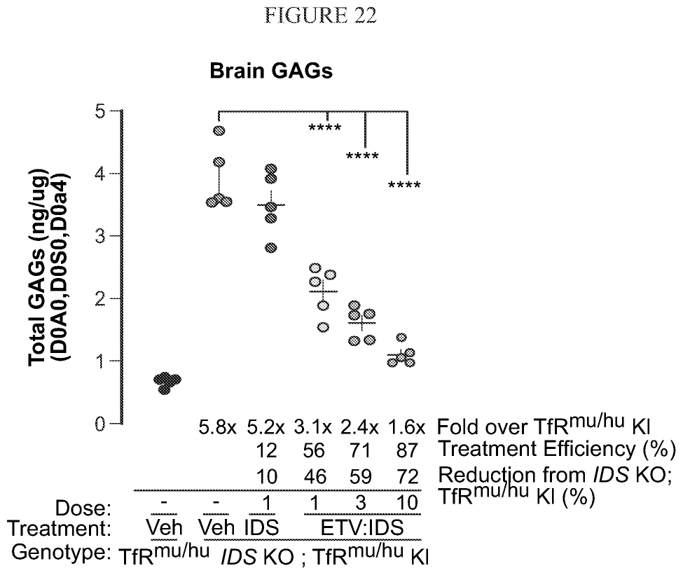

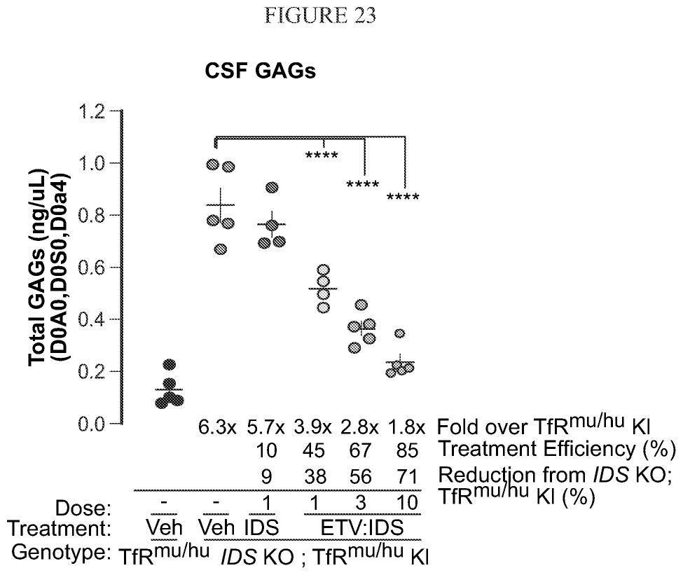

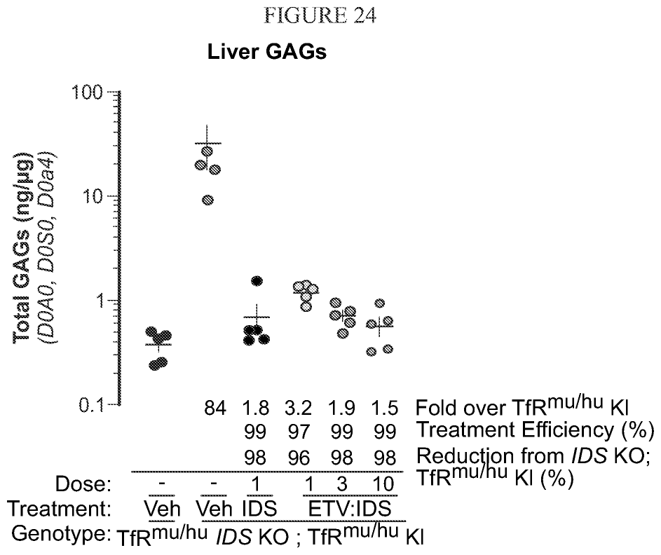

GAGs refer to the sum of heparan sulfate (D0A0, D0S0) and dermatan sulfate (D0a4) derived disaccharides. GAG levels in brain from IDS KO; TfR^{mu/hu} KI mice were measured 7 days after the last dose of ETV:IDS or idursulfase and compared to vehicle treatment and TfR^{mu/hu} KI mice. Brain GAG values decreased with increased doses of ETV:IDS and resulted in a treatment efficiency of 56%, 71%, and 87% from vehicle treated IDS KO; TfR^{mu/hu} KI mice for 1, 3, and 10 mg/kg, respectively while idursulfase resulted in a 12% treatment efficiency at a dose of 1 mg/kg (FIG. 22). The same observation was observed for CSF. CSF GAG values decreased with increasing doses of ETV:IDS and resulted in a treatment efficiency of 45%, 67%, and 85% from vehicle treated IDS KO; TfR^{mu/hu} KI mice for 1, 3, and 10 mg/kg, respectively while idursulfase resulted in a 10% treatment efficiency at a dose of 1 mg/kg (FIG. 23). Brain and CSF GAGs exhibited a high correlation across dosages (Spearman r=0.8976) supporting the utility of measuring changes in CSF GAG as a surrogate for changes in brain GAG. Levels of liver GAGs showed near complete correction at all dose levels of ETV:IDS (treatment efficiency of 97%, 99%, and 99% from vehicle treated IDS KO; TfR^{mu/hu} KI mice for 1, 3, and 10 mg/kg, respectively) and was comparable to idursulfase (treatment efficiency of 99%) at a dose of 1 mg/kg (FIG. 24).

### Brain and CSF Lysosomal Lipids

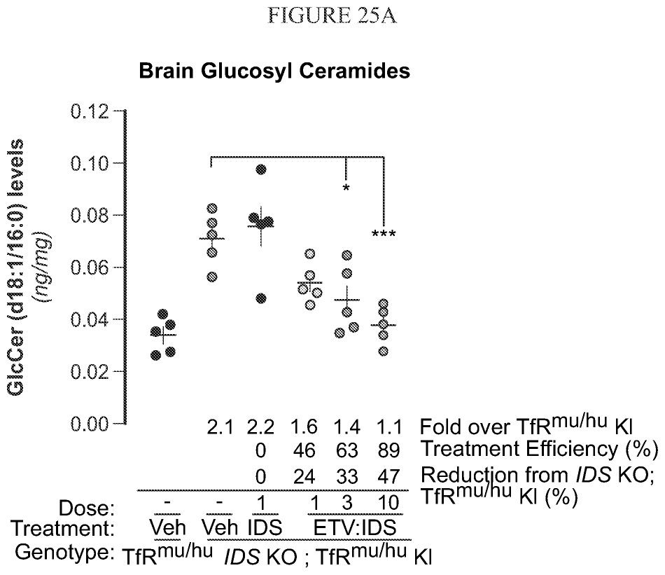

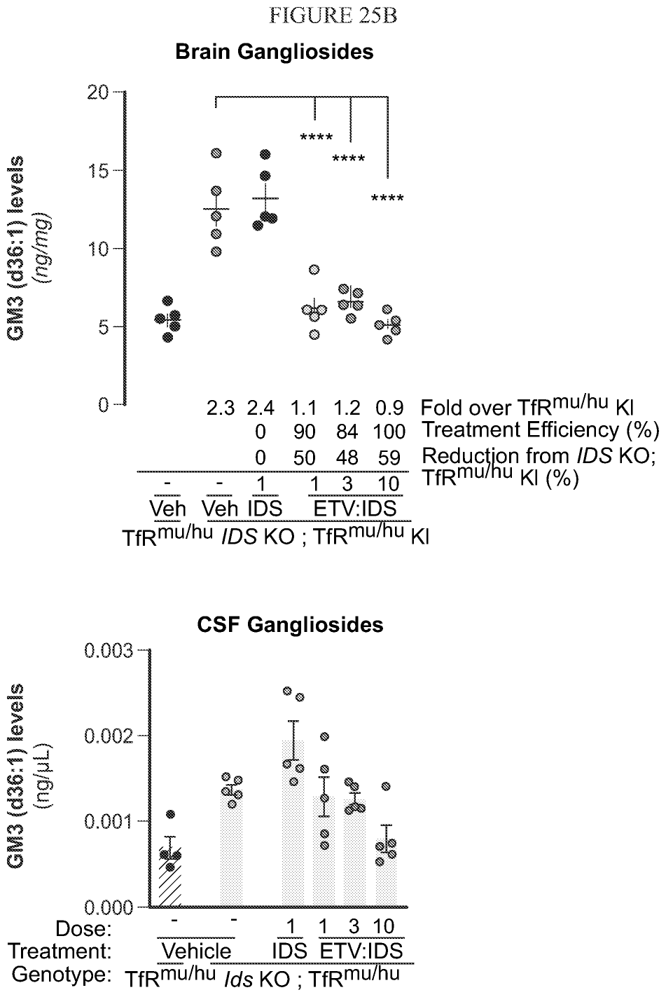

Lysosomal dysfunction, as assessed by measuring levels of accumulated lysosomal lipids, also showed a robust dose-dependent reduction following ETV:IDS treatment, with some lysosomal lipids (i.e. gangliosides) reaching levels comparable to TfR^{mu/hu} KI mice. For instance, GM3 reduction observed in brain at 7 days post the last dose of ETV:IDS resulted in a Fold over TfR^{mu/hu} KI of 1.1×, 1.2×, and 0.9× at 1, 3, and 10 mg/kg, respectively and a Percent treatment efficiency of 90%, 84%, and 100% for 1, 3, and 10 mg/kg, respectively. Brain BMP levels resulted in a treatment efficiency of 76% for all dose levels. In contrast, a dose of 1 mg/kg idursulfase did not have any change in lysosomal lipid accumulation, resulting in a treatment efficiency of 0% (FIGS. 25A-25C). There was also a dose dependent trend showing a reduction in CSF lysosomal levels of glucosylceramides, gangliosides and BMP following ETV:IDS treatment. FIG. 25B (bottom panel) illustrates a reduction in CSF levels of a representative ganglioside species, (d36:1).

### Brain Trem2

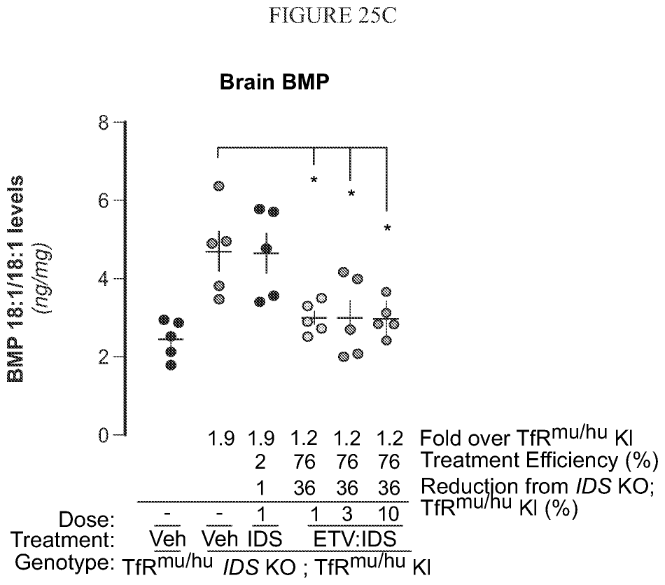

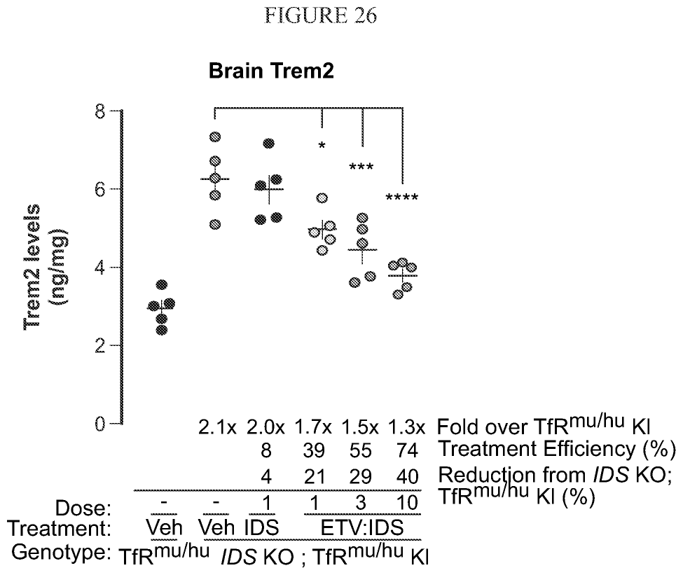

A dose dependent reduction in brain Trem2 levels (FIG. 26), a biomarker of activated microglia and neuroinflammation, was observed. Levels of brain Trem2 decreased with increasing doses of ETV:IDS and resulted in a treatment efficiency of 39%, 55%, and 74% from vehicle treated IDS KO; TfR^{mu/hu} KI mice for 1, 3, and 10 mg/kg, respectively while idursulfase resulted in a treatment efficiency of only 8%. A strong correlation between brain Trem2 and GAG levels (Spearman r=0.8736) was also observed.

### Serum, Brain, and Liver Concentrations

ETV:IDS serum, brain and liver concentrations at 2 hours post-dose on Day 1 (and Day 22 in serum) increased with increasing doses (data not shown). ETV:IDS mean brain concentrations after a single dose of 1, 3, or 10 mg/kg were 32.5-124-fold greater than idursulfase mean brain concentrations following a 1 mg/kg dose (data not shown).

### SUMMARY

These data show that intravenous administration of ETV:IDS for 4 weekly doses results in a robust and dose-dependent reduction in CNS and peripheral GAGs, as well as downstream markers of disease, such as lysosomal lipids and markers of neuroinflammation. In contrast, four weekly doses of idursulfase caused no significant change in brain GAG levels, lysosomal lipids, or Trem2. Restoration of some downstream markers of disease (i.e. gangliosides) to levels similar to IDS WT at doses that do not lead to complete correction of brain GAGs were observed. Additionally, brain and CSF GAGs exhibited a high correlation across dosages, supporting the utility of measuring changes in CSF GAG as a surrogate for changes in brain GAG.

### Example 8. Effect of Peripheral Administration of ETV:IDS on GAG and Neurofilament Light Chain (Nf-L) in 8-Week Old IDS KO×TfR^{muhu} Mice

The effect of 13 weekly IV doses (1 and 3 mg/kg) of ETV:IDS on GAG, lysosomal lipids, and neurofilament light chain (Nf-L) in IDS KO×TfR^{muhu} mice was examined.

### Materials and Methods

### Animal Care

Mice were housed under a 12-hour light/dark cycle and had access to water and standard rodent diet (LabDiet® #25502, Irradiated) ad libitum.

### Mouse Strains

The IDS KO×TfR^{mu/hu} mice and TfR^{mu/hu} KI mice used in this study are described in Example 1 above. All mice used in this study were males.

### Administration and Sample Collection

8-week old IDS KO; TfR^{mu/hu} KI mice were administered 0 (vehicle), 1 mg/kg, or 3 mg/kg ETV:IDS intravenously (IV) via the tail vein (see, Example 6 for ETV:IDS). Eight (8)-week old TfR^{mu/hu} KI mice (IDS WT), injected with vehicle, were used as non-disease controls. The ETV:IDS or vehicle was administered once weekly for a period of 13 weeks. All animals were euthanized 7 days following their last dose. Animal that were observed to have dose reactions, received 10-20 mg/kg diphenhydramine (intraperitoneally) IP, just prior to ETV:IDS administration on subsequent dose occasions.

Each animal was observed twice daily (a.m. and p.m.), except on weekends where animals were observed once in the a.m. Body weights were taken and recorded prior to the first dose and once weekly thereafter, prior to dosing.

In-life and terminal serum samples were collected in manner similar to that described in Examples 7 and 1, respectively. Additionally, prior to scheduled necropsy, approximately 250 microliters of blood were also collected via cardiac puncture for hematology. Blood was collected in EDTA tubes (Sarstedt Microvette 500 K3E) and slowly inverted 10 times. Terminal CSF samples were collected as described in Example 1. Brain and liver tissue samples were also collected as described in Example 1. In addition, urine was collected and chilled immediately prior to termination. Urine samples were then stored in a freezer, set to maintain −60 to −80° C., for urine biomarker analysis.

### Quantification of GAGs

Brain and liver tissues were prepared for quantification of GAGs (e.g. heparan sulfate (HS) and dermatan sulfate (DS)) as described in Example 1. Prior to LCMS assay to quantify GAGs, protein lysates (from tissue) or CSF, urine, or serum were mixed with a combination of Heparinases I, II, III, and Choidriotinase B. The digests were mixed with acetonitrile and analyzed by LCMS. LCMS assay to quantify GAGs was carried out as described in Example 1.

### Methods for CSF and Serum Analysis of Nf-L

Nf-L levels in serum and CSF were measured as described in Example 2.

### Histopathology

Representative samples were embedded in paraffin and stained for Hematoxylin and Eosin (H&E). Tissues were evaluated by light microscopy.

### Results

### Pharmacodynamics

### Brain, CSF, Liver and Urine GAGs

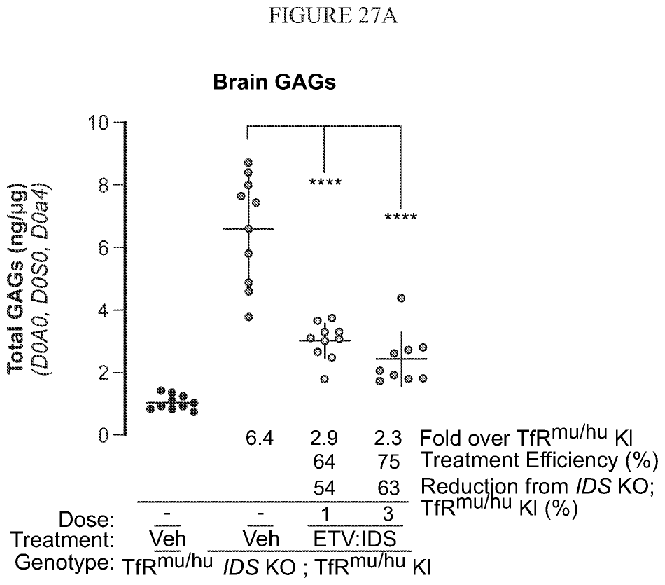

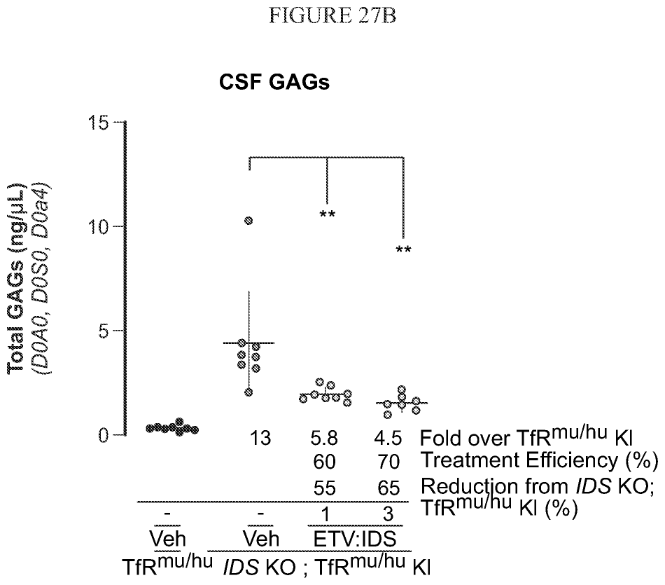

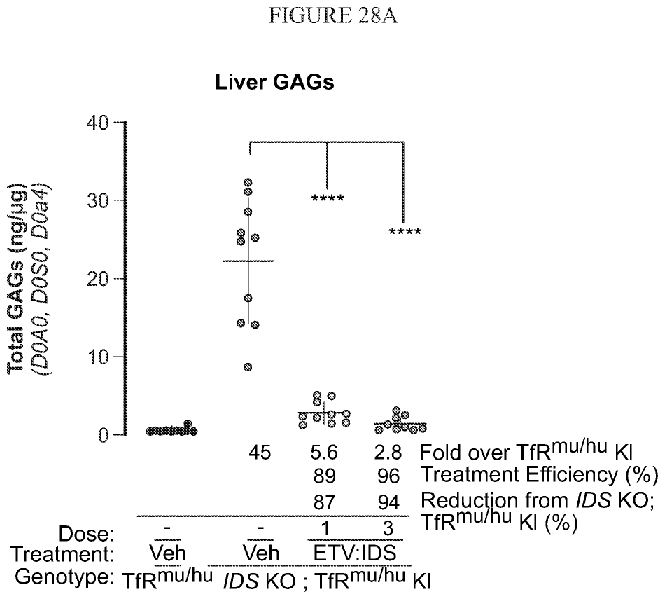

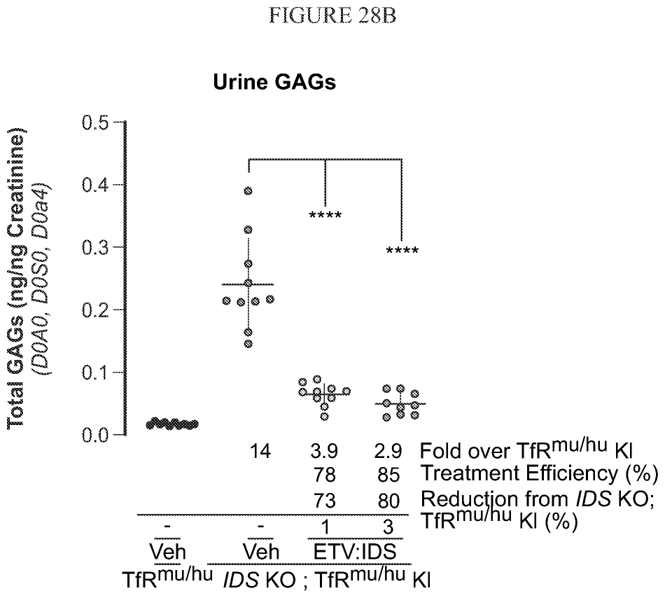

GAGs refer to the sum of heparan sulfate (D0A0, D0S0) and dermatan sulfate (D0a4) derived disaccharides. GAG levels in brain from IDS KO; TfR^{mu/hu} KI mice were measured 7 days after the last dose of ETV:IDS and compared to vehicle treatment and TfR^{mu/hu} mice. Brain GAG values decreased with increasing doses of ETV:IDS and resulted in a treatment efficiency of 64% and 75% from vehicle treated IDS KO; TfR^{mu/hu} KI mice for 1 and 3 mg/kg, respectively (FIG. 27A). The same observation was observed for CSF. CSF GAG values decreased with increasing doses of ETV:IDS and resulted in a treatment efficiency of 60% and 70% from vehicle treated IDS KO; TfR^{mu/hu} KI mice for 1 and 3 mg/kg, respectively (FIG. 27B). Brain and CSF GAGs exhibited a high correlation across dosages supporting the utility of measuring changes in CSF GAGs as a surrogate for changes in brain (data not shown). Levels of liver GAGs showed near complete correction at all dose levels of ETV:IDS (treatment efficiency of 98% and 96% from vehicle treated IDS KO; TfR^{mu/hu} KI mice for 1 and 3 mg/kg, respectively) (FIG. 28A). GAG levels in urine were also reduced following 13 weekly treatments with ETV:IDS. The treatment efficiency of creatinine normalized GAG levels were 78% and 85% with 1 and 3 mg/kg, respectively (FIG. 28B).

### CSF Neurofilament Light

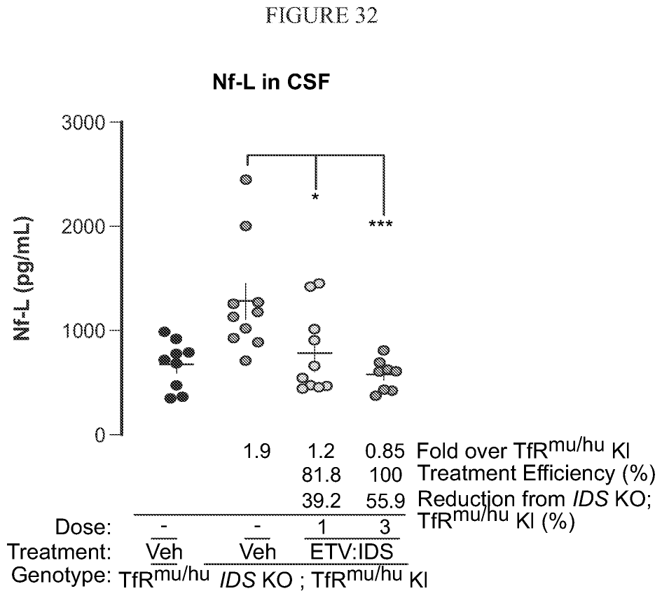

Building evidence suggests that CSF and plasma Neurofilament light chain (Nf-L) levels can serve as a maker of neuronal health and injury in preclinical models as well as neurodegenerative diseases (Bacioglu et al., Neuron 91, 56-66 (2016)). In a mouse model of Hunter syndrome, an elevation of Nf-L levels in the CSF and serum was observed (FIGS. 31A-31C; and FIG. 32). The ability of a long-term treatment with ETV:IDS to reverse these changes was evaluated and complete normalization of CSF Nf-L was observed in IDS KO; TfR^{mu/hu} KI treated with ETV:IDS. The treatment efficiency of Nf-L correction was 82% and 117% at 1 and 3 mg/kg, respectively, bringing the levels of Nf-L down to that observed in IDS WT controls (FIG. 32).

### Brain and CSF Lysosomal Lipids

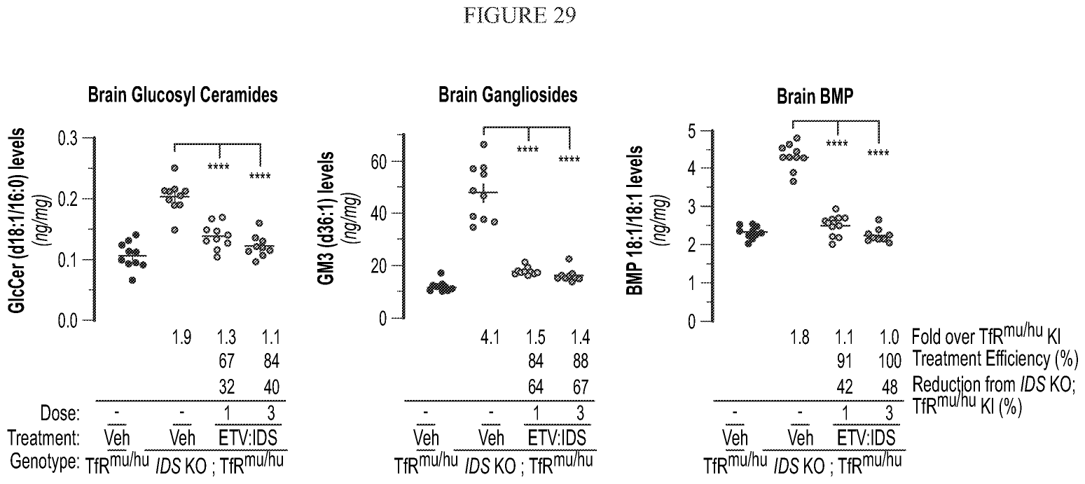

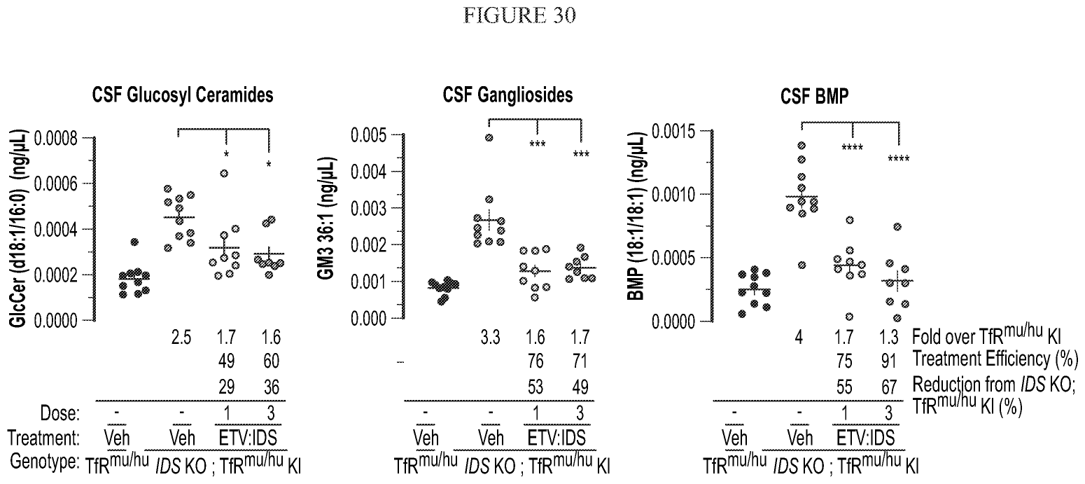

Lysosomal dysfunction in IDS KO; TfR^{mu/hu} KI is assessed by measuring levels of accumulated lysosomal lipids in brain and CSF. Following 13 weekly doses of ETV:IDS, robust correction of lysosomal dysfunction was observed, with some lysosomal lipids such as gangliosides and BMP, reaching levels comparable to TfR^{mu/hu} KI mice. For instance, BMP reduction observed in the brain at 7 days post the last dose of ETV:IDS resulted in levels no different from TfR^{mu/hu} KI mice at 1 and 3 mg/kg. The percent treatment efficiency was >88% at 3 mg/kg for gangliosides and BMP, and 67% and 84% at 1 and 3 mg/kg, respectively, for glucosyl ceramides (FIG. 29). ETV:IDS treatment resulted in a significant reduction in lysosomal lipid levels in the CSF, with treatment efficiencies reaching up to 91% as with BMP (FIG. 30). Brain and CSF lysosomal lipids exhibited a high correlation across dosages supporting the utility of measuring changes in CSF pathway biomarkers as a surrogate for changes in brain (data not shown).

### Body Weights

IDS KO; TfR^{mu/hu} KI weighed more at the initiation of dosing than TfR^{mu/hu} KI mice, and IDS KO; TfR^{mu/hu} KI vehicle treated mice gained more weight than TfR^{mu/hu} KI vehicle treated mice for the duration of the study. Mice treated with ETV:IDS, initially lost weight, and subsequently, gained less weight and weighed significantly less (beginning at Week 4) than the vehicle treated disease model. No significant differences in weight gain between the KO mice treated with 1 and 3 mg/kg ETV:IDS compared to the TfR^{mu/hu} KI group were observed.

### Hematology

Red blood cell parameters (HCT, RBC, HGB) in mice treated with 1 mg/kg or 3 mg/kg ETV:IDS were higher than the disease model and approximated TfR^{mu/hu} KI non-diseased control mice. A similar trend to normalization was observed in neutrophils. PLT numbers were mildly elevated compared to both disease and non-diseased vehicle controls.

### Histopathology

No ETV:IDS-related histopathologic effect was observed in the tissues examined. A pharmacologic effect on neuronal vacuoles (presumptive GAG-filled lysosomes) was observed. Neuronal vacuolation within the vestibular and/or cerebellar nuclei of the brain was consistently present only in vehicle control IDS KO; TfR^{mu/hu} KI animals. No similar neuronal vacuolation was evident in TfR^{mu/hu} KI animals or in IDS KO; TfR^{mu/hu} KI animals administered 1 or 3 mg/kg ETV:IDS.

### SUMMARY

These data demonstrate that ETV:IDS, dosed peripherally in mice via IV administration for 13 weekly doses, can alleviate CNS disease burden, as demonstrated by a reduction in primary storage product (GAGs) in the CNS and periphery, as well as downstream biomarkers of disease. Markers of lysosomal function (glycosyl ceramides, gangliosides, and BMP) and neuronal injury (Nf-L), are restored to control levels. These data also show a high degree of correlation between CSF and brain GAG levels, as well as CSF and brain lysosomal lipid levels following ETV:IDS treatment, thus suggesting the CSF biomarker changes reflect changes in the brain.

Additionally, safety evaluation of IDS KO; TfR^{mu/hu} KI mice demonstrated improvement of parameters associated with disease with ETV:IDS treatment. For instance, weekly IV doses of ETV:IDS at 1 and 3 mg/kg corrected the increased body weight and weight gain that is normally observed in the IDS KO; TfR^{mu/hu} KI mice and restored hematology parameters to non-disease control levels.

### Example 9. ETV:IDS Rescues Neurobehavioral and Skeletal Phenotypes in 4.5-Month Old IDS KO×TfR^{muhu} Mice

ETV:IDS was administered to a mouse model of MPS II that expresses the chimeric human/mouse transferrin receptor (“IDS KO; TfR^{mu/hu} KI mice”). As described herein, administration of ETV:IDS can reduce accumulation of GAGs in brain, CSF, and peripheral tissues. In this Example, the ability of ETV:IDS to correct motor skills, sensorimotor gating, and learning and memory in IDS KO; TfR^{mu/hu} KI mice was evaluated.

### Materials and Methods

### Animal Care and Mouse Strains

Mice were housed under a 12-hour light/dark cycle and had access to water and standard rodent diet (LabDiet® #25502, Irradiated) ad libitum. The IDS KO×TfR^{mu/hu} mice and TfR^{mu/hu} KI mice used in this study are described in Example 1 above. All mice used in this study were males and were approximately 4.5 months of age at the start of dosing and 9 months at necropsy.

### Administration and Sample Collection

IDS KO; TfR^{mu/hu} KI mice (n=21 per group) were administered 17 weekly doses of 3 mg/kg of a representative ETV:IDS protein via intraperitoneal injection (ETV:IDS 35.23.2; see, Example 6). Vehicle treated TfR^{mu/hu} (n=20 per group) and Ids KO; TfR^{mu/hu} mice (n=19 per group) served as the non-disease and disease comparator group, respectively. All animals were euthanized 7 days following their last dose.

Each animal was observed once daily. Body weights were taken and recorded prior to the first dose and once weekly thereafter, prior to dosing.

In-life and terminal serum samples were collected in manner similar to that described in Examples 1 and 7, respectively. Additionally, prior to scheduled necropsy, approximately 250 microliters of blood were also collected via cardiac puncture for hematology. Blood was collected in EDTA tubes (Sarstedt Microvette 500 K3E) and slowly inverted 10 times. Terminal CSF samples were collected as described in Example 1. Brain and liver tissue samples were also collected as described in Example 1. In addition, urine was collected and chilled immediately prior to termination. Urine samples were then stored in a freezer, set to maintain −60 to −80° C., for urine biomarker analysis.

### Quantification of GAGs

Tissue and fluids were processed for GAG analysis as described in Example 1 with the following modifications. Ten (10) μg of total protein lysate was used for liver and brain for subsequent HS/DS digestion. Digestion was carried out in a PCR plate in a total volume of 62 μL (all tissue lysates and biofluids). Lysates or body fluids (3 μL of mouse CSF) were mixed with Heparinases I, II, III, and Chondriotinase B in digestion buffer with internal standard mix of HS and DS (20 ng total per sample). The digests were mixed with acetonitrile and analyzed by mass spectrometry. Individual disaccharide species were identified based on their retention times and MRM transitions using commercially available reference standards (Iduron Ltd). GAGs were quantified by the peak area ratio of D0A0, D0S0, and D0a4 to the internal standard using Analyst 1.7.1 or MultiQuant 3.0.2 (Sciex). Reported GAG amounts were normalized to total protein levels as measured by a BCA assay (Pierce), and interpolated against a calibration curve. For HS/DS calibration curves and QCs, pure standards for D0a4 (DS/CS), D0A0 (HS), and D0S0 (HS) were dissolved in acetonitrile:water 50/50 (v/v) to generate a 1 mg/mL stock. An eight-point dilution curve in matrix was generated, at 5, 10, 20, 100, 1000, 5000, 9000 to 10000 ng/mL. Three levels of QC samples were prepared at 15, 300 and 7500 ng/mL. The calibration curve standards and QCs went directly to enzymatic digestion, together with biological samples, but without adding enzyme, and the following steps and run by LC-MS/MS as described in Example 1. Additionally, one lot of pooled CSF was used as Matrix QC and digested at the same time with study samples to monitor the digestion consistency.

### Methods for CSF and Serum Analysis of Nf-L

Nf-L levels in serum and CSF were measured as described in Example 2.

### Behavioral Assays

All behavioral assays were run from week 11 to week 15 of dosing. All testing and analysis were performed by an experimenter blinded to the genotype and treatment.

Motor coordination and balance. The rotarod test was used to assess motor coordination and balance. The training phase, the first day of testing, consisted of 3 individual trials with an inter-trial interval between 15-20 minutes. During the trial, mice were placed on the rotarod apparatus that was set to a constant speed of 16 rotations per minute (rpm). On the second and third day of testing, mice were placed on the rotarod apparatus with the rod rotating at an accelerated speed, from 4 rpm to 40 rpm, increasing by 3.6 rpm every 30 seconds. The mice were given 2 sessions of 3 trials each, one in the morning and one in the afternoon, for a total of 6 trials per day. The trial ended when the mouse fell off the rod or when 5 minutes had elapsed.
Motor coordination and agility. The pole test assay was used to assess agility and coordination. Mice were gently placed facing down atop a vertical acrylic pole 50 cm in length with a rough surface. The base of the pole was secured onto a home cage and padded with bedding. Three trials were performed for a maximum of 20 seconds per trial, and the latency for the mouse to climb down as well as slides and falls were recorded. If the mouse fell from a pole after secure placement, the animal was assessed a latency of 20 seconds. If the mouse slid less than 20% of the pole length, their latency was adjusted to 1.25 times the total latency. If the mouse slid 20-50% of the pole, they were assigned a latency of 15 seconds. Latency to descend the pole was averaged across the 3 trials for each mouse.
Gait analysis. Gait analysis was assessed using Mouse Specifics DigiGait Treadmill (Framingham, Mass.). Mice were placed inside the DigiGait enclosure, and the treadmill was started at an initial speed of 15 cm/second. The gait analysis required 5-10 seconds of continuous running video acquisition. Multiple attempts per animal could be required to get continuous running, with a maximum of 5 minutes acquisition time at each of the test speeds (15, 20, and 30 cm/second). If a mouse was unable to keep up with the treadmill, the mouse was gently guided to the front end of the walking compartment by gently pushing them forward with the back panel. In addition to analyzing the gait of the animals, the number of animals in each treatment group that were able to successfully completed each speed was recorded.
Startle inhibition. Pre-pulse inhibition (Kinder Scientific, Poway, Calif.) was used to assess if a weak auditory signal, shortly preceding a loud sound, can attenuate an animal's startle response. Testing was performed in a small isolated restraining chamber placed inside a sound attenuating cubicle, free from external movement and noise. On the day of the test, mice were placed in the sound attenuating chamber and given 5 minutes to acclimate to the restraining chamber and 64 dB background noise before stimulus presentations begin. Mice were then exposed to a series of acoustic startle stimuli for 20 minutes in which some stimuli were preceded by a weaker acoustic stimulus, or pre-pulse (pp), at random intervals. Trials with no auditory stimulus were also included to provide a measure of baseline activity. The test is comprised of a total of 80 trials randomly selected from the following list: 24 trials of 40 ms at 120 dB, and 14 trials each of the 4 dB pp 40 ms at 120 dB, 15 dB pp 40 ms at 120 dB, 26 dB pp 40 ms at 120 dB, and no stimulus trials. The interstimulus interval (ISI) is variable with a mean of 15 seconds, and a range of 8-22 seconds. The percentage of pre-pulse inhibition was calculated using the following formula: 100*((mean of 40 ms at 120 dB trials−mean of pre-pulse trial type)/(mean of 40 ms at 120 dB)).
Responsiveness to acoustic stimuli. The acoustic startle threshold test was used to measure an animal's reflexive responses to sudden loud acoustic stimuli. Testing was performed in a small isolated chamber inside a sound attenuating cubicle, free from external movement and noise. On the day of testing, mice were placed into the restraining chamber and given 5 minutes to acclimate to the restraining chamber and 64 dB background noise before acoustic stimulus testing began. Mice were then exposed to a series of acoustic pulses at varying intensities for approximately 15 minutes at random intervals. Trials with no auditory stimulus were also included to provide a measure of baseline startle activity. The test was comprised of a total of 70 trials that are randomly selected from the following list: 10 trials each of 40 ms at 120 dB, 40 ms at 110 dB, 40 ms at 100 dB, 40 ms at 90 dB, 40 ms at 80 dB, 40 ms at 70 dB, and no stimulus trials. The ISI is variable with a mean of 15 seconds, and a range of 8-22 seconds.
Visual-spatial learning and memory. The active place avoidance task was used to measure visual-spatial learning and memory. Testing was performed using the BioSignal Corp. Place Avoidance system and Tracker software (Acton, Mass.). Distinct black and white visual cues were placed on the walls of the room surrounding the apparatus. The grid arena was rotated at 1 rpm during the trials such that the animal must actively navigate against the rotation of the arena to avoid entry into an aversive zone. On day 1 (habituation), a stationary 600 wedge fixed to the external configuration of the spatial cues of the arena was designated as the aversive zone. Entries into this aversive zone did not result in shock delivery during the 10-minute habituation trial and movement within the arena was used to establish a baseline of activity prior to the start of avoidance training. On days 2-4, animals were given a single 10-minute trial per day with the current source (shock) activated. Any time the mouse entered the aversive zone, a mild shock of 0.2 mA current source lasting 500 milliseconds was delivered every 1.5 seconds until the mouse left the zone. On day 5, the 10-minute trial was split into two 5-minute phases. The first phase was the probe test in which the current source was turned off in order to examine the animal's pure spatial memory for the aversive zone without the presence of the unconditioned stimulus. After 5 minutes had elapsed, the current source was turned on only when the animal had clearly left the aversive zone. This reinstatement phase continued for the remaining 5 minutes of the session and provided a measure of working memory if a priming shock was delivered.
Supraspinal nociception. The hot plate test was used to measure supraspinal nociception and was measured on a black anodized aluminum plate. The surface of the hot plate was heated to a constant temperature of 52° C. During testing, mice were placed in a clear cylindrical enclosure placed on top of the hot plate. The latency to respond with either a hind paw lick, hind paw flick, or jump, whichever came first, was measured. Each animal was once tested once and removed after a clear response was observed. To prevent injury, the maximum latency was 30 seconds.
Visual acuity. The visual cliff test was used to measure an animal's visual acuity. The apparatus consisted of two areas, a shallow and a deep side. The shallow side of the cliff was covered with a squared black checker laminated pattern on the floor and walls while the deep side of the cliff was a transparent area suspended 101 cm above the floor to create the illusion of a cliff. For the step-off phase, mice were placed on a platform elevated 2.5 cm above the floor of the apparatus, directly straddling the shallow and deep side of the cliff. The test consisted of 4 trials with a maximum latency of 300 seconds. The latency to step off the platform and the direction of the step off was recorded.

### Facial Scoring

The facial profile of each animal was assessed qualitatively at necropsy and animals were given a score based on the severity of their shortened snouts. All analysis was performed by an experimenter blinded to the phenotype and treatment.

### Histology

The skull base, containing the tympanic bullae, from three mice per group were used for light microscopic evaluation. Briefly, after the brain was removed, skulls were immersion fixed in 4% paraformaldehyde (PFA) and post-fixed in 70% ethyl alcohol. Skulls were decalcified in Immunocal Decalcifier Solution for two days, rinsed in water and then processed and embedded in paraffin. Five (5) micron thick sections were cut at 100 micron intervals through the tympanic bullae, stained with hematoxylin and eosin (H&E), and cover slipped for light microscopic evaluation.

### Ex-Vivo Bone Micro-CT Imaging and Analysis

At necropsy, the pelvis and hindlimbs were immersion fixed in 4% paraformaldehyde in 1×PBS before being transferred to 70% ethyl alcohol for micro-CT imaging. For micro-CT imaging, a Bruker SkyScan-1176 micro-CT scanner was used (X-Ray source: 90 kV/25 W, X-Ray detector: 4000×2672 pixels). A calibration phantom was included during scanning and used for calibration of measurements post scanning. Region of interest selection for trabecular and cortical bone analysis in femur and tibia was based on the American Society of Bone and Mineral Research (ASBMR) published guidelines (Bouxsein, 2010). The following scan parameters were used: X-ray source 60 kV/25 W, In plane resolution 18 um. For image analysis, the following parameters were measured for the femur: trabecular bone mineral density (BMD), trabecular bone mineral content (BMC), trabecular bone volume (BV), trabecular thickness, trabecular separation, trabecular number, cortical thickness, cortical periosteal perimeter, cortical endosteal perimeter, cortical BMD, cortical BMC, and cortical BV.

### Statistical Analysis

Data was expressed as means±SEM and statistical analysis was either performed in GraphPad Prism 8 or imported into R statistical computing software. For assessment of GAG and Nf-L levels, analysis was performed using one-way ANOVA with Tukey's multiple comparison test. Serum and CSF Nf-L correlation analysis was performed using nonparametric Spearman's correlation coefficient. The accelerated treadmill assay was analyzed using Fisher's exact test when one or more groups had less than 5 animals in a condition, otherwise the chi-square test was used. The latency to descend a pole in the pole test was averaged across 3 trials and a linear mixed-effects model was used to account for multiple trials conducted on all subjects. The analysis of pre-pulse inhibition was performed using a linear model. The acoustic startle threshold test was analyzed using a linear mixed-effects model to account for the within subject correlation obtained from having repeated measures of the same subject on different levels of auditory stimulus. For the active place avoidance test, a linear mixed-effects model was used to account for the within subject correlation obtained from having repeated measures of the same subject at different stages of the experiment. The analysis of trial 3 and the reinstatement test of that APA task was performed using a Wilcoxon sum rank test. Animal performance on the APA task was analyzed by Fisher's exact test. The analysis of the hot plate test was performed using a nonparametric Wilcoxon rank sum test. The analysis of the visual cliff test was performed using a chi-square test. Facial score analysis was performed using Fisher exact test. Quantitative analysis from micro-CT scans was performed using one-way ANOVA with Tukey's multiple comparison test.

### Results

### 1. Systemic Administration of ETV:IDS Reduces CNS GAGs and Neurofilament-Light Levels

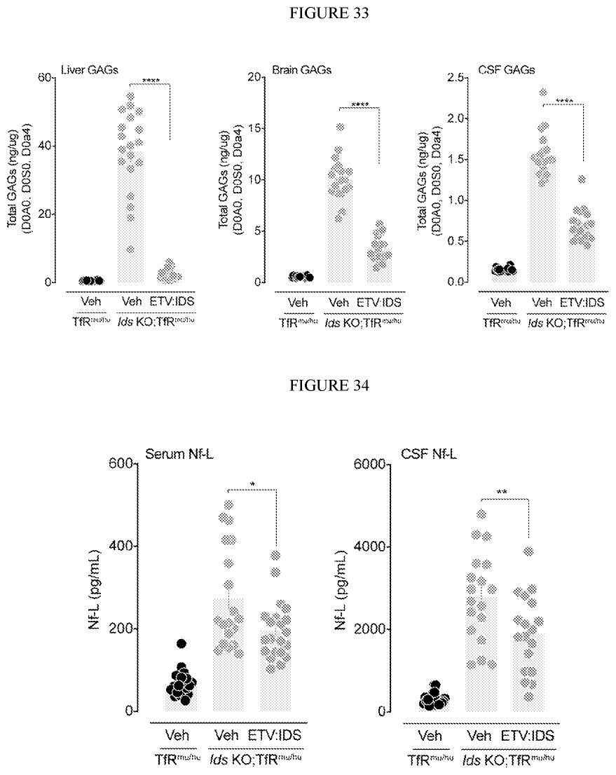

IDS KO; TfR^{mu/hu} received weekly doses of vehicle or 3 mg/kg ETV:IDS via intraperitoneal injection for 17 weeks. Age-matched, vehicle-treated TfR^{mu/hu} mice served as a non-disease control group. GAG levels in liver, brain, and CSF were assessed seven days following the last dose. Total GAGs were determined as the sum of the major HS (D0A0, D0S0) and DS (D0a4) derived disaccharides. ETV:IDS was effective at reducing GAGs in peripheral organs such as the liver as well as effective at lowering substrates in the brain and CSF (FIG. 33). ETV:IDS was also found to be effective at reducing Nf-L in IDS KO; TfR^{mu/hu} mice when treatment was initiated at a stage in disease progression when CSF Nf-L levels are significantly more elevated (4.5 months of age), which is more representative of when treatment onset might be initiated in MPS II human patients. After 17 weeks of treatment, ETV:IDS significantly reduced Nf-L levels by ˜30% in both serum and CSF of IDS KO; TfR^{mu/hu} (FIG. 34), demonstrating that ETV:IDS could still exert partial neuroprotection despite the late treatment onset. Levels of Nf-L in serum and CSF were positively correlated following ETV:IDS treatment (data not shown), suggesting that evaluation of Nf-L in serum can be explored as a CNS treatment responsive biomarker. Overall, these data confirm that ETV:IDS can reduce substrate accumulation in both the CNS and peripheral tissues and is efficacious at lowering a marker of neuroaxonal degeneration.

### 2. Systemic Administration of ETV:IDS Rescues Neurobehavioral Phenotypes

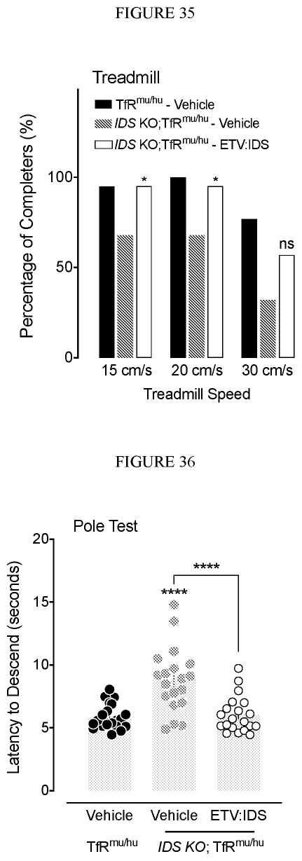

The IDS KO mouse model recapitulates several biological and pathological aspects of MPS II, which include severe impairment in gross and fine motor skills. Minor motor phenotypes have previously been described in the IDS KO mice, but limited data are available on potential cognitive dysfunction in this model. Multiple behavioral domains were compared between IDS KO; TfR^{mu/hu} and TfR^{mu/hu} mice at between 4 and 8 months of age, and clear differences were observed, as illustrated by evaluations with accelerated treadmill assay, the pre-pulse inhibition assay, and the active place avoidance (APA) assay. Weekly dosing of ETV:IDS initiated at 4.5 months of age was able to reduce the identified behavioral deficits in IDS KO; TfR^{mu/hu} mice. To assess motor skills, mouse performance in the rotarod assay was measured, and IDS KO; TfR^{mu/hu} mice did not show any deficits in this task, indicating no major alteration of coordination and balance (data not shown). Consistent with this observation, IDS KO; TfR^{mu/hu} mice displayed normal gait on the treadmill (data not shown). However, at faster speeds on the treadmill, not all IDS KO; TfR^{mu/hu} mice could complete the task, which could indicate either a physical inability to run fast or a lack of motivation to complete a more challenging task (FIG. 35). Remarkably, ETV:IDS treatment improved the performance of IDS KO; TfR^{mu/hu} mice, as demonstrated by the increased number of mice completing the running task despite treadmill acceleration (FIG. 35). To further explore the motor skills and agility of the mice, their performance was measured in the pole test, and it was observed that ETV:IDS treatment also normalized performance of IDS KO; TfR^{mu/hu} mice relative to that of non-disease controls (FIG. 36).

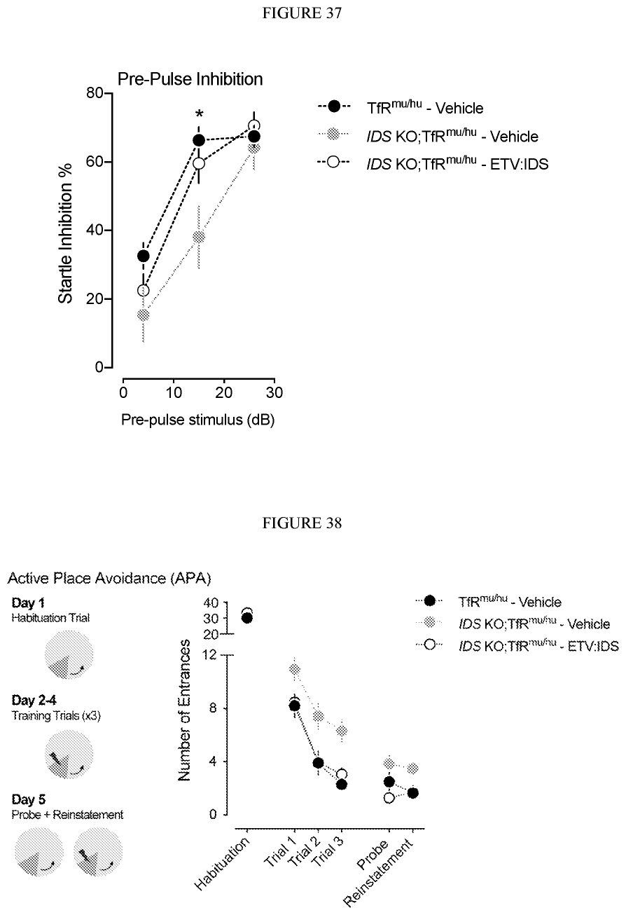

In addition to attention deficits, neuronopathic MPS II patients may present perseverative and repetitive behaviors that suggest abnormalities in inhibitory function. In mice, both attention and inhibitory function can be assessed by measuring sensorimotor gating using the pre-pulse inhibition paradigm (PPI). The PPI assay measures the ability to inhibit the startle response to a strong stimulus preceded by a weaker stimulus. It was observed that IDS KO; TfR^{mu/hu} mice showed a significant reduction in pre-pulse startle inhibition compared to control mice, demonstrating a deficit in sensorimotor gating (FIG. 37). This deficit was fully rescued by ETV:IDS in the PPI assay (FIG. 37). To verify that this effect could not be attributed to differences in hearing sensitivity in the IDS KO; TfR^{mu/hu} mice, the mice were tested by the acoustic startle threshold test. The IDS KO; TfR^{mu/hu} mice did not show any deficits in the response to broad-band noise in this test (data not shown), suggesting that the beneficial effect of ETV:IDS observed in the PPI reflects an improvement of sensorimotor gating, not simply auditory sensitivity.

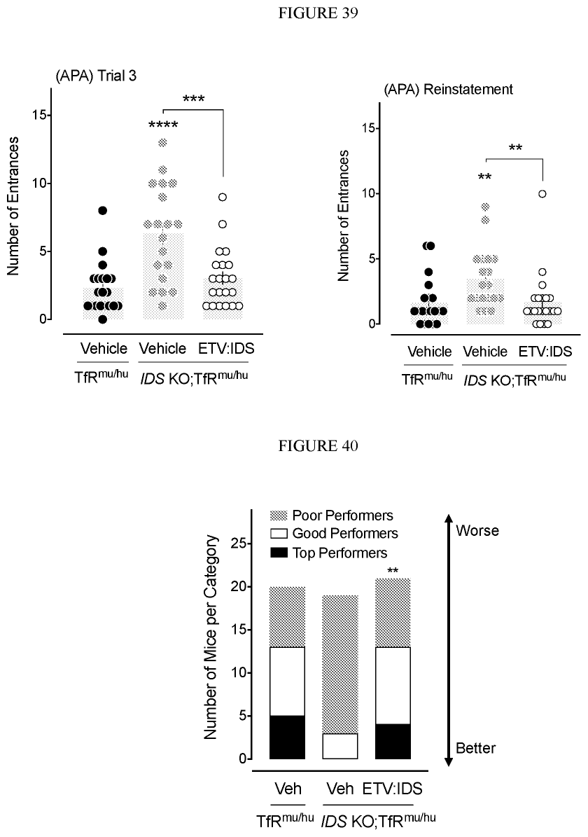

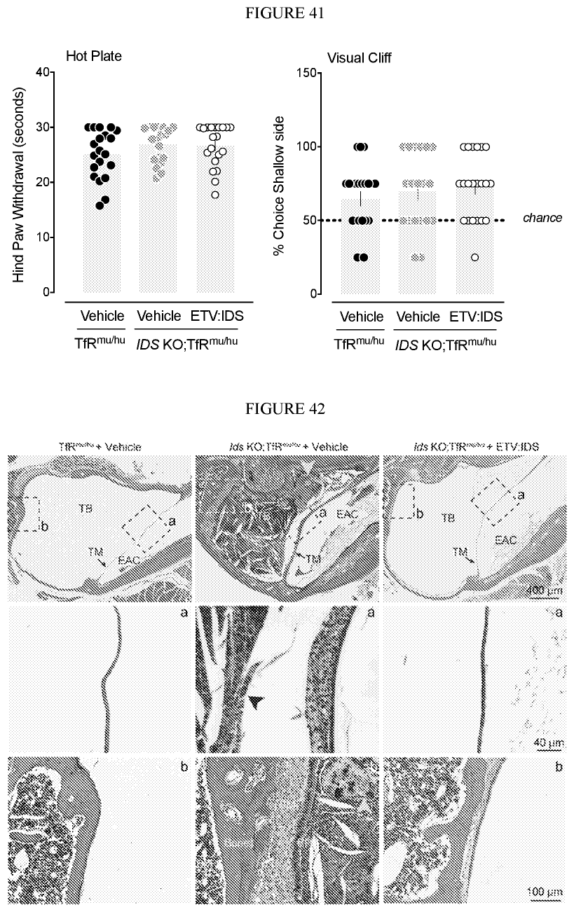

Finally, to determine if IDS KO; TfR^{mu/hu} mice have cognitive deficits, their spatial learning and memory was tested in the active place avoidance (APA) assay. The APA is a hippocampus-dependent task in which the mice learn to use distal visual cues to avoid a zone associated with an aversive stimulus (mild electric shock) while the arena is continuously rotating. IDS KO; TfR^{mu/hu} mice were found to have impaired spatial learning, as shown by the higher number of entrances in the aversive zone (AZ) relative to the control cohort during the learning trials and re-instatement trial (FIGS. 38-40). Remarkably, systemic administration of ETV:IDS normalized performance of these mice to levels similar to control mice in the APA assay (FIGS. 38-40). To verify that the protective effect of ETV:IDS in the APA was not caused by differences in pain sensitivity or visual function, the three experimental groups were tested and found to have similar pain response in the hot plate assay and proper avoidance of the aversive location in the visual cliff assay (FIG. 41). Altogether, the results of the APA assay demonstrate that administration of ETV:IDS normalized spatial learning and memory deficits in the IDS KO; TfR^{mu/hu} mice.

Accordingly, in addition to reducing peripheral and CNS GAG accumulation and Nf-L, a marker of neuronal injury, administration of ETV:IDS corrected multiple neurobehavioral deficits including motor skills, agility, sensorimotor gating, and learning and memory in the MPS II mouse model.

### 3. Systemic Administration of ETV:IDS Corrects Auricular and Skeletal Phenotypes in IDS KO; TfR^{mu/hu} Mice

Progressive conductive and sensorineural hearing loss, resulting from GAG accumulation in the middle ear and recurrent otitis, are common in MPS II patients. The impact of ETV:IDS on the ear canal of an MPS II mouse model was thus evaluated. The external auditory canal and middle ear (tympanic membrane and bulla) were evaluated bilaterally from three animals per group. Compared to vehicle treated TfR^{mu/hu} control mice, marked middle ear effusion and/or chronic otitis media were observed in the tympanic bulla of IDS KO; TfR^{mu/hu} mice (FIG. 42), consistent with previously described phenotypes. Submucosal edema and/or fibrosis with occasional polyp formation were also evident in 5 out of the 6 ear canals assessed (FIG. 42, panel b). IDS KO; TfR^{mu/hu} mice treated with ETV:IDS demonstrated dramatic improvement and generally displayed normal external and middle ear morphology (FIG. 42). Additionally, mild submucosal fibrosis was observed in only 1 out of the 6 ear canals assessed. These data show that chronic ETV:IDS treatment improved middle ear effusion and otitis media, suggesting the potential to improve hearing function, even when hearing deficits have already progressed.

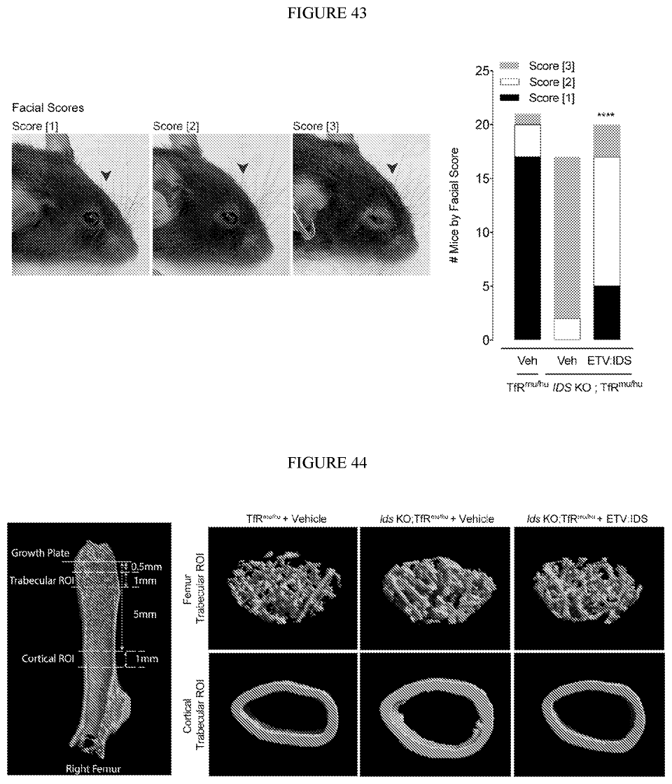

Musculoskeletal abnormalities are present in multiple MPS disorders, including MPS II. The mouse model of MPS II displays skeletal abnormalities similar to what is observed in MPS II patients with skeletal changes appearing early in the disease process and progressively worsening as the animals age. Standard-of-care enzyme replacement therapies and hematopoietic stem cell transplantation (HSCT) therapies have limited beneficial impact on the skeleton if not initiated early, thus, the effect of ETV:IDS treatment on the skeletal phenotypes in IDS KO; TfR^{mu/hu} mice was investigated. At 9 months of age, vehicle treated IDS KO; TfR^{mu/hu} mice displayed short, broadened snouts compared to the non-diseased TfR^{mu/hu} control mice. However, the facial morphology of IDS KO; TfR^{mu/hu} mice treated with ETV:IDS displayed an intermediate phenotype and, in some cases, looked indistinguishable from TfR^{mu/hu} control mice (FIG. 43). At necropsy, the facial profiles of each animal were blindly scored based on the qualitative assessment of the severity of their shortened, broadened snouts with a score of 3 representing severe abnormal skull development. TfR^{mu/hu} control mice had more facial scores of 1 and 2 while those of the vehicle treated IDS KO; TfR^{mu/hu} were almost all exclusively given a score of 3. IDS KO; TfR^{mu/hu} mice treated with ETV:IDS had more facial scores of 1 and 2, showing an improvement in the animals facial abnormalities following ETV:IDS delivery (FIG. 43).

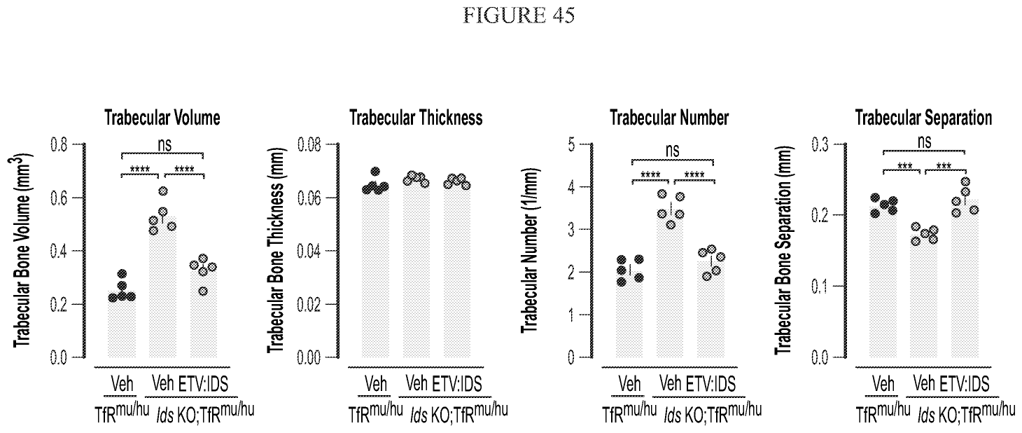

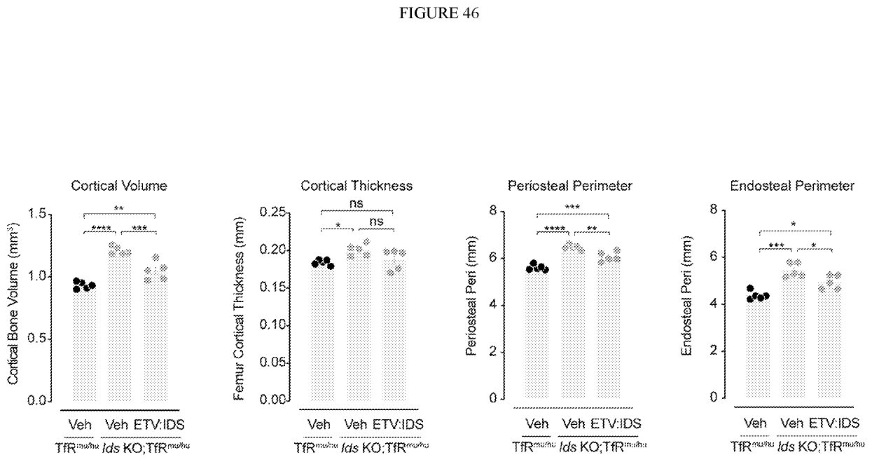

The effect of ETV:IDS on skeletal phenotypes was also investigated using micro-CT to acquire images of the femur and conduct quantitative image analysis of trabecular and cortical bone. Increased trabecular density and thicker cortical bone in the vehicle treated IDS KO; TfR^{mu/hu} mice compared to controls was evident. These abnormalities were corrected by ETV:IDS. Following ETV:IDS treatment, the trabecular and cortical bone of IDS KO; TfR^{mu/hu} mice looked similar to vehicle treated TfR^{mu/hu} controls (FIG. 44). A quantitative analyses on multiple cortical and trabecular morphometric parameters was also carried out. Quantitative analysis demonstrated a significant increase in the total amount of trabecular bone (volume) and a decrease in mean trabecular separation in vehicle treated IDS KO; TfR^{mu/hu} mice compared to controls. This is consistent with an increase in the number of trabeculae, which was significantly reduced following ETV:IDS treatment, approaching values observed in TfR^{mu/hu} control mice (FIG. 45). Femur cortical bone measurements also showed that cortical bone volume, cortical thickness, as well as endosteal and periosteal diameter were significantly increased in vehicle treated IDS KO; TfR^{mu/hu} mice compared to controls and were reduced following ETV:IDS treatment (FIG. 46). The degree of changes observed between the IDS KO; TfR^{mu/hu} mice and controls, as well as in the ETV:IDS treatment group, are greater in cancellous bone (trabeculae) compared to compact (cortical) bone, which is consistent with the relatively higher bone turnover rate of small trabecula. Overall, these data indicate that chronic ETV:IDS treatment can correct skeletal abnormalities when skeletal disease has already progressed.

The totality of data disclosed herein demonstrate that systemic administration of ETV:IDS is efficacious in rescuing MPS II-relevant neurobehavioral and skeletal abnormalities. Furthermore, these effects are observed in older cohorts of mice after neurobehavioral deficits, auricular dysfunction, skeletal abnormalities, and extensive neuroaxonal injury is ongoing and/or has already progressed.

### Example 10. An Observational Study Investigating Potential Treatment-Responsive CNS-Biomarkers in Subjects with Hunter Syndrome (MPSII)

A two-part, prospective, multicenter observational study is performed to assess clinical and biomarker features of MPS II to improve the understanding of biomarker measures and their intra- and inter-patient variability, and to explore their relationships to clinical features and progression of MPS II. This information is used to inform the design of future interventional clinical trials.

### Objectives

### Primary Objective:

To assess GAG levels, including within-patient and between-patient variability, in blood and urine.

### Secondary Objectives:

To assess HS and DS levels in the cerebrospinal fluid (CSF).
To assess levels of potential disease related or treatment-responsive biomarkers in blood, urine and/or CSF samples.
   Total GAGs, HS, DS, Neurofilament light chain (Nf-L), lysosomal lipids, lysosomal proteins, cytokines, and soluble triggering receptor expressed on myeloid cells 2 (sTREM2).
To explore correlations between disease related biomarkers and clinical measures of disease severity (neurocognitive, clinical, functional, and quality of life (QoL) assessments).

### Methods

### Study Design

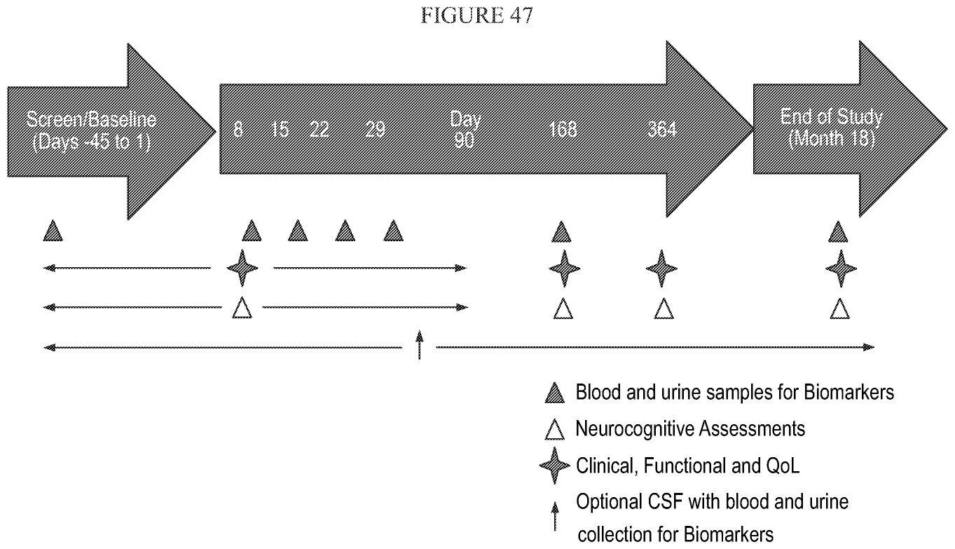

In part 1 of the study, approximately 20 patients with MPS II aged 2 through 10 years are enrolled: 10 or more patients aged 2 through 10 years; and 7 or more patients with nMPS II. Urine and blood samples for biomarker assessment and clinical evaluations of disease severity are measured at regular intervals for up to 18 months (see, FIG. 47). A one-time optional CSF sample may also be collected for biomarkers.

In part 2 of the study, approximately 8 patients with MPS II aged 2 through 30 are enrolled: 4 or more with nMPS II; patients scheduled to undergo general anesthesia for non-study related medical procedure. A single collection of CSF, urine and blood are performed for biomarker assessment. Optionally clinical evaluations of disease severity are performed.

Inclusion Criteria Study participants must have a confirmed diagnosis of MPS II based on the following: documented mutation in the IDS gene; and reduced IDS activity in plasma, WBCs and/or skin fibroblasts (≤10% of the lower limit of normal). For the nMPS II subgroups, study participants must have: a developmental quotient (DQ)<85 and/or a decline of at least 7.5 points in DQ, assessed at least 6 months apart; or have the same genetic mutation as a blood relative with confirmed nMPS II.

### Exclusion Criteria

Criteria for exclusion from the study include the following conditions or events: unstable medical condition; the subject has received any CNS-targeted MPS II investigational therapy (e.g, intrathecal IDS, transferrin or insulin receptor-mediated IDS delivery to CNS, or stem cell transplantation) within the previous 6 months; the subject has received MPS II gene therapy at any time; or the subject has a mutation of other genes that are known to be associated with developmental delay, seizures or other significant CNS disorders.

### Biomarker, Neurocognitive, Clinical, Functional and Quality of Life Assessments

Assessments within each of the following categories are performed as indicated.

Biomarker Assessments: Total Urine GAGs; HS and DS levels in blood, urine and CSF; Nf-L in blood and CSF; Lysosomal lipids [sphingolipids, gangliosides and bis(monoacylglycerol)phosphate (BMP)] in blood, urine, and CSF; Cytokines in blood, urine and CSF; and sTREM2 levels in CSF.

Neurocognitive Assessments: Vineland Adaptive Behavior Scales, Second Edition (VABS-II); Scales of Infant and Toddler Development, Third Edition (BSID-III) or Kaufman Assessment Battery for Children, Second Edition (KABC-II), as determined by algorithm

Clinical Assessments: Adverse events and Patient Diary; Medical History and Demographic data; Physical Examination; and Safety Laboratory Tests (chemistry, hematology and urinalysis, and CSF cell count, protein and glucose).

Functional Assessments: Individualized Educational Plans (IEPs); Individualized Family Service Plan (IFSPS); and Audiology Assessment, Auditory Brainstem Response (ABR)

Quality of Life Assessments: Child Health Questionnaire Parent Form 28 (CHQ-PF28) or Infant and Toddler Quality of Life (ITQOL); and Pediatric Quality of Life Inventory Family Impact Module (PEDSQL-FIM)

### Example 11. ETV:IDS Significantly Reduces CSF GAGs in MPS II Patients: A Study to Determine the Safety, Pharmacokinetics, and Pharmacodynamics of ETV:IDS in Pediatric Subjects with Hunter Syndrome

A study was performed to assess the safety, pharmacokinetics (PK), and pharmacodynamics (PD) of ETV:IDS (ETV:IDS 35.23.2; see, Example 6), an investigational central nervous system (CNS)-penetrant enzyme replacement therapy (ERT), designed to treat both the peripheral and CNS manifestations of Hunter syndrome (MPS II).

### Objectives

To assess the safety and tolerability of ETV:IDS in pediatric subjects with Hunter syndrome.
To assess the PK and PD of ETV:IDS in pediatric subjects with Hunter syndrome.
To assess the effects of ETV:IDS on GAG levels (HS and DS levels) in cerebrospinal fluid (CSF), serum and urine samples.
To assess the effects of ETV:IDS on the levels of biomarkers of lysosomal function, inflammation and neuronal injury (e.g., Nf-L) in serum and/or CSF samples.
To assess the effects of ETV:IDS on clinical measures of disease severity (e.g., using neurocognitive, clinical, functional, and quality of life (QoL) assessments).

### Results

Cohort A completed enrollment of five (5) male subjects with neuronopathic MIPS II having an age range of 5 to 8 years. Race/ethnicity of the subjects included Asian, White, Black and Hispanic. All subjects were on a standard-of-care (SOC) treatment (e.g., Elaprase) for at least two years prior to initiation of treatment with ETV:IDS. Four of the five subjects had anti-drug antibodies against IDS at baseline, with titers ranging from 189 to greater than 11 million. All subjects received at least twelve (12) weekly doses of ETV:IDS, of which at least four of the five subjects have escalated to the highest (30 mg/kg) dose level. ETV:IDS was generally well tolerated at the doses tested (3 to 30 mg/kg). All subjects have continued the study and there have been no discontinuations or dose reductions. All treatment emergent adverse events were mild or moderate in severity. The most frequently observed adverse events were mild or moderate infusion-related reactions consistent with other enzyme replacement therapies (see, Wraith, J. E. 2006. Journal of Inherited Metabolic Disease 29(2-3):442-447; Tylki-Szymanska and Jurecka. 2015. Expert Opinion on Orphan Drugs 3(11):1241-1253), reporting 69% of patients receiving other enzyme replacement therapies were reported to have hypersensitivity reactions). In particular, three of the five subjects had mild or moderate infusion-related reactions. One SAE was reported, which was based on hospitalization for observation of a moderate infusion-related reaction (fever) at dose level 7.5 mg/kg, which resolved in less than 24 hours with administration of acetaminophen and diphenhydramine. Two of five subjects had anemia considered related to blood collection by the investigator. One weekly dose at the 7.5 mg/kg dose level was skipped due to anemia.

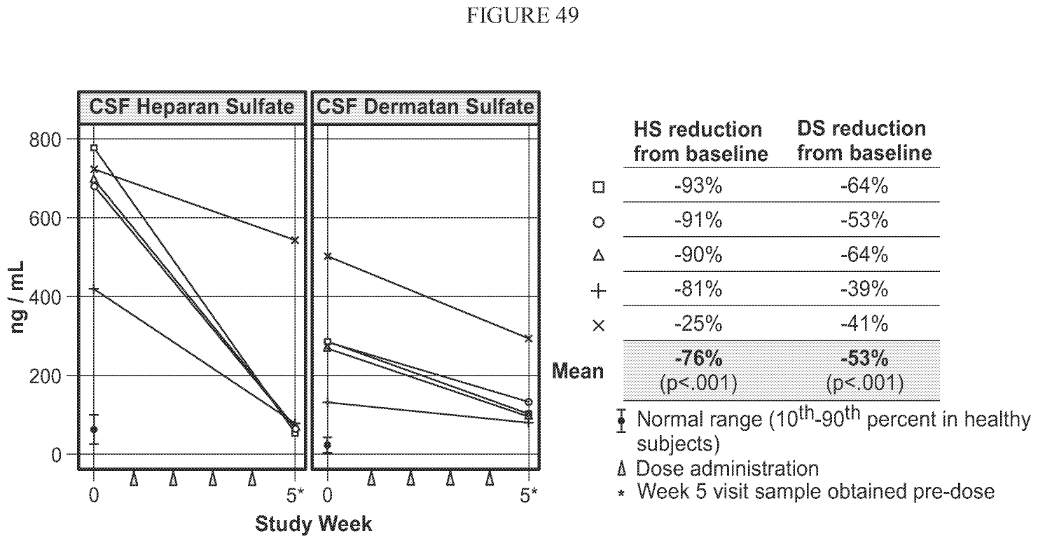

CSF heparan sulfate (HS) levels in the subjects was calculated as the sum of disaccharides D0A0, D0A6, D0S0, and D2S6. As illustrated in FIG. 49, the subjects in Cohort A achieved a statistically significant reduction of CSF HS levels after four (4) weekly doses of ETV:IDS (two 3 mg/kg doses plus two 7.5 mg/kg doses) with a mean reduction from baseline (HS level prior to administration of ETV:IDS) of 76% (p<0.004). Four of five subjects achieved normalization of CSF HS levels with corresponding reductions of 93%, 91%, 90%, and 81% from baseline. The remaining subject experienced a 25% reduction in CSF HS levels relative to baseline. Also as illustrated in FIG. 49, the subjects in Cohort A achieved a statistically significant reduction of CSF dermatan sulfate (DS) after four (4) weekly doses of ETV:IDS with a mean reduction from baseline (DS level prior to administration of ETV:IDS) of 53% (p<0.001). The subjects achieved individual CSF DS reductions of 64%, 64%, 53%, 41%, and 39% from baseline. Total urine GAGs were monitored weekly for all subjects after switching from SOC treatment. Total urine GAG levels, monitored by colorimetric assay, were stable or decreased in all subjects after initiating ETV:IDS, supporting safety of continued dosing.

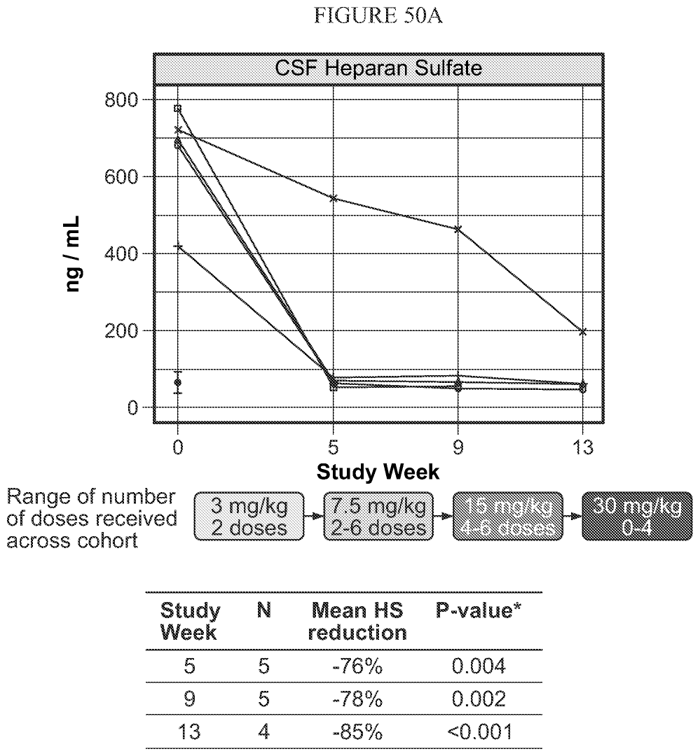

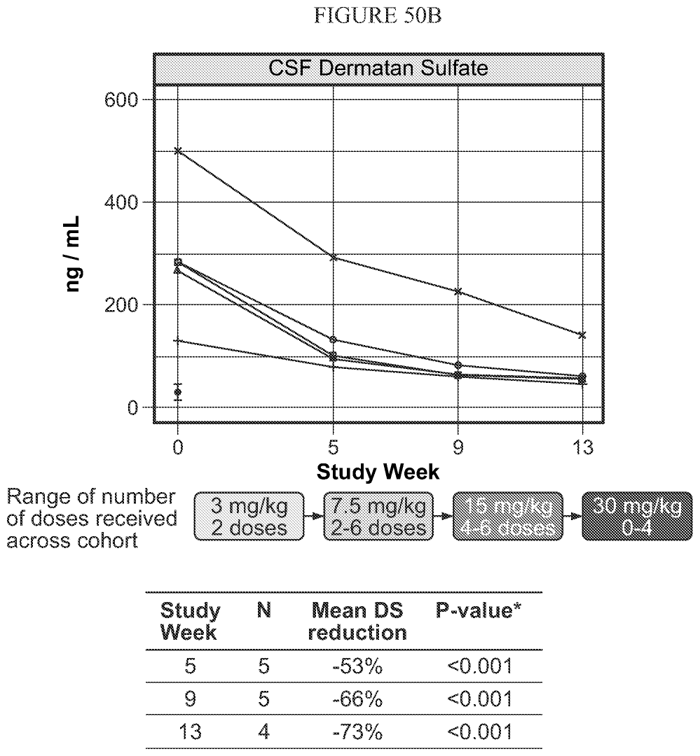

After 12 weekly doses of ETV:IDS, the subjects in Cohort A continued to achieve a statistically significant reduction of CSF HS levels, with a mean reduction from baseline (HS level prior to administration of ETV:IDS) of 85% (p<0.001), with sustained normalization of CSF HS in 4 of 5 subjects (FIG. 50A; normal range indicated by (black circle) determined using 30 adult CSF samples (range 10-90 percentile); adult and child CSF GAG levels are similar (Hendriksz et al., Mol. Genet. Metab. Rep., 5:103-106 (2015))). The remaining subject, who showed a continued reduction in CSF HS from week 5 (25%) to week 13 (73%), had a pre-existing ADA titer against IDS of greater than 11 million, which was 10²-10⁶-fold higher than the titers in the other four subjects.

After 12 weekly doses of ETV:IDS, the subjects in Cohort A continued to achieve a statistically significant reduction of CSF DS levels, with a mean reduction from baseline (DS level prior to administration of ETV:IDS) of 73% (p<0.001) (FIG. 50B).

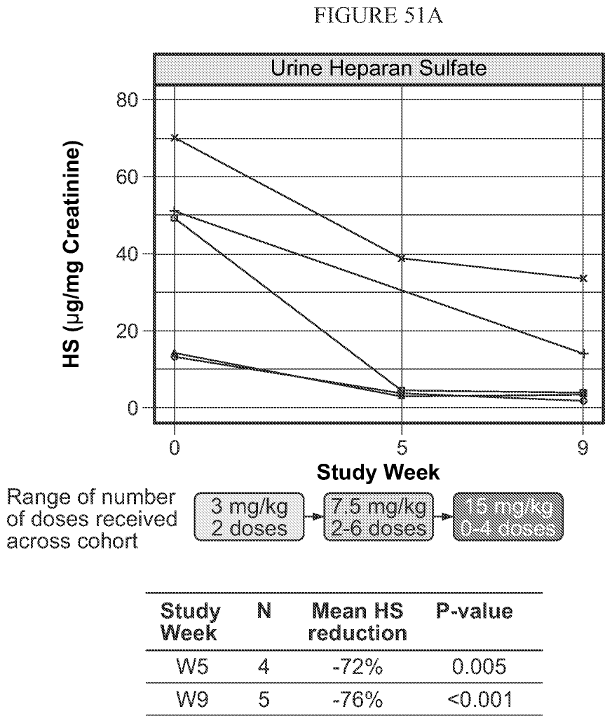

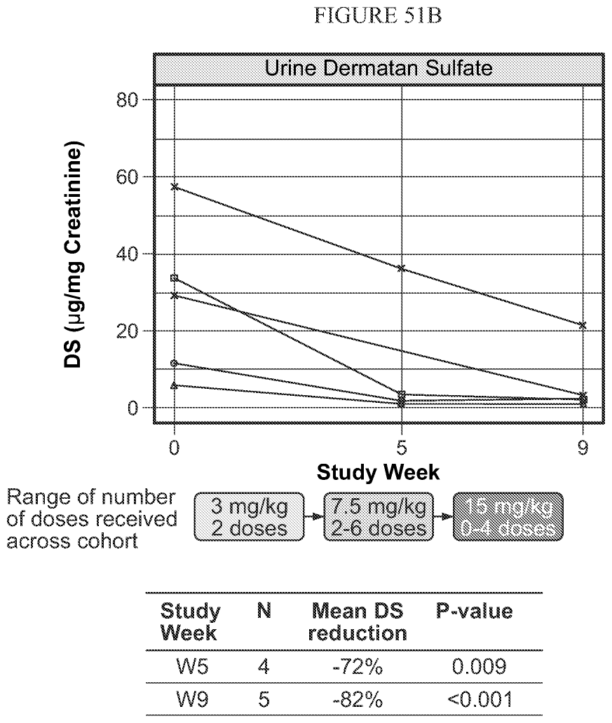

Total urine HS and DS levels decreased after switching from SOC treatment to treatment with ETV:IDS. As illustrated in FIGS. 51A-51B, the subjects in Cohort A achieved a statistically significant reduction of urine HS and DS after four (4) weekly doses of ETV:IDS, with a mean reduction from baseline (HS/DS levels prior to administration of ETV:IDS) of 72% (p=0.005) and 72% (p=0.009), respectively. Sustained urine HS and DS reductions were observed at week 9 (mean HS reduction of 76% (p<0.001) and mean DS reduction of 82% (p<0.001)) (FIGS. 51A-51B).

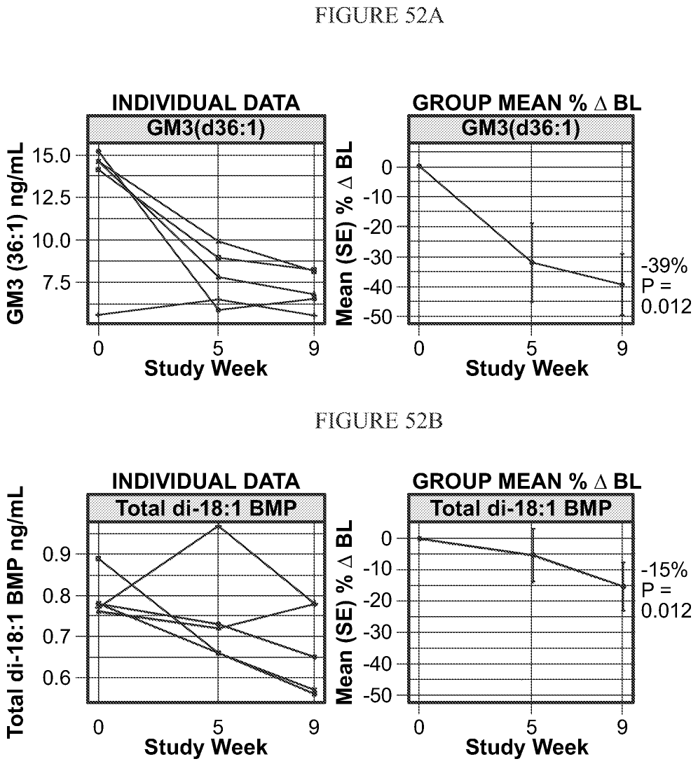

Certain lysosomal lipids are increased in the CSF of MPS II patients, including GM3 and BMP. To evaluate the effect of ETV:IDS on lysosomal function, the levels of GM3 and BMP in the CSF from the Cohort A subjects were examined. As shown in FIG. 52, GM3 declined in 4 of 5 subjects, with a mean decline of 39% at week 9 (p=0.012). BMP declined in 3 of 5 subjects, with a mean decline of 15% at week 9 (p=0.12) (FIG. 52). The reduction in CSF GM3 and the trend in reduction in BMP supports effects of ETV:IDS on lysosomal function.

These data support the use of a once weekly administration of ETV:IDS for the treatment of the neurocognitive and physical manifestations of Hunter syndrome.

### Methods

### Study Design

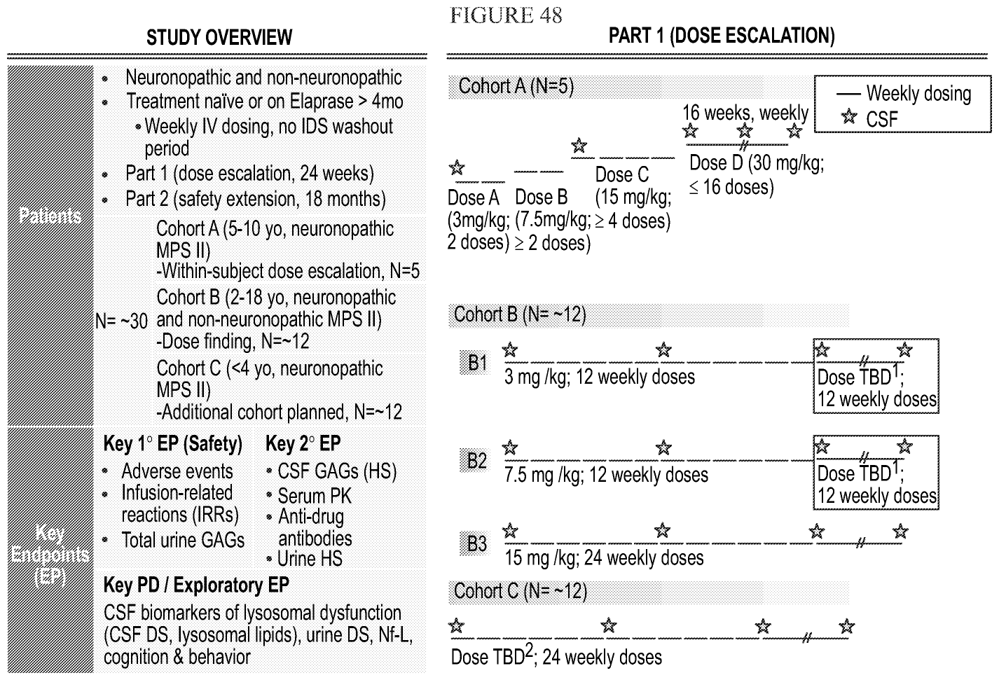

The study has three staggered cohorts: subjects with neuronopathic MPS II aged 5 to 10 years (Cohort A); subjects with MPS II, either neuronopathic or non-neuronopathic, aged 2 to 18 years (Cohort B); and subjects with neuronopathic MPS II who are less than 4 years of age (Cohort C) (see, FIG. 48). The study includes an ascending-dose stage to assess the safety, tolerability, PK, and PD of ETV:IDS over approximately 24 weeks. In particular, ETV:IDS is intravenously administered to the subjects weekly using the dose escalation. The duration of ETV:IDS infusion is approximately 3 hours. For Cohort A, the dose escalation of ETV:IDS was: 3 mg/kg (“Dose A,” 2 weekly infusions), 7.5 mg/kg (“Dose B,” at least 2 weekly infusions); 15 mg/kg (“Dose C,” at least 4 weekly infusions), and 30 mg/kg (“Dose D,” weekly until completion). Cohort B includes three sub-cohorts with starting doses of 3 mg/kg (B1), 7.5 mg/kg (B2), and 15 mg/kg (B3). Subjects within sub-cohorts B1 and B2 receive 12 weekly doses of the starting dose followed by an evaluated dose or doses, with the possibility of one or more dose escalations. The evaluated dose(s) does not exceed 30 mg/kg and is based on the Cohort A/Cohort B safety and biomarker results (e.g., a dose used for at least two weeks in Cohort A that is deemed safe and generally well tolerated) (see, FIG. 48). The dose or doses for use in Cohort C is a dose evaluated in Cohort A and Cohort B and is determined based on safety and biomarker results from those cohorts.

### Inclusion Criteria

Study participants must have a confirmed diagnosis of MPS II. Subjects enrolled in Cohort A are aged 5-10 years with neuronopathic MPS II. Subjects enrolled in Cohort B are aged 2 to 18 years with non-neuronopathic MPS II, neuronopathic MPS II, or unknown phenotype. Subjects enrolling in Cohort C are less than 4 years of age with neuronopathic MPS II. For subjects receiving intravenous iduronate 2-sulfastase (IDS) ERT, they are required to have tolerated a minimum of 4 months of therapy during the period immediately prior to screening.

### Exclusion Criteria

Criteria for exclusion from the study include the following conditions or events: 1) unstable or poorly controlled medical condition(s) or significant medical or psychological comorbidity or comorbidities that may interfere with safe participation in the study or interpretation of study assessments; 2) use of any CNS-targeted MPS II ERT within 3 months before study start for subjects aged ≥5 years, and within 6 months before study start for subjects aged <5 years; 3) use of IDS gene therapy or stem cell therapy at any time; 4) clinically significant thrombocytopenia, other clinically significant coagulation abnormality, or significant active bleeding, or required treatment with an anticoagulant or more than two antiplatelet agents; 5) contraindication for lumbar punctures; 6) have a clinically significant history of stroke, status epilepticus, head trauma with loss of consciousness, or any CNS disease that is not MPS II related within 1 year of screening; 7) have had a ventriculoperitoneal (VP) shunt placed, or any other brain surgery, or have a clinically significant VP shunt malfunction within 30 days of screening; or 8) have any clinically significant CNS trauma or disorder that, in the opinion of the investigator, may interfere with assessment of study endpoints or make participation in the study unsafe.

### CSF and Urine Sample Collection

CSF and urine samples were collected at scheduled time-points. For the results illustrated in FIGS. 49, 50(A,B), and 52(A,B), CSF samples were collected by lumbar puncture prior to first dose and approximately 1 week after the 4^{th}, 8^{th} and/or 12^{th} doses of ETV:IDS. For the results illustrated in FIG. 51(A,B), urine samples were collected prior to first dose and approximately 1 week after the 4^{th} and 8^{th} dose of ETV:IDS.

### LCMS Assay for GAGs

Quantification of GAGs (e.g., HS and DS) in CSF and urine was performed by liquid chromatography using a method similar to that previously described (Pan et al. 2018. Bioanalysis 10(11):825-838; Wang et al. 2018. Biomedical Chromatography 32:e4294 (12 pages)).

### LCMS Assay for BMP and Gangliosides

BMP analyses of CSF were performed by a validated clinical laboratory. Ganglioside analyses of CSF were performed using a method similar to that described in Example 1.

### Pharmacokinetics

Blood samples were collected for the analysis of ETV:IDS serum concentrations at scheduled time-points. PK parameters of ETV:IDS were derived by noncompartmental analysis of the serum concentration-time data, as data allow. PK parameters may include but are not limited to the following: Cmax; Trough concentration (Cmin); Tmax; Area under the concentration-time curve from time zero to the time of last quantifiable concentration (_{AUC0-last}); Area under the concentration-time curve over a dosing interval (AUC_{0-T}); Apparent terminal elimination rate constant (λz); Apparent terminal elimination t½; and Accumulation ratio.

### Safety Assessments

Safety was evaluated using one or more of the assessments described above, as well as the following: Frequency and severity of adverse events (AEs), including infusion-related reactions (IRRs), which include allergic reactions and anaphylaxis; Vital sign measurements; Physical examinations, including neurological examinations; Safety laboratory assessments (including hematology, serum clinical chemistry, urinalysis, and coagulation); Urine total GAG concentrations (normalized to creatinine); Characterization of immunogenicity of ETV:IDS in serum, as measured by the incidence of anti-drug antibodies (ADAs) during the study relative to baseline; and Use of concomitant medications.

### Biomarker, Neurocognitive, Clinical, Functional and Quality of Life Assessments

Assessments similar to those described in Example 10 were performed. These assessments are used to measure outcomes, as detailed below.

### Outcome Measures

### Primary Outcome Measure:

1. Incidence and severity of adverse events (AEs) including infusion-related reactions (IRRs)
2. Urine total glycosaminoglycan (GAG) concentrations

### Secondary Outcome Measure:

3. Change from baseline in cerebrospinal fluid (CSF) of heparan sulfate
4. PK parameter: Maximum observed concentration (Cmax) of ETV:IDS in serum
5. PK parameter: Trough concentration (Cmin) of ETV:IDS in serum
6. PK parameter: Time to maximum observed concentration (Tmax) of ETV:IDS in serum
7. PK parameter: Area under the concentration-time curve from time zero to the time of last quantifiable concentration (AUC[0-last]) of ETV:IDS in serum
8. PK parameter: Area under the concentration-time curve over a dosing interval (AUC[0-τ]) of ETV:IDS in serum
9. PK parameter: Apparent terminal elimination rate constant (λz) of ETV:IDS in serum
10. PK parameter: Apparent terminal elimination t½ of ETV:IDS in serum
11. PK parameter: Accumulation ratio of ETV:IDS in serum
12. Characterization of immunogenicity of ETV:IDS in serum, as measured by the incidence of anti-drug antibodies (ADAs) relative to baseline
13. Change from baseline in urine concentration of heparan sulfate (HS)

### Other Pre-Specified Outcome Measures:

14. Change from baseline in CSF of dermatan sulfate (DS)
15. Change from baseline in urine concentration of DS
16. Change from baseline in serum concentrations of HS and DS
17. Change from baseline in levels of CSF biomarkers of lysosomal function
18. Liver and spleen volumes as assessed by ultrasound
19. Change from baseline in Clinical Global Impression-Severity (CGI-S) score
20. Clinical Global Impression-Improvement (CGI-I) score
21. Change from baseline in Parent/Caregiver Global Impression-Severity (PGI-S) score
22. Parent/Caregiver Global Impression-Improvement (PGI-I) score
23. Change from baseline in Activities of Daily Living for Hunter Syndrome (ADL-HS) score
24. Change from baseline in stool consistency, per the Bristol Stool Scale
25. Change from baseline in Six-Minute Walk Test (6MWT) for subjects ≥6 years old who are able to follow instructions at baseline
26. Change from baseline in the toileting abilities percentage (TAP), as measured by the Toileting Abilities Survey (TAS)
27. Change from baseline in auditory brainstem response (ABR) parameters
28. Change from baseline in the cognitive-domain and the language- and motor-domain score and scores on either the Bayley Scales of Infant and Toddler Development (BSID-III) or the nonverbal index of the Kaufman Assessment Battery for Children (KABC-II)
29. Change from baseline in the composite age-equivalent score and standard scores on the VABS-II
30. Change from baseline in Infant and Toddler Quality of Life Questionnaire (ITQOL) for subjects <5 years of age
31. Change from baseline in Childhood Health Questionnaire Parent Form (CHQ-PF28) for subjects ≥5 years of age
32. Change from baseline in Pediatric Quality of Life Inventory Family Impact Module (PedsQL-FIM)

### Statistical Analysis

The statistical analysis is performed using R Software (v. 3.6.2). Continuous variables are summarized using descriptive statistics (number of participants, mean, standard deviation [SD], median, minimum, maximum, and 95% confidence interval [CI] where applicable.) P-values were determined by T-test comparison of mean percent change from baseline.

### Tables

TABLE 1
Summary of GlcCer levels in brains of IDS
KO mice treated with ETV:IDS (fold over WT)

| | KO + | KO + | KO + |
|---|---|---|---|
| | Vehicle | ETV:IDS | Idursulfase |
| GlcCer (d18:1, 16:0) | 2.2 | 1.2 | 1.8 |
| GlcCer (d18:1, 18:0) | 1.5 | 1.0 | 1.4 |
| GlcCer (d18:2, 18:0) | 1.6 | 0.9 | 1.3 |
| GlcCer (d18:1, 20:0) | 1.6 | 1.1 | 1.4 |
| GlcCer (d18:2, 20:0) | 1.7 | 1.1 | 1.4 |
| GlcCer (d18:1, 22:0) | 2.0 | 1.1 | 1.8 |
| GlcCer (d18:1, 22:1) | 1.2 | 1.1 | 1.1 |
| GlcCer (d18:2, 22:0) | 1.1 | 1.0 | 1.3 |
| GlcCer (d18:1, 24:1) | 1.6 | 1.1 | 1.4 |
| GlcCer (d18:1, 24:0) | 1.9 | 1.1 | 1.6 |

TABLE 2
Lipid levels in brains of IDS KO mice treated with
vehicle, ETV:IDS or Elaprase (fold changes over WT)

| | Fold changes over WT | | | |
|---|---|---|---|---|
| | | 0.9 | 1.1 | |
| | KO + | KO + | KO + | |
| | Vehicle | ETV:IDS | Elaprase | WT |
| PA(16:0_18:1) | 1.01652815 | 0.87805028 | 1.02614953 | 1 |
| PA(18:0_18:1) | 0.9803871 | 0.89061561 | 1.01854752 | 1 |
| PA(18:1_18:1) | 1.01723588 | 0.8753273 | 1.01906474 | 1 |
| PA(18:0_20:4) | 1.05587055 | 0.95439223 | 1.0287576 | 1 |
| PA(18:1_22:6) | 1.03000727 | 0.98349165 | 1.00662459 | 1 |
| PA(18:0_22:6) | 0.99319049 | 0.92980276 | 0.93442667 | 1 |
| PE(P-18:0/18:1) | 0.98195031 | 0.95324202 | 1.02518226 | 1 |
| PE(P-18:0/18:2) | 0.97225052 | 0.89209895 | 0.93871825 | 1 |
| PE(P-16:0/20:4) | 0.99144839 | 0.94499411 | 0.92862372 | 1 |
| PE(P-18:0/20:4) | 0.96754718 | 0.89442024 | 0.91564924 | 1 |
| PE(P-16:0/22:6) | 0.92008888 | 0.85984114 | 0.87469497 | 1 |
| PE(P-18:0/22:6) | 0.99775035 | 0.95455118 | 0.96148973 | 1 |
| Sulfatide C16:0 | 0.90576849 | 0.79127831 | 0.76195962 | 1 |
| Sulfatide C18:0 | 0.81280069 | 0.75779574 | 0.68197925 | 1 |
| Sulfatide C18:0(OH) | 0.76031924 | 0.75628052 | 0.64675377 | 1 |
| Sulfatide C24:0 | 0.75780648 | 0.70713297 | 0.66131545 | 1 |
| Sulfatide C24:0(OH) | 0.78833907 | 0.72508863 | 0.74860619 | 1 |
| Sulfatide C24:1 | 0.821473 | 0.74990649 | 0.75471324 | 1 |
| Sulfatide C24:1(OH) | 0.67258164 | 0.62567179 | 0.57409235 | 1 |
| GM3(d34:1) | 2.09393928 | 1.16672118 | 1.98170536 | 1 |
| GM3(d36:1) | 3.74157091 | 1.08458273 | 2.97575794 | 1 |
| GM3(d38:1) | 5.52033941 | 0.99911522 | 4.11985175 | 1 |
| GM3(d40:1) | 1.42159849 | 1.03491677 | 1.39585927 | 1 |
| GD3(d34:1) | 1.46726848 | 0.96014081 | 1.31353994 | 1 |
| GD3(d36:1) | 1.46699639 | 0.86224602 | 1.21726307 | 1 |
| GD3(d38:1) | 1.53724473 | 0.82158939 | 1.20878056 | 1 |
| GD3(d40:1) | 0.49744039 | 0.55827773 | 0.76669619 | 1 |
| GD3(d42:2) | 1.23998054 | 1.00287447 | 1.21684885 | 1 |
| GD3(d42:1) | 1.37177496 | 1.05759888 | 1.30756718 | 1 |
| GD1a/b(d36:1) | 0.88470817 | 0.82107431 | 0.81837911 | 1 |
| GD1a/b(d38:1) | 0.94221971 | 0.82198461 | 0.86416076 | 1 |
| GT1b(d36:1) | 0.93359111 | 0.81370939 | 0.85646637 | 1 |
| GT1b(d38:1) | 1.01291833 | 1.05855164 | 0.98541269 | 1 |
| LBPA(20:4/20:4) | 0.91555026 | 0.74507558 | 0.88416882 | 1 |
| LBPA(22:6/22:6) | 1.36257845 | 0.96610388 | 1.31658696 | 1 |
| LBPA(18:1/18:1) | 1.69980961 | 0.93424803 | 1.60274415 | 1 |
| Palmitic acid | 0.98744714 | 0.98336985 | 0.9050388 | 1 |
| Palmitoleic acid | 1.0337731 | 1.07564952 | 0.8971913 | 1 |
| Stearic acid | 0.96048221 | 0.92211752 | 0.86237988 | 1 |
| Oleic acid | 1.04267865 | 1.07932738 | 0.94252135 | 1 |
| Linoleic acid | 1.16554943 | 1.23446712 | 1.06289268 | 1 |
| Linolenic acid | 1.09587266 | 1.14758313 | 1.00817176 | 1 |
| Arahidonic acid | 0.96304418 | 0.93138432 | 0.88614686 | 1 |
| EPA | 1.15835012 | 1.09293496 | 1.11325942 | 1 |
| DHA | 1.12990573 | 1.13617975 | 1.05917735 | 1 |
| LPE(P-16:0) | 1.31137183 | 1.23195216 | 1.37622222 | 1 |
| LPE(P-18:0) | 1.30059624 | 1.16733271 | 1.29181659 | 1 |
| LPE(P-18:1) | 1.25418938 | 1.07523775 | 1.2800414 | 1 |
| LPE(16:0) | 1.17836852 | 1.02214629 | 1.18935927 | 1 |
| LPE(18:0) | 1.20717572 | 1.07020161 | 1.19516724 | 1 |
| LPE(18:1) | 1.25977631 | 1.16098986 | 1.34700962 | 1 |
| LPI(16:0) | 1.1754397 | 1.01479906 | 1.20396379 | 1 |
| LPI(18:0) | 1.22645413 | 1.00320543 | 1.22476213 | 1 |
| LPI(20:4) | 1.08781434 | 1.07354529 | 1.1529951 | 1 |
| LPG(16:0) | 1.20599747 | 1.0439152 | 1.21749983 | 1 |
| LPG(18:0) | 1.20955994 | 0.99977385 | 1.15689786 | 1 |
| LPG(18:1) | 1.19938507 | 1.04431583 | 1.21637775 | 1 |
| LPG(20:4) | 1.19976807 | 0.98041911 | 1.16595786 | 1 |
| CL(72:8) | 1.31239964 | 1.17287219 | 1.50060034 | 1 |
| Cholesterol Sulfate | 0.80540874 | 0.94340786 | 0.91029938 | 1 |
| PG(16:0_18:1) | 0.92567469 | 0.83442845 | 0.90850807 | 1 |
| PG(18:0_18:1) | 1.02612596 | 0.96264443 | 0.99514232 | 1 |
| PG(18:1/18:1) | 0.95920271 | 0.89717417 | 0.94675624 | 1 |
| PG(18:0_20:4) | 0.90789033 | 0.82978451 | 0.87514211 | 1 |
| PI(18:0_18:1) | 1.17804687 | 1.06396153 | 1.13113519 | 1 |
| PI(18:1/18:1) | 1.14059806 | 1.00809425 | 1.11351988 | 1 |
| PI(16:0_20:4) | 1.20957205 | 1.10714204 | 1.14720781 | 1 |
| PI(18:0_20:4) | 1.17971223 | 1.09279463 | 1.13849017 | 1 |
| PI(16:0_22:6) | 1.18060688 | 1.0109285 | 1.15474382 | 1 |
| PI(18:0_22:6) | 1.30588528 | 1.1193819 | 1.2830753 | 1 |
| PI(20:4/20:4) | 1.31581237 | 1.26493195 | 1.45542681 | 1 |
| PS(18:0_18:1) | 1.03679611 | 0.90917125 | 0.94646579 | 1 |
| PS(18:0_20:4) | 1.06956884 | 0.94321053 | 0.99958824 | 1 |
| PS(16:0_22:6) | 1.01389572 | 0.87820865 | 0.92402097 | 1 |
| PS(18:1_22:6) | 1.03170698 | 0.93486711 | 0.95345599 | 1 |
| PS(18:0_22:6) | 0.92376879 | 0.84379138 | 0.87852082 | 1 |
| Sphingosine | 1.20956582 | 1.03139557 | 1.07103473 | 1 |
| Sphinganine | 1.12625109 | 0.95945537 | 1.00866548 | 1 |
| Cer(d18:1/16:0) | 1.1898435 | 1.2702808 | 1.09898379 | 1 |
| Cer(d18:1/18:0) | 1.05719808 | 1.04709156 | 0.93672262 | 1 |
| Cer(d18:1/24:0) | 1.07960504 | 0.97523544 | 0.92010478 | 1 |
| Cer(d18:1/24:1) | 0.82995192 | 0.81205788 | 0.87781267 | 1 |
| Cer(d18:0/16:0 | 1.40270677 | 1.30051111 | 1.13522744 | 1 |
| Cer(d18:0/18:0) | 0.98462219 | 0.92661852 | 0.93865067 | 1 |
| Cer(d18:0/24:0) | 0.99180462 | 0.98741525 | 0.97143202 | 1 |
| Cer(d18:0/24:1) | 1.11123432 | 1.04202586 | 1.0700886 | 1 |
| SM(d18:1/16:0) | 1.12700306 | 1.0423671 | 1.07524893 | 1 |
| SM(d18:1/18:0) | 0.92305936 | 0.85541522 | 0.83048724 | 1 |
| SM(d18:1/24:0) | 1.05515306 | 0.88080112 | 1.00315475 | 1 |
| SM(d18:1/24:1) | 1.12213595 | 1.03280034 | 1.03584333 | 1 |
| SM(d18:1/0:0) | 0.91345559 | 0.75717864 | 1.0337506 | 1 |
| LacCer(d18:1/16:0) | 0.89628608 | 0.86982524 | 0.84127411 | 1 |
| LacCer(d18:1/18:0) | 0.85680143 | 0.77685157 | 0.805254 | 1 |
| LacCer(d18:1/24:0) | 0.86945705 | 0.79822554 | 0.78436148 | 1 |
| LacCer(d18:1/24:1) | 1.1604392 | 0.90198172 | 0.9768989 | 1 |
| LPC(16:0) | 1.11016041 | 1.1168508 | 1.2442346 | 1 |
| LPC(18:0) | 1.10803656 | 1.07688874 | 1.1572643 | 1 |
| LPC(18:1) | 1.1154886 | 1.04813535 | 1.24491264 | 1 |
| LPC(20:4) | 0.98466789 | 0.96068998 | 1.1325393 | 1 |
| LPC(22:6) | 0.9658907 | 0.86998883 | 1.06140777 | 1 |
| LPC(26:0) | 0.88498543 | 0.67243113 | 0.9152565 | 1 |
| LPC(24:0) | 1.09363028 | 0.85617717 | 1.07651749 | 1 |
| LPC(26:1) | 1.20767361 | 0.88836639 | 1.16334611 | 1 |
| LPC(24:1) | 1.08223641 | 0.82913148 | 1.09880877 | 1 |
| LPC(16:1) | 1.0717371 | 1.00744658 | 1.19339107 | 1 |
| PC(36:1) | 1.00006753 | 0.93268207 | 0.94070597 | 1 |
| PC(36:2) | 0.99239587 | 0.9360556 | 0.95517289 | 1 |
| PC(36:4) | 0.96248027 | 0.91666785 | 0.9224405 | 1 |
| PC(38:4) | 0.96130284 | 0.92655592 | 0.92290748 | 1 |
| PC(38:6) | 0.96207788 | 0.90828018 | 0.92381277 | 1 |
| PC(40:4) | 0.91798169 | 0.91136589 | 0.86172822 | 1 |
| PC(40:6) | 0.94836904 | 0.90006372 | 0.91057844 | 1 |
| PC(O-18:0/2:0) | 1.14562106 | 1.02053195 | 1.19829164 | 1 |
| PE(36:1) | 1.06114562 | 1.0189909 | 1.05259418 | 1 |
| PE(36:2) | 1.15485876 | 1.04526236 | 1.1343485 | 1 |
| PE(36:4) | 1.04833954 | 1.03567807 | 1.06728264 | 1 |
| PE(38:4) | 1.04079742 | 0.99455675 | 1.0226952 | 1 |
| PE(38:6) | 1.07401418 | 0.99212159 | 1.06265553 | 1 |
| PE(40:4) | 1.04695373 | 1.04931574 | 1.01557279 | 1 |
| PE(40:6) | 1.03972968 | 0.98870268 | 1.04136612 | 1 |
| Cholesterol | 0.91803474 | 0.83017672 | 0.90636493 | 1 |
| CE(16:1) | 0.79707665 | 1.03142836 | 0.94106694 | 1 |
| CE(18:1) | 0.71507121 | 1.0606394 | 0.76736776 | 1 |
| CE(18:2) | 0.86833261 | 0.90755089 | 0.79528551 | 1 |
| CE(20:4) | 1.42343634 | 1.50925476 | 1.39206153 | 1 |
| CE(20:5) | 0.47822541 | 0.98426688 | 0.52619281 | 1 |
| CE(22:6) | 0.46977302 | 1.13646633 | 0.45746034 | 1 |
| TG 52:4/18:1 | 0.9439894 | 3.79244261 | 0.66349662 | 1 |
| TG 52:3/18:1 | 1.08787116 | 3.46853351 | 0.85350626 | 1 |
| TG 54:2/18:0 | 1.07559143 | 1.06097724 | 1.02031321 | 1 |
| TG 52:5/20:4 | 0.95111046 | 0.94243599 | 0.95376133 | 1 |
| TG 54:6/20:4 | 0.95099702 | 1.37444033 | 0.88391879 | 1 |
| TG 54:7/20:4 | 0.97464894 | 1.54022919 | 0.85627423 | 1 |
| TG 56:4/20:4 | 0.73673358 | 0.61495212 | 0.66053781 | 1 |
| TG 56:6/20:4 | 0.9621839 | 0.868923 | 0.9856356 | 1 |
| TG 56:7/20:4 | 0.92092128 | 1.1335638 | 0.87873015 | 1 |
| TG 58:5/20:4 | 0.93020084 | 0.82193456 | 0.94840377 | 1 |
| TG 58:7/20:4 | 0.93722573 | 0.85237293 | 0.89052547 | 1 |
| TG 58:8/22:6 | 1.07576583 | 0.94100942 | 0.99773868 | 1 |
| TG 60:7/22:6 | 0.96749854 | 0.73417824 | 0.82130776 | 1 |
| TG 60:8/22:6 | 1.05266868 | 0.92069031 | 0.9919752 | 1 |
| Sphingosine-1- | 0.81893112 | 0.73370827 | 0.74586929 | 1 |
| phosphate | | | | |
| GB3(d18:1/16:0) | 2.70726534 | 2.32101997 | 1.24086224 | 1 |
| GB3(d18:1/18:0) | 1.72741991 | 0.82100977 | 1.42007533 | 1 |
| GB3(d18:1/24:0) | 1.24531193 | 0.90109689 | 0.98417536 | 1 |
| GB3(d18:1/24:1) | 1.23974776 | 1.99474236 | 1.70175418 | 1 |
| Glu-Cholesterol | 1.16546978 | 1.0592315 | 1.28977423 | 1 |
| Glu-Sitosterol | 0.93789182 | 0.96273937 | 1.05635854 | 1 |
| MG(18:1) | 1.22564038 | 1.32477308 | 1.08297302 | 1 |
| MG(20:4) | 1.37288278 | 0.96352326 | 1.16845401 | 1 |
| MG(18:0) | 0.92851907 | 0.92927545 | 0.90605412 | 1 |
| MG(16:0) | 0.99556967 | 1.03136026 | 0.95593567 | 1 |
| MG(22:6) | 1.33933106 | 1.05594249 | 1.15561146 | 1 |
| DG(18:0_18:1) | 1.0158107 | 0.9205636 | 1.01847638 | 1 |
| DG(18:1/18:1) | 1.07022061 | 0.95162384 | 1.15577943 | 1 |
| DG(16:0_20:4) | 1.05625574 | 0.82770657 | 1.16973462 | 1 |
| DG(18:0_20:4) | 1.06677225 | 0.84833167 | 1.16790474 | 1 |
| DG(18:0_22:6) | 1.15219477 | 0.95279647 | 1.31616862 | 1 |
| DG(18:1_20:4) | 1.03513365 | 0.86622511 | 1.20009423 | 1 |
| GlcCer(d18:1/16:0) | 2.22163513 | 1.19149946 | 1.78189812 | 1 |
| GlcCer(d18:1/18:0) | 1.46205981 | 0.95576955 | 1.36166575 | 1 |
| GlcCer(d18:2/18:0) | 1.57032387 | 0.91791498 | 1.31665568 | 1 |
| GlcCer(d18:1/20:0) | 1.59944265 | 1.10082448 | 1.43078499 | 1 |
| GlcCer(d18:2/20:0) | 1.70565767 | 1.0775347 | 1.39732541 | 1 |
| GlcCer(d18:1/22:0) | 2.02843272 | 1.13859199 | 1.75002022 | 1 |
| GlcCer(d18:1/22:1) | 1.18073058 | 1.0920974 | 1.0967809 | 1 |
| GlcCer(d18:2/22:0) | 1.10416862 | 1.0231995 | 1.30810189 | 1 |
| GlcCer(d18:1/24:1) | 1.63769649 | 1.0627572 | 1.42013123 | 1 |
| GlcCer(d18:1/24:0) | 1.90800362 | 1.10331771 | 1.62785015 | 1 |
| GalCer(d18:1/16:0) | 0.95377573 | 1.03579944 | 0.98731752 | 1 |
| GalCer(d18:1/18:0) | 1.08295723 | 1.0711189 | 1.08097307 | 1 |
| GalCer(d18:2/18:0) | 1.10558046 | 1.07030437 | 1.02805261 | 1 |
| GalCer(d18:1/20:0) | 0.9834055 | 1.01205304 | 0.99950882 | 1 |
| GalCer(d18:2/20:0) | 0.90135811 | 0.91012739 | 0.86083355 | 1 |
| GalCer(d18:1/22:0) | 0.95195268 | 0.96098222 | 0.97624086 | 1 |
| GalCer(d18:1/22:1) | 0.8802212 | 0.95477018 | 0.91442636 | 1 |
| GalCer(d18:2/22:0) | 0.87455475 | 0.84087909 | 0.83798295 | 1 |
| GalCer(d18:1/24:1) | 0.94958635 | 0.91499795 | 0.92923241 | 1 |
| GalCer(d18:1/24:0) | 0.96166861 | 0.90660743 | 0.93744839 | 1 |
| GalCer | 0.8915232 | 0.79920332 | 0.74682448 | 1 |
| alpha(d18:1/16:0) | | | | |
| GluSph | 1.10548038 | 0.94259932 | 0.70868688 | 1 |
| GalSph | 0.9856999 | 0.97263443 | 0.84839691 | 1 |
| 5-HETE | 1.05260938 | 1.24918007 | 0.97156757 | 1 |
| 12-HETE | 1.26557201 | 1.39758942 | 0.90968976 | 1 |
| 15-HETE | 1.38726117 | 1.56716266 | 1.18427095 | 1 |
| 6k-PGF1alpha | 1.11581922 | 1.26819321 | 1.00650322 | 1 |
| TxB2 | 1.661162 | 1.72217815 | 1.45288251 | 1 |
| PGF2alpha | 1.13462868 | 1.35655273 | 1.05179143 | 1 |
| PGE2 | 1.57913809 | 1.51872651 | 1.36194441 | 1 |
| 5,15-DiHETE | 1.47866435 | 1.4028389 | 1.13105795 | 1 |
| 5-iPF2alpha | 1.44800938 | 1.37299339 | 1.42011554 | 1 |
| PGD2 | 1.7946429 | 1.33883673 | 1.4750862 | 1 |
| 9-HOTrE | 1.18369056 | 1.09028544 | 1.13822769 | 1 |
| 9-OxoODE | 1.02194862 | 1.28526776 | 1.00290607 | 1 |
| 13-OxoODE | 0.85385685 | 0.86595781 | 0.84143578 | 1 |
| 9(10)-EpOME | 0.98699842 | 0.95158158 | 1.00759544 | 1 |
| 9-HODE | 1.36011268 | 1.51848008 | 1.16511187 | 1 |
| 13-HODE | 1.14124554 | 1.26747678 | 0.96222687 | 1 |

### Informal Sequence Listing

| SEQ ID | | |
|---|---|---|
| NO: | Sequence | Description |
| 1 | APELLGGPSVFLFPPKPKDTLMISRTPEVTCVVVDVSHEDPEVKFNWYVD | Wild-type human |
| | GVEVHNAKTKPREEQYNSTYRVVSVLTVLHQDWLNGKEYKCKVSNKALPA | Fc sequence |
| | PIEKTISKAKGQPREPQVYTLPPSRDELTKNQVSLTCLVKGFYPSDIAVE | Positions 231-447 |
| | WESNGQPENNYKTTPPVLDSDGSFFLYSKLTVDKSRWQQGNVFSCSVMHE | EU index |
| | ALHNHYTQKSLSLSPGK | numbering |
| 2 | APELLGGPSVFLFPPKPKDTLMISRTPEVTCVVVDVSHEDPEVKFNWYVD | CH2 domain |
| | GVEVHNAKTKPREEQYNSTYRVVSVLTVLHQDWLNGKEYKCKVSNKALPA | sequence |
| | PIEKTISKAK | Positions 231-340 |
| | | EU index |
| | | numbering |
| 3 | GQPREPQVYTLPPSRDELTKNQVSLTCLVKGFYPSDIAVEWESNGQPENN | CH3 domain |
| | YKTTPPVLDSDGSFFLYSKLTVDKSRWQQGNVFSCSVMHEALHNHYTQKS | sequence |
| | LSLSPGK | Positions 341-447 |
| | | EU index |
| | | numbering |
| 4 | APELLGGPSVFLFPPKPKDTLMISRTPEVTCVVVDVSHEDPEVKFNWYVD | Clone CH3C.35 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVE | |
| | WESYGTEWSSYKTTPPVLDSDGSFFLYSKLTVTKSEWQQGFVFSCSVMHE | |
| | ALHNHYTQKSLSLSPGK | |
| 5 | APELLGGPSVFLFPPKPKDTLMISRTPEVTCVVVDVSHEDPEVKFNWYVD | Clone CH3C.35.13 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESLGHVWAVYKTTPPVLDSDGSFFLYSKLTVPKSTWQQGWVFSCSVMHE | |
| | ALHNHYTQKSLSLSPGK | |
| 6 | APELLGGPSVFLFPPKPKDTLMISRTPEVTCVVVDVSHEDPEVKFNWYVD | Clone CH3C.35.14 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVE | |
| | WESLGHVWAVYQTTPPVLDSDGSFFLYSKLTVPKSTWQQGWVFSCSVMHE | |
| | ALHNHYTQKSLSLSPGK | |
| 7 | APELLGGPSVFLFPPKPKDTLMISRTPEVTCVVVDVSHEDPEVKFNWYVD | Clone CH3C.35.15 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESLGHVWAVYQTTPPVLDSDGSFFLYSKLTVPKSTWQQGWVFSCSVMHE | |
| | ALHNHYTQKSLSLSPGK | |
| 8 | APELLGGPSVFLFPPKPKDTLMISRTPEVTCVVVDVSHEDPEVKFNWYVD | Clone CH3C.35.16 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESLGHVWVNQKTTPPVLDSDGSFFLYSKLTVPKSTWQQGWVFSCSVMHE | |
| | ALHNHYTQKSLSLSPGK | |
| 9 | APELLGGPSVFLFPPKPKDTLMISRTPEVTCVVVDVSHEDPEVKFNWYVD | Clone CH3C.35.17 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVE | |
| | WESLGHVWVNQQTTPPVLDSDGSFFLYSKLTVPKSTWQQGWVFSCSVMHE | |
| | ALHNHYTQKSLSLSPGK | |
| 10 | APELLGGPSVFLFPPKPKDTLMISRTPEVTCVVVDVSHEDPEVKFNWYVD | Clone CH3C.35.18 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESLGHVWVNQQTTPPVLDSDGSFFLYSKLTVPKSTWQQGWVFSCSVMHE | |
| | ALHNHYTQKSLSLSPGK | |
| 11 | APELLGGPSVFLFPPKPKDTLMISRTPEVTCVVVDVSHEDPEVKFNWYVD | Clone CH3C.35.19 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESYGTEWSSYKTTPPVLDSDGSFFLYSKLTVTKSEWQQGFVFSCSVMHE | |
| | ALHNHYTQKSLSLSPGK | |
| 12 | APELLGGPSVFLFPPKPKDTLMISRTPEVTCVVVDVSHEDPEVKFNWYVD | Clone CH3C.35.20 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVE | |
| | WESYGTEWSSYKTTPPVLDSDGSFFLYSKLTVTKEEWQQGFVFSCSVMHE | |
| | ALHNHYTQKSLSLSPGK | |
| 13 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESYGTEWSSYKTTPPVLDSDGSFFLYSKLTVTKEEWQQGFVFSCSVMHE | |
| | ALHNHYTQKSLSLSPGK | |
| 14 | APELLGGPSVFLFPPKPKDTLMISRTPEVTCVVVDVSHEDPEVKFNWYVD | Clone CH3C.35.22 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESYGTEWSNYKTTPPVLDSDGSFFLYSKLTVTKSEWQQGFVFSCSVMHE | |
| | ALHNHYTQKSLSLSPGK | |
| 15 | APELLGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVE | |
| | WESYGTEWSNYKTTPPVLDSDGSFFLYSKLTVTKEEWQQGFVFSCSVMHE | |
| | ALHNHYTQKSLSLSPGK | |
| 16 | APELLGGPSVFLFPPKPKDTLMISRTPEVTCVVVDVSHEDPEVKFNWYVD | Clone CH3C.35.24 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESYGTEWSNYKTTPPVLDSDGSFFLYSKLTVTKEEWQQGFVFSCSVMHE | |
| | ALHNHYTQKSLSLSPGK | |
| 17 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.N163 |
| | PIEKTISKAKGQPREPQVYTLPPSRDELTKNQVSLTCLVKGFYPSDIAVE | |
| | WESYGTEWSNYKTTPPVLDSDGSFFLYSKLTVTKSEWQQGFVFSCSVMHE | |
| | ALHNHYTQKSLSLSPGK | |
| 18 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.K165Q |
| | PIEKTISKAKGQPREPQVYTLPPSRDELTKNQVSLTCLVKGFYPSDIAVE | |
| | WESYGTEWSSYQTTPPVLDSDGSFFLYSKLTVTKSEWQQGFVFSCSVMHE | |
| | ALHNHYTQKSLSLSPGK | |
| 19 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.N163. |
| | PIEKTISKAKGQPREPQVYTLPPSRDELTKNQVSLTCLVKGFYPSDIAVE | K165Q |
| | WESYGTEWSNYQTTPPVLDSDGSFFLYSKLTVTKSEWQQGFVFSCSVMHE | |
| | ALHNHYTQKSLSLSPGK | |
| 20 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.1 |
| | PIEKTISKAKGQPREPQVYTLPPSRDELTKNQVSLTCLVKGFYPSDIAVL | |
| | WESYGTEWSSYKTTPPVLDSDGSFFLYSKLTVTKSEWQQGFVFSCSVMHE | |
| | ALHNHYTQKSLSLSPGK | |
| 21 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.2 |
| | PIEKTISKAKGQPREPQVYTLPPSRDELTKNQVSLTCLVKGFYPSDIAVL | |
| | WESYGTEWSSYRTTPPVLDSDGSFFLYSKLTVTKSEWQQGFVFSCSVMHE | |
| | ALHNHYTQKSLSLSPGK | |
| 22 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.3 |
| | PIEKTISKAKGQPREPQVYTLPPSRDELTKNQVSLTCLVKGFYPSDIAVL | |
| | WESYGTEWSSYRTTPPVLDSDGSFFLYSKLTVTREEWQQGFVFSCSVMHE | |
| | ALHNHYTQKSLSLSPGK | |
| 23 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.4 |
| | PIEKTISKAKGQPREPQVYTLPPSRDELTKNQVSLTCLVKGFYPSDIAVL | |
| | WESYGTEWSSYRTTPPVLDSDGSFFLYSKLTVTGEEWQQGFVFSCSVMHE | |
| | ALHNHYTQKSLSLSPGK | |
| 24 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.5 |
| | PIEKTISKAKGQPREPQVYTLPPSRDELTKNQVSLTCLVKGFYPSDIAVL | |
| | WESYGTEWSSYRTTPPVLDSDGSFFLYSKLTVTREEWQQGFVFSCWVMHE | |
| | ALHNHYTQKSLSLSPGK | |
| 25 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.6 |
| | PIEKTISKAKGQPREPQVYTLPPSRDELTKNQVSLTCLVKGFYPSDIAVL | |
| | WESYGTEWSSYRTTPPVLDSDGSFFLYSKLTVTKEEWQQGFVFSCWVMHE | |
| | ALHNHYTQKSLSLSPGK | |
| 26 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.7 |
| | PIEKTISKAKGQPREPQVYTLPPSRDELTKNQVSLTCLVKGFYPSDIAVL | |
| | WESYGTEWSSYRTTPPVLDSDGSFFLYSKLTVTREEWQQGFVFTCWVMHE | |
| | ALHNHYTQKSLSLSPGK | |
| 27 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.8 |
| | PIEKTISKAKGQPREPQVYTLPPSRDELTKNQVSLTCLVKGFYPSDIAVL | |
| | WESYGTEWSSYRTTPPVLDSDGSFFLYSKLTVTREEWQQGFVFTCGVMHE | |
| | ALHNHYTQKSLSLSPGK | |
| 28 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.9 |
| | PIEKTISKAKGQPREPQVYTLPPSRDELTKNQVSLTCLVKGFYPSDIAVL | |
| | WESYGTEWSSYRTTPPVLDSDGSFFLYSKLTVTREEWQQGFVFECWVMHE | |
| | ALHNHYTQKSLSLSPGK | |
| 29 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.10 |
| | PIEKTISKAKGQPREPQVYTLPPSRDELTKNQVSLTCLVKGFYPSDIAVL | |
| | WESYGTEWSSYRTTPPVLDSDGSFFLYSKLTVTREEWQQGFVFKCWVMHE | |
| | ALHNHYTQKSLSLSPGK | |
| 30 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.11 |
| | PIEKTISKAKGQPREPQVYTLPPSRDELTKNQVSLTCLVKGFYPSDIAVL | |
| | WESYGTEWSSYRTTPPVLDSDGSFFLYSKLTVTPEEWQQGFVFKCWVMHE | |
| | ALHNHYTQKSLSLSPGK | |
| 31 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.12 |
| | PIEKTISKAKGQPREPQVYTLPPSRDELTKNQVSLTCLVKGFYPSDIAVW | |
| | WESYGTEWSSYRTTPPVLDSDGSFFLYSKLTVTREEWQQGFVFSCSVMHE | |
| | ALHNHYTQKSLSLSPGK | |
| 32 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.13 |
| | PIEKTISKAKGQPREPQVYTLPPSRDELTKNQVSLTCLVKGFYPSDIAVW | |
| | WESYGTEWSSYRTTPPVLDSDGSFFLYSKLTVTGEEWQQGFVFSCSVMHE | |
| | ALHNHYTQKSLSLSPGK | |
| 33 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.14 |
| | PIEKTISKAKGQPREPQVYTLPPSRDELTKNQVSLTCLVKGFYPSDIAVW | |
| | WESYGTEWSSYRTTPPVLDSDGSFFLYSKLTVTREEWQQGFVFTCWVMHE | |
| | ALHNHYTQKSLSLSPGK | |
| 34 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.15 |
| | PIEKTISKAKGQPREPQVYTLPPSRDELTKNQVSLTCLVKGFYPSDIAVW | |
| | WESYGTEWSSYRTTPPVLDSDGSFFLYSKLTVTGEEWQQGFVFTCWVMHE | |
| | ALHNHYTQKSLSLSPGK | |
| 35 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.16 |
| | PIEKTISKAKGQPREPQVYTLPPSRDELTKNQVSLTCLVKGFYPSDIAVW | |
| | WESYGTEWSSYRTTPPVLDSDGSFFLYSKLTVTREEWQQGFVFTCGVMHE | |
| | ALHNHYTQKSLSLSPGK | |
| 36 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLTCLVKGFYPSDIAVL | |
| | WESYGTEWSSYKTTPPVLDSDGSFFLYSKLTVTKEEWQQGFVFSCSVMHE | |
| | ALHNHYTQKSLSLSPGK | |
| 37 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.18 |
| | PIEKTISKAKGQPREPQVYTLPPSRDELTKNQVSLTCLVKGFYPSDIAVL | |
| | WESYGTEWSSYRTTPPVLDSDGSFFLYSKLTVTKEEWQQGFVFSCSVMHE | |
| | ALHNHYTQKSLSLSPGK | |
| 38 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 |
| | PIEKTISKAKGQPREPQVYTLPPSRDELTKNQVSLTCLVKGFYPSDIAVE | |
| | WESFGTEWSSYKTTPPVLDSDGSFFLYSKLTVTKEEWQQGFVFSCSVMHE | |
| | ALHNHYTQKSLSLSPGK | |
| 39 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.2 |
| | PIEKTISKAKGQPREPQVYTLPPSRDELTKNQVSLTCLVKGFYPSDIAVE | |
| | WESYGTEWASYKTTPPVLDSDGSFFLYSKLTVTKEEWQQGFVFSCSVMHE | |
| | ALHNHYTQKSLSLSPGK | |
| 40 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.3 |
| | PIEKTISKAKGQPREPQVYTLPPSRDELTKNQVSLTCLVKGFYPSDIAVE | |
| | WESYGTEWVSYKTTPPVLDSDGSFFLYSKLTVTKEEWQQGFVFSCSVMHE | |
| | ALHNHYTQKSLSLSPGK | |
| 41 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.4 |
| | PIEKTISKAKGQPREPQVYTLPPSRDELTKNQVSLTCLVKGFYPSDIAVE | |
| | WESYGTEWSSYKTTPPVLDSDGSFFLYSKLTVSKEEWQQGFVFSCSVMHE | |
| | ALHNHYTQKSLSLSPGK | |
| 42 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.5 |
| | PIEKTISKAKGQPREPQVYTLPPSRDELTKNQVSLTCLVKGFYPSDIAVE | |
| | WESFGTEWASYKTTPPVLDSDGSFFLYSKLTVTKEEWQQGFVFSCSVMHE | |
| | ALHNHYTQKSLSLSPGK | |
| 43 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.6 |
| | PIEKTISKAKGQPREPQVYTLPPSRDELTKNQVSLTCLVKGFYPSDIAVE | |
| | WESFGTEWVSYKTTPPVLDSDGSFFLYSKLTVTKEEWQQGFVFSCSVMHE | |
| | ALHNHYTQKSLSLSPGK | |
| 44 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.a.1 |
| | PIEKTISKAKGQPREPQVYTLPPSRDELTKNQVSLTCLVKGFYPSDIAVW | |
| | WESFGTEWSSYKTTPPVLDSDGSFFLYSKLTVTKEEWQQGFVFSCSVMHE | |
| | ALHNHYTQKSLSLSPGK | |
| 45 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.a.2 |
| | PIEKTISKAKGQPREPQVYTLPPSRDELTKNQVSLTCLVKGFYPSDIAVW | |
| | WESYGTEWASYKTTPPVLDSDGSFFLYSKLTVTKEEWQQGFVFSCSVMHE | |
| | ALHNHYTQKSLSLSPGK | |
| 46 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.a.3 |
| | PIEKTISKAKGQPREPQVYTLPPSRDELTKNQVSLTCLVKGFYPSDIAVW | |
| | WESYGTEWVSYKTTPPVLDSDGSFFLYSKLTVTKEEWQQGFVFSCSVMHE | |
| | ALHNHYTQKSLSLSPGK | |
| 47 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.a.4 |
| | PIEKTISKAKGQPREPQVYTLPPSRDELTKNQVSLTCLVKGFYPSDIAVW | |
| | WESYGTEWSSYKTTPPVLDSDGSFFLYSKLTVSKEEWQQGFVFSCSVMHE | |
| | ALHNHYTQKSLSLSPGK | |
| 48 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.a.5 |
| | PIEKTISKAKGQPREPQVYTLPPSRDELTKNQVSLTCLVKGFYPSDIAVW | |
| | WESFGTEWASYKTTPPVLDSDGSFFLYSKLTVTKEEWQQGFVFSCSVMHE | |
| | ALHNHYTQKSLSLSPGK | |
| 49 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.a.6 |
| | PIEKTISKAKGQPREPQVYTLPPSRDELTKNQVSLTCLVKGFYPSDIAVW | |
| | WESFGTEWVSYKTTPPVLDSDGSFFLYSKLTVTKEEWQQGFVFSCSVMHE | |
| | ALHNHYTQKSLSLSPGK | |
| 50 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.1 |
| | PIEKTISKAKGQPREPQVYTLPPSRDELTKNQVSLTCLVKGFYPSDIAVE | |
| | WESFGTEWSNYKTTPPVLDSDGSFFLYSKLTVTKEEWQQGFVFSCSVMHE | |
| | ALHNHYTQKSLSLSPGK | |
| 51 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 |
| | PIEKTISKAKGQPREPQVYTLPPSRDELTKNQVSLTCLVKGFYPSDIAVE | |
| | WESYGTEWANYKTTPPVLDSDGSFFLYSKLTVTKEEWQQGFVFSCSVMHE | |
| | ALHNHYTQKSLSLSPGK | |
| 52 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 |
| | PIEKTISKAKGQPREPQVYTLPPSRDELTKNQVSLTCLVKGFYPSDIAVE | |
| | WESYGTEWVNYKTTPPVLDSDGSFFLYSKLTVTKEEWQQGFVFSCSVMHE | |
| | ALHNHYTQKSLSLSPGK | |
| 53 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 |
| | PIEKTISKAKGQPREPQVYTLPPSRDELTKNQVSLTCLVKGFYPSDIAVE | |
| | WESYGTEWSNYKTTPPVLDSDGSFFLYSKLTVSKEEWQQGFVFSCSVMHE | |
| | ALHNHYTQKSLSLSPGK | |
| 54 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.5 |
| | PIEKTISKAKGQPREPQVYTLPPSRDELTKNQVSLTCLVKGFYPSDIAVE | |
| | WESFGTEWANYKTTPPVLDSDGSFFLYSKLTVTKEEWQQGFVFSCSVMHE | |
| | ALHNHYTQKSLSLSPGK | |
| 55 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.6 |
| | PIEKTISKAKGQPREPQVYTLPPSRDELTKNQVSLTCLVKGFYPSDIAVE | |
| | WESFGTEWVNYKTTPPVLDSDGSFFLYSKLTVTKEEWQQGFVFSCSVMHE | |
| | ALHNHYTQKSLSLSPGK | |
| 56 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.24.1 |
| | PIEKTISKAKGQPREPQVYTLPPSRDELTKNQVSLTCLVKGFYPSDIAVW | |
| | WESFGTEWSNYKTTPPVLDSDGSFFLYSKLTVTKEEWQQGFVFSCSVMHE | |
| | ALHNHYTQKSLSLSPGK | |
| 57 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.24.2 |
| | PIEKTISKAKGQPREPQVYTLPPSRDELTKNQVSLTCLVKGFYPSDIAVW | |
| | WESYGTEWANYKTTPPVLDSDGSFFLYSKLTVTKEEWQQGFVFSCSVMHE | |
| | ALHNHYTQKSLSLSPGK | |
| 58 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.24.3 |
| | PIEKTISKAKGQPREPQVYTLPPSRDELTKNQVSLTCLVKGFYPSDIAVW | |
| | WESYGTEWVNYKTTPPVLDSDGSFFLYSKLTVTKEEWQQGFVFSCSVMHE | |
| | ALHNHYTQKSLSLSPGK | |
| 59 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.24.4 |
| | PIEKTISKAKGQPREPQVYTLPPSRDELTKNQVSLTCLVKGFYPSDIAVW | |
| | WESYGTEWSNYKTTPPVLDSDGSFFLYSKLTVSKEEWQQGFVFSCSVMHE | |
| | ALHNHYTQKSLSLSPGK | |
| 60 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.24.5 |
| | PIEKTISKAKGQPREPQVYTLPPSRDELTKNQVSLTCLVKGFYPSDIAVW | |
| | WESFGTEWANYKTTPPVLDSDGSFFLYSKLTVTKEEWQQGFVFSCSVMHE | |
| | ALHNHYTQKSLSLSPGK | |
| 61 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.24.6 |
| | PIEKTISKAKGQPREPQVYTLPPSRDELTKNQVSLTCLVKGFYPSDIAVW | |
| | WESFGTEWVNYKTTPPVLDSDGSFFLYSKLTVTKEEWQQGFVFSCSVMHE | |
| | ALHNHYTQKSLSLSPGK | |
| 62 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.1 |
| | PIEKTISKAKGQPREPQVYTLPPSRDELTKNQVSLTCLVKGFYPSDIAVL | |
| | WESFGTEWSSYKTTPPVLDSDGSFFLYSKLTVTKEEWQQGFVFSCSVMHE | |
| | ALHNHYTQKSLSLSPGK | |
| 63 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLTCLVKGFYPSDIAVL | |
| | WESYGTEWASYKTTPPVLDSDGSFFLYSKLTVTKEEWQQGFVFSCSVMHE | |
| | ALHNHYTQKSLSLSPGK | |
| 64 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.3 |
| | PIEKTISKAKGQPREPQVYTLPPSRDELTKNQVSLTCLVKGFYPSDIAVL | |
| | WESYGTEWVSYKTTPPVLDSDGSFFLYSKLTVTKEEWQQGFVFSCSVMHE | |
| | ALHNHYTQKSLSLSPGK | |
| 65 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.4 |
| | PIEKTISKAKGQPREPQVYTLPPSRDELTKNQVSLTCLVKGFYPSDIAVL | |
| | WESYGTEWSSYKTTPPVLDSDGSFFLYSKLTVSKEEWQQGFVFSCSVMHE | |
| | ALHNHYTQKSLSLSPGK | |
| 66 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.5 |
| | PIEKTISKAKGQPREPQVYTLPPSRDELTKNQVSLTCLVKGFYPSDIAVL | |
| | WESFGTEWASYKTTPPVLDSDGSFFLYSKLTVTKEEWQQGFVFSCSVMHE | |
| | ALHNHYTQKSLSLSPGK | |
| 67 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.6 |
| | PIEKTISKAKGQPREPQVYTLPPSRDELTKNQVSLTCLVKGFYPSDIAVL | |
| | WESFGTEWVSYKTTPPVLDSDGSFFLYSKLTVTKEEWQQGFVFSCSVMHE | |
| | ALHNHYTQKSLSLSPGK | |
| 68 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.N390 |
| | PIEKTISKAKGQPREPQVYTLPPSRDELTKNQVSLTCLVKGFYPSDIAVE | |
| | WESYGTEWSNYKTTPPVLDSDGSFFLYSKLTVTKSEWQQGFVFSCSVMHE | |
| | ALHNHYTQKSLSLSPGK | |
| 69 | MPPPRTGRGLLWLGLVLSSVCVALGSETQANSTTDALNVLLIIVDDLRPS | Full-length human |
| | LGCYGDKLVRSPNIDQLASHSLLFQNAFAQQAVCAPSRVSFLTGRRPDTT | IDS polypeptide |
| | RLYDFNSYWRVHAGNFSTIPQYFKENGYVTMSVGKVFHPGISSNHTDDSP | sequence |
| | YSWSFPPYHPSSEKYENTKTCRGPDGELHANLLCPVDVLDVPEGTLPDKQ | |
| | STEQAIQLLEKMKTSASPFFLAVGYHKPHIPFRYPKEFQKLYPLENITLA | |
| | PDPEVPDGLPPVAYNPWMDIRQREDVQALNISVPYGPIPVDFQRKIRQSY | |
| | FASVSYLDTQVGRLLSALDDLQLANSTIIAFTSDHGWALGEHGEWAKYSN | |
| | FDVATHVPLIFYVPGRTASLPEAGEKLFPYLDPFDSASQLMEPGRQSMDL | |
| | VELVSLFPTLAGLAGLQVPPRCPVPSFHVELCREGKNLLKHFRFRDLEED | |
| | PYLPGNPRELIAYSQYPRPSDIPQWNSDKPSLKDIKIMGYSIRTIDYRYT | |
| | VWVGFNPDEFLANFSDIHAGELYFVDSDPLQDHNMYNDSQGGDLFQLLMP | |
| 70 | TDALNVLLIIVDDLRPSLGCYGDKLVRSPNIDQLASHSLLFQNAFAQQAV | Mature human IDS |
| | CAPSRVSFLTGRRPDTTRLYDFNSYWRVHAGNFSTIPQYFKENGYVTMSV | polypeptide |
| | GKVFHPGISSNHTDDSPYSWSFPPYHPSSEKYENTKTCRGPDGELHANLL | sequence |
| | CPVDVLDVPEGTLPDKQSTEQAIQLLEKMKTSASPFFLAVGYHKPHIPFR | |
| | YPKEFQKLYPLENITLAPDPEVPDGLPPVAYNPWMDIRQREDVQALNISV | |
| | PYGPIPVDFQRKIRQSYFASVSYLDTQVGRLLSALDDLQLANSTIIAFTS | |
| | DHGWALGEHGEWAKYSNFDVATHVPLIFYVPGRTASLPEAGEKLFPYLDP | |
| | FDSASQLMEPGRQSMDLVELVSLFPTLAGLAGLQVPPRCPVPSFHVELCR | |
| | EGKNLLKHFRFRDLEEDPYLPGNPRELIAYSQYPRPSDIPQWNSDKPSLK | |
| | DIKIMGYSIRTIDYRYTVWVGFNPDEFLANFSDIHAGELYFVDSDPLQDH | |
| | NMYNDSQGGDLFQLLMP | |
| 71 | EPKSCDKTHTCPPCP | Human IgG1 hinge |
| | | amino acid |
| | | sequence |
| 72 | MMDQARSAFSNLFGGEPLSYTRFSLARQVDGDNSHVEMKLAVDEEENADN | Human transferrin |
| | NTKANVTKPKRCSGSICYGTIAVIVFFLIGFMIGYLGYCKGVEPKTECER | receptor protein |
| | LAGTESPVREEPGEDFPAARRLYWDDLKRKLSEKLDSTDFTGTIKLLNEN | 1 (TFR1) |
| | SYVPREAGSQKDENLALYVENQFREFKLSKVWRDQHFVKIQVKDSAQNSV | |
| | IIVDKNGRLVYLVENPGGYVAYSKAATVTGKLVHANFGTKKDFEDLYTPV | |
| | NGSIVIVRAGKITFAEKVANAESLNAIGVLIYMDQTKFPIVNAELSFFGH | |
| | AHLGTGDPYTPGFPSFNHTQFPPSRSSGLPNIPVQTISRAAAEKLFGNME | |
| | GDCPSDWKTDSTCRMVTSESKNVKLTVSNVLKEIKILNIFGVIKGFVEPD | |
| | HYVVVGAQRDAWGPGAAKSGVGTALLLKLAQMFSDMVLKDGFQPSRSIIF | |
| | ASWSAGDFGSVGATEWLEGYLSSLHLKAFTYINLDKAVLGTSNFKVSASP | |
| | LLYTLIEKTMQNVKHPVTGQFLYQDSNWASKVEKLTLDNAAFPFLAYSGI | |
| | PAVSFCFCEDTDYPYLGTTMDTYKELIERIPELNKVARAAAEVAGQFVIK | |
| | LTHDVELNLDYERYNSQLLSFVRDLNQYRADIKEMGLSLQWLYSARGDFF | |
| | RATSRLTTDFGNAEKTDRFVMKKLNDRVMRVEYHFLSPYVSPKESPFRHV | |
| | FWGSGSHTLPALLENLKLRKQNNGAFNETLFRNQLALATWTIQGAANALS | |
| | GDVWDIDNEE | |
| 73 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob |
| | PIEKTISKAKGQPREPQVYTLPPSRDELTKNQVSLWCLVKGFYPSDIAVW | mutation |
| | WESYGTEWSSYKTTPPVLDSDGSFFLYSKLTVTKEEWQQGFVFSCSVMHE | |
| | ALHNHYTQKSLSLSPGK | |
| 74 | APEAAGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob and |
| | PIEKTISKAKGQPREPQVYTLPPSRDELTKNQVSLWCLVKGFYPSDIAVW | LALA mutations |
| | WESYGTEWSSYKTTPPVLDSDGSFFLYSKLTVTKEEWQQGFVFSCSVMHE | |
| | ALHNHYTQKSLSLSPGK | |
| 75 | APELLGGPSVFLFPPKPKDTLYITREPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob and YTE |
| | PIEKTISKAKGQPREPQVYTLPPSRDELTKNQVSLWCLVKGFYPSDIAVW | mutations |
| | WESYGTEWSSYKTTPPVLDSDGSFFLYSKLTVTKEEWQQGFVFSCSVMHE | |
| | ALHNHYTQKSLSLSPGK | |
| 76 | APEAAGGPSVFLFPPKPKDTLYITREPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob, LALA, |
| | PIEKTISKAKGQPREPQVYTLPPSRDELTKNQVSLWCLVKGFYPSDIAVW | and YTE mutations |
| | WESYGTEWSSYKTTPPVLDSDGSFFLYSKLTVTKEEWQQGFVFSCSVMHE | |
| | ALHNHYTQKSLSLSPGK | |
| 77 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole mutations |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSVMHE | |
| | ALHNHYTQKSLSLSPGK | |
| 78 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole and LALA |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPGK | |
| 79 | APELLGGPSVFLFPPKPKDTLYITREPEVTCVVVDVSHEDPEVKFNWYVD | Fc sequence with |
| | GVEVHNAKTKPREEQYNSTYRVVSVLTVLHQDWLNGKEYKCKVSNKALPA | hole and YTE |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPGK | |
| 80 | APEAAGGPSVFLFPPKPKDTLYITREPEVTCVVVDVSHEDPEVKFNWYVD | Fc sequence with |
| | GVEVHNAKTKPREEQYNSTYRVVSVLTVLHQDWLNGKEYKCKVSNKALPA | hole, LALA, and |
| | PIEKTISKAKGQPREPQVYTLPPSRDELTKNQVSLSCAVKGFYPSDIAVE | YTE mutations |
| | WESNGQPENNYKTTPPVLDSDGSFFLVSKLTVDKSRWQQGNVFSCSVMHE | |
| | ALHNHYTQKSLSLSPGK | |
| 81 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with hole |
| | PIEKTISKAKGQPREPQVYTLPPSRDELTKNQVSLSCAVKGFYPSDIAVW | mutations |
| | WESYGTEWSSYKTTPPVLDSDGSFFLVSKLTVTKEEWQQGFVFSCSVMHE | |
| | ALHNHYTQKSLSLSPGK | |
| 82 | APEAAGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with hole and |
| | PIEKTISKAKGQPREPQVYTLPPSRDELTKNQVSLSCAVKGFYPSDIAVW | LALA mutations |
| | WESYGTEWSSYKTTPPVLDSDGSFFLVSKLTVTKEEWQQGFVFSCSVMHE | |
| | ALHNHYTQKSLSLSPGK | |
| 83 | APELLGGPSVFLFPPKPKDTLYITREPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with hole and YTE |
| | PIEKTISKAKGQPREPQVYTLPPSRDELTKNQVSLSCAVKGFYPSDIAVW | mutations |
| | WESYGTEWSSYKTTPPVLDSDGSFFLVSKLTVTKEEWQQGFVFSCSVMHE | |
| | ALHNHYTQKSLSLSPGK | |
| 84 | APEAAGGPSVFLFPPKPKDTLYITREPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with hole, LALA, |
| | PIEKTISKAKGQPREPQVYTLPPSRDELTKNQVSLSCAVKGFYPSDIAVW | and YTE mutations |
| | WESYGTEWSSYKTTPPVLDSDGSFFLVSKLTVTKEEWQQGFVFSCSVMHE | |
| | ALHNHYTQKSLSLSPGK | |
| 85 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | knob mutation |
| | PIEKTISKAKGQPREPQVYTLPPSRDELTKNQVSLWCLVKGFYPSDIAVE | |
| | WESNGQPENNYKTTPPVLDSDGSFFLYSKLTVDKSRWQQGNVFSCSVMHE | |
| | ALHNHYTQKSLSLSPGK | |
| 86 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | knob and LALA |
| | PIEKTISKAKGQPREPQVYTLPPSRDELTKNQVSLWCLVKGFYPSDIAVE | mutations |
| | WESNGQPENNYKTTPPVLDSDGSFFLYSKLTVDKSRWQQGNVFSCSVMHE | |
| | ALHNHYTQKSLSLSPGK | |
| 87 | APELLGGPSVFLFPPKPKDTLYITREPEVTCVVVDVSHEDPEVKFNWYVD | Fc sequence with |
| | GVEVHNAKTKPREEQYNSTYRVVSVLTVLHQDWLNGKEYKCKVSNKALPA | knob and YTE |
| | PIEKTISKAKGQPREPQVYTLPPSRDELTKNQVSLWCLVKGFYPSDIAVE | mutations |
| | WESNGQPENNYKTTPPVLDSDGSFFLYSKLTVDKSRWQQGNVFSCSVMHE | |
| | ALHNHYTQKSLSLSPGK | |
| 88 | APEAAGGPSVFLFPPKPKDTLYITREPEVTCVVVDVSHEDPEVKFNWYVD | Fc sequence with |
| | GVEVHNAKTKPREEQYNSTYRVVSVLTVLHQDWLNGKEYKCKVSNKALPA | knob, LALA, and |
| | PIEKTISKAKGQPREPQVYTLPPSRDELTKNQVSLWCLVKGFYPSDIAVE | YTE mutations |
| | WESNGQPENNYKTTPPVLDSDGSFFLYSKLTVDKSRWQQGNVFSCSVMHE | |
| | ALHNHYTQKSLSLSPGK | |
| 89 | DKTHTCPPCP | Portion of human |
| | | IgG1 hinge |
| | | sequence |
| 90 | SETQANSTTD ALNVLLIIVD DLRPSLGCYG DKLVRSPNID | IDS sequence |
| | QLASHSLLFQ NAFAQQAVCA PSRVSFLTGR RPDTTRLYDF | (cysteine |
| | NSYWRVHAGN FSTIPQYFKE NGYVTMSVGK VFHPGISSNH | modified to |
| | TDDSPYSWSF PPYHPSSEKY ENTKTCRGPD GELHANLLCP | formylglycine |
| | VDVLDVPEGT LPDKQSTEQA IQLLEKMKTS ASPFFLAVGY | double |
| | HKPHIPFRYP KEFQKLYPLE NITLAPDPEV PDGLPPVAYN | underlined) |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF QLLMP | |
| 91 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (cysteine |
| | HKPHIPFRYP KEFQKLYPLE NITLAPDPEV PDGLPPVAYN | modified to |
| | PWMDIRQRED VQALNISVPY GPIPVDFQRK IRQSYFASVS | formylglycine |
| | YLDTQVGRLL SALDDLQLAN STIIAFTSDH GWALGEHGEW | double |
| | AKYSNFDVAT HVPLIFYVPG RTASLPEAGE KLFPYLDPFD | underlined) and |
| | SASQLMEPGR QSMDLVELVS LFPTLAGLAG LQVPPRCPVP | hole and LALA |
| | SFHVELCREG KNLLKHFRFR DLEEDPYLPG NPRELIAYSQ | mutations |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVWDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 92 | DKTHTCPPCPAPEAAGGPSVFLFPPKPKDTLMISRTPEVTCVVVDVSHED | Clone CH3C.35.21 |
| | PEVKFNWYVDGVEVHNAKTKPREEQYNSTYRVVSVLTVLHQDWLNGKEYK | with knob and |
| | CKVSNKALPAPIEKTISKAKGQPREPQVYTLPPSRDELTKNQVSLWCLVK | LALA mutations |
| | GFYPSDIAVWWESYGTEWSSYKTTPPVLDSDGSFFLYSKLTVTKEEWQQG | and portion of |
| | FVFSCSVMHEALHNHYTQKSLSLSPGK | human IgG1 hinge |
| | | sequence |
| 93 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (cysteine |
| | HKPHIPFRYP KEFQKLYPLE NITLAPDPEV PDGLPPVAYN | modified to |
| | PWMDIRQRED VQALNISVPY GPIPVDFQRK IRQSYFASVS | formylglycine |
| | YLDTQVGRLL SALDDLQLAN STIIAFTSDH GWALGEHGEW | double |
| | AKYSNFDVAT HVPLIFYVPG RTASLPEAGE KLFPYLDPFD | underlined) and |
| | SASQLMEPGR QSMDLVELVS LFPTLAGLAG LQVPPRCPVP | hole mutations |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 94 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (cysteine |
| | HKPHIPFRYP KEFQKLYPLE NITLAPDPEV PDGLPPVAYN | modified to |
| | PWMDIRQRED VQALNISVPY GPIPVDFQRK IRQSYFASVS | formylglycine |
| | YLDTQVGRLL SALDDLQLAN STIIAFTSDH GWALGEHGEW | double |
| | AKYSNFDVAT HVPLIFYVPG RTASLPEAGE KLFPYLDPFD | underlined) and |
| | SASQLMEPGR QSMDLVELVS LFPTLAGLAG LQVPPRCPVP | knob mutation |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLWCLVK | |
| | GFYPSDIAVE WESNGQPENN YKTTPPVLDS DGSFFLYSKL | |
| | TVDKSRWQQG NVFSCSVMHE ALHNHYTQKS LSLSPGK | |
| 95 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLWCLVKGFYPSDIAVL | with knob and |
| | WESYGTEWSSYKTTPPVLDSDGSFFLYSKLTVTKEEWQQGFVFSCSVMHE | LALA mutations |
| | ALHNHYTQKSLSLSPGK | |
| 96 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLWCLVKGFYPSDIAVE | knob mutation |
| | WESFGTEWSSYKTTPPVLDSDGSFFLYSKLTVTKEEWQQGFVFSCSVMHE | |
| | ALHNHYTQKSLSLSPGK | |
| 97 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLWCLVKGFYPSDIAVE | knob and LALA |
| | WESFGTEWSSYKTTPPVLDSDGSFFLYSKLTVTKEEWQQGFVFSCSVMHE | mutations |
| | ALHNHYTQKSLSLSPGK | |
| 98 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.20.1 with |
| | PIEKTISKAKGQPREPQVYTLPPSRDELTKNQVSLWCLVKGFYPSDIAVE | knob and LALAPG |
| | WESFGTEWSSYKTTPPVLDSDGSFFLYSKLTVTKEEWQQGFVFSCSVMHE | mutations |
| | ALHNHYTQKSLSLSPGK | |
| 99 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLWCLVKGFYPSDIAVE | knob and YTE |
| | WESFGTEWSSYKTTPPVLDSDGSFFLYSKLTVTKEEWQQGFVFSCSVMHE | mutations |
| | ALHNHYTQKSLSLSPGK | |
| 100 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLWCLVKGFYPSDIAVE | knob, LALA, and |
| | WESFGTEWSSYKTTPPVLDSDGSFFLYSKLTVTKEEWQQGFVFSCSVMHE | YTE mutations |
| | ALHNHYTQKSLSLSPGK | |
| 101 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.20.1 with |
| | PIEKTISKAKGQPREPQVYTLPPSRDELTKNQVSLWCLVKGFYPSDIAVE | knob, LALAPG, and |
| | WESFGTEWSSYKTTPPVLDSDGSFFLYSKLTVTKEEWQQGFVFSCSVMHE | YTE mutations |
| | ALHNHYTQKSLSLSPGK | |
| 102 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLSCAVKGFYPSDIAVE | hole mutations |
| | WESFGTEWSSYKTTPPVLDSDGSFFLVSKLTVTKEEWQQGFVFSCSVMHE | |
| | ALHNHYTQKSLSLSPGK | |
| 103 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLSCAVKGFYPSDIAVE | hole and LALA |
| | WESFGTEWSSYKTTPPVLDSDGSFFLVSKLTVTKEEWQQGFVFSCSVMHE | mutations |
| | ALHNHYTQKSLSLSPGK | |
| 104 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.20.1 with |
| | PIEKTISKAKGQPREPQVYTLPPSRDELTKNQVSLSCAVKGFYPSDIAVE | hole and LALAPG |
| | WESFGTEWSSYKTTPPVLDSDGSFFLVSKLTVTKEEWQQGFVFSCSVMHE | mutations |
| | ALHNHYTQKSLSLSPGK | |
| 105 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLSCAVKGFYPSDIAVE | hole and YTE |
| | WESFGTEWSSYKTTPPVLDSDGSFFLVSKLTVTKEEWQQGFVFSCSVMHE | mutations |
| | ALHNHYTQKSLSLSPGK | |
| 106 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.20.1 with |
| | PIEKTISKAKGQPREPQVYTLPPSRDELTKNQVSLSCAVKGFYPSDIAVE | hole, LALA, and |
| | WESFGTEWSSYKTTPPVLDSDGSFFLVSKLTVTKEEWQQGFVFSCSVMHE | YTE mutations |
| | ALHNHYTQKSLSLSPGK | |
| 107 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.20.1 with |
| | PIEKTISKAKGQPREPQVYTLPPSRDELTKNQVSLSCAVKGFYPSDIAVE | hole, LALAPG, and |
| | WESFGTEWSSYKTTPPVLDSDGSFFLVSKLTVTKEEWQQGFVFSCSVMHE | YTE mutations |
| | ALHNHYTQKSLSLSPGK | |
| 108 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob mutation |
| | WESYGTEWANYKTTPPVLDSDGSFFLYSKLTVTKEEWQQGFVFSCSVMHE | |
| | ALHNHYTQKSLSLSPGK | |
| 109 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and LALA |
| | WESYGTEWANYKTTPPVLDSDGSFFLYSKLTVTKEEWQQGFVFSCSVMHE | mutations |
| | ALHNHYTQKSLSLSPGK | |
| 110 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob and LALAPG |
| | WESYGTEWANYKTTPPVLDSDGSFFLYSKLTVTKEEWQQGFVFSCSVMHE | mutations |
| | ALHNHYTQKSLSLSPGK | |
| 111 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and YTE |
| | WESYGTEWANYKTTPPVLDSDGSFFLYSKLTVTKEEWQQGFVFSCSVMHE | mutations |
| | ALHNHYTQKSLSLSPGK | |
| 112 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob, LALA, and |
| | WESYGTEWANYKTTPPVLDSDGSFFLYSKLTVTKEEWQQGFVFSCSVMHE | YTE mutations |
| | ALHNHYTQKSLSLSPGK | |
| 113 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob, LALAPG, and |
| | WESYGTEWANYKTTPPVLDSDGSFFLYSKLTVTKEEWQQGFVFSCSVMHE | YTE mutations |
| | ALHNHYTQKSLSLSPGK | |
| 114 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole mutations |
| | WESYGTEWANYKTTPPVLDSDGSFFLVSKLTVTKEEWQQGFVFSCSVMHE | |
| | ALHNHYTQKSLSLSPGK | |
| 115 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole and LALA |
| | WESYGTEWANYKTTPPVLDSDGSFFLVSKLTVTKEEWQQGFVFSCSVMHE | mutations |
| | ALHNHYTQKSLSLSPGK | |
| 116 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLSCAVKGFYPSDIAVE | hole and LALAPG |
| | WESYGTEWANYKTTPPVLDSDGSFFLVSKLTVTKEEWQQGFVFSCSVMHE | mutations |
| | ALHNHYTQKSLSLSPGK | |
| 117 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole and YTE |
| | WESYGTEWANYKTTPPVLDSDGSFFLVSKLTVTKEEWQQGFVFSCSVMHE | mutations |
| | ALHNHYTQKSLSLSPGK | |
| 118 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLSCAVKGFYPSDIAVE | hole, LALA, and |
| | WESYGTEWANYKTTPPVLDSDGSFFLVSKLTVTKEEWQQGFVFSCSVMHE | YTE mutations |
| | ALHNHYTQKSLSLSPGK | |
| 119 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLSCAVKGFYPSDIAVE | hole, LALAPG, and |
| | WESYGTEWANYKTTPPVLDSDGSFFLVSKLTVTKEEWQQGFVFSCSVMHE | YTE mutations |
| | ALHNHYTQKSLSLSPGK | |
| 120 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLWCLVKGFYPSDIAVE | knob mutation |
| | WESYGTEWVNYKTTPPVLDSDGSFFLYSKLTVTKEEWQQGFVFSCSVMHE | |
| | ALHNHYTQKSLSLSPGK | |
| 121 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLWCLVKGFYPSDIAVE | knob and LALA |
| | WESYGTEWVNYKTTPPVLDSDGSFFLYSKLTVTKEEWQQGFVFSCSVMHE | mutations |
| | ALHNHYTQKSLSLSPGK | |
| 122 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.3 with |
| | PIEKTISKAKGQPREPQVYTLPPSRDELTKNQVSLWCLVKGFYPSDIAVE | knob and LALAPG |
| | WESYGTEWVNYKTTPPVLDSDGSFFLYSKLTVTKEEWQQGFVFSCSVMHE | mutations |
| | ALHNHYTQKSLSLSPGK | |
| 123 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLWCLVKGFYPSDIAVE | knob and YTE |
| | WESYGTEWVNYKTTPPVLDSDGSFFLYSKLTVTKEEWQQGFVFSCSVMHE | mutations |
| | ALHNHYTQKSLSLSPGK | |
| 124 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLWCLVKGFYPSDIAVE | knob, LALA, and |
| | WESYGTEWVNYKTTPPVLDSDGSFFLYSKLTVTKEEWQQGFVFSCSVMHE | YTE mutations |
| | ALHNHYTQKSLSLSPGK | |
| 125 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.3 with |
| | PIEKTISKAKGQPREPQVYTLPPSRDELTKNQVSLWCLVKGFYPSDIAVE | knob, LALAPG, and |
| | WESYGTEWVNYKTTPPVLDSDGSFFLYSKLTVTKEEWQQGFVFSCSVMHE | YTE mutations |
| | ALHNHYTQKSLSLSPGK | |
| 126 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLSCAVKGFYPSDIAVE | hole mutations |
| | WESYGTEWVNYKTTPPVLDSDGSFFLVSKLTVTKEEWQQGFVFSCSVMHE | |
| | ALHNHYTQKSLSLSPGK | |
| 127 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLSCAVKGFYPSDIAVE | hole and LALA |
| | WESYGTEWVNYKTTPPVLDSDGSFFLVSKLTVTKEEWQQGFVFSCSVMHE | mutations |
| | ALHNHYTQKSLSLSPGK | |
| 128 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.3 with |
| | PIEKTISKAKGQPREPQVYTLPPSRDELTKNQVSLSCAVKGFYPSDIAVE | hole and LALAPG |
| | WESYGTEWVNYKTTPPVLDSDGSFFLVSKLTVTKEEWQQGFVFSCSVMHE | mutations |
| | ALHNHYTQKSLSLSPGK | |
| 129 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLSCAVKGFYPSDIAVE | hole and YTE |
| | WESYGTEWVNYKTTPPVLDSDGSFFLVSKLTVTKEEWQQGFVFSCSVMHE | mutations |
| | ALHNHYTQKSLSLSPGK | |
| 130 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.3 with |
| | PIEKTISKAKGQPREPQVYTLPPSRDELTKNQVSLSCAVKGFYPSDIAVE | hole, LALA, and |
| | WESYGTEWVNYKTTPPVLDSDGSFFLVSKLTVTKEEWQQGFVFSCSVMHE | YTE mutations |
| | ALHNHYTQKSLSLSPGK | |
| 131 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.3 with |
| | PIEKTISKAKGQPREPQVYTLPPSRDELTKNQVSLSCAVKGFYPSDIAVE | hole, LALAPG, and |
| | WESYGTEWVNYKTTPPVLDSDGSFFLVSKLTVTKEEWQQGFVFSCSVMHE | YTE mutations |
| | ALHNHYTQKSLSLSPGK | |
| 132 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLWCLVKGFYPSDIAVE | knob mutation |
| | WESYGTEWSNYKTTPPVLDSDGSFFLYSKLTVSKEEWQQGFVFSCSVMHE | |
| | ALHNHYTQKSLSLSPGK | |
| 133 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLWCLVKGFYPSDIAVE | knob and LALA |
| | WESYGTEWSNYKTTPPVLDSDGSFFLYSKLTVSKEEWQQGFVFSCSVMHE | mutations |
| | ALHNHYTQKSLSLSPGK | |
| 134 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.4 with |
| | PIEKTISKAKGQPREPQVYTLPPSRDELTKNQVSLWCLVKGFYPSDIAVE | knob and LALAPG |
| | WESYGTEWSNYKTTPPVLDSDGSFFLYSKLTVSKEEWQQGFVFSCSVMHE | mutations |
| | ALHNHYTQKSLSLSPGK | |
| 135 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLWCLVKGFYPSDIAVE | knob and YTE |
| | WESYGTEWSNYKTTPPVLDSDGSFFLYSKLTVSKEEWQQGFVFSCSVMHE | mutations |
| | ALHNHYTQKSLSLSPGK | |
| 136 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLWCLVKGFYPSDIAVE | knob, LALA, and |
| | WESYGTEWSNYKTTPPVLDSDGSFFLYSKLTVSKEEWQQGFVFSCSVMHE | YTE mutations |
| | ALHNHYTQKSLSLSPGK | |
| 137 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.4 with |
| | PIEKTISKAKGQPREPQVYTLPPSRDELTKNQVSLWCLVKGFYPSDIAVE | knob, LALAPG, and |
| | WESYGTEWSNYKTTPPVLDSDGSFFLYSKLTVSKEEWQQGFVFSCSVMHE | YTE mutations |
| | ALHNHYTQKSLSLSPGK | |
| 138 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLSCAVKGFYPSDIAVE | hole mutations |
| | WESYGTEWSNYKTTPPVLDSDGSFFLVSKLTVSKEEWQQGFVFSCSVMHE | |
| | ALHNHYTQKSLSLSPGK | |
| 139 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLSCAVKGFYPSDIAVE | hole and LALA |
| | WESYGTEWSNYKTTPPVLDSDGSFFLVSKLTVSKEEWQQGFVFSCSVMHE | mutations |
| | ALHNHYTQKSLSLSPGK | |
| 140 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.4 with |
| | PIEKTISKAKGQPREPQVYTLPPSRDELTKNQVSLSCAVKGFYPSDIAVE | hole and LALAPG |
| | WESYGTEWSNYKTTPPVLDSDGSFFLVSKLTVSKEEWQQGFVFSCSVMHE | mutations |
| | ALHNHYTQKSLSLSPGK | |
| 141 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLSCAVKGFYPSDIAVE | hole and YTE |
| | WESYGTEWSNYKTTPPVLDSDGSFFLVSKLTVSKEEWQQGFVFSCSVMHE | mutations |
| | ALHNHYTQKSLSLSPGK | |
| 142 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.4 with |
| | PIEKTISKAKGQPREPQVYTLPPSRDELTKNQVSLSCAVKGFYPSDIAVE | hole, LALA, and |
| | WESYGTEWSNYKTTPPVLDSDGSFFLVSKLTVSKEEWQQGFVFSCSVMHE | YTE mutations |
| | ALHNHYTQKSLSLSPGK | |
| 143 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.4 with |
| | PIEKTISKAKGQPREPQVYTLPPSRDELTKNQVSLSCAVKGFYPSDIAVE | hole, LALAPG, and |
| | WESYGTEWSNYKTTPPVLDSDGSFFLVSKLTVSKEEWQQGFVFSCSVMHE | YTE mutations |
| | ALHNHYTQKSLSLSPGK | |
| 144 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob |
| | WESYGTEWASYKTTPPVLDSDGSFFLYSKLTVTKEEWQQGFVFSCSVMHE | mutation |
| | ALHNHYTQKSLSLSPGK | |
| 145 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALA mutations |
| | ALHNHYTQKSLSLSPGK | |
| 146 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALAPG mutations |
| | ALHNHYTQKSLSLSPGK | |
| 147 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob and YTE |
| | WESYGTEWASYKTTPPVLDSDGSFFLYSKLTVTKEEWQQGFVFSCSVMHE | mutations |
| | ALHNHYTQKSLSLSPGK | |
| 148 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob, LALA, |
| | WESYGTEWASYKTTPPVLDSDGSFFLYSKLTVTKEEWQQGFVFSCSVMHE | and YTE mutations |
| | ALHNHYTQKSLSLSPGK | |
| 149 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLWCLVKGFYPSDIAVL | with knob, |
| | WESYGTEWASYKTTPPVLDSDGSFFLYSKLTVTKEEWQQGFVFSCSVMHE | LALAPG, and YTE |
| | ALHNHYTQKSLSLSPGK | mutations |
| 150 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLSCAVKGFYPSDIAVL | with hole |
| | WESYGTEWASYKTTPPVLDSDGSFFLVSKLTVTKEEWQQGFVFSCSVMHE | mutations |
| | ALHNHYTQKSLSLSPGK | |
| 151 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLSCAVKGFYPSDIAVL | with hole and |
| | WESYGTEWASYKTTPPVLDSDGSFFLVSKLTVTKEEWQQGFVFSCSVMHE | LALA mutations |
| | ALHNHYTQKSLSLSPGK | |
| 152 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLSCAVKGFYPSDIAVL | with hole and |
| | WESYGTEWASYKTTPPVLDSDGSFFLVSKLTVTKEEWQQGFVFSCSVMHE | LALAPG mutations |
| | ALHNHYTQKSLSLSPGK | |
| 153 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLSCAVKGFYPSDIAVL | with hole and YTE |
| | WESYGTEWASYKTTPPVLDSDGSFFLVSKLTVTKEEWQQGFVFSCSVMHE | mutations |
| | ALHNHYTQKSLSLSPGK | |
| 154 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLSCAVKGFYPSDIAVL | with hole, LALA, |
| | WESYGTEWASYKTTPPVLDSDGSFFLVSKLTVTKEEWQQGFVFSCSVMHE | and YTE mutations |
| | ALHNHYTQKSLSLSPGK | |
| 155 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLSCAVKGFYPSDIAVL | with hole, |
| | WESYGTEWASYKTTPPVLDSDGSFFLVSKLTVTKEEWQQGFVFSCSVMHE | LALAPG, and YTE |
| | ALHNHYTQKSLSLSPGK | mutations |
| 156 | APELLGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with knob |
| | PIEKTISKAKGQPREPQVYTLPPSRDELTKNQVSLWCLVKGFYPSDIAVE | mutation |
| | WESYGTEWSNYKTTPPVLDSDGSFFLYSKLTVTKEEWQQGFVFSCSVMHE | |
| | ALHNHYTQKSLSLSPGK | |
| 157 | APEAAGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with knob and |
| | PIEKTISKAKGQPREPQVYTLPPSRDELTKNQVSLWCLVKGFYPSDIAVE | LALA mutations |
| | WESYGTEWSNYKTTPPVLDSDGSFFLYSKLTVTKEEWQQGFVFSCSVMHE | |
| | ALHNHYTQKSLSLSPGK | |
| 158 | APEAAGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALGA | with knob and |
| | PIEKTISKAKGQPREPQVYTLPPSRDELTKNQVSLWCLVKGFYPSDIAVE | LALAPG mutations |
| | WESYGTEWSNYKTTPPVLDSDGSFFLYSKLTVTKEEWQQGFVFSCSVMHE | |
| | ALHNHYTQKSLSLSPGK | |
| 159 | APELLGGPSVFLFPPKPKDTLYITREPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with knob and YTE |
| | PIEKTISKAKGQPREPQVYTLPPSRDELTKNQVSLWCLVKGFYPSDIAVE | mutations |
| | WESYGTEWSNYKTTPPVLDSDGSFFLYSKLTVTKEEWQQGFVFSCSVMHE | |
| | ALHNHYTQKSLSLSPGK | |
| 160 | APEAAGGPSVFLFPPKPKDTLYITREPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with knob, LALA, |
| | PIEKTISKAKGQPREPQVYTLPPSRDELTKNQVSLWCLVKGFYPSDIAVE | and YTE mutations |
| | WESYGTEWSNYKTTPPVLDSDGSFFLYSKLTVTKEEWQQGFVFSCSVMHE | |
| | ALHNHYTQKSLSLSPGK | |
| 161 | APEAAGGPSVFLFPPKPKDTLYITREPEVTCVVVDVSHEDPEVKFNWYVD | Clone CH3C.35.23 |
| | GVEVHNAKTKPREEQYNSTYRVVSVLTVLHQDWLNGKEYKCKVSNKALGA | with knob, |
| | PIEKTISKAKGQPREPQVYTLPPSRDELTKNQVSLWCLVKGFYPSDIAVE | LALAPG, and YTE |
| | WESYGTEWSNYKTTPPVLDSDGSFFLYSKLTVTKEEWQQGFVFSCSVMHE | mutations |
| | ALHNHYTQKSLSLSPGK | |
| 162 | APELLGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with hole |
| | PIEKTISKAKGQPREPQVYTLPPSRDELTKNQVSLSCAVKGFYPSDIAVE | mutations |
| | WESYGTEWSNYKTTPPVLDSDGSFFLVSKLTVTKEEWQQGFVFSCSVMHE | |
| | ALHNHYTQKSLSLSPGK | |
| 163 | APEAAGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with hole and |
| | PIEKTISKAKGQPREPQVYTLPPSRDELTKNQVSLSCAVKGFYPSDIAVE | LALA mutations |
| | WESYGTEWSNYKTTPPVLDSDGSFFLVSKLTVTKEEWQQGFVFSCSVMHE | |
| | ALHNHYTQKSLSLSPGK | |
| 164 | APEAAGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALGA | with hole and |
| | PIEKTISKAKGQPREPQVYTLPPSRDELTKNQVSLSCAVKGFYPSDIAVE | LALAPG mutations |
| | WESYGTEWSNYKTTPPVLDSDGSFFLVSKLTVTKEEWQQGFVFSCSVMHE | |
| | ALHNHYTQKSLSLSPGK | |
| 165 | APELLGGPSVFLFPPKPKDTLYITREPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with hole and YTE |
| | PIEKTISKAKGQPREPQVYTLPPSRDELTKNQVSLSCAVKGFYPSDIAVE | |
| | WESYGTEWSNYKTTPPVLDSDGSFFLVSKLTVTKEEWQQGFVFSCSVMHE | mutations |
| | ALHNHYTQKSLSLSPGK | |
| 166 | APEAAGGPSVFLFPPKPKDTLYITREPEVTCVVVDVSHEDPEVKFNWYVD | Clone CH3C.35.23 |
| | GVEVHNAKTKPREEQYNSTYRVVSVLTVLHQDWLNGKEYKCKVSNKALPA | with hole, LALA, |
| | PIEKTISKAKGQPREPQVYTLPPSRDELTKNQVSLSCAVKGFYPSDIAVE | and YTE mutations |
| | WESYGTEWSNYKTTPPVLDSDGSFFLVSKLTVTKEEWQQGFVFSCSVMHE | |
| | ALHNHYTQKSLSLSPGK | |
| 167 | APEAAGGPSVFLFPPKPKDTLYITREPEVTCVVVDVSHEDPEVKFNWYVD | Clone CH3C.35.23 |
| | GVEVHNAKTKPREEQYNSTYRVVSVLTVLHQDWLNGKEYKCKVSNKALGA | with hole, |
| | PIEKTISKAKGQPREPQVYTLPPSRDELTKNQVSLSCAVKGFYPSDIAVE | LALAPG, and YTE |
| | WESYGTEWSNYKTTPPVLDSDGSFFLVSKLTVTKEEWQQGFVFSCSVMHE | mutations |
| | ALHNHYTQKSLSLSPGK | |
| 168 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.21.17.2 |
| | CKVSNKALPAPIEKTISKAKGQPREPQVYTLPPSRDELTKNQVSLWCLVK | with knob and |
| | GFYPSDIAVLWESYGTEWASYKTTPPVLDSDGSFFLYSKLTVTKEEWQQG | LALA mutations |
| | FVFSCSVMHEALHNHYTQKSLSLSPGK | and portion of |
| | | human IgG1 hinge |
| | | sequence |
| 169 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALPAPIEKTISKAKGQPREPQVYTLPPSRDELTKNQVSLWCLVK | knob and LALA |
| | GFYPSDIAVEWESYGTEWANYKTTPPVLDSDGSFFLYSKLTVTKEEWQQG | mutations and |
| | FVFSCSVMHEALHNHYTQKSLSLSPGK | portion of human |
| | | IgG1 hinge |
| | | sequence |
| 170 | SETQANSTTD ALNVLLIIVD DLRPSLGCYG DKLVRSPNID | IDS sequence |
| | QLASHSLLFQ NAFAQQAVCA PSRVSFLTGR RPDTTRLYDF | |
| | NSYWRVHAGN FSTIPQYFKE NGYVTMSVGK VFHPGISSNH | |
| | TDDSPYSWSF PPYHPSSEKY ENTKTCRGPD GELHANLLCP | |
| | VDVLDVPEGT LPDKQSTEQA IQLLEKMKTS ASPFFLAVGY | |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF QLLMP | |
| 171 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole and LALA |
| | HKPHIPFRYP KEFQKLYPLE NITLAPDPEV PDGLPPVAYN | mutations |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 172 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole mutations |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 173 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | knob mutation |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLWCLVK | |
| | GFYPSDIAVE WESNGQPENN YKTTPPVLDS DGSFFLYSKL | |
| | TVDKSRWQQG NVFSCSVMHE ALHNHYTQKS LSLSPGK | |
| 174 | SETQANSTTD ALNVLLIIVD DLRPSLGCYG DKLVRSPNID | IDS sequence |
| | QLASHSLLFQ NAFAQQAVfGA PSRVSFLTGR RPDTTRLYDF | (formylglycine |
| | NSYWRVHAGN FSTIPQYFKE NGYVTMSVGK VFHPGISSNH | residue ″fG″ |
| | TDDSPYSWSF PPYHPSSEKY ENTKTCRGPD GELHANLLCP | double |
| | VDVLDVPEGT LPDKQSTEQA IQLLEKMKTS ASPFFLAVGY | underlined) |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF QLLMP | |
| 175 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | hole and LALA |
| | SASQLMEPGR QSMDLVELVS LFPTLAGLAG LQVPPRCPVP | mutations |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 176 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | hole mutations |
| | SASQLMEPGR QSMDLVELVS LFPTLAGLAG LQVPPRCPVP | |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 177 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | knob mutation |
| | SASQLMEPGR QSMDLVELVS LFPTLAGLAG LQVPPRCPVP | |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLWCLVK | |
| | GFYPSDIAVE WESNGQPENN YKTTPPVLDS DGSFFLYSKL | |
| | TVDKSRWQQG NVFSCSVMHE ALHNHYTQKS LSLSPGK | |
| 178 | NSVIIVDKNGRLVYLVENPGGYVAYSKAATVTGKLVHANFGTKKDFEDLY | Human TfR apical |
| | TPVNGSIVIVRAGKITFAEKVANAESLNAIGVLIYMDQTKFPIVNAELSF | domain |
| | FGHAHLGTGDPYTPGFPSFNHTQFPPSRSSGLPNIPVQTISRAAAEKLFG | |
| | NMEGDCPSDWKTDSTCRMVTSESKNVKLTVS | |
| 179 | GGGGS | Glycine-rich |
| | | linker |
| 180 | GGGGSGGGGS | Glycine-rich |
| | | linker |
| 181 | HHHHHH | Hexahistidine tag |
| 182 | YxTEWSS | Library motif |
| 183 | APELLGGPSVFLFPPKPKDTLMISRTPEVTCVVVDVSHEDPEVKFNWYVD | Wild-type human |
| | GVEVHNAKTKPREEQYNSTYRVVSVLTVLHQDWLNGKEYKCKVSNKALPA | Fc sequence |
| | PIEKTISKAKGQPREPQVYTLPPSRDELTKNQVSLTCLVKGFYPSDIAVE | positions 231-446 |
| | WESNGQPENNYKTTPPVLDSDGSFFLYSKLTVDKSRWQQGNVFSCSVMHE | EU index |
| | ALHNHYTQKSLSLSPG | numbering |
| 184 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESYGTEWSSYKTTPPVLDSDGSFFLYSKLTVTKEEWQQGFVFSCSVMHE | |
| | ALHNHYTQKSLSLSPG | |
| 185 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob |
| | PIEKTISKAKGQPREPQVYTLPPSRDELTKNQVSLWCLVKGFYPSDIAVW | mutation |
| | WESYGTEWSSYKTTPPVLDSDGSFFLYSKLTVTKEEWQQGFVFSCSVMHE | |
| | ALHNHYTQKSLSLSPG | |
| 186 | APEAAGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob and |
| | PIEKTISKAKGQPREPQVYTLPPSRDELTKNQVSLWCLVKGFYPSDIAVW | LALA mutations |
| | WESYGTEWSSYKTTPPVLDSDGSFFLYSKLTVTKEEWQQGFVFSCSVMHE | |
| | ALHNHYTQKSLSLSPG | |
| 187 | APELLGGPSVFLFPPKPKDTLYITREPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob and YTE |
| | PIEKTISKAKGQPREPQVYTLPPSRDELTKNQVSLWCLVKGFYPSDIAVW | mutations |
| | WESYGTEWSSYKTTPPVLDSDGSFFLYSKLTVTKEEWQQGFVFSCSVMHE | |
| | ALHNHYTQKSLSLSPG | |
| 188 | APEAAGGPSVFLFPPKPKDTLYITREPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob, LALA, |
| | PIEKTISKAKGQPREPQVYTLPPSRDELTKNQVSLWCLVKGFYPSDIAVW | and YTE mutations |
| | WESYGTEWSSYKTTPPVLDSDGSFFLYSKLTVTKEEWQQGFVFSCSVMHE | |
| | ALHNHYTQKSLSLSPG | |
| 189 | DKTHTCPPCPAPEAAGGPSVFLFPPKPKDTLMISRTPEVTCVVVDVSHED | Clone CH3C.35.21 |
| | PEVKFNWYVDGVEVHNAKTKPREEQYNSTYRVVSVLTVLHQDWLNGKEYK | with knob and |
| | CKVSNKALPAPIEKTISKAKGQPREPQVYTLPPSRDELTKNQVSLWCLVK | LALA mutations |
| | GFYPSDIAVWWESYGTEWSSYKTTPPVLDSDGSFFLYSKLTVTKEEWQQG | and portion of |
| | FVFSCSVMHEALHNHYTQKSLSLSPG | human IgGI hinge |
| | | sequence |
| 190 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLTCLVKGFYPSDIAVL | |
| | WESYGTEWSSYKTTPPVLDSDGSFFLYSKLTVTKEEWQQGFVFSCSVMHE | |
| | ALHNHYTQKSLSLSPG | |
| 191 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLWCLVKGFYPSDIAVL | with knob and |
| | WESYGTEWSSYKTTPPVLDSDGSFFLYSKLTVTKEEWQQGFVFSCSVMHE | LALA mutations |
| | ALHNHYTQKSLSLSPG | |
| 192 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 |
| | PIEKTISKAKGQPREPQVYTLPPSRDELTKNQVSLTCLVKGFYPSDIAVE | |
| | WESYGTEWANYKTTPPVLDSDGSFFLYSKLTVTKEEWQQGFVFSCSVMHE | |
| | ALHNHYTQKSLSLSPG | |
| 193 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob mutation |
| | WESYGTEWANYKTTPPVLDSDGSFFLYSKLTVTKEEWQQGFVFSCSVMHE | |
| | ALHNHYTQKSLSLSPG | |
| 194 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and LALA |
| | WESYGTEWANYKTTPPVLDSDGSFFLYSKLTVTKEEWQQGFVFSCSVMHE | mutations |
| | ALHNHYTQKSLSLSPG | |
| 195 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob and LALAPG |
| | WESYGTEWANYKTTPPVLDSDGSFFLYSKLTVTKEEWQQGFVFSCSVMHE | mutations |
| | ALHNHYTQKSLSLSPG | |
| 196 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and YTE |
| | WESYGTEWANYKTTPPVLDSDGSFFLYSKLTVTKEEWQQGFVFSCSVMHE | mutations |
| | ALHNHYTQKSLSLSPG | |
| 197 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob, LALA, and |
| | WESYGTEWANYKTTPPVLDSDGSFFLYSKLTVTKEEWQQGFVFSCSVMHE | YTE mutations |
| | ALHNHYTQKSLSLSPG | |
| 198 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob, LALAPG, and |
| | WESYGTEWANYKTTPPVLDSDGSFFLYSKLTVTKEEWQQGFVFSCSVMHE | YTE mutations |
| | ALHNHYTQKSLSLSPG | |
| 199 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALPAPIEKTISKAKGQPREPQVYTLPPSRDELTKNQVSLWCLVK | knob and LALA |
| | GFYPSDIAVEWESYGTEWANYKTTPPVLDSDGSFFLYSKLTVTKEEWQQG | mutations and |
| | FVFSCSVMHEALHNHYTQKSLSLSPG | portion of human |
| | | IgG1 hinge |
| | | sequence |
| 200 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLTCLVKGFYPSDIAVL | |
| | WESYGTEWASYKTTPPVLDSDGSFFLYSKLTVTKEEWQQGFVFSCSVMHE | |
| | ALHNHYTQKSLSLSPG | |
| 201 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob |
| | WESYGTEWASYKTTPPVLDSDGSFFLYSKLTVTKEEWQQGFVFSCSVMHE | mutation |
| | ALHNHYTQKSLSLSPG | |
| 202 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALA mutations |
| | ALHNHYTQKSLSLSPG | |
| 203 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALAPG mutations |
| | ALHNHYTQKSLSLSPG | |
| 204 | APELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob and YTE |
| | WESYGTEWASYKTTPPVLDSDGSFFLYSKLTVTKEEWQQGFVFSCSVMHE | mutations |
| | ALHNHYTQKSLSLSPG | |
| 205 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob, LALA, |
| | WESYGTEWASYKTTPPVLDSDGSFFLYSKLTVTKEEWQQGFVFSCSVMHE | and YTE mutations |
| | ALHNHYTQKSLSLSPG | |
| 206 | APEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLWCLVKGFYPSDIAVL | with knob, |
| | WESYGTEWASYKTTPPVLDSDGSFFLYSKLTVTKEEWQQGFVFSCSVMHE | LALAPG, and YTE |
| | ALHNHYTQKSLSLSPG | mutations |
| 207 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.21.17.2 |
| | CKVSNKALPAPIEKTISKAKGQPREPQVYTLPPSRDELTKNQVSLWCLVK | with knob and |
| | GFYPSDIAVLWESYGTEWASYKTTPPVLDSDGSFFLYSKLTVTKEEWQQG | LALA mutations |
| | FVFSCSVMHEALHNHYTQKSLSLSPG | and portion of |
| | | human IgG1 hinge |
| | | sequence |
| 208 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole mutations |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSVMHE | |
| | ALHNHYTQKSLSLSPG | |
| 209 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole and LALA |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPG | |
| 210 | APELLGGPSVFLFPPKPKDTLYITREPEVTCVVVDVSHEDPEVKFNWYVD | Fc sequence with |
| | GVEVHNAKTKPREEQYNSTYRVVSVLTVLHQDWLNGKEYKCKVSNKALPA | hole and YTE |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPG | |
| 211 | APEAAGGPSVFLFPPKPKDTLYITREPEVTCVVVDVSHEDPEVKFNWYVD | Fc sequence with |
| | GVEVHNAKTKPREEQYNSTYRVVSVLTVLHQDWLNGKEYKCKVSNKALPA | hole, LALA, and |
| | PIEKTISKAKGQPREPQVYTLPPSRDELTKNQVSLSCAVKGFYPSDIAVE | YTE mutations |
| | WESNGQPENNYKTTPPVLDSDGSFFLVSKLTVDKSRWQQGNVFSCSVMHE | |
| | ALHNHYTQKSLSLSPG | |
| 212 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole mutations |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 213 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole and LALA |
| | HKPHIPFRYP KEFQKLYPLE NITLAPDPEV PDGLPPVAYN | mutations |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 214 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (cysteine |
| | HKPHIPFRYP KEFQKLYPLE NITLAPDPEV PDGLPPVAYN | modified to |
| | PWMDIRQRED VQALNISVPY GPIPVDFQRK IRQSYFASVS | formylglycine |
| | YLDTQVGRLL SALDDLQLAN STIIAFTSDH GWALGEHGEW | double |
| | AKYSNFDVAT HVPLIFYVPG RTASLPEAGE KLFPYLDPFD | underlined) and |
| | SASQLMEPGR QSMDLVELVS LFPTLAGLAG LQVPPRCPVP | hole mutations |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 215 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (cysteine |
| | HKPHIPFRYP KEFQKLYPLE NITLAPDPEV PDGLPPVAYN | modified to |
| | PWMDIRQRED VQALNISVPY GPIPVDFQRK IRQSYFASVS | formylglycine |
| | YLDTQVGRLL SALDDLQLAN STIIAFTSDH GWALGEHGEW | double |
| | AKYSNFDVAT HVPLIFYVPG RTASLPEAGE KLFPYLDPFD | underlined) and |
| | SASQLMEPGR QSMDLVELVS LFPTLAGLAG LQVPPRCPVP | hole and LALA |
| | SFHVELGREG KNLLKHFRFR DLEEDPYLPG NPRELIAYSQ | mutations |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 216 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | hole mutations |
| | SASQLMEPGR QSMDLVELVS LFPTLAGLAG LQVPPRCPVP | |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 217 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | hole and LALA |
| | SASQLMEPGR QSMDLVELVS LFPTLAGLAG LQVPPRCPVP | mutations |
| | SFHVELGREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

What is claimed is:

1. A method of treating Hunter syndrome in a subject in need thereof, comprising administering to the subject a therapeutically effective dose of a pharmaceutical composition comprising a protein, wherein administration of the pharmaceutical composition reduces levels of a glycosaminoglycan (GAG) in the cerebrospinal fluid (CSF) of the subject to a baseline level measured in a healthy subject or a subject that does not have Hunter syndrome, and wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

2. The method of claim 1, wherein the therapeutically effective dose is from about 3 mg/kg to about 30 mg/kg of protein.

3. The method of claim 2, wherein the therapeutically effective dose is about 3 mg/kg of protein.

4. The method of claim 2, wherein the therapeutically effective dose is about 7.5 mg/kg of protein.

5. The method of claim 2, wherein the therapeutically effective dose is about 15 mg/kg of protein.

6. The method of claim 2, wherein the therapeutically effective dose is about 30 mg/kg of protein.

7. The method of any one of claims 1-6, wherein the pharmaceutical composition is administered weekly.

8. The method of any one of claims 1-7, wherein the pharmaceutical composition further comprises a pharmaceutically acceptable excipient.

9. The method of any one of claims 1-8, wherein the second Fc polypeptide specifically binds to the transferrin receptor.

10. The method of any one of claims 1-9, wherein the IDS amino acid sequence comprises an amino acid sequence having at least 90% identity to SEQ ID NO:70.

11. The method of claim 10, wherein the IDS amino acid sequence comprises a sequence selected from the group consisting of SEQ ID NOs:70, 90, 170 and 174.

12. The method of any one of claims 1-11, wherein the first Fc polypeptide linked to the IDS amino acid sequence comprises the amino acid sequence of SEQ ID NO: 91, 171 or 175.

13. The method of any one of claims 1-11, wherein the first Fc polypeptide linked to the IDS amino acid sequence comprises the amino acid sequence of SEQ ID NO: 213, 215, or 217.

14. The method of any one of claims 1-13, wherein the IDS amino acid sequence is linked to the N-terminus of the first Fc polypeptide.

15. The method of any one of claims 1-14, wherein the second Fc polypeptide comprises:
a. Leu at position 380;
b. Ala at position 389; and
c. Ser at position 390.

16. The method of any one of claims 1-14, wherein the second Fc polypeptide comprises:
a. Glu at position 380;
b. Ala at position 389; and
c. Asn at position 390.

17. The method of any one of claims 1-14, wherein the second Fc polypeptide comprises:
a. Trp at position 380;
b. Ser at position 389; and
c. Ser at position 390.

18. The method of any one of claims 1-14, wherein the second Fc polypeptide comprises:
a. Leu at position 380;
b. Ser at position 389; and
c. Ser at position 390.

19. The method of any one of claims 1-9, wherein the first Fc polypeptide linked to the IDS amino acid sequence comprises SEQ ID NO: 175; and the second Fc polypeptide comprises SEQ ID NO: 92.

20. The method of any one of claims 1-9, wherein the first Fc polypeptide linked to the IDS amino acid sequence comprises SEQ ID NO: 217; and the second Fc polypeptide comprises SEQ ID NO: 189.

21. The method of any one of claims 1-9, wherein the first Fc polypeptide linked to the IDS amino acid sequence comprises SEQ ID NO: 175; and the second Fc polypeptide comprises SEQ ID NO: 95.

22. The method of claim 21, wherein the second Fc polypeptide further comprises SEQ ID NO: 89, wherein SEQ ID NO: 89 is attached to the N-terminus of SEQ ID NO:95.

23. The method of any one of claims 1-9, wherein the first Fc polypeptide linked to the IDS amino acid sequence comprises SEQ ID NO: 217; and the second Fc polypeptide comprises SEQ ID NO: 191.

24. The method of claim 23, wherein the second Fc polypeptide further comprises SEQ ID NO: 89, wherein SEQ ID NO: 89 is attached to the N-terminus of SEQ ID NO:191.

25. The method of any one of claims 1-9, wherein the first Fc polypeptide linked to the IDS amino acid sequence comprises SEQ ID NO: 175; and the second Fc polypeptide comprises SEQ ID NO: 168.

26. The method of any one of claims 1-9, wherein the first Fc polypeptide linked to the IDS amino acid sequence comprises SEQ ID NO: 217; and the second Fc polypeptide comprises SEQ ID NO: 207.

27. The method of any one of claims 1-9, wherein the first Fc polypeptide linked to the IDS amino acid sequence comprises SEQ ID NO: 175; and the second Fc polypeptide comprises SEQ ID NO: 169.

28. The method of any one of claims 1-9, wherein the first Fc polypeptide linked to the IDS amino acid sequence comprises SEQ ID NO: 217; and the second Fc polypeptide comprises SEQ ID NO: 199.

29. The method of any one of claims 1-28, wherein the administration of the pharmaceutical composition reduces levels of one or more analytes in the CSF of the subject by at least about 10%, 15%, 20%, 25%, or 30%, wherein the reduction is relative to the level of the corresponding one or more analytes in the subject prior to the administration, wherein the one or more analytes are selected from the group consisting of neurofilament light (Nf-L), soluble triggering receptor expressed on myeloid cells 2 (sTREM2), a bis(monoacylglycerol) phosphate (BMP), a ganglioside, and a sphingolipid.

30. The method of claim 29, wherein the administration of the pharmaceutical composition reduces levels of the one or more analytes in the CSF of the subject to baseline levels, measured in a healthy subject or in a subject that does not have Hunter syndrome.

31. The method of any one of claims 1-30, wherein the administration of the pharmaceutical composition reduces levels of one or more analytes in the serum of the subject by at least about 30%, 40%, 50%, 60%, or 70% relative to the level of the corresponding one or more analytes in the serum of the subject prior to the administration, wherein the one or more analytes are selected from the group consisting of a GAG and Nf-L.

32. The method of claim 31, wherein the administration of the pharmaceutical composition reduces levels of a GAG in the serum of the subject to baseline levels, measured in a healthy subject or in a subject that does not have Hunter syndrome.

33. The method of claim 31, wherein the administration of the pharmaceutical composition reduces levels of Nf-L in the serum of the subject to baseline levels, measured in a healthy subject or in a subject that does not have Hunter syndrome.

34. The method of any one of claims 1-33, wherein the administration of the pharmaceutical composition reduces levels of a GAG in the urine of the subject by at least about 30%, 40%, 50%, 60%, or 70% relative to the level of the GAG in the urine of the subject prior to the administration.

35. The method of claim 34, wherein the administration of the pharmaceutical composition reduces levels of a GAG in the urine of the subject to baseline levels, measured in a healthy subject or in a subject that does not have Hunter syndrome.

36. The method of any one of claims 1-35, wherein the administration of the pharmaceutical composition improves or maintains the subject's toileting abilities percentage (TAP) relative to a baseline level measured for the subject prior to administration of the pharmaceutical composition.

37. The method of any one of claims 1-36, wherein the administration of the pharmaceutical composition improves or maintains the subject's qualitative individualized educational plan (IEP) trajectories in cognitive categories or behavioral categories relative to a baseline level measured for the subject prior to administration of the pharmaceutical composition.

38. The method of any one of claims 1-37, wherein the administration of the pharmaceutical composition improves or maintains the subject's hearing relative to a baseline level measured for the subject prior to administration of the pharmaceutical composition, as assessed by an auditory brainstem response (ABR) assessment or a standard hearing test.

39. The method of any one of claims 1-38, wherein the administration of the pharmaceutical composition changes the subject's liver volume relative to a baseline level measured for the subject prior to administration of the pharmaceutical composition, as assessed by MRI or ultrasound.

40. The method of any one of claims 1-39, wherein the administration of the pharmaceutical composition changes the subject's spleen volume relative to a baseline level measured for the subject prior to administration of the pharmaceutical composition, as assessed by MRI or ultrasound.

41. The method of any one of claims 1-40, wherein the subject is a male subject.

42. The method of any one of claims 1-41, wherein the subject is from about 2 to 18 years of age.

43. The method of any one of claims 1-41, wherein the subject is from about 2 to 10 years of age.

44. The method of any one of claims 1-41, wherein the subject is from about 5 to 10 years of age.

45. The method of any one of claims 1-41, wherein the subject is less than 4 years of age.

46. The method of any one of claims 1-45, wherein the subject has a weight of ≥15 kg.

47. The method of any one of claims 1-45, wherein the subject has a weight of ≥19 kg.

48. The method of any one of claims 1-47, wherein the subject has a development quotient (DQ)<85.

49. The method of any one of claims 1-48, wherein the subject has a decline of at least 10 points in development quotient (DQ) within a period of about 6 months or longer.

50. The method of any one of claims 1-49, wherein the subject has neuronopathic Hunter syndrome (nMPS II) or has a same genetic mutation in the IDS gene as a blood relative with confirmed neuronopathic Hunter syndrome (nMPS II).

51. The method of any one of claims 1-50, wherein the pharmaceutical composition is administered to the subject intravenously.

52. The method of claim 51, wherein the subject does not experience an infusion-related reaction (IRR) upon administration of the pharmaceutical composition.

53. The method of claim 52, wherein IRR is an allergic reaction.

54. The method of claim 52, wherein IRR is anaphylaxis.

55. The method of any one of claims 52-54, wherein the pharmaceutical composition is administered to the subject without pretreatment or co-administration of medication for an infusion-related reaction (IRR).

56. The method of claim 51, wherein the pharmaceutical composition is administered to the subject with pretreatment or co-administration of medication for an infusion-related reaction (IRR).

57. The method of claim 55 or 56, wherein the medication for infusion-related reaction is one or more selected from the group consisting of: an anti-histamine, an anti-pyretic, and a corticosteroid.

58. The method of any one of claims 1-57, wherein the urine total GAG concentration (normalized to creatinine) does not increase relative to a baseline level after administration of the pharmaceutical composition, wherein the baseline level is measured for the subject prior to administration of the pharmaceutical composition.

59. The method of any one of claims 1-57, wherein the urine total GAG concentration (normalized to creatinine) decreases relative to a baseline level after administration of the pharmaceutical composition, wherein the baseline level is measured for the subject prior to administration of the pharmaceutical composition.

60. The method of claim 58 or 59, wherein the stabilization or decrease in urine total GAG concentration in the subject supports safety of the administered dose.

61. The method of any one of claims 1-60, wherein the incidence of anti-drug antibodies (ADAs) does not increase relative to a baseline level after administration of the pharmaceutical composition, wherein the baseline level is measured for the subject prior to administration of the pharmaceutical composition.

62. The method of any one of claims 1-60, wherein the incidence of anti-drug antibodies (ADAs) increases by less than 10% relative to a baseline level after administration of the pharmaceutical composition, wherein the baseline level is measured for the subject prior to administration of the pharmaceutical composition.

63. The method of any one of claims 1-62, wherein the frequency of serious adverse events (SAEs) is less than about 1/1000.

64. The method of any one of claims 1-63, wherein the administration of the pharmaceutical composition reduces levels of a glycosaminoglycan (GAG) in the CSF of the subject by at least 50%, 60%, 70%, 75%, 80%, 85%, or 90% after at least 4 weekly doses, wherein the reduction is relative to the CSF levels of the GAG in the subject prior to administration.

65. The method of claim 64, wherein the administration of the pharmaceutical composition reduces levels of the glycosaminoglycan (GAG) in the CSF of the subject by at least 50%, 60%, 70%, 75%, 80%, 85%, or 90% after 4 weekly doses.

66. The method of any one of claims 1-63, wherein the administration of the pharmaceutical composition reduces levels of a glycosaminoglycan (GAG) in the CSF of the subject by at least 50%, 60%, 70%, 75%, 80%, 85%, or 90% after at least 8 weekly doses, wherein the reduction is relative to the CSF levels of the GAG in the subject prior to administration.

67. The method of claim 66, wherein the administration of the pharmaceutical composition reduces levels of the glycosaminoglycan (GAG) in the CSF of the subject by at least 50%, 60%, 70%, 75%, 80%, 85%, or 90% after 8 weekly doses.

68. The method of any one of claims 1-63, wherein the administration of the pharmaceutical composition reduces levels of a glycosaminoglycan (GAG) in the CSF of the subject by at least 50%, 60%, 70%, 75%, 80%, 85%, or 90% after at least 12 weekly doses, wherein the reduction is relative to the CSF levels of the GAG in the subject prior to administration.

69. The method of claim 68, wherein the administration of the pharmaceutical composition reduces levels of the glycosaminoglycan (GAG) in the CSF of the subject by at least 50%, 60%, 70%, 75%, 80%, 85%, or 90% after 12 weekly doses.

70. The method of any one of claims 1-69, wherein the administration of the pharmaceutical composition reduces levels of a glycosaminoglycan (GAG) in the urine of the subject by at least 50%, 60%, 70%, 75%, 80%, 85%, or 90% after at least 4 weekly doses, wherein the reduction is relative to the urine levels of the GAG in the subject prior to administration.

71. The method of claim 70, wherein the administration of the pharmaceutical composition reduces levels of the glycosaminoglycan (GAG) in the urine of the subject by at least 50%, 60%, 70%, 75%, 80%, 85%, or 90% after 4 weekly doses.

72. The method of any one of claims 1-69, wherein the administration of the pharmaceutical composition reduces levels of a glycosaminoglycan (GAG) in the urine of the subject by at least 50%, 60%, 70%, 75%, 80%, 85%, or 90% after at least 8 weekly doses, wherein the reduction is relative to the urine levels of the GAG in the subject prior to administration.

73. The method of claim 72, wherein the administration of the pharmaceutical composition reduces levels of the glycosaminoglycan (GAG) in the urine of the subject by at least 50%, 60%, 70%, 75%, 80%, 85%, or 90% after 8 weekly doses.

74. The method of any one of claims 70-73, wherein administration of the pharmaceutical composition reduces levels of the glycosaminoglycan (GAG) in the urine of the subject to a baseline level measured in a healthy subject or a subject that does not have Hunter syndrome.

75. The method of any one of claims 64-74, wherein the GAG is heparan sulfate.

76. The method of claim 75, wherein administration of the pharmaceutical composition reduces levels heparan sulfate in the CSF of the subject by at least 80% after at least 4 weekly doses, wherein the reduction is relative to the CSF heparan sulfate levels in the subject prior to administration.

77. The method of claim 75, wherein administration of the pharmaceutical composition reduces levels heparan sulfate in the CSF of the subject by at least 70% after at least 12 weekly doses, wherein the reduction is relative to the CSF heparan sulfate levels in the subject prior to administration.

78. The method of any one of claims 64-74, wherein the GAG is dermatan sulfate.

79. The method of any one of claims 1-78, wherein the administration of the pharmaceutical composition reduces levels of a ganglioside in the CSF of the subject by at least 10%, 15%, 20%, 25%, 30%, 40%, 50%, 60%, 70%, 80%, or 90% after at least 8 weekly doses, wherein the reduction is relative to the CSF levels of the ganglioside in the subject prior to administration.

80. The method of claim 79, wherein the administration of the pharmaceutical composition reduces levels of the ganglioside in the CSF of the subject by at least 10%, 15%, 20%, 25%, 30%, 40%, 50%, 60%, 70%, 80%, or 90% after 8 weekly doses.

81. The method of claim 79 or 80, wherein the ganglioside is GM3.

82. The method of any one of claims 1-81, wherein the administration of the pharmaceutical composition reduces levels of a BMP in the CSF of the subject by at least 10%, 15%, 20%, 25%, 30%, 40%, 50%, 60%, 70%, 80%, or 90% after at least 8 weekly doses, wherein the reduction is relative to the CSF levels of the BMP in the subject prior to administration.

83. The method of claim 82, wherein the administration of the pharmaceutical composition reduces levels of the BMP in the CSF of the subject by at least 10%, 15%, 20%, 25%, 30%, 40%, 50%, 60%, 70%, 80%, or 90% after 8 weekly doses.

84. The method of claim 82 or 83, wherein the BMP is a BMP di 18:1 species.

85. The method of any one of claims 1-84, wherein prior to the administration of the pharmaceutical composition, the subject had received recombinant idursulfase enzyme replacement therapy.

86. The method of any one of claims 1-85, wherein the subject had pre-existing anti-drug antibodies against IDS prior to administration of the pharmaceutical composition.

87. The method of claim 86, wherein the subject's titer of anti-drug antibodies against IDS ranges from 189 to greater than 11 million.

88. The method of claim 87, wherein the subject's titer of anti-drug antibodies against IDS is greater than 11 million.

89. A method of treating Hunter syndrome in a subject in need thereof, comprising administering to the subject a protein at a weekly dose of about 3 mg/kg, and wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

90. A method of treating Hunter syndrome in a subject in need thereof, comprising administering to the subject a protein at a weekly dose of about 7.5 mg/kg, and wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

91. A method of treating Hunter syndrome in a subject in need thereof, comprising administering to the subject a protein at a weekly dose of about 15 mg/kg, and wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

92. A method of treating Hunter syndrome in a subject in need thereof, comprising administering to the subject a protein at a weekly dose of about 30 mg/kg, and wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 89;
vii. Ser or Asn at position 390;
viii. Thr at position 413;
ix. Glu at position 415;
x. Glu at position 416; and
xi. Phe at position 421.

93. The method of any one of claims 89-92, wherein the protein is comprised in a pharmaceutical composition that further comprises a pharmaceutically acceptable excipient.

94. A method of resolving an infusion-related reaction (IRR) in a subject receiving treatment for Hunter syndrome, comprising administering one or more agents selected from the group consisting of an anti-histamine, an anti-pyretic, and a corticosteroid, wherein the subject is being administered or was administered a pharmaceutical composition comprising a protein at a dose of at least about 7.5 mg/kg of protein, and wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 89;
vii. Ser or Asn at position 390;
viii. Thr at position 413;
ix. Glu at position 415;
x. Glu at position 416; and
xi. Phe at position 421.

95. The method of claim 94, wherein the IRR is a fever.

96. The method of claim 94 or 95, wherein method of resolving the IRR comprises administering to the subject acetaminophen and diphenhydramine.

97. A method of treating a neurobehavioral deficit, an auditory deficit and/or a musculoskeletal abnormality in a subject with Hunter syndrome, comprising administering to the subject a therapeutically effective dose of a pharmaceutical composition comprising a protein, wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

98. The method of claim 97, wherein the therapeutically effective dose comprises an amount of the protein that is activity equivalent to a standard of care dose for recombinant idursulfase.

99. The method of claim 97, wherein the therapeutically effective dose comprises an amount of the protein that is activity equivalent to a standard of care dose for recombinant idursulfase-beta.

100. The method of claim 97 or 98, wherein the therapeutically effective dose is from about 3 mg/kg to about 30 mg/kg of protein.

101. The method of any one of claims 97-100, wherein the pharmaceutical composition further comprises a pharmaceutically acceptable excipient.

102. The method of any one of claims 97-101, wherein the second Fc polypeptide specifically binds to the transferrin receptor.

103. The method of any one of claims 97-102, wherein the IDS amino acid sequence comprises an amino acid sequence having at least 90% identity to SEQ ID NO:70.

104. The method of claim 103, wherein the IDS amino acid sequence comprises a sequence selected from the group consisting of SEQ ID NOs:70, 90, 170 and 174.

105. The method of any one of claims 97-104, wherein the first Fc polypeptide linked to the IDS amino acid sequence comprises the amino acid sequence of SEQ ID NO: 91, 171 or 175.

106. The method of any one of claims 97-104, wherein the first Fc polypeptide linked to the IDS amino acid sequence comprises the amino acid sequence of SEQ ID NO: 213, 215, or 217.

107. The method of any one of claims 97-106, wherein the IDS amino acid sequence is linked to the N-terminus of the first Fc polypeptide.

108. The method of any one of claims 97-107, wherein the second Fc polypeptide comprises:
a. Leu at position 380;
b. Ala at position 389; and
c. Ser at position 390.

109. The method of any one of claims 97-107, wherein the second Fc polypeptide comprises:
a. Glu at position 380;
b. Ala at position 389; and
c. Asn at position 390.

110. The method of any one of claims 97-107, wherein the second Fc polypeptide comprises:
a. Trp at position 380;
b. Ser at position 389; and
c. Ser at position 390.

111. The method of any one of claims 97-107, wherein the second Fc polypeptide comprises:
a. Leu at position 380;
b. Ser at position 389; and
c. Ser at position 390.

112. The method of any one of claims 97-102, wherein the first Fc polypeptide linked to the IDS amino acid sequence comprises SEQ ID NO: 175; and the second Fc polypeptide comprises SEQ ID NO: 92.

113. The method of any one of claims 97-102, wherein the first Fc polypeptide linked to the IDS amino acid sequence comprises SEQ ID NO: 217; and the second Fc polypeptide comprises SEQ ID NO: 189.

114. The method of any one of claims 97-102, wherein the first Fc polypeptide linked to the IDS amino acid sequence comprises SEQ ID NO: 175; and the second Fc polypeptide comprises SEQ ID NO: 95.

115. The method of claim 114, wherein the second Fc polypeptide further comprises SEQ ID NO: 89, wherein SEQ ID NO: 89 is attached to the N-terminus of SEQ ID NO:95.

116. The method of any one of claims 97-102, wherein the first Fc polypeptide linked to the IDS amino acid sequence comprises SEQ ID NO: 217; and the second Fc polypeptide comprises SEQ ID NO: 191.

117. The method of claim 116, wherein the second Fc polypeptide further comprises SEQ ID NO: 89, wherein SEQ ID NO: 89 is attached to the N-terminus of SEQ ID NO:191.

118. The method of any one of claims 97-102, wherein the first Fc polypeptide linked to the IDS amino acid sequence comprises SEQ ID NO: 175; and the second Fc polypeptide comprises SEQ ID NO: 168.

119. The method of any one of claims 97-102, wherein the first Fc polypeptide linked to the IDS amino acid sequence comprises SEQ ID NO: 217; and the second Fc polypeptide comprises SEQ ID NO: 207.

120. The method of any one of claims 97-102, wherein the first Fc polypeptide linked to the IDS amino acid sequence comprises SEQ ID NO: 175; and the second Fc polypeptide comprises SEQ ID NO: 169.

121. The method of any one of claims 97-102, wherein the first Fc polypeptide linked to the IDS amino acid sequence comprises SEQ ID NO: 217; and the second Fc polypeptide comprises SEQ ID NO: 199.

122. The method of any one of claims 97-121, which is a method of treating a neurobehavioral deficit.

123. The method of claim 122, wherein the neurobehavioral deficit is a motor skill deficit.

124. The method of claim 123, wherein the motor skill deficit is a fine motor skill deficit.

125. The method of claim 123, wherein the motor skill deficit is a gross motor skill deficit.

126. The method of claim 122, wherein the neurobehavioral deficit is an agility deficit.

127. The method of claim 122, wherein the neurobehavioral deficit is a cognitive deficit.

128. The method of claim 127, wherein the cognitive deficit is a learning or memory deficit.

129. The method of claim 122, wherein the neurobehavioral deficit is a sensorimotor gating deficit.

130. The method of any one of claims 97-121, which is a method of treating an auditory deficit.

131. The method of claim 130, wherein administration of the pharmaceutical composition reduces middle ear effusion and/or otitis media in the subject, relative to a baseline level.

132. The method of claim 131, wherein the baseline level of a parameter is the parameter level for the subject prior to administration of the pharmaceutical composition.

133. The method of any one of claims 97-121, which is a method of treating a musculoskeletal abnormality.

134. The method of claim 133, wherein the musculoskeletal abnormality is a skeletal abnormality.

135. The method of any one of claims 97-134, wherein the administration of the pharmaceutical composition reduces levels of one or more analytes in the CSF of the subject by at least about 10%, 15%, 20%, 25%, or 30%, wherein the reduction is relative to the level of the corresponding one or more analytes in the subject prior to the administration, wherein the one or more analytes are selected from the group consisting of a glycosaminoglycan (GAG), neurofilament light (Nf-L), soluble triggering receptor expressed on myeloid cells 2 (sTREM2), a bis(monoacylglycerol) phosphate (BMP), a ganglioside, and a sphingolipid.

136. A pharmaceutical composition comprising a protein for use in a method of treating Hunter syndrome, the method comprising administering a therapeutically effective dose of the pharmaceutical composition to a subject in need thereof, wherein administration of the pharmaceutical composition reduces levels of a glycosaminoglycan (GAG) in the CSF of the subject to a baseline level measured in a healthy subject or a subject that does not have Hunter syndrome, and wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

137. Use of a protein in the preparation of a medicament for treating Hunter syndrome by administering a therapeutically effective dose of the medicament to a subject in need thereof, wherein administration of the medicament reduces levels of a glycosaminoglycan (GAG) in the CSF of the subject to a baseline level measured in a healthy subject or a subject that does not have Hunter syndrome, and wherein the protein comprises:
a. a first Fc polypeptide linked to an iduronate 2-sulfatase (IDS) amino acid sequence, an IDS variant amino acid sequence, or a catalytically active fragment thereof, and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

138. A pharmaceutical composition comprising a protein for use in a method of treating a neurobehavioral deficit, an auditory deficit and/or a musculoskeletal deficit associated with Hunter syndrome, the method comprising administering a therapeutically effective dose of the pharmaceutical composition to a subject in need thereof, wherein the protein comprises:
a. a first Fc polypeptide linked to an iduronate 2-sulfatase (IDS) amino acid sequence, an IDS variant amino acid sequence, or a catalytically active fragment thereof; and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

139. Use of a protein in the preparation of a medicament for treating a neurobehavioral deficit, an auditory deficit and/or a musculoskeletal deficit associated with Hunter syndrome, by administering a therapeutically effective dose of the medicament to a subject in need thereof, wherein the protein comprises:
a. a first Fc polypeptide linked to an iduronate 2-sulfatase (IDS) amino acid sequence, an IDS variant amino acid sequence, or a catalytically active fragment thereof, and
b. a second Fc polypeptide comprising the following amino acid residues, according to EU numbering:
i. Trp, Leu, or Glu at position 380;
ii. Tyr at position 384;
iii. Thr at position 386;
iv. Glu at position 387;
v. Trp at position 388;
vi. Ser or Ala at position 389;
vii. Ser or Asn at position 390;
viii. Thr at position 413;
ix. Glu at position 415;
x. Glu at position 416; and
xi. Phe at position 421.

140. The pharmaceutical composition of claim 136 or 138 or the use of claim 137 or 139, wherein the therapeutically effective dose is from about 3 mg/kg to about 30 mg/kg of protein.
